SEQUENCE LISTING (1547 sequences)

SEQ ID NO: 1 - protein, 9 aa, synthetic construct
GYTFTDYNI

SEQ ID NO: 2 - protein, 8 aa, synthetic construct
INPNYDST

SEQ ID NO: 3 - protein, 11 aa, synthetic construct
CARGNSWYFDV

SEQ ID NO: 4 - protein, 5 aa, synthetic construct
SSVRY

SEQ ID NO: 5 - skipped

SEQ ID NO: 6 - protein, 10 aa, synthetic construct
CQQWSSNPLT

SEQ ID NO: 7 - protein, 9 aa, synthetic construct
GYTFTDYNL

SEQ ID NO: 8 - protein, 8 aa, synthetic construct
INPNYDST

SEQ ID NO: 9 - protein, 11 aa, synthetic construct
CARGNSWYFDV

SEQ ID NO: 10 - protein, 5 aa, synthetic construct
SSVRY

SEQ ID NO: 11 - skipped

SEQ ID NO: 12 - protein, 10 aa, synthetic construct
CQQWSSNPLT

SEQ ID NO: 13 - protein, 5 aa, synthetic construct
DYIMH

SEQ ID NO: 14 - protein, 9 aa, synthetic construct
WVDPEYGST

SEQ ID NO: 15 - protein, 9 aa, synthetic construct
DDGSYSPFD

SEQ ID NO: 16 - protein, 11 aa, synthetic construct
KASENVVTYVS

SEQ ID NO: 17 - protein, 7 aa, synthetic construct
GASNRYT

SEQ ID NO: 18 - protein, 8 aa, synthetic construct
GQGYSYPY

SEQ ID NO: 19 - protein, 5 aa, synthetic construct
DYIMH

SEQ ID NO: 20 - protein, 9 aa, synthetic construct
WVDPEYGST

SEQ ID NO: 21 - protein, 9 aa, synthetic construct
DDGSYSPFD

SEQ ID NO: 22 - protein, 11 aa, synthetic construct
RASENVVTYVS

SEQ ID NO: 23 - protein, 7 aa, synthetic construct
GASNRYT

SEQ ID NO: 24 - protein, 8 aa, synthetic construct
GQGYSYPY

SEQ ID NO: 25 - protein, 5 aa, synthetic construct
TFGMS

SEQ ID NO: 26 - protein, 17 aa, synthetic construct
WINTYSGVPTYADDFKG

SEQ ID NO: 27 - protein, 6 aa, synthetic construct
RSNFAY

SEQ ID NO: 28 - protein, 15 aa, synthetic construct
RASESVDSSGNSFMH

SEQ ID NO: 29 - protein, 7 aa, synthetic construct
RASNLES

SEQ ID NO: 30 - protein, 9 aa, synthetic construct
QQSNEDPWT

SEQ ID NO: 31 - protein, 7 aa, synthetic construct
GYTFTTF

SEQ ID NO: 32 - protein, 6 aa, synthetic construct
NTYSGV

SEQ ID NO: 33 - protein, 6 aa, synthetic construct
RSNFAY

SEQ ID NO: 34 - protein, 15 aa, synthetic construct
RASESVDSSGNSFMH

SEQ ID NO: 35 - protein, 7 aa, synthetic construct
RASNLES

SEQ ID NO: 36 - protein, 9 aa, synthetic construct
QQSNEDPWT

SEQ ID NO: 37 - protein, 10 aa, synthetic construct
GYTFTTFGMS

SEQ ID NO: 38 - protein, 10 aa, synthetic construct
WINTYSGVPT

SEQ ID NO: 39 - protein, 6 aa, synthetic construct
RSNFAY

SEQ ID NO: 40 - protein, 15 aa, synthetic construct
RASESVDSSGNSFMH

SEQ ID NO: 41 - protein, 7 aa, synthetic construct
RASNLES

SEQ ID NO: 42 - protein, 9 aa, synthetic construct
QQSNEDPWT

SEQ ID NO: 43 - protein, 7 aa, synthetic construct
GYTFTTF

SEQ ID NO: 44 - protein, 8 aa, synthetic construct
INTYSGVP

SEQ ID NO: 45 - protein, 8 aa, synthetic construct
ARRSNFAY

SEQ ID NO: 46 - protein, 10 aa, synthetic construct
ESVDSSGNSF

SEQ ID NO: 47 - skipped

SEQ ID NO: 48 - protein, 10 aa, synthetic construct
CQQSNEDPWT

SEQ ID NO: 49 - protein, 7 aa, synthetic construct
GYTFTTF

SEQ ID NO: 50 - protein, 6 aa, synthetic construct
NTYSGV

SEQ ID NO: 51 - protein, 6 aa, synthetic construct
RSNFAY

SEQ ID NO: 52 - protein, 15 aa, synthetic construct
RASESLTASGNSFMH

SEQ ID NO: 53 - protein, 7 aa, synthetic construct
RASNLES

SEQ ID NO: 54 - protein, 9 aa, synthetic construct
QQSRHVNWT

SEQ ID NO: 55 - protein, 7 aa, synthetic construct
GYTFTTF

SEQ ID NO: 56 - protein, 6 aa, synthetic construct
NTYSGV

SEQ ID NO: 57 - protein, 6 aa, synthetic construct
RSNFAY

SEQ ID NO: 58 - protein, 15 aa, synthetic construct
RASQTVDSSGNSFMH

SEQ ID NO: 59 - protein, 7 aa, synthetic construct
LGNRLES

SEQ ID NO: 60 - protein, 9 aa, synthetic construct
QQSNEDPWT

SEQ ID NO: 61 - protein, 7 aa, synthetic construct
GYTFTTF

SEQ ID NO: 62 - protein, 6 aa, synthetic construct
NTYSGV

SEQ ID NO: 63 - protein, 6 aa, synthetic construct
RSNFAY

SEQ ID NO: 64 - protein, 15 aa, synthetic construct
RASESLTASGNSFMH

SEQ ID NO: 65 - protein, 7 aa, synthetic construct
RASNLES

SEQ ID NO: 66 - protein, 9 aa, synthetic construct
QQSRHVNWT

SEQ ID NO: 67 - protein, 5 aa, synthetic construct
TFGMS

SEQ ID NO: 68 - protein, 17 aa, synthetic construct
WINTYSGVPHYAQGFTG

SEQ ID NO: 69 - protein, 6 aa, synthetic construct
RSNFAY

SEQ ID NO: 70 - protein, 15 aa, synthetic construct
RASESLTASGNSFMH

SEQ ID NO: 71 - protein, 7 aa, synthetic construct
RASNLES

SEQ ID NO: 72 - protein, 9 aa, synthetic construct
QQSRHVNWT

SEQ ID NO: 73 - protein, 8 aa, synthetic construct
GYTFTTFG

SEQ ID NO: 74 - protein, 8 aa, synthetic construct
INTYSGVP

SEQ ID NO: 75 - protein, 8 aa, synthetic construct
ARRSNFAY

SEQ ID NO: 76 - protein, 10 aa, synthetic construct
ESLTASGNSF

SEQ ID NO: 77 - skipped

SEQ ID NO: 78 - protein, 9 aa, synthetic construct
QQSRHVNWT

SEQ ID NO: 79 - protein, 5 aa, synthetic construct
TYAMS

SEQ ID NO: 80 - protein, 17 aa, synthetic construct
IISDGGDATFYADSVKG

SEQ ID NO: 81 - protein, 10 aa, synthetic construct
DGTSAAAFDS

SEQ ID NO: 82 - protein, 11 aa, synthetic construct
QASQDITNFLN

SEQ ID NO: 83 - protein, 7 aa, synthetic construct
DASTLQT

SEQ ID NO: 84 - protein, 9 aa, synthetic construct
QQSDSYPIT

SEQ ID NO: 85 - protein, 5 aa, synthetic construct
TYAMS

SEQ ID NO: 86 - protein, 17 aa, synthetic construct
IISDGGDATVYADSVKG

SEQ ID NO: 87 - protein, 10 aa, synthetic construct
DGTSAAAFDS

SEQ ID NO: 88 - protein, 11 aa, synthetic construct
QASQDITNFLN

SEQ ID NO: 89 - protein, 7 aa, synthetic construct
DASTLQT

SEQ ID NO: 90 - protein, 9 aa, synthetic construct
QQSDSYPIT

SEQ ID NO: 91 - protein, 5 aa, synthetic construct
TYAMS

SEQ ID NO: 92 - protein, 17 aa, synthetic construct
IISDGGDATVYADSVKG

SEQ ID NO: 93 - protein, 10 aa, synthetic construct
DGTSAAAFDS

SEQ ID NO: 94 - protein, 11 aa, synthetic construct
QASQDITNFLN

SEQ ID NO: 95 - protein, 7 aa, synthetic construct
DASTLQT

SEQ ID NO: 96 - protein, 9 aa, synthetic construct
QQSDSYPIT

SEQ ID NO: 97 - protein, 5 aa, synthetic construct
TYAMS

SEQ ID NO: 98 - protein, 17 aa, synthetic construct
IISDGGDATFYADSVKG

SEQ ID NO: 99 - protein, 10 aa, synthetic construct
DGTSAAAFDS

SEQ ID NO: 100 - protein, 11 aa, synthetic construct
QASQDITNFLN

SEQ ID NO: 101 - protein, 7 aa, synthetic construct
DASRRRT

SEQ ID NO: 102 - protein, 9 aa, synthetic construct
QQSDSYPIT

SEQ ID NO: 103 - protein, 5 aa, synthetic construct
TYAMS

SEQ ID NO: 104 - protein, 17 aa, synthetic construct
IISDGGDATVYADSVKG

SEQ ID NO: 105 - protein, 10 aa, synthetic construct
DGTSAAAFDS

SEQ ID NO: 106 - protein, 11 aa, synthetic construct
QASQDITNFLN

SEQ ID NO: 107 - protein, 7 aa, synthetic construct
DASRRRT

SEQ ID NO: 108 - protein, 9 aa, synthetic construct
QQSDSYPIT

SEQ ID NO: 109 - protein, 5 aa, synthetic construct
TYAMS

SEQ ID NO: 110 - protein, 17 aa, synthetic construct
IISDGGDATVYADSVKG

SEQ ID NO: 111 - protein, 10 aa, synthetic construct
DGTSAAAFDS

SEQ ID NO: 112 - protein, 11 aa, synthetic construct
HASQDITNFLN

SEQ ID NO: 113 - protein, 7 aa, synthetic construct
DASTLQT

SEQ ID NO: 114 - protein, 9 aa, synthetic construct
QQSDSYPIT

SEQ ID NO: 115 - protein, 5 aa, synthetic construct
TYAMS

SEQ ID NO: 116 - protein, 17 aa, synthetic construct
IISDGGDATVYADSVKG

SEQ ID NO: 117 - protein, 10 aa, synthetic construct
DRTSAAAFDS

SEQ ID NO: 118 - protein, 11 aa, synthetic construct
QASQDITNFLN

SEQ ID NO: 119 - protein, 7 aa, synthetic construct
DASTLQT

SEQ ID NO: 120 - protein, 9 aa, synthetic construct
QQSDSYPIT

SEQ ID NO: 121 - protein, 5 aa, synthetic construct
TYAMS

SEQ ID NO: 122 - protein, 16 aa, synthetic construct
ISDGGDATVYADSVKG

SEQ ID NO: 123 - protein, 10 aa, synthetic construct
DGTSAAAFDS

SEQ ID NO: 124 - protein, 11 aa, synthetic construct
QASQDITNFLN

SEQ ID NO: 125 - protein, 7 aa, synthetic construct
DASTLQT

SEQ ID NO: 126 - protein, 9 aa, synthetic construct
QQSDSYPIT

SEQ ID NO: 127 - protein, 5 aa, synthetic construct
TYAMS

SEQ ID NO: 128 - protein, 17 aa, synthetic construct
IISDGGDATVYADSVKG

SEQ ID NO: 129 - protein, 10 aa, synthetic construct
DGTSAAAFDS

SEQ ID NO: 130 - protein, 11 aa, synthetic construct
QASQDITNFLN

SEQ ID NO: 131 - protein, 7 aa, synthetic construct
DASTLQT

SEQ ID NO: 132 - protein, 9 aa, synthetic construct
QQSDSYPIT

SEQ ID NO: 133 - protein, 5 aa, synthetic construct
TYAMS

SEQ ID NO: 134 - protein, 17 aa, synthetic construct
IISDGGDVTVYADSVKG

SEQ ID NO: 135 - protein, 10 aa, synthetic construct
DRTSAAAFDS

SEQ ID NO: 136 - protein, 11 aa, synthetic construct
QASQDITNFLN

SEQ ID NO: 137 - protein, 7 aa, synthetic construct
DASTLQT

SEQ ID NO: 138 - protein, 9 aa, synthetic construct
QQSDSYPIT

SEQ ID NO: 139 - protein, 5 aa, synthetic construct
TYAMS

SEQ ID NO: 140 - protein, 17 aa, synthetic construct
IISDGGDATVYADSVKG

SEQ ID NO: 141 - protein, 10 aa, synthetic construct
DRTSAAAFDS

SEQ ID NO: 142 - protein, 11 aa, synthetic construct
QASQDITNFLN

SEQ ID NO: 143 - protein, 7 aa, synthetic construct
DASTLQT

SEQ ID NO: 144 - protein, 9 aa, synthetic construct
QQSDSYPIT

SEQ ID NO: 145 - protein, 5 aa, synthetic construct
TYAMS

SEQ ID NO: 146 - protein, 17 aa, synthetic construct
IISDGGDATVYADSVKG

SEQ ID NO: 147 - protein, 10 aa, synthetic construct
DGTSAAAFDS

SEQ ID NO: 148 - protein, 11 aa, synthetic construct
QASQDITNFLN

SEQ ID NO: 149 - protein, 7 aa, synthetic construct
DASTLQT

SEQ ID NO: 150 - protein, 9 aa, synthetic construct
QQSDSYPIT

SEQ ID NO: 151 - protein, 5 aa, synthetic construct
SYAMT

SEQ ID NO: 152 - protein, 17 aa, synthetic construct
GISGGGAATFYADSVKG

SEQ ID NO: 153 - protein, 10 aa, synthetic construct
QSQYVGGFDY

SEQ ID NO: 154 - protein, 14 aa, synthetic construct
TGSSSNIGAGYGVH

SEQ ID NO: 155 - protein, 7 aa, synthetic construct
GNTNRPS

SEQ ID NO: 156 - protein, 11 aa, synthetic construct
QSYDSSLSGWV

SEQ ID NO: 157 - protein, 5 aa, synthetic construct
SYAMT

SEQ ID NO: 158 - protein, 17 aa, synthetic construct
GISGGGAATFYADSVKG

SEQ ID NO: 159 - protein, 10 aa, synthetic construct
QSQYVGGFDY

SEQ ID NO: 160 - protein, 14 aa, synthetic construct
TGSSSNIGAGYGVH

SEQ ID NO: 161 - protein, 7 aa, synthetic construct
GNTNRPS

SEQ ID NO: 162 - protein, 11 aa, synthetic construct
QSYDSSLSGWV

SEQ ID NO: 163 - protein, 5 aa, synthetic construct
SYAMT

SEQ ID NO: 164 - protein, 17 aa, synthetic construct
GISGGGAATFYADSVKG

SEQ ID NO: 165 - protein, 10 aa, synthetic construct
QSQYVGGFDY

SEQ ID NO: 166 - protein, 14 aa, synthetic construct
TGSSSNFGAGYGVH

SEQ ID NO: 167 - protein, 7 aa, synthetic construct
GNTNRPS

SEQ ID NO: 168 - protein, 11 aa, synthetic construct
QSYDSSLSGWV

SEQ ID NO: 169 - protein, 5 aa, synthetic construct
SYAMT

SEQ ID NO: 170 - protein, 17 aa, synthetic construct
GISGDGAATFYADSVKG

SEQ ID NO: 171 - protein, 10 aa, synthetic construct
QSQYVGGFDY

SEQ ID NO: 172 - protein, 14 aa, synthetic construct
TGSSSNFGAGYGVH

SEQ ID NO: 173 - protein, 7 aa, synthetic construct
GNTNRPS

SEQ ID NO: 174 - protein, 11 aa, synthetic construct
QSYDSSLSGWV

SEQ ID NO: 175 - protein, 5 aa, synthetic construct
SYAMS

SEQ ID NO: 176 - protein, 17 aa, synthetic construct
GISAGGGETFYADSVKG

SEQ ID NO: 177 - protein, 10 aa, synthetic construct
VHPISYGFDI

SEQ ID NO: 178 - protein, 11 aa, synthetic construct
QASQDIKKYLN

SEQ ID NO: 179 - protein, 7 aa, synthetic construct
DASTLQT

SEQ ID NO: 180 - protein, 9 aa, synthetic construct
QQSDNTPVT

SEQ ID NO: 181 - protein, 5 aa, synthetic construct
SYAIH

SEQ ID NO: 182 - protein, 17 aa, synthetic construct
VISSDGGYKNYADSVKG

SEQ ID NO: 183 - protein, 6 aa, synthetic construct
DRQIGD

SEQ ID NO: 184 - protein, 11 aa, synthetic construct
RASHGISNYLA

SEQ ID NO: 185 - protein, 7 aa, synthetic construct
AASTLQS

SEQ ID NO: 186 - protein, 8 aa, synthetic construct
QQYNTYRT

SEQ ID NO: 187 - protein, 5 aa, synthetic construct
TYAMT

SEQ ID NO: 188 - protein, 17 aa, synthetic construct
AISDSGGDTFYADSVKG

SEQ ID NO: 189 - protein, 10 aa, synthetic construct
DGTYAARFDY

SEQ ID NO: 190 - protein, 11 aa, synthetic construct
QASQDISNYLN

SEQ ID NO: 191 - protein, 7 aa, synthetic construct
DASTLET

SEQ ID NO: 192 - protein, 9 aa, synthetic construct
QQSDSFPIT

SEQ ID NO: 193 - protein, 5 aa, synthetic construct
NFGIS

SEQ ID NO: 194 - protein, 17 aa, synthetic construct
GIVPVLGIATYAQKFQG

SEQ ID NO: 195 - protein, 11 aa, synthetic construct
GSAWYDGSFQY

SEQ ID NO: 196 - protein, 12 aa, synthetic construct
RASQSVSSTFLA

SEQ ID NO: 197 - protein, 7 aa, synthetic construct
DASTRAT

SEQ ID NO: 198 - protein, 9 aa, synthetic construct
QQYDSWPFT

SEQ ID NO: 199 - protein, 5 aa, synthetic construct
SYGVS

SEQ ID NO: 200 - protein, 17 aa, synthetic construct
RIVPVFGIANYAQKFQG

SEQ ID NO: 201 - protein, 14 aa, synthetic construct
QSPYVTYSSYYFDY

SEQ ID NO: 202 - protein, 12 aa, synthetic construct
RASQSVSNNFLA

SEQ ID NO: 203 - protein, 7 aa, synthetic construct
DASTRAT

SEQ ID NO: 204 - protein, 9 aa, synthetic construct
QQYGSWPIT

SEQ ID NO: 205 - protein, 5 aa, synthetic construct
NYGFS

SEQ ID NO: 206 - protein, 17 aa, synthetic construct
GIVPVFGIATYAQKFQG

SEQ ID NO: 207 - protein, 14 aa, synthetic construct
DYSYYPDDPRYFEY

SEQ ID NO: 208 - protein, 12 aa, synthetic construct
RASQSVSSTFLA

SEQ ID NO: 209 - protein, 7 aa, synthetic construct
AASSRAT

SEQ ID NO: 210 - protein, 9 aa, synthetic construct
QQYGSWPLT

SEQ ID NO: 211 - protein, 5 aa, synthetic construct
SYTMG

SEQ ID NO: 212 - protein, 16 aa, synthetic construct
FISSSGHTYYANWAKG

SEQ ID NO: 213 - protein, 15 aa, synthetic construct
EGGYGGYDYTGIFNL

SEQ ID NO: 214 - protein, 11 aa, synthetic construct
QATESISSWLA

SEQ ID NO: 215 - protein, 7 aa, synthetic construct
GASTLES

SEQ ID NO: 216 - protein, 12 aa, synthetic construct
QQGYIYTNVDNT

SEQ ID NO: 217 - protein, 5 aa, synthetic construct
SYTMG

SEQ ID NO: 218 - protein, 16 aa, synthetic construct
FISSSGHTYYANWAKG

SEQ ID NO: 219 - protein, 15 aa, synthetic construct
DGGYGGYDYTGIFNL

SEQ ID NO: 220 - protein, 11 aa, synthetic construct
QATESISSWLA

SEQ ID NO: 221 - protein, 7 aa, synthetic construct
GASTLES

SEQ ID NO: 222 - protein, 12 aa, synthetic construct
QQGYIYTNVDNT

SEQ ID NO: 223 - protein, 5 aa, synthetic construct
SYGVN

SEQ ID NO: 224 - protein, 16 aa, synthetic construct
GINTGGSTYYANWAKG

SEQ ID NO: 225 - protein, 12 aa, synthetic construct
TSGNNVYNYFTL

SEQ ID NO: 226 - protein, 11 aa, synthetic construct
QASQSIPSLLA

SEQ ID NO: 227 - protein, 7 aa, synthetic construct
APSTLAS

SEQ ID NO: 228 - protein, 13 aa, synthetic construct
QSYYYGDNTYNNI

SEQ ID NO: 229 - protein, 5 aa, synthetic construct
TYDIN

SEQ ID NO: 230 - protein, 16 aa, synthetic construct
IIYTGGITNFANWAKG

SEQ ID NO: 231 - protein, 16 aa, synthetic construct
GGYDSEGYVYPDAFDP

SEQ ID NO: 232 - protein, 11 aa, synthetic construct
QASESISNLLA

SEQ ID NO: 233 - protein, 7 aa, synthetic construct
RASILTS

SEQ ID NO: 234 - protein, 12 aa, synthetic construct
QHGYTGTNVQNV

SEQ ID NO: 235 - protein, 5 aa, synthetic construct
NYAMG

SEQ ID NO: 236 - protein, 16 aa, synthetic construct
SRRTDGITYYANWAEG

SEQ ID NO: 237 - protein, 12 aa, synthetic construct
DVGGEGGWYFNL

SEQ ID NO: 238 - protein, 11 aa, synthetic construct
QASQSINIYLA

SEQ ID NO: 239 - protein, 7 aa, synthetic construct
DASKLAS

SEQ ID NO: 240 - protein, 8 aa, synthetic construct
QQGINNIG

SEQ ID NO: 241 - protein, 5 aa, synthetic construct
NYAMG

SEQ ID NO: 242 - protein, 16 aa, synthetic construct
SRRTDGITYYANWAEG

SEQ ID NO: 243 - protein, 12 aa, synthetic construct
DVGGDGGWYFNL

SEQ ID NO: 244 - protein, 11 aa, synthetic construct
QASQSINIYLA

SEQ ID NO: 245 - protein, 7 aa, synthetic construct
DASKLAS

SEQ ID NO: 246 - protein, 8 aa, synthetic construct
QQGINNIG

SEQ ID NO: 247 - protein, 5 aa, synthetic construct
SYAMG

SEQ ID NO: 248 - protein, 16 aa, synthetic construct
DISTSGNAYYATWVKG

SEQ ID NO: 249 - protein, 12 aa, synthetic construct
ADYGGETYAFDP

SEQ ID NO: 250 - protein, 11 aa, synthetic construct
QASQSISSYLN

SEQ ID NO: 251 - protein, 7 aa, synthetic construct
SASTLAS

SEQ ID NO: 252 - protein, 12 aa, synthetic construct
QQGYSDSNIDNV

SEQ ID NO: 253 - protein, 5 aa, synthetic construct
SHHMI

SEQ ID NO: 254 - protein, 17 aa, synthetic construct
IIDAGSGSTYYASWAKG

SEQ ID NO: 255 - protein, 13 aa, synthetic construct
GGLTESLGTYFDL

SEQ ID NO: 256 - protein, 11 aa, synthetic construct
QASESIDSGLA

SEQ ID NO: 257 - protein, 7 aa, synthetic construct
DSSTLAS

SEQ ID NO: 258 - protein, 15 aa, synthetic construct
QSNYDTGSSVYDWGS

SEQ ID NO: 259 - protein, 5 aa, synthetic construct
DYFMT

SEQ ID NO: 260 - protein, 16 aa, synthetic construct
IINTGGDSYYATWAKG

SEQ ID NO: 261 - protein, 15 aa, synthetic construct
DTGYGGYDYAGSFDP

SEQ ID NO: 262 - protein, 11 aa, synthetic construct
QASENINSWLA

SEQ ID NO: 263 - protein, 7 aa, synthetic construct
EASKLAS

SEQ ID NO: 264 - protein, 12 aa, synthetic construct
QQGYIYIDVGNI

SEQ ID NO: 265 - protein, 6 aa, synthetic construct
VSYWIC

SEQ ID NO: 266 - protein, 17 aa, synthetic construct
CTDGGDGSSYYASWVNG

SEQ ID NO: 267 - protein, 8 aa, synthetic construct
DRSDVFNL

SEQ ID NO: 268 - protein, 11 aa, synthetic construct
QAGQSIDSNLA

SEQ ID NO: 269 - protein, 7 aa, synthetic construct
RASTLAS

SEQ ID NO: 270 - protein, 14 aa, synthetic construct
QSFYVTISAMVDYP

SEQ ID NO: 271 - protein, 5 aa, synthetic construct
RYAMA

SEQ ID NO: 272 - protein, 16 aa, synthetic construct
YIDTGDSTYYATWAKG

SEQ ID NO: 273 - protein, 7 aa, synthetic construct
VGVRMYL

SEQ ID NO: 274 - protein, 11 aa, synthetic construct
QASQSISSYLS

SEQ ID NO: 275 - protein, 7 aa, synthetic construct
RASTLES

SEQ ID NO: 276 - protein, 12 aa, synthetic construct
QCGYYGGSYIGA

SEQ ID NO: 277 - protein, 5 aa, synthetic construct
SYGIS

SEQ ID NO: 278 - protein, 17 aa, synthetic construct
YIYPDYGSTDYATWVNG

SEQ ID NO: 279 - protein, 16 aa, synthetic construct
GYASSSGYYDPKYFGL

SEQ ID NO: 280 - protein, 11 aa, synthetic construct
RASEDIESYLA

SEQ ID NO: 281 - protein, 7 aa, synthetic construct
DASDLAS

SEQ ID NO: 282 - protein, 12 aa, synthetic construct
QHGFYTSRSDSV

SEQ ID NO: 283 - protein, 5 aa, synthetic construct
SYDMS

SEQ ID NO: 284 - protein, 16 aa, synthetic construct
YIWSSGSAYYATWAEG

SEQ ID NO: 285 - protein, 9 aa, synthetic construct
RYVGSSYDT

SEQ ID NO: 286 - protein, 13 aa, synthetic construct
QSSQSVSSNNYLS

SEQ ID NO: 287 - protein, 7 aa, synthetic construct
AASYLAS

SEQ ID NO: 288 - protein, 11 aa, synthetic construct
LGDYDNDIDHA

SEQ ID NO: 289 - protein, 5 aa, synthetic construct
SYAMG

SEQ ID NO: 290 - protein, 16 aa, synthetic construct
FIDTGGSTYYANWAKG

SEQ ID NO: 291 - protein, 7 aa, synthetic construct
VGARMYL

SEQ ID NO: 292 - protein, 11 aa, synthetic construct
QASQSISNLLA

SEQ ID NO: 293 - protein, 7 aa, synthetic construct
RASTLES

SEQ ID NO: 294 - protein, 12 aa, synthetic construct
QCSYYGGSYIGA

SEQ ID NO: 295 - protein, 5 aa, synthetic construct
RYYMS

SEQ ID NO: 296 - protein, 17 aa, synthetic construct
YIDPIFGNTYYASWVNG

SEQ ID NO: 297 - protein, 15 aa, synthetic construct
DGDAGYDGYGYGTDL

SEQ ID NO: 298 - protein, 11 aa, synthetic construct
QASENIYSGLA

SEQ ID NO: 299 - protein, 7 aa, synthetic construct
SAFTLAS

SEQ ID NO: 300 - protein, 13 aa, synthetic construct
QTYYYGSVTYFNA

SEQ ID NO: 301 - protein, 5 aa, synthetic construct
SHYMI

SEQ ID NO: 302 - protein, 17 aa, synthetic construct
IITSSDYIYYARWAKGR

SEQ ID NO: 303 - protein, 12 aa, synthetic construct
YNYDDDGELFNL

SEQ ID NO: 304 - protein, 13 aa, synthetic construct
QSSQSIDANNDLA

SEQ ID NO: 305 - protein, 7 aa, synthetic construct
LASKLAS

SEQ ID NO: 306 - protein, 11 aa, synthetic construct
LGGYDDDADNT

SEQ ID NO: 307 - protein, 6 aa, synthetic construct
NNYYMC

SEQ ID NO: 308 - protein, 18 aa, synthetic construct
CIYPSIVGPTYYANWAKG

SEQ ID NO: 309 - protein, 12 aa, synthetic construct
DRYDDYGDYFNL

SEQ ID NO: 310 - protein, 11 aa, synthetic construct
QASQSIYNYLS

SEQ ID NO: 311 - protein, 7 aa, synthetic construct
YASTLAS

SEQ ID NO: 312 - protein, 14 aa, synthetic construct
QSNSGVNGNRYGNA

SEQ ID NO: 313 - protein, 8 aa, synthetic construct
SYSFTDYN

SEQ ID NO: 314 - protein, 16 aa, synthetic construct
IFPKYGTTSYNQKFKG

SEQ ID NO: 315 - protein, 11 aa, synthetic construct
ATDGGTWYFDV

SEQ ID NO: 316 - protein, 5 aa, synthetic construct
SSVTY

SEQ ID NO: 317 - protein, 11 aa, synthetic construct
LTSNLASGVPA

SEQ ID NO: 318 - protein, 9 aa, synthetic construct
QQWSSNPPT

SEQ ID NO: 319 - protein, 8 aa, synthetic construct
SYSFTDYN

SEQ ID NO: 320 - protein, 16 aa, synthetic construct
IFPKYGTTSYAQKLQG

SEQ ID NO: 321 - protein, 11 aa, synthetic construct
ATDGGTWYFDV

SEQ ID NO: 322 - protein, 5 aa, synthetic construct
SSVTY

SEQ ID NO: 323 - protein, 11 aa, synthetic construct
LTSNLASGVPS

SEQ ID NO: 324 - protein, 9 aa, synthetic construct
QQWSSNPPT

SEQ ID NO: 325 - protein, 8 aa, synthetic construct
GYTFTDYY

SEQ ID NO: 326 - protein, 16 aa, synthetic construct
INPNDGGTTYSQKFKG

SEQ ID NO: 327 - protein, 18 aa, synthetic construct
AREGNYYAYDVRVWYFDV

SEQ ID NO: 328 - protein, 6 aa, synthetic construct
QDIITY

SEQ ID NO: 329 - protein, 11 aa, synthetic construct
STSSLNSGVPS

SEQ ID NO: 330 - protein, 9 aa, synthetic construct
QQYSELPYT

SEQ ID NO: 331 - protein, 8 aa, synthetic construct
GYTFTDYY

SEQ ID NO: 332 - protein, 16 aa, synthetic construct
INPNDGGTTYAQKFQG

SEQ ID NO: 333 - protein, 18 aa, synthetic construct
AREGNYYAYDVRVWYFDV

SEQ ID NO: 334 - protein, 6 aa, synthetic construct
QDIITY

SEQ ID NO: 335 - protein, 11 aa, synthetic construct
STSSLNSGVPS

SEQ ID NO: 336 - protein, 9 aa, synthetic construct
QQYSELPYT

SEQ ID NO: 337 - protein, 8 aa, synthetic construct
GFTFSDAW

SEQ ID NO: 338 - protein, 18 aa, synthetic construct
VRNKANNHATYYAESVKG

SEQ ID NO: 339 - protein, 16 aa, synthetic construct
TRSVGGYGTTYWYFDV

SEQ ID NO: 340 - protein, 12 aa, synthetic construct
QNLLNSGNQKNY

SEQ ID NO: 341 - protein, 11 aa, synthetic construct
GASTRESGVPD

SEQ ID NO: 342 - protein, 9 aa, synthetic construct
QSEHSYPYT

SEQ ID NO: 343 - protein, 8 aa, synthetic construct
GFTFSDAW

SEQ ID NO: 344 - protein, 18 aa, synthetic construct
VRNKANNHATYYAASVKG

SEQ ID NO: 345 - protein, 16 aa, synthetic construct
TRSVGGYGTTYWYFDV

SEQ ID NO: 346 - protein, 12 aa, synthetic construct
QNLLNSGNQKNY

SEQ ID NO: 347 - protein, 11 aa, synthetic construct
GASTRESGVPD

SEQ ID NO: 348 - protein, 9 aa, synthetic construct
QSEHSYPYT

SEQ ID NO: 349 - protein, 8 aa, synthetic construct
GDTFTSYS

SEQ ID NO: 350 - protein, 8 aa, synthetic construct
IITILDDT

SEQ ID NO: 351 - protein, 21 aa, synthetic construct
AGVYEGPNSEGSYDDYGFLKH

SEQ ID NO: 352 - protein, 6 aa, synthetic construct
ESVAND

SEQ ID NO: 353 - skipped

SEQ ID NO: 354 - protein, 11 aa, synthetic construct
QQYANWPPRET

SEQ ID NO: 355 - protein, 8 aa, synthetic construct
GDTFTSYS

SEQ ID NO: 356 - protein, 8 aa, synthetic construct
IITILDDT

SEQ ID NO: 357 - protein, 21 aa, synthetic construct
AGVYEGPNSEGSYDDYGFLKH

SEQ ID NO: 358 - protein, 6 aa, synthetic construct
ESVAND

SEQ ID NO: 359 - skipped

SEQ ID NO: 360 - protein, 11 aa, synthetic construct
QQYANWPPRET

SEQ ID NO: 361 - protein, 8 aa, synthetic construct
GDTFTSYS

SEQ ID NO: 362 - protein, 8 aa, synthetic construct
IITDLDDT

SEQ ID NO: 363 - protein, 21 aa, synthetic construct
AGVYEGPNSEGSYDDYGFLKH

SEQ ID NO: 364 - protein, 6 aa, synthetic construct
ESGAND

SEQ ID NO: 365 - skipped

SEQ ID NO: 366 - protein, 11 aa, synthetic construct
QQYANWPPRGT

SEQ ID NO: 367 - protein, 8 aa, synthetic construct
GDTFTSYS

SEQ ID NO: 368 - protein, 8 aa, synthetic construct
IITILDDT

SEQ ID NO: 369 - protein, 21 aa, synthetic construct
AGVYEGPNSEGSYDDYGFLKH

SEQ ID NO: 370 - protein, 6 aa, synthetic construct
ESVAND

SEQ ID NO: 371 - skipped

SEQ ID NO: 372 - protein, 11 aa, synthetic construct
QQYTNWPPRET

SEQ ID NO: 373 - protein, 8 aa, synthetic construct
GDTFTSYS

SEQ ID NO: 374 - protein, 8 aa, synthetic construct
IITILDDT

SEQ ID NO: 375 - protein, 21 aa, synthetic construct
AGVYEGPNSEGSYDDYGFLKH

SEQ ID NO: 376 - protein, 6 aa, synthetic construct
ESVAND

SEQ ID NO: 377 - skipped

SEQ ID NO: 378 - protein, 11 aa, synthetic construct
QQYAIWPPRET

SEQ ID NO: 379 - protein, 8 aa, synthetic construct
GDTFTSYS

SEQ ID NO: 380 - protein, 8 aa, synthetic construct
IITILDDT

SEQ ID NO: 381 - protein, 21 aa, synthetic construct
AGVYEGPNSEGSYDDYGFLKH

SEQ ID NO: 382 - protein, 6 aa, synthetic construct
RSVAND

SEQ ID NO: 383 - skipped

SEQ ID NO: 384 - protein, 11 aa, synthetic construct
QQYAIWPPRET

SEQ ID NO: 385 - protein, 8 aa, synthetic construct
GDTFTSYS

SEQ ID NO: 386 - protein, 8 aa, synthetic construct
IITILDDT

SEQ ID NO: 387 - protein, 21 aa, synthetic construct
AGVYEGPNSEGSYDDYGFLKH

SEQ ID NO: 388 - protein, 6 aa, synthetic construct
ESVANN

SEQ ID NO: 389 - skipped

SEQ ID NO: 390 - protein, 11 aa, synthetic construct
QQYARWPPTET

SEQ ID NO: 391 - protein, 8 aa, synthetic construct
GDTFTSYS

SEQ ID NO: 392 - protein, 8 aa, synthetic construct
IITIPDDT

SEQ ID NO: 393 - protein, 21 aa, synthetic construct
AGVYEGPNSEGSYDDYGFLKH

SEQ ID NO: 394 - protein, 6 aa, synthetic construct
ESVATD

SEQ ID NO: 395 - skipped

SEQ ID NO: 396 - protein, 11 aa, synthetic construct
QQYDNWPPRET

SEQ ID NO: 397 - protein, 8 aa, synthetic construct
GDTFTSYS

SEQ ID NO: 398 - protein, 8 aa, synthetic construct
IITILDDT

SEQ ID NO: 399 - protein, 21 aa, synthetic construct
AGVYEGPNSEGSYDDYGFLKH

SEQ ID NO: 400 - protein, 6 aa, synthetic construct
ESVRND

SEQ ID NO: 401 - skipped

SEQ ID NO: 402 - protein, 11 aa, synthetic construct
QQYANWPPHET

SEQ ID NO: 403 - protein, 8 aa, synthetic construct
GDTFTSYS

SEQ ID NO: 404 - protein, 8 aa, synthetic construct
IITIDDDT

SEQ ID NO: 405 - protein, 21 aa, synthetic construct
AGVYEGPNSEGSYDDYGFYKH

SEQ ID NO: 406 - protein, 6 aa, synthetic construct
ESVGND

SEQ ID NO: 407 - skipped

SEQ ID NO: 408 - protein, 11 aa, synthetic construct
QQYANWPPRET

SEQ ID NO: 409 - protein, 8 aa, synthetic construct
GDTFTSYS

SEQ ID NO: 410 - protein, 8 aa, synthetic construct
IITILDDS

SEQ ID NO: 411 - protein, 21 aa, synthetic construct
AGVYEGPNSEGSYDDYGFLKH

SEQ ID NO: 412 - protein, 6 aa, synthetic construct
ESVAND

SEQ ID NO: 413 - skipped

SEQ ID NO: 414 - protein, 11 aa, synthetic construct
QQYANWPWRET

SEQ ID NO: 415 - protein, 8 aa, synthetic construct
GDTFTSYS

SEQ ID NO: 416 - protein, 8 aa, synthetic construct
IITIDDDT

SEQ ID NO: 417 - protein, 21 aa, synthetic construct
AGVYEGPNSEGSYDDYGFYKH

SEQ ID NO: 418 - protein, 6 aa, synthetic construct
ESVAND

SEQ ID NO: 419 - skipped

SEQ ID NO: 420 - protein, 11 aa, synthetic construct
QQGANWPPRET

SEQ ID NO: 421 - protein, 8 aa, synthetic construct
GDTFTSYS

SEQ ID NO: 422 - protein, 8 aa, synthetic construct
IITIYDDT

SEQ ID NO: 423 - protein, 21 aa, synthetic construct
AGVYEGPNSEGSYDDYGFFKH

SEQ ID NO: 424 - protein, 6 aa, synthetic construct
ESVAND

SEQ ID NO: 425 - skipped

SEQ ID NO: 426 - protein, 11 aa, synthetic construct
QQYANWPPRET

SEQ ID NO: 427 - protein, 8 aa, synthetic construct
GDTFTSYS

SEQ ID NO: 428 - protein, 8 aa, synthetic construct
IITILDDT

SEQ ID NO: 429 - protein, 21 aa, synthetic construct
AGVYEGPNSEGSYDDYGFLKY

SEQ ID NO: 430 - protein, 6 aa, synthetic construct
ESVAVD

SEQ ID NO: 431 - skipped

SEQ ID NO: 432 - protein, 11 aa, synthetic construct
QQYANKPPRET

SEQ ID NO: 433 - protein, 8 aa, synthetic construct
GDTFTSYS

SEQ ID NO: 434 - protein, 8 aa, synthetic construct
IITILDDT

SEQ ID NO: 435 - protein, 21 aa, synthetic construct
AGVYEGPNSEGSYDDYGALKH

SEQ ID NO: 436 - protein, 6 aa, synthetic construct
ESVAND

SEQ ID NO: 437 - skipped

SEQ ID NO: 438 - protein, 11 aa, synthetic construct
QQYANWPPRET

SEQ ID NO: 439 - protein, 8 aa, synthetic construct
GDTFTSYS

SEQ ID NO: 440 - protein, 8 aa, synthetic construct
IITDLDDT

SEQ ID NO: 441 - protein, 21 aa, synthetic construct
AGVYEGPNSEGSYDDYGFLKH

SEQ ID NO: 442 - protein, 6 aa, synthetic construct
ESGAND

SEQ ID NO: 443 - skipped

SEQ ID NO: 444 - protein, 11 aa, synthetic construct
QQYANWPPRGT

SEQ ID NO: 445 - protein, 8 aa, synthetic construct
GDTFTSYS

SEQ ID NO: 446 - protein, 8 aa, synthetic construct
IITDLDDT

SEQ ID NO: 447 - protein, 21 aa, synthetic construct
AGVYEGPNSEGSYDDYGQLKH

SEQ ID NO: 448 - protein, 6 aa, synthetic construct
ESVAND

SEQ ID NO: 449 - skipped

SEQ ID NO: 450 - protein, 11 aa, synthetic construct
QQYANWPPRET

SEQ ID NO: 451 - protein, 8 aa, synthetic construct
GDTFTSYS

SEQ ID NO: 452 - protein, 8 aa, synthetic construct
IITILDDT

SEQ ID NO: 453 - protein, 21 aa, synthetic construct
AGVYEGPNSEGSYDDYGQLKH

SEQ ID NO: 454 - protein, 6 aa, synthetic construct
ESVAND

SEQ ID NO: 455 - skipped

SEQ ID NO: 456 - protein, 11 aa, synthetic construct
QQYANTPPRET

SEQ ID NO: 457 - protein, 8 aa, synthetic construct
GDTFTSYS

SEQ ID NO: 458 - protein, 8 aa, synthetic construct
IITDLDDT

SEQ ID NO: 459 - protein, 21 aa, synthetic construct
AGVYEGPNSEGSYDDYGFLKH

SEQ ID NO: 460 - protein, 6 aa, synthetic construct
ESVAND

SEQ ID NO: 461 - skipped

SEQ ID NO: 462 - protein, 11 aa, synthetic construct
QQYANWPPRET

SEQ ID NO: 463 - protein, 8 aa, synthetic construct
GDTFTSYS

SEQ ID NO: 464 - protein, 8 aa, synthetic construct
IITILDDT

SEQ ID NO: 465 - protein, 21 aa, synthetic construct
AGVYEGPNSEGSYDDYGFLKH

SEQ ID NO: 466 - protein, 6 aa, synthetic construct
ESVGND

SEQ ID NO: 467 - skipped

SEQ ID NO: 468 - protein, 11 aa, synthetic construct
QQYANWPPRET

SEQ ID NO: 469 - protein, 8 aa, synthetic construct
GDTFTSYS

SEQ ID NO: 470 - protein, 8 aa, synthetic construct
IITIDDDT

SEQ ID NO: 471 - protein, 21 aa, synthetic construct
AGVYEGPNSEGSYDDYGQLKH

SEQ ID NO: 472 - protein, 6 aa, synthetic construct
ESVGND

SEQ ID NO: 473 - skipped

SEQ ID NO: 474 - protein, 11 aa, synthetic construct
QQYANVPPRET

SEQ ID NO: 475 - protein, 8 aa, synthetic construct
GDTFTSYS

SEQ ID NO: 476 - protein, 8 aa, synthetic construct
IITIDDDT

SEQ ID NO: 477 - protein, 21 aa, synthetic construct
AGVYEGPNSEGSYDDYGQLKH

SEQ ID NO: 478 - protein, 6 aa, synthetic construct
ESVGND

SEQ ID NO: 479 - skipped

SEQ ID NO: 480 - protein, 11 aa, synthetic construct
QQYANDPPRET

SEQ ID NO: 481 - protein, 8 aa, synthetic construct
GYTFTDLG

SEQ ID NO: 482 - protein, 8 aa, synthetic construct
INTHTGMP

SEQ ID NO: 483 - protein, 10 aa, synthetic construct
ARDHPLGLDY

SEQ ID NO: 484 - protein, 10 aa, synthetic construct
QSVDVNGVSY

SEQ ID NO: 485 - skipped

SEQ ID NO: 486 - protein, 9 aa, synthetic construct
QQSREDPYT

SEQ ID NO: 487 - protein, 8 aa, synthetic construct
GYTFTDLG

SEQ ID NO: 488 - protein, 8 aa, synthetic construct
INTHTGEP

SEQ ID NO: 489 - protein, 10 aa, synthetic construct
TRDHPLGLDY

SEQ ID NO: 490 - protein, 10 aa, synthetic construct
QSVDVNGVSY

SEQ ID NO: 491 - skipped

SEQ ID NO: 492 - protein, 9 aa, synthetic construct
QQSREDPYT

SEQ ID NO: 493 - protein, 8 aa, synthetic construct
GYTFTDLG

SEQ ID NO: 494 - protein, 8 aa, synthetic construct
INTHTGEP

SEQ ID NO: 495 - protein, 10 aa, synthetic construct
TRDHPLGLDY

SEQ ID NO: 496 - protein, 10 aa, synthetic construct
QSVDVNGLSY

SEQ ID NO: 497 - skipped

SEQ ID NO: 498 - protein, 9 aa, synthetic construct
QQSREDPYT

SEQ ID NO: 499 - protein, 8 aa, synthetic construct
GYTFTDLG

SEQ ID NO: 500 - protein, 8 aa, synthetic construct
INTHTGKP

SEQ ID NO: 501 - protein, 10 aa, synthetic construct
TRDHPLGLDY

SEQ ID NO: 502 - protein, 10 aa, synthetic construct
QSVDVNGVSY

SEQ ID NO: 503 - skipped

SEQ ID NO: 504 - protein, 9 aa, synthetic construct
QQSREDPYT

SEQ ID NO: 505 - protein, 8 aa, synthetic construct
GYTFTDLG

SEQ ID NO: 506 - protein, 8 aa, synthetic construct
INTQTGEP

SEQ ID NO: 507 - protein, 10 aa, synthetic construct
TRDHPLGLDY

SEQ ID NO: 508 - protein, 10 aa, synthetic construct
QSVDVAGVSY

SEQ ID NO: 509 - skipped

SEQ ID NO: 510 - protein, 9 aa, synthetic construct
QQAREDPYT

SEQ ID NO: 511 - protein, 8 aa, synthetic construct
GYTFTDLG

SEQ ID NO: 512 - protein, 8 aa, synthetic construct
INTQTGEP

SEQ ID NO: 513 - protein, 10 aa, synthetic construct
ARDHPLGLDY

SEQ ID NO: 514 - protein, 10 aa, synthetic construct
QSVDVNGVSY

SEQ ID NO: 515 - skipped

SEQ ID NO: 516 - protein, 9 aa, synthetic construct
QQSREDPYT

SEQ ID NO: 517 - protein, 8 aa, synthetic construct
GYTFTDLG

SEQ ID NO: 518 - protein, 8 aa, synthetic construct
INTHTGEP

SEQ ID NO: 519 - protein, 10 aa, synthetic construct
TRDHPLGLDY

SEQ ID NO: 520 - protein, 10 aa, synthetic construct
QSVDVNGVSY

SEQ ID NO: 521 - skipped

SEQ ID NO: 522 - protein, 9 aa, synthetic construct
QQSREDPYT

SEQ ID NO: 523 - protein, 8 aa, synthetic construct
GYTFTDLG

SEQ ID NO: 524 - protein, 8 aa, synthetic construct
INTHTGEP

SEQ ID NO: 525 - protein, 10 aa, synthetic construct
ARDHPLGLDY

SEQ ID NO: 526 - protein, 10 aa, synthetic construct
QSVDVNGVSY

SEQ ID NO: 527 - skipped

SEQ ID NO: 528 - protein, 9 aa, synthetic construct
QQSREDPYT

SEQ ID NO: 529 - protein, 8 aa, synthetic construct
GYTFTDLG

SEQ ID NO: 530 - protein, 8 aa, synthetic construct
INTITGEP

SEQ ID NO: 531 - protein, 10 aa, synthetic construct
TRDHPLGLDY

SEQ ID NO: 532 - protein, 10 aa, synthetic construct
QSLDVNGVSY

SEQ ID NO: 533 - skipped

SEQ ID NO: 534 - protein, 9 aa, synthetic construct
QQSREDPYT

SEQ ID NO: 535 - protein, 8 aa, synthetic construct
GYTFTDLG

SEQ ID NO: 536 - protein, 8 aa, synthetic construct
INTHTGEP

SEQ ID NO: 537 - protein, 10 aa, synthetic construct
TRDHPLGLDY

SEQ ID NO: 538 - protein, 10 aa, synthetic construct
QSVDVNGVSY

SEQ ID NO: 539 - skipped

SEQ ID NO: 540 - protein, 9 aa, synthetic construct
QQNREDPYT

SEQ ID NO: 541 - protein, 8 aa, synthetic construct
GYTFTDLG

SEQ ID NO: 542 - protein, 8 aa, synthetic construct
INTITGEP

SEQ ID NO: 543 - protein, 10 aa, synthetic construct
TRDHPLGLDY

SEQ ID NO: 544 - protein, 10 aa, synthetic construct
QSVDVNGVSY

SEQ ID NO: 545 - skipped

SEQ ID NO: 546 - protein, 9 aa, synthetic construct
QQSREDPYT

SEQ ID NO: 547 - protein, 8 aa, synthetic construct
GYTFTDLG

SEQ ID NO: 548 - protein, 8 aa, synthetic construct
INTHTGEP

SEQ ID NO: 549 - protein, 10 aa, synthetic construct
TRDHPLGLDY

SEQ ID NO: 550 - protein, 10 aa, synthetic construct
QSVDVNGVSY

SEQ ID NO: 551 - skipped

SEQ ID NO: 552 - protein, 9 aa, synthetic construct
QQAREDPYT

SEQ ID NO: 553 - protein, 8 aa, synthetic construct
GFNIRDTY

SEQ ID NO: 554 - protein, 8 aa, synthetic construct
IDPDNGDT

SEQ ID NO: 555 - protein, 13 aa, synthetic construct
SRDLADGYNAFDR

SEQ ID NO: 556 - protein, 6 aa, synthetic construct
QDVSTA

SEQ ID NO: 557 - skipped

SEQ ID NO: 558 - protein, 9 aa, synthetic construct
QQPTYTPPT

SEQ ID NO: 559 - protein, 8 aa, synthetic construct
GFNIRDTF

SEQ ID NO: 560 - protein, 8 aa, synthetic construct
IDPDNGDT

SEQ ID NO: 561 - protein, 13 aa, synthetic construct
SREGEDGYNAWDL

SEQ ID NO: 562 - protein, 6 aa, synthetic construct
QDVSTA

SEQ ID NO: 563 - skipped

SEQ ID NO: 564 - protein, 9 aa, synthetic construct
QQPPYTPPT

SEQ ID NO: 565 - protein, 8 aa, synthetic construct
GFNIRATY

SEQ ID NO: 566 - protein, 8 aa, synthetic construct
IDPTNGDT

SEQ ID NO: 567 - protein, 13 aa, synthetic construct
TREGDDGYNAWDK

SEQ ID NO: 568 - protein, 6 aa, synthetic construct
QDVSTA

SEQ ID NO: 569 - skipped

SEQ ID NO: 570 - protein, 9 aa, synthetic construct
QQPSYTPPT

SEQ ID NO: 571 - protein, 14 aa, synthetic construct
FSDYYMSWVRQAPG

SEQ ID NO: 572 - protein, 19 aa, synthetic construct
ANINTDGSEKYYLDSVKGR

SEQ ID NO: 573 - protein, 10 aa, synthetic construct
AREEYGAFDI

SEQ ID NO: 574 - protein, 14 aa, synthetic construct
CSGSSSNIGSNTVN

SEQ ID NO: 575 - protein, 7 aa, synthetic construct
DNNKRPS

SEQ ID NO: 576 - protein, 13 aa, synthetic construct
CQSFDRGLSGSIV

SEQ ID NO: 577 - protein, 14 aa, synthetic construct
FSSYAMHWVRQAPG

SEQ ID NO: 578 - protein, 19 aa, synthetic construct
SAISGGATTTYYADSVKGR

SEQ ID NO: 579 - protein, 13 aa, synthetic construct
AKGGTGDPYYFDY

SEQ ID NO: 580 - protein, 15 aa, synthetic construct
CTGSSSNIGAGYDVH

SEQ ID NO: 581 - protein, 7 aa, synthetic construct
RNNQRPS

SEQ ID NO: 582 - protein, 12 aa, synthetic construct
CAARDDGLSGPV

SEQ ID NO: 583 - protein, 14 aa, synthetic construct
FSNAWMSWVRQAPG

SEQ ID NO: 584 - protein, 19 aa, synthetic construct
SSISSASGYIYYGDSVKGR

SEQ ID NO: 585 - protein, 12 aa, synthetic construct
ARGTLYGDFDEF

SEQ ID NO: 586 - protein, 14 aa, synthetic construct
CSGSSSNIGNNAVN

SEQ ID NO: 587 - protein, 7 aa, synthetic construct
GNTNRPS

SEQ ID NO: 588 - protein, 13 aa, synthetic construct
CQSYDSSLSGYVV

SEQ ID NO: 589 - protein, 14 aa, synthetic construct
FSSNEMSWIRQAPG

SEQ ID NO: 590 - protein, 18 aa, synthetic construct
SVIYSGGSTYYADSVKGR

SEQ ID NO: 591 - protein, 12 aa, synthetic construct
ARREGWLVPFDY

SEQ ID NO: 592 - protein, 14 aa, synthetic construct
CSGSSSNIGSNTVN

SEQ ID NO: 593 - protein, 7 aa, synthetic construct
GNIIRPS

SEQ ID NO: 594 - protein, 13 aa, synthetic construct
CQSFDTTLSGSIV

SEQ ID NO: 595 - protein, 14 aa, synthetic construct
FSRYWMHWVRQVPG

SEQ ID NO: 596 - protein, 19 aa, synthetic construct
SGISDSGVVTYYADSVKGR

SEQ ID NO: 597 - protein, 10 aa, synthetic construct
ARAQSVAFDI

SEQ ID NO: 598 - protein, 15 aa, synthetic construct
CSGSSSNIGAGHDVH

SEQ ID NO: 599 - protein, 7 aa, synthetic construct
YDDLLPS

SEQ ID NO: 600 - protein, 12 aa, synthetic construct
CAAWDDSLSGWV

SEQ ID NO: 601 - protein, 14 aa, synthetic construct
FSSYAMSWVRQAPG

SEQ ID NO: 602 - protein, 19 aa, synthetic construct
SVISGSGGSTYYADAVKGR

SEQ ID NO: 603 - protein, 10 aa, synthetic construct
TTDSGSGSYL

SEQ ID NO: 604 - protein, 15 aa, synthetic construct
CTGSSSNIGAGYDVH

SEQ ID NO: 605 - protein, 7 aa, synthetic construct
SNNQRPS

SEQ ID NO: 606 - protein, 12 aa, synthetic construct
CAAWDDSLNGPV

SEQ ID NO: 607 - protein, 14 aa, synthetic construct
FSSNYMSWVRQAPG

SEQ ID NO: 608 - protein, 19 aa, synthetic construct
SVISGSGGSTYYADSVKGR

SEQ ID NO: 609 - protein, 9 aa, synthetic construct
ARDRGWFDP

SEQ ID NO: 610 - protein, 14 aa, synthetic construct
CSGSRSNIDNSYVS

SEQ ID NO: 611 - protein, 7 aa, synthetic construct
RNNQRPS

SEQ ID NO: 612 - protein, 12 aa, synthetic construct
CATWDDSLSGPV

SEQ ID NO: 613 - protein, 14 aa, synthetic construct
FSRHAMNWVRQAPG

SEQ ID NO: 614 - protein, 19 aa, synthetic construct
SSISTGSSYIDYADSVKGR

SEQ ID NO: 615 - protein, 13 aa, synthetic construct
AREKGHYYYGMDV

SEQ ID NO: 616 - protein, 15 aa, synthetic construct
CTGSSSNIGAGYDVH

SEQ ID NO: 617 - protein, 7 aa, synthetic construct
GNSYRPS

SEQ ID NO: 618 - protein, 13 aa, synthetic construct
CQSYDTSLSAYVV

SEQ ID NO: 619 - protein, 14 aa, synthetic construct
FSNAWMSWVRQAPG

SEQ ID NO: 620 - protein, 19 aa, synthetic construct
SAISVSGINTYYADSVKGR

SEQ ID NO: 621 - protein, 11 aa, synthetic construct
ARDTGSLGVDY

SEQ ID NO: 622 - protein, 14 aa, synthetic construct
CSGSSSNIGSNTVN

SEQ ID NO: 623 - protein, 7 aa, synthetic construct
RNNQRPS

SEQ ID NO: 624 - protein, 12 aa, synthetic construct
CQSYDSSLSISV

SEQ ID NO: 625 - protein, 14 aa, synthetic construct
SSSYWMSWVRQAPG

SEQ ID NO: 626 - protein, 19 aa, synthetic construct
SAISGSGGSTYYADSVKGR

SEQ ID NO: 627 - protein, 11 aa, synthetic construct
AREYSGYEFDF

SEQ ID NO: 628 - protein, 15 aa, synthetic construct
CTGSSSNIGARSDVH

SEQ ID NO: 629 - protein, 7 aa, synthetic construct
GNRNRPS

SEQ ID NO: 630 - protein, 13 aa, synthetic construct
CQSFDRGLSGSIV

SEQ ID NO: 631 - protein, 14 aa, synthetic construct
SSSYWMSWVRQAPG

SEQ ID NO: 632 - protein, 19 aa, synthetic construct
SAISGSGGSTYYADSVKGR

SEQ ID NO: 633 - protein, 11 aa, synthetic construct
AREYSGYEFDF

SEQ ID NO: 634 - protein, 15 aa, synthetic construct
CTGSSSNIGARSDVH

SEQ ID NO: 635 - protein, 7 aa, synthetic construct
GNRNRPS

SEQ ID NO: 636 - protein, 13 aa, synthetic construct
CQSFDRGLSGSIV

SEQ ID NO: 637 - protein, 14 aa, synthetic construct
FSSNYMSWVRQAPG

SEQ ID NO: 638 - protein, 19 aa, synthetic construct
SSISSSSSYIYYADSVKGR

SEQ ID NO: 639 - protein, 11 aa, synthetic construct
ARDRGRTGTDY

SEQ ID NO: 640 - protein, 14 aa, synthetic construct
CSGTTSNIGSYAVN

SEQ ID NO: 641 - protein, 7 aa, synthetic construct
GNINRPS

SEQ ID NO: 642 - protein, 12 aa, synthetic construct
CQSYDSSLSASL

SEQ ID NO: 643 - protein, 14 aa, synthetic construct
FSSYAMSWVRQAPG

SEQ ID NO: 644 - protein, 19 aa, synthetic construct
STIIGSGANTWYADSVKGR

SEQ ID NO: 645 - protein, 13 aa, synthetic construct
ARHEGYYYYGMDV

SEQ ID NO: 646 - protein, 15 aa, synthetic construct
CTGSSSNIGAGYVVH

SEQ ID NO: 647 - protein, 7 aa, synthetic construct
GNSNRPS

SEQ ID NO: 648 - protein, 12 aa, synthetic construct
CAAWDDSLNGRV

SEQ ID NO: 649 - protein, 5 aa, synthetic construct
GYNMN

SEQ ID NO: 650 - protein, 17 aa, synthetic construct
KIDPYYGGTDYNLRFKD

SEQ ID NO: 651 - protein, 13 aa, synthetic construct
EPNYGSNDWYFDV

SEQ ID NO: 652 - protein, 11 aa, synthetic construct
RASQDIDNYLN

SEQ ID NO: 653 - protein, 7 aa, synthetic construct
YTSILHS

SEQ ID NO: 654 - protein, 9 aa, synthetic construct
QQGNTLPWT

SEQ ID NO: 655 - protein, 5 aa, synthetic construct
ESIIH

SEQ ID NO: 656 - protein, 17 aa, synthetic construct
WFYPGSDNINYNEKFKD

SEQ ID NO: 657 - protein, 10 aa, synthetic construct
HEGPYVYFDY

SEQ ID NO: 658 - protein, 11 aa, synthetic construct
KASQDVSTAVA

SEQ ID NO: 659 - protein, 8 aa, synthetic construct
YWTSTRHT

SEQ ID NO: 660 - protein, 9 aa, synthetic construct
QHHYSTPYT

SEQ ID NO: 661 - protein, 5 aa, synthetic construct
SYWMQ

SEQ ID NO: 662 - protein, 17 aa, synthetic construct
AIYPGDGDSRYIQKFKG

SEQ ID NO: 663 - protein, 9 aa, synthetic construct
DGNYYAMDY

SEQ ID NO: 664 - protein, 12 aa, synthetic construct
TASSSVSSTYLH

SEQ ID NO: 665 - protein, 7 aa, synthetic construct
STSNLAS

SEQ ID NO: 666 - protein, 9 aa, synthetic construct
HQYHRSPPT

SEQ ID NO: 667 - protein, 5 aa, synthetic construct
DTYMH

SEQ ID NO: 668 - protein, 17 aa, synthetic construct
RIDPANGYTEYDPKFQD

SEQ ID NO: 669 - protein, 5 aa, synthetic construct
TQLYY

SEQ ID NO: 670 - protein, 12 aa, synthetic construct
TASSSVSSTYLH

SEQ ID NO: 671 - protein, 7 aa, synthetic construct
STSNLAS

SEQ ID NO: 672 - protein, 9 aa, synthetic construct
HQYHRSPLT

SEQ ID NO: 673 - protein, 5 aa, synthetic construct
VYGVN

SEQ ID NO: 674 - protein, 16 aa, synthetic construct
MIWGDGSTAYNSALKS

SEQ ID NO: 675 - protein, 9 aa, synthetic construct
DGRRYALDY

SEQ ID NO: 676 - protein, 15 aa, synthetic construct
RASESVDSYGDSFLH

SEQ ID NO: 677 - protein, 7 aa, synthetic construct
RASNLDS

SEQ ID NO: 678 - protein, 9 aa, synthetic construct
QQSNEDPYT

SEQ ID NO: 679 - protein, 117 aa, synthetic construct
EVQLQQFGAELVKPGASVKISCKASGYTFTDYNIDWVKQSHGKSLEWIGDINPNYDSTSYSQKFRGKATLTVDKSSSTAYMELRSLTSEDTAVYYCARGNSWYFDVWGAGTTVTVSS

SEQ ID NO: 680 - protein, 106 aa, synthetic construct
QIVLTQSPALMSASPGEKVTMTCSASSSVRYMYWYQQKPRSSPKPWIYLTSNLASGVPARFSGSGSGTSYSLTISSMEAEDAATYYCQQWSSNPLTFGAGTKLELK

SEQ ID NO: 681 - protein, 117 aa, synthetic construct
EVQLQQFGAELVKPGASVKISCKASGYTFTDYNLDWVKQSHGKSLEWIGDINPNYDSTSYSQKFRGKATLTVDKSSSTAYMELRSLTSEDTAVYYCARGNSWYFDVWGAGTTVTVSS

SEQ ID NO: 682 - protein, 106 aa, synthetic construct; identical to SEQ ID NO: 680

SEQ ID NO: 683 - protein, 119 aa, synthetic construct
EVQLQQSGPEVGKPGASVKISCKASGYTFTDYIMHWVKQSPGQGLEWIGWVDPEYGSTDYAEKFKKRATLTADTSTNTAYIELSSLTSEDTATYFCARDDGSYSPFDYWGQGVMVTVSS

SEQ ID NO: 684 - protein, 107 aa, synthetic construct
NIVMTQSPSSLSASVGDRVTITCKASENVVTYVSWYQQKPEKAPKLLIYGASNRYTGVPDRFTGSGSATDFTLTISSLQAEDFADYHCGQGYSYPYTFGGGTKVEIK

SEQ ID NO: 685 - protein, 119 aa, synthetic construct
EVQLVQSGAEVKKPGASVKISCKASGYTFTDYIMHWVKQAPGQGLEWIGWVDPEYGSTDYAEKFKKRATLTADTSTNTAYIELSSLTSEDTATYFCARDDGSYSPFDYWGQGVLVTVSS

SEQ ID NO: 686 - protein, 107 aa, synthetic construct
NIVMTQSPSSLSASVGDRVTITCKASENVVTYVSWYQQKPGKAPKLLIYGASNRYTGVPDRFTGSGSATDFTLTISSLQPEDFADYHCGQGYSYPYTFGGGTKVEIK

SEQ ID NO: 687 - protein, 119 aa, synthetic construct
EVQLVQSGAEVKKPGASVKVSCKASGYTFTDYIMHWVRQAPGQGLEWIGWVDPEYGSTDYAEKFKKRATLTADTSTNTAYIELSSLTSEDTATYFCARDDGSYSPFDYWGQGTLVTVSS

SEQ ID NO: 688 - protein, 107 aa, synthetic construct
DIQMTQSPSSLSASVGDRVTITCKASENVVTYVSWYQQKPGKAPKLLIYGASNRYTGVPDRFSGSGSATDFTLTISSLQPEDFADYHCGQGYSYPYTFGGGTKVEIK

SEQ ID NO: 689 - protein, 119 aa, synthetic construct
EVQLVQSGAEVKKPGASVKVSCKASGYTFTDYIMHWVRQAPGQGLEWIGWVDPEYGSTDYAEKFKKRVTMTADTSTNTAYMELSSLTSEDTATYFCARDDGSYSPFDYWGQGTLVTVSS

SEQ ID NO: 690 - protein, 107 aa, synthetic construct
DIQMTQSPSSLSASVGDRVTITCRASENVVTYVSWYQQKPGKAPKLLIYGASNRYTGVPDRFSGSGSATDFTLTISSLQPEDFADYHCGQGYSYPYTFGGGTKVEIK

SEQ ID NO: 691 - protein, 119 aa, synthetic construct
EVQLVQSGAEVKKPGASVKVSCKASGYTFTDYIMHWVRQAPGQGLEWIGWVDPEYGSTDYAEKFKKRVTMTADTSTNTAYMELSSLRSEDTAVYYCARDDGSYSPFDYWGQGTLVTVSS

SEQ ID NO: 692 - protein, 107 aa, synthetic construct
DIQMTQSPSSLSASVGDRVTITCKASENVVTYVSWYQQKPGKAPKLLIYGASNRYTGVPDRFSGSGSATDFTLTISSLQPEDFATYHCGQGYSYPYTFGGGTKVEIK

SEQ ID NO: 693 - protein, 119 aa, synthetic construct
QVQLVQSGAEVKKPGASVKVSCKASGYTFTDYIMHWVRQAPGQGLEWMGWVDPEYGSTDYAEKFKKRVTMTRDTSTSTVYMELSSLRSEDTAVYYCARDDGSYSPFDYWGQGTLVTVSS

SEQ ID NO: 694 - protein, 107 aa, synthetic construct
DIQMTQSPSSLSASVGDRVTITCKASENVVTYVSWYQQKPGKAPKLLIYGASNRYTGVPSRFSGSGSGTDFTLTISSLQPEDFATYYCGQGYSYPYTFGGGTKVEIK

SEQ ID NO: 695 - protein, 115 aa, synthetic construct
QIQLVQSGPELKKPGETVKISCKASGYTFTTFGMSWVKQAPGKGLKWMGWINTYSGVPTYADDFKGRFAFSLETSASTAYLQINNLKNEDTATYFCARRSNFAYWGQGTLVTVSA

SEQ ID NO: 696 - protein, 111 aa, synthetic construct
DIVLTQSPASLAVSLGQRATISCRASESVDSSGNSFMHWYQQKAGQSFKLLIYRASNLESGIPARFSGSGSRTDFTLTINPVEADDVATYYCQQSNEDPWTFGGGTKLEIK

SEQ ID NO: 697 - protein, 115 aa, synthetic construct
QVQLVQSGSELKKPGASVKVSCKASGYTFTTFGMSWVRQAPGQGLEWMGWINTYSGVPTYAQGFTGRFVFSLDTSVSTAYLQISSLKAEDTAVYFCARRSNFAYWGQGTLVTVSS

SEQ ID NO: 698 - protein, 111 aa, synthetic construct
DIVLTQSPDSLAVSLGERATINCRASESVDSSGNSFMHWYQQKPGQPPKLLIYRASNLESGVPDRFSGSGSRTDFTLTISSLQAEDVAVYYCQQSNEDPWTFGGGTKVEIK

SEQ ID NO: 699 - protein, 444 aa, synthetic construct
QIQLVQSGPELKKPGETVKISCKASGYTFTTFGMSWVKQAPGKGLKWMGWINTYSGVPTYADDFKGRFAFSLETSASTAYLQINNLKNEDTATYFCARRSNFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 700 - protein, 218 aa, synthetic construct
DIVLTQSPASLAVSLGQRATISCRASESVDSSGNSFMHWYQQKAGQSFKLLIYRASNLESGIPARFSGSGSRTDFTLTINPVEADDVATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 701 - protein, 444 aa, synthetic construct
QVQLVQSGSELKKPGASVKVSCKASGYTFTTFGMSWVRQAPGQGLEWMGWINTYSGVPTYAQGFTGRFVFSLDTSVSTAYLQISSLKAEDTAVYFCARRSNFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 702 - protein, 218 aa, synthetic construct
DIVLTQSPDSLAVSLGERATINCRASESLTASGNSFMHWYQQKPGQPPKLLIYRASNLESGVPDRFSGSGSRTDFTLTISSLQAEDVAVYYCQQSRHVNWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 703 - protein, 115 aa, synthetic construct; identical to SEQ ID NO: 697

SEQ ID NO: 704 - protein, 111 aa, synthetic construct
DIVLTQSPDSLAVSLGERATINCRASESLTASGNSFMHWYQQKPGQPPKLLIYRASNLESGVPDRFSGSGSRTDFTLTISSLQAEDVAVYYCQQSRHVNWTFGGGTKVEIK

SEQ ID NO: 705 - protein, 444 aa, synthetic construct
QVQLVQSGSELKKPGASVKVSCKASGYTFTTFGMSWVRQAPGQGLEWMGWINTYSGVPHYAQGFTGRFVFSLDTSVSTAYLQISSLKAEDTAVYFCARRSNFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 706 - protein, 218 aa, synthetic construct
DIVLTQSPDSLAVSLGERATINCRASQTVDSSGNSFMHWYQQKPGQPPKLLIYLGNRLESGVPDRFSGSGSRTDFTLTISSLQAEDVAVYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 707 - protein, 115 aa, synthetic construct
QVQLVQSGSELKKPGASVKVSCKASGYTFTTFGMSWVRQAPGQGLEWMGWINTYSGVPHYAQGFTGRFVFSLDTSVSTAYLQISSLKAEDTAVYFCARRSNFAYWGQGTLVTVSS

SEQ ID NO: 708 - protein, 111 aa, synthetic construct
DIVLTQSPDSLAVSLGERATINCRASQTVDSSGNSFMHWYQQKPGQPPKLLIYLGNRLESGVPDRFSGSGSRTDFTLTISSLQAEDVAVYYCQQSNEDPWTFGGGTKVEIK

SEQ ID NO: 709 - protein, 444 aa, synthetic construct; identical to SEQ ID NO: 705

SEQ ID NO: 710 - protein, 218 aa, synthetic construct; identical to SEQ ID NO: 702

SEQ ID NO: 711 - protein, 115 aa, synthetic construct; identical to SEQ ID NO: 707

SEQ ID NO: 712 - protein, 111 aa, synthetic construct; identical to SEQ ID NO: 704

SEQ ID NO: 713 - protein, 119 aa, synthetic construct
QVQLLESGGGLVQPGGSLRLSCAASGFTFSTYAMSWVRQAPGKGLEWVSIISDGGDATFYADSVKGRFTISRDNSKNTLYLQMNSLRAEDTAVYYCARDGTSAAAFDSWGQGTLVTVSS

SEQ ID NO: 714 - protein, 107 aa, synthetic construct
DIQMTQSPSSLSASVGDRVTITCQASQDITNFLNWYQQKPGKAPKLLIYDASTLQTGVPSRFSGSGSGTDFTFTISSLQPEDIATYYCQQSDSYPITFGQGTKVEIK

SEQ ID NO: 715 - protein, 119 aa, synthetic construct
QVQLLESGGGLVQPGGSLRLSCAASGFTFSTYAMSWIRQAPGKGLEWVSIISDGGDATVYADSVKGRFTISRDNSKNTLYLQMNSLRAEDTAVYYCARDGTSAAAFDSWGQGTLVTVSS

SEQ ID NO: 716 - protein, 107 aa, synthetic construct; identical to SEQ ID NO: 714

SEQ ID NO: 717 - protein, 119 aa, synthetic construct
QVQLLESGGGLVQPGGSLRLSCAASGFTFSTYAMSWIRQAPGKGLEWVSIISDGGDATVYADSVKGRFTISRDNNRNTLYLQMNSLRAEDTAVYYCARDGTSAAAFDSWGQGTLVTVSS

SEQ ID NO: 718 - protein, 107 aa, synthetic construct; identical to SEQ ID NO: 714

SEQ ID NO: 719 - protein, 119 aa, synthetic construct; identical to SEQ ID NO: 713

SEQ ID NO: 720 - protein, 107 aa, synthetic construct
DIQMTQSPSSLSASVGDRVTITCQASQDITNFLNWYQQKPGKAPKLLIYDASRRRTGVPSRFSGSGSGTDFTFTISSLQPEDIATYYCQQSDSYPITFGQGTKVEIK

SEQ ID NO: 721 - protein, 119 aa, synthetic construct; identical to SEQ ID NO: 717

SEQ ID NO: 722 - protein, 107 aa, synthetic construct
DIQMTQSPSSLSASVGDRVTITCQASQDITNFLNWYQQKPGKAPKLLIYDASRRRAGVPSRFSGSGSGTDFTFTISSLQPEDIATYYCQQSDSYPITFGQGTKVEIK

SEQ ID NO: 723 - protein, 119 aa, synthetic construct; identical to SEQ ID NO: 717

SEQ ID NO: 724 - protein, 107 aa, synthetic construct
DIQMTQSPSSLSASVGDRVTITCHASQDITNFLNWYQQKPGKAPKLLIYDASTLQTGVPSRFSGSGSGTDFTFTISSLQPEDIATYYCQQSDSYPITFGQGTKVEIK

SEQ ID NO: 725 - protein, 119 aa, synthetic construct
QVQLLESGGGLVQPGGSLRLSYAASGFTFSTYAMSWIRQAPGKGLEWVSIISDGGDATVYADSVKGRFTISRDNSKNTLYLQMNSLRAEDTAVYYCARDRTSAAAFDSWGQGTLVTVSS

SEQ ID NO: 726 - protein, 107 aa, synthetic construct; identical to SEQ ID NO: 714

SEQ ID NO: 727 - protein, 119 aa, synthetic construct
QVQLLESGGGLVQPGGSLRLSCAVSGFTFSTYAMSWIRQAPGKGLEWVSIISDGGDATVYADSVKGRFTISRDNSKNTLYLQMNSLRAEDTAVYYWARDGTSAAAFDSWGQGTLVTVSS

SEQ ID NO: 728 - protein, 107 aa, synthetic construct; identical to SEQ ID NO: 714

SEQ ID NO: 729 - protein, 119 aa, synthetic construct
QVQLLESGGGLVQPGDSLRLSCAASGFTFSTYAMSWIRQAPGKGLEWVSIISDGGDATVYADSVKGRFTISRDNNRNTLYLQMNSLRAEDTAVYYCARDGTSAAAFDSWGQGTLVTVSS

SEQ ID NO: 730 - protein, 107 aa, synthetic construct
DIQMTQSPSSLSASVGDRVTITCQASQDITNFLNWYQQKPGKAPKLLIYDASTLQTGVPSRFSGSGSGTNFTFTISSLQPEDIATYYCQQSDSYPITFGQGTKVEIK

SEQ ID NO: 731 - protein, 119 aa, synthetic construct
QVQLLESVGGLVQPGGSLRLSCAVSGFTFSTYAMSWIRQAPGKGLEWVSIISDGGDVTVYADSVKGRFTISRDNSKNTLYLQMNSLRAEDTAVYYCARDRTSAAAFDSWGQGTLVSDSS

SEQ ID NO: 732 - protein, 107 aa, synthetic construct
DIQMTQSPSSLSASVGDRVTITCQASQDITNFLNWYQQKPGKAPKLLIYDASTLQTGVPSRFSGSGSGTDFNFTISSLQPEDIATYYCQQSDSYPITFGQGTKVEIK

SEQ ID NO: 733 - protein, 119 aa, synthetic construct
QVQLLESGGGLVQPGGSLRLSYAASGFTFSTYAMSWIRQAPRKGLEWVSIISDGGDATVYADSVKGRFTISRDNSKNTLYLQMNSLRAEDTAVYYCARDRTSAAAFDSWGQGTLVTVSS

SEQ ID NO: 734 - protein, 107 aa, synthetic construct; identical to SEQ ID NO: 714

SEQ ID NO: 735 - protein, 119 aa, synthetic construct; identical to SEQ ID NO: 727

SEQ ID NO: 736 - protein, 107 aa, synthetic construct; identical to SEQ ID NO: 714

SEQ ID NO: 737 - protein, 119 aa, synthetic construct
QVQLLESGGGLVQPGGSLRLSCAASGFSFTSYAMTWVRQAPGKGLEWVSGISGGGAATFYADSVKGRFTISRDNSKNTLYLQMNSLRAEDTAVYYCARQSQYVGGFDYWGQGTLVTVSS

SEQ ID NO: 738 - protein, 111 aa, synthetic construct
QSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVL

SEQ ID NO: 739 - protein, 119 aa, synthetic construct; identical to SEQ ID NO: 737

SEQ ID NO: 740 - protein, 111 aa, synthetic construct
QSVLTQPPSVSGAPGQRVTISCTGSSSNIGAGYGVHWYQQLPGTAPKLLIHGNTNRPSGVPDRFSGFKSGTSASLAITGLQAEDEADYYCQSYDSSLSGWVFGGGTKLTVL

SEQ ID NO: 741 - protein, 119 aa, synthetic construct; identical to SEQ ID NO: 737

SEQ ID NO: 742 - protein, 111 aa, synthetic construct
QSVLTQPPSVSGAPGQRVTISCTGSSSNFGAGYGVHWYQQLPGTAPKLLIHGNTNRPSGVPDRFSGFKSGTSASLAITGLQAEDEADYYCQSYDSSLSGWVFGGGTKLTVL

SEQ ID NO: 743 - protein, 119 aa, synthetic construct
QVQLLESGGGLVQPGGSLRLSCAASGFSFTSYAMTWVRQAPGKGLEWVSGISGDGAATFYADSVKGRFTISRDNSKNTLYLQMNSLRAEDTAVYYCARQSQYVGGFDYWGQGTLVTVSS

SEQ ID NO: 744 - protein, 111 aa, synthetic construct; identical to SEQ ID NO: 742

SEQ ID NO: 745 - protein, 119 aa, synthetic construct
QVQLLESGGGLVQPGGSLRLSCAASGFSFSSYAMSWVRQAPGKGLEWVSGISAGGGETFYADSVKGRFTISRDNSKNTLYLQMNSLRAEDTAVYYCARVHPISYGFDIWGQGTLVTVSS

SEQ ID NO: 746 - protein, 107 aa, synthetic construct
DIQMTQSPSSLSASVGDRVTITCQASQDIKKYLNWYQQKPGKAPKLLIYDASTLQTGVPSRFSGSGSGTDFTFTISSLQPEDIATYYCQQSDNTPVTFGQGTKVEIK

SEQ ID NO: 747 - protein, 115 aa, synthetic construct
QVQLVESGGGVVQPGRSLRLSCAASGFTFSSYAIHWVRQAPGKGLEWVAVISSDGGYKNYADSVKGRFTISRDNSKNTLYLQMDSLRAEDTAVYYCAKDRQIGDLGQGTLVTVSS

SEQ ID NO: 748 - protein, 106 aa, synthetic construct
DVVMTQSPSFLSASVGDRVTITCRASHGISNYLAWYQQKPGKAPKLLIYAASTLQSGVPSRFSGSGSGTEFTLTISSLQPDDFATYYCQQYNTYRTFGQGTKVEIK

SEQ ID NO: 749 - protein, 119 aa, synthetic construct
QVQLLESGGGLVQPGGSLRLSCAASGFTFNTYAMTWVRQAPGKGLEWVSAISDSGGDTFYADSVKGRFTISRDNSKNTLYLQMNSLRAEDTAVYYCARDGTYAARFDYWGQGTLVTVSS

SEQ ID NO: 750 - protein, 107 aa, synthetic construct
DIQMTQSPSSLSASVGDRVTITCQASQDISNYLNWYQQKPGKAPKLLIYDASTLETGVPSRFSGSGSGTDFTFTISSLQPEDIATYYCQQSDSFPITFGQGTKVEIK

SEQ ID NO: 751 - protein, 120 aa, synthetic construct
QVQLVQSGAEVKKPGSSVKVSCKASGDSFNNFGISWVRQAPGQGLEWMGGIVPVLGIATYAQKFQGRVTITADESTSTAYMELSSLRSEDTAVYYCARGSAWYDGSFQYWGQGTLVTVSS

SEQ ID NO: 752 - protein, 108 aa, synthetic construct
EIVLTQSPGTLSLSPGERATLSCRASQSVSSTFLAWYQQKPGQAPRLLIYDASTRATGIPDRFSGSDSGTDFTLTISRLEPEDFAVYYCQQYDSWPFTFGQGTKVEIK

SEQ ID NO: 753 - protein, 123 aa, synthetic construct
QVQLVQSGAEVKKPGSSVKVSCKASGDTFSSYGVSWVRQAPGQGLEWMGRIVPVFGIANYAQKFQGRVTITADESTSTAYMELSSLRSEDTAVYYCARQSPYVTYSSYYFDYWGQGTLVTVSS

SEQ ID NO: 754 - protein, 108 aa, synthetic construct
EIVLTQSPGTLSLSPGERATLSCRASQSVSNNFLAWYQQKPGQAPRLLIYDASTRATGIPDRFSGSGSGTDFTLTISRLEPEDFAVYYCQQYGSWPITFGQGTKVEIK

SEQ ID NO: 755 - protein, 123 aa, synthetic construct
QVQLVQSGAEVKKPGSSVKVSCKASGDTFSNYGFSWVRQAPGQGLEWMGGIVPVFGIATYAQKFQGRVTITADESTSTAYMELSSLRSEDTAVYYCARDYSYYPDDPRYFEYWGQGTLVTVSS

SEQ ID NO: 756 - protein, 108 aa, synthetic construct
EIVLTQSPGTLSLSPGERATLSCRASQSVSSTFLAWYQQKPGQAPRLLIYAASSRATGIPDRFSGSGSGTDFTLTISRLEPEDFAVYYCQQYGSWPLTFGQGTKVEIK

SEQ ID NO: 757 - protein, 123 aa, synthetic construct
EVQLVESGGGLVQPGGSLRLSCAASGIDLSSYTMGWVRQAPGKGLEWVGFISSSGHTYYANWAKGRFTISRDNSKNTLYLQMNSLRAEDTAVYYCAREGGYGGYDYTGIFNLWGQGTLVTVSS

SEQ ID NO: 758 - protein, 110 aa, synthetic construct
DIQMTQSPSTLSASVGDRVTITCQATESISSWLAWYQQKPGKAPKLLIYGASTLESGVPSRFSGSGSGTEFTLTISSLQPDDFATYYCQQGYIYTNVDNTFGGGTKVEIK

SEQ ID NO: 759 - protein, 123 aa, synthetic construct
EVQLVESGGGLVQPGGSLRLSCAASGIDLSSYTMGWVRQAPGKGLEWVGFISSSGHTYYANWAKGRFTISRDNSKNTLYLQMNSLRAEDTAVYYCARDGGYGGYDYTGIFNLWGQGTLVTVSS

SEQ ID NO: 760 - protein, 110 aa, synthetic construct; identical to SEQ ID NO: 758

SEQ ID NO: 761 - protein, 120 aa, synthetic construct
EVQLVESGGGLVQPGGSLRLSCAASGFSLNSYGVNWVRQAPGKGLEWVGGINTGGSTYYANWAKGRFTISRDNSKNTLYLQMNSLRAEDTAVYYCARTSGNNVYNYFTLWGQGTLVTVSS

SEQ ID NO: 762 - protein, 111 aa, synthetic construct
DIQMTQSPSSLSASVGDRVTITCQASQSIPSLLAWYQQKPGKAPKLLIYAPSTLASGVPSRFSGSGSGTDFTLTISSLQPEDFATYYCQSYYYGDNTYNNIFGGGTKVEIK

SEQ ID NO: 763 - protein, 124 aa, synthetic construct
EVQLVESGGGLVQPGGSLRLSCAASGFSLSTYDINWVRQAPGKGLEWVGIIYTGGITNFANWAKGRFTISRDNSKNTLYLQMNSLRAEDTAVYYCARGGYDSEGYVYPDAFDPWGQGTLVTVSS

SEQ ID NO: 764 - protein, 110 aa, synthetic construct
DIQMTQSPSSLSASVGDRVTITCQASESISNLLAWYQQKPGKAPKLLIYRASILTSGVPSRFSGSGSGTDFTLTISSLQPEDFATYYCQHGYTGTNVQNVFGGGTKVEIK

SEQ ID NO: 765 - protein, 120 aa, synthetic construct
EVQLVESGGGLVQPGGSLRLSCAASGIDLSNYAMGWVRQAPGKGLEWVGSRRTDGITYYANWAEGRFTISRDNSKNTLYLQMNSLRAEDTAVYYCGRDVGGEGGWYFNLWGQGTLVTVSS

SEQ ID NO: 766 - protein, 106 aa, synthetic construct
DIQMTQSPSTLSASVGDRVTITCQASQSINIYLAWYQQKPGKAPKLLIYDASKLASGVPSRFSGSGSGTEFTLTISSLQPDDFATYYCQQGINNIGFGGGTKVEIK

SEQ ID NO: 767 - protein, 120 aa, synthetic construct
EVQLVESGGGLVQPGGSLRLSCAASGIDLSNYAMGWVRQAPGKGLEWVGSRRTDGITYYANWAEGRFTISRDNSKNTLYLQMNSLRAEDTAVYYCGRDVGGDGGWYFNLWGQGTLVTVSS

SEQ ID NO: 768 - protein, 106 aa, synthetic construct; identical to SEQ ID NO: 766

SEQ ID NO: 769 - protein, 120 aa, synthetic construct
EVQLVESGGGLVQPGGSLRLSCAASGIDLSSYAMGWVRQAPGKGLEWVGDISTSGNAYYATWVKGRFTISRDNSKNTLYLQMNSLRAEDTAVYYCARADYGGETYAFDPWGQGTLVTVSS

SEQ ID NO: 770 - protein, 110 aa, synthetic construct
DIQMTQSPSSLSASVGDRVTITCQASQSISSYLNWYQQKPGKAPKLLIYSASTLASGVPSRFSGSGSGTDFTLTISSLQPEDFATYYCQQGYSDSNIDNVFGGGTKVEIK

SEQ ID NO: 771 - protein, 122 aa, synthetic construct
EVQLVESGGGLVQPGGSLRLSCAASGFSLSSHHMIWVRQAPGKGLEWVGIIDAGSGSTYYASWAKGRFTISRDNSKNTLYLQMNSLRAEDTAVYYCARGGLTESLGTYFDLWGQGTLVTVSS

SEQ ID NO: 772 - protein, 113 aa, synthetic construct
DIQMTQSPSSLSASVGDRVTITCQASESIDSGLAWYQQKPGKAPKLLIYDSSTLASGVPSRFSGSGSGTDFTLTISSLQPEDFATYYCQSNYDTGSSVYDWGSFGGGTKVEIK

SEQ ID NO: 773 - protein, 123 aa, synthetic construct
EVQLVESGGGLVQPGGSLRLSCAASGFSLSDYFMTWVRQAPGKGLEWVGIINTGGDSYYATWAKGRFTISRDNSKNTLYLQMNSLRAEDTAVYYCARDTGYGGYDYAGSFDPWGQGTLVTVSS

SEQ ID NO: 774 - protein, 110 aa, synthetic construct
DIQMTQSPSSVSASVGDRVTITCQASENINSWLAWYQQKPGKAPKLLIYEASKLASGVPSRFSGSGSGTDFTLTISSLQPEDFATYYCQQGYIYIDVGNIFGGGTKVEIK

SEQ ID NO: 775 - protein, 118 aa, synthetic construct
EVQLVESGGGLVQPGGSLRLSCAASGFSFSVSYWICWVRQAPGKGLEWVACTDGGDGSSYYASWVNGRFTISRDNSKNTLYLQMNSLRAEDTAVYYCARDRSDVFNLWGQGTLVTVSS

SEQ ID NO: 776 - protein, 112 aa, synthetic construct
DIQMTQSPSSLSASVGDRVTITCQAGQSIDSNLAWYQQKPGKAPKLLIYRASTLASGVPSRFSGSGSGTDFTLTISSLQPEDFATYYCQSFYVTISAMVDYPFGGGTKVEIK

SEQ ID NO: 777 - protein, 115 aa, synthetic construct
EVQLLESGGGLVQPGGSLRLSCAASGIDLSRYAMAWVRQAPGKGLEWVGYIDTGDSTYYATWAKGRFTISRDNSKNTLYLQMNSLRAEDTAVYYCTNVGVRMYLWGQGTLVTVSS

SEQ ID NO: 778 - protein, 110 aa, synthetic construct
DIQMTQSPSSLSASVGDRVTITCQASQSISSYLSWYQQKPGKAPKLLIYRASTLESGVPSRFSGSGSGTDFTLTISSLQPEDFATYYCQCGYYGGSYIGAFGGGTKVEIK

SEQ ID NO: 779 - protein, 125 aa, synthetic construct
EVQLVESGGGLVQPGGSLRLSCAASGIDFSSYGISWVRQAPGKGLEWVAYIYPDYGSTDYATWVNGRFTISLDNSKNTLYLQMNSLRAEDTAVYYCASGYASSSGYYDPKYFGLWGQGTLVTVSS

SEQ ID NO: 780 - protein, 110 aa, synthetic construct
DIQMTQSPSSLSASVGDRVTITCRASEDIESYLAWYQQKPGKAPKLLIYDASDLASGVPSRFSGSGSGTDFTLTISSLQPEDFATYYCQHGFYTSRSDSVFGGGTKVEIK

SEQ ID NO: 781 - protein, 117 aa, synthetic construct
EVQLVESGGGLVQPGGSLRLSCAASGFSLSSYDMSWVRQAPGKGLEWVGYIWSSGSAYYATWAEGRFTISRDNSKNTLYLQMNSLRAEDTAVYYCARRYVGSSYDTWGQGTLVTVSS

SEQ ID NO: 782 - protein, 111 aa, synthetic construct
DIQMTQSPSSLSASVGDRVTITCQSSQSVSSNNYLSWYQQKPGKAPKLLIYAASYLASGVPSRFSGSGSGTDFTLTISSLQPEDFATYYCLGDYDNDIDHAFGGGTKVEIK

SEQ ID NO: 783 - protein, 115 aa, synthetic construct
EVQLVESGGGLVQPGGSLRLSCAASGIDLSSYAMGWVRQAPGKGLEWVGFIDTGGSTYYANWAKGRFTISRDNSKNTLYLQMNSLRAEDTAVYYCANVGARMYLWGQGTLVTVSS

SEQ ID NO: 784 - protein, 110 aa, synthetic construct
DIQMTQSPSSLSASVGDRVTITCQASQSISNLLAWYQQKPGKAPKLLIYRASTLESGVPSRFSGSGSGTDFTLTISSLQPEDFATYYCQCSYYGGSYIGAFGGGTKVEIK

SEQ ID NO: 785 - protein, 124 aa, synthetic construct
EVQLVESGGGLVQPGGSLRLSCAASGFDFSRYYMSWVRQAPGKGLEWVGYIDPIFGNTYYASWVNGRFTISSDNAKNSLYLQMNSLRAEDTAVYYCARDGDAGYDGYGYGTDLWGQGTLVTVSS

SEQ ID NO: 786 - protein, 111 aa, synthetic construct
DIQMTQSPSSLSASVGDRVTITCQASENIYSGLAWYQQKPGKVPKLLIVSAFTLASGVPSRFSGSGSGTDFTLTISSLQPEDVATYYCQTYYYGSVTYFNAFGGGTKVEIK

SEQ ID NO: 787 - protein, 120 aa, synthetic construct
EVQLVESGGGLVQPGGSLRLSCAASGFSLNSHYMIWVRQAPGKGLEWVGIITSSDYIYYARWAKGRFTISRDNSKNTLYLQMNSLRAEDTAVYYCARYNYDDDGELFNLWGQGTLVTVSS

SEQ ID NO: 788 - protein, 111 aa, synthetic construct
DIQMTQSPSSLSASVGDRVTITCQSSQSIDANNDLAWYQQKPGKAPKLLIYLASKLASGVPSRFSGSGSGTDFTLTISSLQPEDFATYYCLGGYDDDADNTFGGGTKVEIK

SEQ ID NO: 789 - protein, 123 aa, synthetic construct
EVQLVESGGGLVQPGGSLRLSCAASGFSFTNNYYMCWVRQAPGKGLEWVGCIYPSIVGPTYYANWAKGRFTISRDNSKNTLYLQMNSLRAEDTAVYYCVRDRYDDYGDYFNLWGQGTLVTVSS

SEQ ID NO: 790 - protein, 112 aa, synthetic construct
DIQMTQSPSSLSASVGDRVTITCQASQSIYNYLSWYQQKPGKAPKRLIYYASTLASGVPSRFSGSGSGTDFTLTISSLQPEDFATYYCQSNSGVNGNRYGNAFGGGTKVEIK

SEQ ID NO: 791 - protein, 128 aa, synthetic construct
EVQLVQSGAEVKKPGSSVKVSCKASGDTFTSYSFNWVRQAPGQGLEWMGRIITILDDTEYAPHLQGRVTITADKSTSTVYLELRNLRSDDTAVYFCAGVYEGPNSEGSYDDYGFLKHWGQGTLVTVSS

SEQ ID NO: 792 - protein, 109 aa, synthetic construct
DIVMTQSPATLSVSPGERATLSCRASESVANDLAWYQQKPGQVPRLLIYGASTRATGVPARFSGSGSGAEFTLTISSLQSEDFAVYYCQQYANWPPRETFGQGTRLEIK

SEQ ID NO: 793 - protein, 128 aa, synthetic construct; identical to SEQ ID NO: 791

SEQ ID NO: 794 - protein, 109 aa, synthetic construct; identical to SEQ ID NO: 792

SEQ ID NO: 795 - protein, 128 aa, synthetic construct
EVQLVQSGAEVKKPGSSVKVSCKASGDTFTSYSWNWVRQAPGQGLEWMGRIITDLDDTEYAPHLQGRVTITADKSTSTVYLELRNLRSDDTAVYFCAGVYEGPNSEGSYDDYGFLKHWGQGTLVTVSS

SEQ ID NO: 796 - protein, 109 aa, synthetic construct
DIVMTQSPATLSVSPGERATLSCRASESGANDLAWYQQKPGQVPRLLCYGASTRATGVPARFSGSGSGAEFTLTISSLQSEDFAVYYCQQYANWPPRGTFGQGTRLEIK

SEQ ID NO: 797 - protein, 128 aa, synthetic construct; identical to SEQ ID NO: 791

SEQ ID NO: 798 - protein, 109 aa, synthetic construct
DIVMTQSPATLSVSPGERATLSCRASESVANDLAWYQQKPGQVPRLLIYGASTRARGVPARFSGSGSGAEFTLTISSLQSEDFAVYYCQQYTNWPPRETFGQGTRLEIK

SEQ ID NO: 799 - protein, 128 aa, synthetic construct; identical to SEQ ID NO: 791

SEQ ID NO: 800 - protein, 109 aa, synthetic construct
DIVMTQSPATLSVSPGERATLSCRASESVANDLAWYQQKPGQVPRLLRYGASTRATGVPARFSGSGSGAEFTLTISSLQSEDFAVYYCQQYAIWPPRETFGQGTRLEIK

SEQ ID NO: 801 - protein, 128 aa, synthetic construct
EVQLVQSGAEVKKPGSSVKVSCKASGDTFTSYSYNWVRQAPGQGLEWMGRIITILDDTEYAPHLQGRVTITADKSTSTVYLELRNLRSDDTAVYFCAGVYEGPNSEGSYDDYGFLKHWGQGTLVTVSS

SEQ ID NO: 802 - protein, 109 aa, synthetic construct
DIVMTQSPATLSVSPGERATLSCRASRSVANDLAWYQQKPGQVPRLLIYGASTRATGVPARFSGSGSGAEFTLTISSLQSEDFAVYYCQQYAIWPPRETFGQGTRLEIK

SEQ ID NO: 803 - protein, 128 aa, synthetic construct; identical to SEQ ID NO: 791

SEQ ID NO: 804 - protein, 109 aa, synthetic construct
DIVMTQSPATLSVSPGERATLSCRASESVANNLAWYQQKPGQVPRLLIYGASTRATGVPARFSGSGSGAEFTLTISSLQSEDFAVYYCQQYARWPPTETFGQGTRLEIK

SEQ ID NO: 805 - protein, 128 aa, synthetic construct
EVQLVQSGAEVKKPGSSVKVSCKASGDTFTSYSFNWVRQAPGQGLEWMGRIITIPDDTEYAPHLQGRVTITADKSTSTVYLELRNLRSDDTAVYFCAGVYEGPNSEGSYDDYGFLKHWGQGTLVTVSS

SEQ ID NO: 806 - protein, 109 aa, synthetic construct
DIVMTQSPATLSVSPGERATLSCRASESVATDLAWYQQKPGQVPRLLIYGASTRATGVPARFSGSGSGAEFTLTISSLQSEDFAVYYCQQYDNWPPRETFGQGTRLEIK

SEQ ID NO: 807 - protein, 128 aa, synthetic construct; identical to SEQ ID NO: 791

SEQ ID NO: 808 - protein, 109 aa, synthetic construct
DIVMTQSPATLSVSPGERATLSCRASESVRNDLAWYQQKPGQVPRLLIYGASTRATGVPARFSGSGSGAEFTLTISSLQSEDFAVYYCQQYANWPPHETFGQGTRLEIK

SEQ ID NO: 809 - protein, 128 aa, synthetic construct
EVQLVQSGAEVKKPGSSVKVSCKASGDTFTSYSFNWVRQAPGQGLEWMGRIITIDDDTEYAPHLQGRVTITADKSTSTVYLELRNLRSDDTAVYFCAGVYEGPNSEGSYDDYGFYKHWGQGTLVTVSS

SEQ ID NO: 810 - protein, 109 aa, synthetic construct
DIVMTQSPATLSVSPGERATLSCRASESVGNDLAWYQQKPGQVPRLLIYGASTRAPGVPARFSGSGSGAEFTLTISSLQSEDFAVYYCQQYANWPPRETFGQGTRLEIK

SEQ ID NO: 811 - protein, 128 aa, synthetic construct
EVQLVQSGAEVKKPGSSVKVSCKASGDTFTSYSFNWVRQAPGQGLEWMGRIITILDDSEYAPHLQGRVTITADKSTSTVYLELRNLRSDDTAVYFCAGVYEGPNSEGSYDDYGFLKHWGQGTLVTVSS

SEQ ID NO: 812 - protein, 109 aa, synthetic construct
DIVMTQSPATLSVSPGERATLSCRASESVANDLAWYQQKPGQVPRLLIYDASTRATGVPARFSGSGSGAEFTLTISSLQSEDFAVYYCQQYANWPWRETFGQGTRLEIK

SEQ ID NO: 813 - protein, 128 aa, synthetic construct
EVQLVQSGAEVKKPGSSVKVSCKASGDTFTSYSYNWVRQAPGQGLEWMGRIITIDDDTEYAPHLQGRVTITADKSTSTVYLELRNLRSDDTAVYFCAGVYEGPNSEGSYDDYGFYKHWGQGTLVTVSS

SEQ ID NO: 814 - protein, 109 aa, synthetic construct
DIVMTQSPATLSVSPGERATLSCRASESVANDLAWYQQKPGQVPRLLIYGASTRATGVPARFSGSGSGAEFTLTISSLQSEDFAVYYCQQGANWPPRETFGQGTRLEIK

SEQ ID NO: 815 - protein, 128 aa, synthetic construct
EVQLVQSGAEVKKPGSSVKVSCKASGDTFTSYSFNWVRQAPGQGLEWMGRIITIYDDTEYAPHLQGRVTITADKSTSTVYLELRNLRSDDTAVYFCAGVYEGPNSEGSYDDYGFFKHWGQGTLVTVSS

SEQ ID NO: 816 - protein, 109 aa, synthetic construct; identical to SEQ ID NO: 792

SEQ ID NO: 817 - protein, 128 aa, synthetic construct
EVQLVQSGAEVKKPGSSVKVSCKASGDTFTSYSFNWVRQAPGQGLEWMGRIITILDDTEYAPHLQGRVTITADKSTSTVYLELRNLRSDDTAVYFCAGVYEGPNSEGSYDDYGFLKYWGQGTLVTVSS

SEQ ID NO: 818 - protein, 109 aa, synthetic construct
DIVMTQSPATLSVSPGERATLSCRASESVAVDLAWYQQKPGQVPRLLIYGASTRATGVPARFSGSGSGAEFTLTISSLQSEDFAVYYCQQYANKPPRETFGQGTRLEIK

SEQ ID NO: 819 - protein, 128 aa, synthetic construct
EVQLVQSGAEVKKPGSSVKVSCKASGDTFTSYSFNWVRQAPGQGLEWMGRIITILDDTEYAPHLQGRVTITADKSTSTVYLELRNLRSDDTAVYFCAGVYEGPNSEGSYDDYGALKHWGQGTLVTVSS

SEQ ID NO: 820 - protein, 109 aa, synthetic construct; identical to SEQ ID NO: 792

SEQ ID NO: 821 - protein, 128 aa, synthetic construct
EVQLVQSGAEVKKPGSSVKVSCKASGDTFTSYSFNWVRQAPGQGLEWMGRIITDLDDTEYAPHLQGRVTITADKSTSTVYLELRNLRSDDTAVYFCAGVYEGPNSEGSYDDYGQLKHWGQGTLVTVSS

SEQ ID NO: 822 - protein, 109 aa, synthetic construct; identical to SEQ ID NO: 792

SEQ ID NO: 823 - protein, 128 aa, synthetic construct
EVQLVQSGAEVKKPGSSVKVSCKASGDTFTSYSFNWVRQAPGQGLEWMGRIITILDDTEYAPHLQGRVTITADKSTSTVYLELRNLRSDDTAVYFCAGVYEGPNSEGSYDDYGQLKHWGQGTLVTVSS

SEQ ID NO: 824 - protein, 109 aa, synthetic construct
DIVMTQSPATLSVSPGERATLSCRASESVANDLAWYQQKPGQVPRLLIYGASTRAPGVPARFSGSGSGAEFTLTISSLQSEDFAVYYCQQYANTPPRETFGQGTRLEIK

SEQ ID NO: 825 - protein, 128 aa, synthetic construct
EVQLVQSGAEVKKPGSSVKVSCKASGDTFTSYSFNWVRQAPGQGLEWMGRIITDLDDTEYAPHLQGRVTITADKSTSTVYLELRNLRSDDTAVYFCAGVYEGPNSEGSYDDYGFLKHWGQGTLVTVSS

SEQ ID NO: 826 - protein, 109 aa, synthetic construct
DIVMTQSPATLSVSPGERATLSCRASESVANDLTWYQQKPGQVPRLLIYGASTRATGVPARFSGSGSGAEFTLTISSLQSEDFAVYYCQQYANWPPRETFGQGTRLEIK

SEQ ID NO: 827 - protein, 128 aa, synthetic construct; identical to SEQ ID NO: 791

SEQ ID NO: 828 - protein, 109 aa, synthetic construct
DIVMTQSPATLSVSPGERATLSCRASESVGNDLAWYQQKPGQVPRLLIYGASTRATGVPARFSGSGSGAEFTLTISSLQSEDFAVYYCQQYANWPPRETFGQGTRLEIK

SEQ ID NO: 829 - protein, 128 aa, synthetic construct
EVQLVQSGAEVKKPGSSVKVSCKASGDTFTSYSFNWVRQAPGQGLEWMGRIITIDDDTEYAPHLQGRVTITADKSTSTVYLELRNLRSDDTAVYFCAGVYEGPNSEGSYDDYGQLKHWGQGTLVTVSS

SEQ ID NO: 830 - protein, 109 aa, synthetic construct
DIVMTQSPATLSVSPGERATLSCRASESVGNDLAWYQQKPGQVPRLLIYGASTRATGVPARFSGSGSGAEFTLTISSLQSEDFAVYYCQQYANVPPRETFGQGTRLEIK

SEQ ID NO: 831 - protein, 128 aa, synthetic construct; identical to SEQ ID NO: 829

SEQ ID NO: 832 - protein, 109 aa, synthetic construct
DIVMTQSPATLSVSPGERATLSCRASESVGNDLAWYQQKPGQVPRLLIYGISTRATGVPARFSGSGSGAEFTLTISSLQSEDFAVYYCQQYANDPPRETFGQGTRLEIK

SEQ ID NO: 833 - protein, 120 aa, synthetic construct
EVQLVESGGGLVQPGGSLRLSCAASGFNIRDTYIHWVRQSPGKGLEWVADIDPDNGDTRYADSVKGRFTISADTSKNTAYLQMNSLRAEDTAIYYCSRDLADGYNAFDRWGQGTLVTVSS

SEQ ID NO: 834 - protein, 107 aa, synthetic construct
DIQMTQSPILLSASVGDRVTITCRASQDVSTAVAWSQQRTNGSPRLLIYSASSLYSGVPSRFSGSRSGTDFTLTISSLQPEDIADYYCQQPTYTPPTFGAGTKVEIK

SEQ ID NO: 835 - protein, 120 aa, synthetic construct
EVQLVESGGGLVQPGGSLRLSCAASGFNIRDTFIHWVRQSPGKGLEWVADIDPDNGDTRYADSVKGRFTISADTSKNTAYLQMNSLRAEDTAIYYCSREGEDGYNAWDLWGQGTLVTVSS

SEQ ID NO: 836 - protein, 107 aa, synthetic construct
DIQMTQSPILLSASVGDRVTITCRASQDVSTAVAWSQQRTNGSPRLLIYSASSLYSGVPSRFSGSRSGTDFTLTISSLQPEDIADYYCQQPPYTPPTFGAGTKVEIK

SEQ ID NO: 837 - protein, 117 aa, synthetic construct
QVQLVQSGAEVKKPGSSVKVSCKASGYTFTDLGIGWVRQAPGQGLEWMGWINTITGEPFYNPKFKGRVTITADKSTSTAYMELSSLRSEDTAVYYCTRDHPLGLDYWGQGTTVTVSS

SEQ ID NO: 838 - protein, 111 aa, synthetic construct
DIVMTQSPDSLAVSLGERATINCRASQSLDVNGVSYLNWYQQKPGQPPKFLIYKASNLESGVPDRFSGSGSGTDFTLTISSLQAEDVAVYYCQQSREDPYTFGGGTKVEIK

SEQ ID NO: 839 - protein, 117 aa, synthetic construct
QVQLVQSGAEVKKPGSSVKVSCKASGYTFTDLGIGWVRQAPGQGLEWMGWINTHTGEPYDNPKFKGRVTITADKSTSTAYMELSSLRSEDTAVYYCTRDHPLGLDYWGQGTTVTVSS

SEQ ID NO: 840 - protein, 111 aa, synthetic construct
DIVMTQSPDSLAVSLGERATINCRSSQSVDVNGVSYLDWYQQKPGQPPKLLITKASNRESGVPDRFSGSGSGTDFTLTISSLQAEDVAVYYCQQNREDPYTFGGGTKVEIK

SEQ ID NO: 841 - protein, 117 aa, synthetic construct; identical to SEQ ID NO: 837

SEQ ID NO: 842 - protein, 111 aa, synthetic construct
DIVMTQSPDSLAVSLGERATINCRASQSVDVNGVSYLNWYQQKPGQPPKFLIYKASNLESGVPDRFSGSGSGTDFTLTISSLQAEDVAVYYCQQSREDPYTFGGGTKVEIK

SEQ ID NO: 843 - protein, 117 aa, synthetic construct
EVQLVQSGPELKKPGASVKVSCKASGYTFTDLGMNWVRQAPGQGLEWMGWINTHTGMPFYADDFKGRFVFSLDTSVSTAYLQISSLKAEDTAVYYCARDHPLGLDYWGQGTLVTVSS

SEQ ID NO: 844 - protein, 111 aa, synthetic construct
DIQVTQSPSSLSASVGDRVTITCITSQSVDVNGVSYLNWYQQKPGKVPKFLIYKASNLRPGVPSRFSGSGSGTDFTLTISSLQPEDVATYYCQQSREDPYTFGQGTKVEIK

SEQ ID NO: 845 - protein, 117 aa, synthetic construct
EVQLVQSGPELKKPGASVKVSCKGSGYTFTDLGINWVRQAPGQGLEWMGWINTHTGEPFYADDFKGRFVFSLDTSVSTAYLQISSLKAEDTAVYYCTRDHPLGLDYWGQGTLVTVSS

SEQ ID NO: 846 - protein, 111 aa, synthetic construct
DIQVTQSPSSLSASVGDRVTITCITSQSVDVNGVSYLNWYQQKPGKVPKFLIYKASRLRSGVPSRFSGSGSGTDFTLTISSLQPEDVATYYCQQSREDPYTFGQGTKVEIK

SEQ ID NO: 847 - protein, 117 aa, synthetic construct
EVQLVQSGPELKKPGASVKVSCKASGYTFTDLGIGWVRQAPGQGLEWMGWINTHTGEPFYADDFKGRFVFSLDTSVSTAYLQISSLKAEDTAVYYCTRDHPLGLDYWGQGTLVTVSS

SEQ ID NO: 848 - protein, 111 aa, synthetic construct
DIQVTQSPSSLSASVGDRVTITCITSQSVDVNGLSYLNWYQQKPGKVTKFLIYKASNLGSGVPSRFSGSGSGTDFTLTISSLQPEDVATYYCQQSREDPYTFGQGTKVEIK

SEQ ID NO: 849 - protein, 117 aa, synthetic construct
EVQLVQSGPELKKPGASVKVSCKASGYTFTDLGMNWVRQAPGQGLEWMGWINTHTGKPFYADDFKGRFVFSLDTSVSTAYLQISSLKAEDTAVYYCTRDHPLGLDYWGQGTLVTVSS

SEQ ID NO: 850 - protein, 111 aa, synthetic construct
DIQVTQSPSSLSASVGDRVTITCITSQSVDVNGVSYLNWYQQKPGKVPKFLIYKASNLRSGVPSRFSGSGSGTDFTLTISSLQPEDVATYYCQQSREDPYTFGQGTKVEIK

SEQ ID NO: 851 - protein, 117 aa, synthetic construct
EVQLVQSGPELKKPGASVKVSCKGSGYTFTDLGMGWVRQAPGQGLEWMGWINTQTGEPFYADDFKGRFVFSLDTSVSTAYLQISSLKAEDTAVYYCTRDHPLGLDYWGQGTLVTVSS

SEQ ID NO: 852 - protein, 111 aa, synthetic construct
DIQVTQSPSSLSASVGDRVTITCITSQSVDVAGVSYLNWYQQKPGKVPKFLIHKASNLRSGVPSRFSGSGSGTDFTLTISSLQPEDVATYYCQQAREDPYTFGQGTKVEIK

SEQ ID NO: 853 - protein, 117 aa, synthetic construct
EVQLVQSGPELKKPGASVKVSCKGSGYTFTDLGMNWVRQAPGQGLEWMGWINTQTGEPFYADDFKGRFVFSLDTSVSTAYLQISSLKAEDTAVYFCARDHPLGLDYWGQGTLVTVSS

SEQ ID NO: 854 - protein, 111 aa, synthetic construct
DIQVTQSPSSLSASVGDRVTITCITSQSVDVNGVSYLNWYQQKPGKVPKFLIYKASILRSGVPSRFSGSGSGTDFTLTISSLQPEDVATYYCQQSREDPYTFGQGTKVEIK

SEQ ID NO: 855 - protein, 117 aa, synthetic construct
EVQLVQSGPELKKPGASVKVSCKGSGYTFTDLGINWVRQAPGQGLEWMGWINTHTGEPFYADDFKGRFVFSLDTSVSTAYLQISSLKAEDTAVYFCTRDHPLGLDYWGQGTLVTVSS

SEQ ID NO: 856 - protein, 111 aa, synthetic construct; identical to SEQ ID NO: 844

SEQ ID NO: 857 - protein, 117 aa, synthetic construct
EVQLVQSGPELKKPGASVKVSCKGSGYTFTDLGINWVRQAPGQGLEWMGWINTHTGEPYYADDFKGRFVFSLDTSVSTAYLQISSLKAEDTAVYYCARDHPLGLDYWGQGTLVTVSS

SEQ ID NO: 858 - protein, 111 aa, synthetic construct
DIQVTQSPSSLSASVGDRVTITCITSQSVDVNGVSYMNWYQQKPGKVPKFLIYKAVNLRPGVPSRFSGSGSGTDFTLTISSLQPEDVATYYCQQSREDPYTFGQGTKVEIK

SEQ ID NO: 859 - protein, 117 aa, synthetic construct
QVQLVQSGAEVKKPGSSVKVSCKGSGYTFTDLGIGWVRQAPGQGLEWMGWINTHTGEPFYNPKFKGRVTITADKSTSTAYMELSSLRSEDTAVYYCTRDHPLGLDYWGQGTTVTVSS

SEQ ID NO: 860 - protein, 111 aa, synthetic construct
DIVMTQSPDSLAVSLGERATINCRSSQSVDVNGVSYLNWYQQKPGQPPKFLIYKASNRESGVPDRFSGSGSGTDFTLTISSLQAEDVAVYYCQQAREDPYTFGGGTKVEIK

SEQ ID NO: 861 - protein, 128 aa, synthetic construct; identical to SEQ ID NO: 795

SEQ ID NO: 862 - protein, 109 aa, synthetic construct; identical to SEQ ID NO: 796

SEQ ID NO: 863 - protein, 120 aa, synthetic construct
EVQLVESGGGLVQPGGSLRLSCAASGFNIRATYIHWVRQSPGKGLEWVADIDPTNGDTRYADSVKGRFTISADTSKNTAYLQMNSLRAEDTAIYYCTREGDDGYNAWDKWGQGTLVTVSS

SEQ ID NO: 864 - protein, 107 aa, synthetic construct
DIQMTQSPILLSASVGDRVTITCRASQDVSTAVAWSQQRTNGSPRLLIYSASSLYSGVPSRFSGSRSGTDFTLTISSLQPEDIADYYSQQPSYTPPTFGAGTKVEIK

SEQ ID NO: 865 - protein, 120 aa, synthetic construct
EVQLVESGGGLVQPGGSLRLSCAASGFNIRAMYIHWVRQSPGKGLEWVADIDPTNGDTRYADSVKGRFTISADTSKNTAYLQMNSLRAEDTAIYYCTREGQDGYNAFDKWGQGTLVTVSS

SEQ ID NO: 866 - protein, 107 aa, synthetic construct
DIQMTQSPILLSASVGDRVTITCRASQDVSTAVAWSQQRTNGSPRLLIYSASSLYSGVPSRFSGSRSGTDFTLTISSLQPEDIADYYCQQHRYTPPTFGAGTKVEIK

SEQ ID NO: 867 - protein, 120 aa, synthetic construct
EVQLVESGGGLVQPGGSLRLSCAASGFNIRATYIHWVRQSPGKGLEWVADISPDNGDTRYADSVKGRFTISADTSKNTAYLQMNSLRAEDTAIYYCSRDLADGYNAFDLWGQGTLVTVSS

SEQ ID NO: 868 - protein, 107 aa, synthetic construct
DIQMTQSPILLSASVGDRVTITCRASQDVSTAVAWSQQRTNGSPRLLIYSASSLYSGVPSRFSGSRSGTDFTLTISSLQPEDIADYYCQQHGYTPPTFGAGTKVEIK

SEQ ID NO: 869 - protein, 120 aa, synthetic construct
EVQLVESGGGLVQPGGSLRLSCAASGFNIRDTYIHWVRQSPGKGLEWVADIDPDNGDTRYADSVKGRFTISADTSKNTAYLQMNSLRAEDTAIYYCTREGADGYNAFDLWGQGTLVTVSS

SEQ ID NO: 870 - protein, 107 aa, synthetic construct
DIQMTQSPILLSASVGDRVTITCRASQDVSTAVAWSQQRTNGSPRLLIYPASSLYSGVPSRFSGSRSGTDFTLTISSLQPEDIADYYCQQPDYTPPTFGAGTKVEIK

SEQ ID NO: 871 - protein, 120 aa, synthetic construct
EVQLVESGGGLVQPGGSLRLSCAASGFNIRAFYIHWVRQSPGKGLEWVADIDPTNGDTRYADSVKGRFTISADTSKNTAYLQMNSLRAEDTAIYYCTREGEDGYNAFDQWGQGTLVTVSS

SEQ ID NO: 872 - protein, 107 aa, synthetic construct
DIQMTQSPILLSASVGDRVTITCRASQDVSTAVAWYQQRTNGSPRLLIYPASSLYSGVPSRFSGSRSGTDFTLTISSLQPEDIADYYCQQPRYTPPTFGAGTKVEIK

SEQ ID NO: 873 - protein, 120 aa, synthetic construct
EVQLVESGGGLVQPGGSLRLSCAASGFNIKDFFIHWVRQAPGKGLEWVADIDPTNGDTRYADSVKGRFTISADTSKNTAYLQMNSLRAEDTAVYYCSREGQDGYNAWDLWGQGTLVTVSS

SEQ ID NO: 874 - protein, 107 aa, synthetic construct
DIQMTQSPSSLSASVGDRVTITCRASQDISTAVAWSQQKPGKAPKLLIYSASSLYSGVPSRFSGSRSGTDFTLTISSLQPEDFATYYCQQPSLTPPTFGQGTKVEIK

SEQ ID NO: 875 - protein, 120 aa, synthetic construct
EVQLVESGGGLVQPGGSLRLSCAASGFNIKDYFIHWVRQAPGKGLEWVADIDPTNGDTRYADSVKGRFTISADTSKNTAYLQMNSLRAEDTAVYYCSREGQDGYNAWDQWGQGTLVTVSS

SEQ ID NO: 876 - protein, 107 aa, synthetic construct
DIQMTQSPSSLSASVGDRVTITCRASQDISTAVAWSQQKPGKAPKLLIYPASSLYSGVPSRFSGSRSGTDFTLTISSLQPEDFATYYCQQHPYTPPTFGQGTKVEIK

SEQ ID NO: 877 - protein, 120 aa, synthetic construct
EVQLVESGGGLVQPGGSLRLSCAASGFNIKAYYIHWVRQAPGKGLEWVADIDPDNGDTRYADSVKGRFTISADTSKNTAYLQMNSLRAEDTAVYYCSREGQDGYNAWDQWGQGTLVTVSS

SEQ ID NO: 878 - protein, 107 aa, synthetic construct
DIQMTQSPSSLSASVGDRVTITCRASQDVSTAVAWSQQKPGKAPKLLIYPASSLYSGVPSRFSGSRSGTDFTLTISSLQPEDFATYYCQQPTLTPPTFGQGTKVEIK

SEQ ID NO: 879 - protein, 120 aa, synthetic construct
EVQLVESGGGLVQPGGSLRLSCAASGFNIRAYYIHWVRQAPGKGLEWVADIDPTNGDTRYADSVKGRFTISADTSKNTAYLQMNSLRAEDTAVYYCSREGQDGYNAWDLWGQGTLVTVSS

SEQ ID NO: 880 - protein, 107 aa, synthetic construct
DIQMTQSPSSLSASVGDRVTITCRASQDVSTAVAWYQQKPGKAPKLLIYPASSLYSGVPSRFSGSRSGTDFTLTISSLQPEDFATYYCQQPGYTPPTFGQGTKVEIK

SEQ ID NO: 881 - protein, 120 aa, synthetic construct
EVQLVESGGGLVQPGGSLRLSCAASGFNIRDYYIHWVRQAPGKGLEWVADIDPTNGDTRYADSVKGRFTISADTSKNTAYLQMNSLRAEDTAVYYCSREGQDGYNAWDLWGQGTLVTVSS

SEQ ID NO: 882 - protein, 107 aa, synthetic construct
DIQMTQSPSSLSASVGDRVTITCRASQDVSTAVAWYQQKPGKAPKLLIYPASSLYSGVPSRFSGSRSGTDFTLTISSLQPEDFATYYCQQHPYTPPTFGQGTKVEIK

SEQ ID NO: 883 - protein, 120 aa, synthetic construct
EVQLVESGGGLVQPGGSLRLSCAASGFNIKDYFIHWVRQAPGKGLEWVADIDPTNGDTRYADSVKGRFTISADTSKNTAYLQMNSLRAEDTAVYYCSREGQDGYNAWDMWGQGTLVTVSS

SEQ ID NO: 884 - protein, 107 aa, synthetic construct
DIQMTQSPSSLSASVGDRVTITCRASQDISTAVAWSQQKPGKAPKLLIYPASSLYSGVPSRFSGSRSGTDFTLTISSLQPEDFATYYCQQPLYTPPTFGQGTKVEIK

SEQ ID NO: 885 - protein, 120 aa, synthetic construct
EVQLVESGGGLVQPGGSLRLSCAASGFNIRDMFIHWVRQSPGKGLEWVADISPDNGDTRYADSVKGRFTISADTSKNTAYLQMNSLRAEDTAIYYCTREGEDGYNAFDMWGQGTLVTVSS

SEQ ID NO: 886 - protein, 107 aa, synthetic construct
DIQMTQSPILLSASVGDRVTITCRASQDVSTAVAWSQQRTNGSPRLLIYSASSLYSGVPSRFSGSRSGTDFTLTISSLQPEDIADYYCQQHAYTPPTFGAGTKVEIK

SEQ ID NO: 887 - protein, 120 aa, synthetic construct
EVQLVESGGGLVQPGGSLRLSCAASGFNIRDTYIHWVRQSPGKGLEWVADIDPTNGDTRYADSVKGRFTISADTSKNTAYLQMNSLRAEDTAIYYCSREMADGYNAFDLWGQGTLVTVSS

SEQ ID NO: 888 - protein, 107 aa, synthetic construct
DIQMTQSPILLSASVGDRVTITCRASQDVSTAVAWSQQRTNGSPRLLIYSASSLYSGVPSRFSGSRSGTDFTLTISSLQPEDIADYYCQQPNYTPPTFGAGTKVEIK

SEQ ID NO: 889 - protein, 120 aa, synthetic construct
EVQLVESGGGLVQPGGSLRLSCAASGFNIRAMYIHWVRQAPGKGLEWVADIDPTNGDTRYADSVKGRFTISADTSKNTAYLQMNSLRAEDTAVYYCTREGQDGYNAFDMWGQGTLVTVSS

SEQ ID NO: 890 - protein, 107 aa, synthetic construct
DIQMTQSPSSLSASVGDRVTITCRASQDISTAVAWSQQKPGKAPKLLIYSASSLYSGVPSRFSGSRSGTDFTLTISSLQPEDFATYYCQQPNLTPPTFGQGTKVEIK

SEQ ID NO: 891 - protein, 128 aa, synthetic construct; identical to SEQ ID NO: 791

SEQ ID NO: 892 - protein, 109 aa, synthetic construct; identical to SEQ ID NO: 792

SEQ ID NO: 893 - protein, 128 aa, synthetic construct; identical to SEQ ID NO: 809

SEQ ID NO: 894 - protein, 109 aa, synthetic construct; identical to SEQ ID NO: 810

SEQ ID NO: 895 - protein, 128 aa, synthetic construct; identical to SEQ ID NO: 815

SEQ ID NO: 896 - protein, 109 aa, synthetic construct; identical to SEQ ID NO: 792

SEQ ID NO: 897 - protein, 128 aa, synthetic construct; identical to SEQ ID NO: 817

SEQ ID NO: 898 - protein, 109 aa, synthetic construct; identical to SEQ ID NO: 818

SEQ ID NO: 899 - protein, 128 aa, synthetic construct; identical to SEQ ID NO: 821

SEQ ID NO: 900 - protein, 109 aa, synthetic construct; identical to SEQ ID NO: 792

SEQ ID NO: 901 - protein, 128 aa, synthetic construct; identical to SEQ ID NO: 829

SEQ ID NO: 902 - protein, 109 aa, synthetic construct; identical to SEQ ID NO: 830

SEQ ID NO: 903 - protein, 120 aa, synthetic construct; identical to SEQ ID NO: 833

SEQ ID NO: 904 - protein, 107 aa, synthetic construct; identical to SEQ ID NO: 834

SEQ ID NO: 905 - protein, 120 aa, synthetic construct; identical to SEQ ID NO: 835

SEQ ID NO: 906 - protein, 107 aa, synthetic construct; identical to SEQ ID NO: 836

SEQ ID NO: 907 - protein, 120 aa, synthetic construct; identical to SEQ ID NO: 865

SEQ ID NO: 908 - protein, 107 aa, synthetic construct; identical to SEQ ID NO: 866

SEQ ID NO: 909 - protein, 120 aa, synthetic construct; identical to SEQ ID NO: 867

SEQ ID NO: 910 - protein, 107 aa, synthetic construct; identical to SEQ ID NO: 868

SEQ ID NO: 911 - protein, 120 aa, synthetic construct; identical to SEQ ID NO: 869

SEQ ID NO: 912 - protein, 107 aa, synthetic construct; identical to SEQ ID NO: 870

SEQ ID NO: 913 - protein, 120 aa, synthetic construct; identical to SEQ ID NO: 871

SEQ ID NO: 914 - protein, 107 aa, synthetic construct; identical to SEQ ID NO: 872

SEQ ID NO: 915 - protein, 120 aa, synthetic construct; identical to SEQ ID NO: 873

SEQ ID NO: 916 - protein, 107 aa, synthetic construct; identical to SEQ ID NO: 874

SEQ ID NO: 917 - protein, 120 aa, synthetic construct; identical to SEQ ID NO: 875

SEQ ID NO: 918 - protein, 107 aa, synthetic construct; identical to SEQ ID NO: 876

SEQ ID NO: 919 - protein, 120 aa, synthetic construct; identical to SEQ ID NO: 877

SEQ ID NO: 920 - protein, 107 aa, synthetic construct; identical to SEQ ID NO: 878

SEQ ID NO: 921 - protein, 120 aa, synthetic construct; identical to SEQ ID NO: 879

SEQ ID NO: 922 - protein, 107 aa, synthetic construct; identical to SEQ ID NO: 880

SEQ ID NO: 923 - protein, 120 aa, synthetic construct; identical to SEQ ID NO: 881

SEQ ID NO: 924 - protein, 107 aa, synthetic construct; identical to SEQ ID NO: 882

SEQ ID NO: 925 - protein, 117 aa, synthetic construct; identical to SEQ ID NO: 839

SEQ ID NO: 926 - protein, 111 aa, synthetic construct; identical to SEQ ID NO: 840

SEQ ID NO: 927 - protein, 117 aa, synthetic construct; identical to SEQ ID NO: 859

SEQ ID NO: 928 - protein, 111 aa, synthetic construct; identical to SEQ ID NO: 860

SEQ ID NO: 929 - protein, 117 aa, synthetic construct; identical to SEQ ID NO: 851

SEQ ID NO: 930 - protein, 111 aa, synthetic construct; identical to SEQ ID NO: 852

SEQ ID NO: 931 - protein, 117 aa, synthetic construct; identical to SEQ ID NO: 853

SEQ ID NO: 932 - protein, 111 aa, synthetic construct; identical to SEQ ID NO: 854

SEQ ID NO: 933 - protein, 120 aa, synthetic construct; identical to SEQ ID NO: 875

SEQ ID NO: 934 - protein, 107 aa, synthetic construct; identical to SEQ ID NO: 876

SEQ ID NO: 935 - protein, 128 aa, synthetic construct; identical to SEQ ID NO: 817

SEQ ID NO: 936 - protein, 109 aa, synthetic construct; identical to SEQ ID NO: 818

SEQ ID NO: 937 - protein, 128 aa, synthetic construct; identical to SEQ ID NO: 821

SEQ ID NO: 938 - protein, 109 aa, synthetic construct; identical to SEQ ID NO: 792

SEQ ID NO: 939 - protein, 128 aa, synthetic construct; identical to SEQ ID NO: 829

SEQ ID NO: 940 - protein, 109 aa, synthetic construct; identical to SEQ ID NO: 830

SEQ ID NO: 941 - protein, 117 aa, synthetic construct
EVQLLESGGGLVQPGGSLRLSCAASGFTFSDYYMSWVRQAPGKGLEWVANINTDGSEKYYLDSVKGRFTISRDNSKNTLYLQMNSLRAEDTAVYYCAREEYGAFDIWGQGTLVTVSS

SEQ ID NO: 942 - protein, 112 aa, synthetic construct
QSVLTQPPSASGTPGQRVTISCSGSSSNIGSNTVNWYQQLPGTAPKLLIYDNNKRPSGVPDRFSGSKSGTSASLAISGLRSEDEADYYCQSFDRGLSGSIVFGGGTKLTVLG

SEQ ID NO: 943 - protein, 120 aa, synthetic construct
EVQLLESGGGLVQPGGSLRLSCAASGFTFSSYAMHWVRQAPGKGLEWVSAISGGATTTYYADSVKGRFTISRDNSKNTLYLQMNSLRAEDTAVYYCAKGGTGDPYYFDYWGQGTLVTVSS

SEQ ID NO: 944 - protein, 112 aa, synthetic construct
QSVLTQPPSASGTPGQRVTISCTGSSSNIGAGYDVHWYQQLPGTAPKLLIYRNNQRPSGVPDRFSGSKSGTSASLAISGLRSEDEADYYCAARDDGLSGPVFGGGTKLTVLG

SEQ ID NO: 945 - protein, 119 aa, synthetic construct
EVQLLESGGGLVQPGGSLRLSCAASGFTFSNAWMSWVRQAPGKGLEWVSSISSASGYIYYGDSVKGRFTISRDNSKNTLYLQMNNLRAEDTAVYYCARGTLYGDFDEFWGQGTLVTVSS

SEQ ID NO: 946 - protein, 112 aa, synthetic construct
QSVLTQPPSASGTPGQRVTISCSGSSSNIGNNAVNWYQQLPGTAPKLLIYGNTNRPSGVPDRFSGSKSGTSASLAISGLRSEDEADYYCQSYDSSLSGYVVFGGGTKLTVLG

SEQ ID NO: 947 - protein, 118 aa, synthetic construct
EVQLLESGGGLVQPGGSLRLSCAASGFTFSSNEMSWIRQAPGKGLEWVSVIYSGGSTYYADSVKGRFTISRDNSKNTLYLQMNSLRAEDTAVYYCARREGWLVPFDYWGQGTLVTVSS

SEQ ID NO: 948 - protein, 112 aa, synthetic construct
QSVLTQPPSASGTPGQRVTISCSGSSSNIGSNTVNWYQQLPGTAPKLLIYGNIIRPSGVPDRFSGSKSGTSASLAISGLRSEDEADYYCQSFDTTLSGSIVFGGGTKLTVLG

SEQ ID NO: 949 - protein, 117 aa, synthetic construct
EVQLLESGGGLVQPGGSLRLSCAASGFTFSRYWMHWVRQVPGKGLEWVSGISDSGVVTYYADSVKGRFTISRDNSKNTLYLQMNSLRAEDTAVYYCARAQSVAFDIWGQGTLVTVSS

SEQ ID NO: 950 - protein, 112 aa, synthetic construct
QSVLTQPPSASGTPGQRVTISCSGSSSNIGAGHDVHWYQQLPGTAPKLLIYYDDLLPSGVPDRFSGSKSGTSASLAISGLRSEDEADYYCAAWDDSLSGWVFGGGTKLTVLG

SEQ ID NO: 951 - protein, 117 aa, synthetic construct
EVQLLESGGGLVQPGGSLRLSCAASGFTFSSYAMSWVRQAPGKGLEWVSVISGSGGSTYYADAVKGRFTISRDNSKNTLYLQMNSLRAEDTAVYYCTTDSGSGSYLWGQGTLVTVSS

SEQ ID NO: 952 - protein, 112 aa, synthetic construct
QSVLTQPPSASGTPGQRVTISCTGSSSNIGAGYDVHWYQQLPGTAPKLLIYSNNQRPSGVPDRFSGSKSGTSASLAISGLRSEDEADYYCAAWDDSLNGPVFGGGTKLTVLG

SEQ ID NO: 953 - protein, 116 aa, synthetic construct
EVQLLESGGGLVQPGGSLRLSCAASGFTFSSNYMSWVRQAPGKGLEWVSVISGSGGSTYYADSVKGRFTISRDNSKNTLYLQMNSLRAEDTAVYYCARDRGWFDPWGQGTLVTVSS

SEQ ID NO: 954 - protein, 111 aa, synthetic construct
QSVLTQPPSASGTPGQRVTISCSGSRSNIDNSYVSWYQQLPGTAPKLLIYRNNQRPSGVPDRFSGSKSGTSASLAISGLRSEDEADYYCATWDDSLSGPVFGGGTKLTVLG

SEQ ID NO: 955 - protein, 120 aa, synthetic construct
EVQLLESGGGLVQPGGSLRLSCAASGFTFSRHAMNWVRQAPGKGLEWVSSISTGSSYIDYADSVKGRFTISRDNSKNTLYLQMNSLRAEDTAVYYCAREKGHYYYGMDVWGQGTLVTVSS

SEQ ID NO: 956 - protein, 113 aa, synthetic construct
QSVLTQPPSASGTPGQRVTISCTGSSSNIGAGYDVHWYQQLPGTAPKLLIYGNSYRPSGVPDRFSGSKSGTSASLAISGLRSEDEADYYCQSYDTSLSAYVVFGGGTKLTVLG

SEQ ID NO: 957 - protein, 118 aa, synthetic construct
EVQLLESGGGLVQPGGSLRLSCAASGFTFSNAWMSWVRQAPGKGLEWVSAISVSGINTYYADSVKGRFTISRDNSKNTLYLQMNSLRAEDTAVYYCARDTGSLGVDYWGQGTLVTVSS

SEQ ID NO: 958 - protein, 111 aa, synthetic construct
QSVLTQPPSASGTPGQRVTISCSGSSSNIGSNTVNWYQQLPGTAPKLLIYRNNQRPSGVPDRFSGSKSGTSASLAISGLRSEDEADYYCQSYDSSLSISVFGGGTKLTVLG

SEQ ID NO: 959 - protein, 118 aa, synthetic construct
EVQLLESGGGLVQPGGSLRLSCAASGFTSSSYWMSWVRQAPGKGLEWVSAISGSGGSTYYADSVKGRFTISRDNSKNTLYLQMNSLRAEDTAVYYCAREYSGYEFDFWGQGTLVTVSS

SEQ ID NO: 960 - protein, 113 aa, synthetic construct
QSVLTQPPSASGTPGQRVTISCTGSSSNIGARSDVHWYQQLPGTAPKLLIYGNRNRPSGVPDRFSGSKSGTSASLAISGLRSEDEADYYCQSFDRGLSGSIVFGGGTKLTVLG

SEQ ID NO: 961 - protein, 118 aa, synthetic construct; identical to SEQ ID NO: 959

SEQ ID NO: 962 - protein, 113 aa, synthetic construct; identical to SEQ ID NO: 960

SEQ ID NO: 963 - protein, 118 aa, synthetic construct
EVQLLESGGGLVQPGGSLRLSCAASGFTFSSNYMSWVRQAPGKGLEWVSSISSSSSYIYYADSVKGRFTISRDNSKNTLYLQMNSLRAEDTAVYYCARDRGRTGTDYWGQGTLVTVSS

SEQ ID NO: 964 - protein, 111 aa, synthetic construct
QSVLTQPPSASGTPGQRVTISCSGTTSNIGSYAVNWYQQLPGTAPKLLIYGNINRPSGVPDRFSGSKSGTSASLAISGLRSEDEADYYCQSYDSSLSASLFGGGTKLTVLG

SEQ ID NO: 965 - protein, 120 aa, synthetic construct
EVQLLESGGGLVQPGGSLRLSCAASGFTFSSYAMSWVRQAPGKGLEWVSTIIGSGANTWYADSVKGRFTISRDNSKNTLYLQMNSLRAEDTAVYYCARHEGYYYYGMDVWGQGTLVTVSS

SEQ ID NO: 966 - protein, 112 aa, synthetic construct
QSVLTQPPSASGTPGQRVTISCTGSSSNIGAGYVVHWYQQLPGTAPKLLIYGNSNRPSGVPDRFSGSKSGTSASLAISGLRSEDEADYYCAAWDDSLNGRVFGGGTKLTVLG

SEQ ID NO: 967 - protein, 122 aa, synthetic construct
EVQLQQSGPEMEKPGASVKISCKASGYSFTGYNMNWVKQSIGKSLEWIGKIDPYYGGTDYNLRFKDKATLTVDKSSSTAYMHLSRLTSEDSAVYYCAREPNYGSNDWYFDVWGAGTTVTVSS

SEQ ID NO: 968 - protein, 108 aa, synthetic construct
DIQVTQTTSSLSASLGDRVTISCRASQDIDNYLNWYQQRPDGTVKLLIYYTSILHSGVPSRFSGSGSRTDFSLTISSLEEEDFATYFCQQGNTLPWTFGGGTKLEFKR

SEQ ID NO: 969 - protein, 119 aa, synthetic construct
KVQLQQSGAELVKPGASVKLSCKASGYTFTESIIHWVKQRSGQGLEWIGWFYPGSDNINYNEKFKDKATLTADKSSSTVYMELTRLTSEDSAVYFCASHEGPYVYFDYWGQGTTLTVSS

SEQ ID NO: 970 - protein, 108 aa, synthetic construct
DIVMTQSHKFMSTSVGDRVSITCKASQDVSTAVAWYQQKPGQSPKLLIYWTSTRHTGVPDRFTGSGSGTDYTLTISSVQAEDLALYYCQHHYSTPYTFGGGTKLEIQR

SEQ ID NO: 971 - protein, 118 aa, synthetic construct
QVQLQQSGAELARPGTSVKLSCKASGYTFTSYWMQWVKQTPGQGLEWIGAIYPGDGDSRYIQKFKGKATLTADKSSSTAYMQLSSLASEDSAVYYCARDGNYYAMDYWGQGTSVTVSS

SEQ ID NO: 972 - protein, 109 aa, synthetic construct
QIVLTQSPAIMSASLGERVTITCTASSSVSSTYLHWYQQKPGSSPKLWIYSTSNLASGVPARFSGSGSGTSYSLTISSMEAEDAATYYCHQYHRSPPTFGAGTKLELKR

SEQ ID NO: 973 - protein, 114 aa, synthetic construct
EVQLQQSGAELVKPGASVKLSCTPSGFNIKDTYMHWVKQRPEQGLEWIGRIDPANGYTEYDPKFQDKATITADTSSNTAYLQLSSLTSEDTAVYYCADTQLYYWGQGTTLTVSS

SEQ ID NO: 974 - protein, 109 aa, synthetic construct
QIVLTQSPAIMSASLGERVTMTCTASSSVSSTYLHWYQQKPGSSPKLWIYSTSNLASGVPARFSGSGSGTSYSLTISNMEAEDAATYYCHQYHRSPLTFGAGTKLELKR

SEQ ID NO: 975 - protein, 117 aa, synthetic construct
QVQLKESGPGLVAPSQSLSITCTVSGFSLTVYGVNWVRQPPGKGLEWLGMIWGDGSTAYNSALKSRLTITKDNSKTQVFLKMNSLQTDDTARYYCARDGRRYALDYWGQGTSVTVSS

SEQ ID NO: 976 - protein, 112 aa, synthetic construct
DIVLTQSPTSLAVSLGQRATISCRASESVDSYGDSFLHWYQQKPGQPPILLIYRASNLDSGIPARFSGSGSRTDFTLTINPVEADDVATYYCQQSNEDPYTFGGGTKLEINR

SEQ ID NO: 977 - protein, 8 aa, synthetic construct
GYTFTDYL

SEQ ID NO: 978 - protein, 8 aa, synthetic construct
VDPEYGST

SEQ ID NO: 979 - protein, 14 aa, synthetic construct
ARDDGSYSPFDYWG

SEQ ID NO: 980 - protein, 6 aa, synthetic construct
QNINKY

SEQ ID NO: 981 - skipped

SEQ ID NO: 982 - protein, 9 aa, synthetic construct
CLQYVNLLT

SEQ ID NO: 983 - protein, 8 aa, synthetic construct
GYTFTDYL

SEQ ID NO: 984 - protein, 8 aa, synthetic construct
VDPEYGST

SEQ ID NO: 985 - protein, 14 aa, synthetic construct
ARDDGSYSPFDYWG

SEQ ID NO: 986 - protein, 6 aa, synthetic construct
QNINKY

SEQ ID NO: 987 - skipped

SEQ ID NO: 988 - protein, 9 aa, synthetic construct
CLQYVNLIT

SEQ ID NO: 989 - protein, 8 aa, synthetic construct
GYTFTDYI

SEQ ID NO: 990 - protein, 8 aa, synthetic construct
VDPEYGST

SEQ ID NO: 991 - protein, 14 aa, synthetic construct
ARDDGSYSPFDYWG

SEQ ID NO: 992 - protein, 6 aa, synthetic construct
QNINKY

SEQ ID NO: 993 - skipped

SEQ ID NO: 994 - protein, 9 aa, synthetic construct
CLQYVNLLT

SEQ ID NO: 995 - protein, 8 aa, synthetic construct
GYTFTDYI

SEQ ID NO: 996 - protein, 8 aa, synthetic construct
VDPEYGST

SEQ ID NO: 997 - protein, 14 aa, synthetic construct
ARDDGSYSPFDYWG

SEQ ID NO: 998 - protein, 6 aa, synthetic construct
QNINKY

SEQ ID NO: 999 - skipped

SEQ ID NO: 1000 - protein, 9 aa, synthetic construct
CLQYVNLIT

SEQ ID NO: 1001 - protein, 8 aa, synthetic construct
GYTFTDYL

SEQ ID NO: 1002 - protein, 8 aa, synthetic construct
VDPEYGST

SEQ ID NO: 1003 - protein, 14 aa, synthetic construct
ARDDGSYSPFDYWG

SEQ ID NO: 1004 - protein, 6 aa, synthetic construct
QNINKY

SEQ ID NO: 1005 - skipped

SEQ ID NO: 1006 - protein, 9 aa, synthetic construct
CLQYVNLLT

SEQ ID NO: 1007 - protein, 8 aa, synthetic construct
GYTFTDYL

SEQ ID NO: 1008 - protein, 8 aa, synthetic construct
VDPEYGST

SEQ ID NO: 1009 - protein, 14 aa, synthetic construct
ARDDGSYSPFDYWG

SEQ ID NO: 1010 - protein, 6 aa, synthetic construct
QNINKY

SEQ ID NO: 1011 - skipped

SEQ ID NO: 1012 - protein, 9 aa, synthetic construct
CLQYVNLIT

SEQ ID NO: 1013 - protein, 8 aa, synthetic construct
GYTFTDYI

SEQ ID NO: 1014 - protein, 8 aa, synthetic construct
VDPEYGST

SEQ ID NO: 1015 - protein, 14 aa, synthetic construct
ARDDGSYSPFDYWG

SEQ ID NO: 1016 - protein, 6 aa, synthetic construct
QNINKY

SEQ ID NO: 1017 - skipped

SEQ ID NO: 1018 - protein, 9 aa, synthetic construct
CLQYVNLLT

SEQ ID NO: 1019 - protein, 8 aa, synthetic construct
GYTFTDYI

SEQ ID NO: 1020 - protein, 8 aa, synthetic construct
VDPEYGST

SEQ ID NO: 1021 - protein, 14 aa, synthetic construct
ARDDGSYSPFDYWG

SEQ ID NO: 1022 - protein, 6 aa, synthetic construct
QNINKY

SEQ ID NO: 1023 - skipped

SEQ ID NO: 1024 - protein, 9 aa, synthetic construct
CLQYVNLIT

SEQ ID NO: 1025 - protein, 7 aa, synthetic construct
GFTFSSY

SEQ ID NO: 1026 - protein, 6 aa, synthetic construct
SSGGSY

SEQ ID NO: 1027 - protein, 14 aa, synthetic construct
QRVDGYSSYWYFDV

SEQ ID NO: 1028 - protein, 10 aa, synthetic construct
SASSSVYYMY

SEQ ID NO: 1029 - protein, 7 aa, synthetic construct
STSNLAS

SEQ ID NO: 1030 - protein, 9 aa, synthetic construct
QQRRNYPYT

SEQ ID NO: 1031 - protein, 14 aa, synthetic construct
FDDYGMSWVRQAPG

SEQ ID NO: 1032 - protein, 18 aa, synthetic construct
SVIYSGGSTYYADSVKGR

SEQ ID NO: 1033 - protein, 16 aa, synthetic construct
CARDRSSSWYRDGMDV

SEQ ID NO: 1034 - protein, 15 aa, synthetic construct
CTGSSSNIGAGYDVH

SEQ ID NO: 1035 - protein, 7 aa, synthetic construct
GNSNRPS

SEQ ID NO: 1036 - protein, 12 aa, synthetic construct
CAAWDDSLSGWV

SEQ ID NO: 1037 - protein, 14 aa, synthetic construct
FDDYGMSWVRQAPG

SEQ ID NO: 1038 - protein, 18 aa, synthetic construct
STIYSGDNAYYGASVRGR

SEQ ID NO: 1039 - protein, 15 aa, synthetic construct
ARVYSSSWRKRAFDI

SEQ ID NO: 1040 - protein, 14 aa, synthetic construct
CSGTSSNIESNTVN

SEQ ID NO: 1041 - protein, 7 aa, synthetic construct
SDNQRPS

SEQ ID NO: 1042 - protein, 12 aa, synthetic construct
CAAWDDSLSGWV

SEQ ID NO: 1043 - protein, 14 aa, synthetic construct
FSDYYMSWIRQAPG

SEQ ID NO: 1044 - protein, 19 aa, synthetic construct
ALIWYDGGNEYYADSVKGR

SEQ ID NO: 1045 - protein, 11 aa, synthetic construct
VRETGNYGMDV

SEQ ID NO: 1046 - protein, 15 aa, synthetic construct
CTGSSSNIGAGYDVH

SEQ ID NO: 1047 - protein, 7 aa, synthetic construct
RNNQRPS

SEQ ID NO: 1048 - protein, 12 aa, synthetic construct
CATWDDRVNGPV

SEQ ID NO: 1049 - protein, 14 aa, synthetic construct
FSDYYMSWIRQAPG

SEQ ID NO: 1050 - protein, 19 aa, synthetic construct
ALIWYDGGNEYYADSVKGR

SEQ ID NO: 1051 - protein, 11 aa, synthetic construct
ARYYGDGGFDP

SEQ ID NO: 1052 - protein, 15 aa, synthetic construct
CTGSSSNIGAGYVVH

SEQ ID NO: 1053 - protein, 7 aa, synthetic construct
SNNQRPS

SEQ ID NO: 1054 - protein, 12 aa, synthetic construct
CAAWDDSLNGPV

SEQ ID NO: 1055 - protein, 14 aa, synthetic construct
FSSNYMSWVRQAPG

SEQ ID NO: 1056 - protein, 19 aa, synthetic construct
ALIWYDGSNKYYADSVKGR

SEQ ID NO: 1057 - protein, 8 aa, synthetic construct
AKDPLFDS

SEQ ID NO: 1058 - protein, 15 aa, synthetic construct
CTGRSSNIGAGYDVH

SEQ ID NO: 1059 - protein, 7 aa, synthetic construct
DNNKRPS

SEQ ID NO: 1060 - protein, 12 aa, synthetic construct
CAAWDDSLNGPV

SEQ ID NO: 1061 - protein, 14 aa, synthetic construct
FNTYSMNWVRQAPG

SEQ ID NO: 1062 - protein, 18 aa, synthetic construct
SVLYSDDDTHYADSVKGR

SEQ ID NO: 1063 - protein, 17 aa, synthetic construct
ARDCGGDCHSGDDAFDI

SEQ ID NO: 1064 - protein, 14 aa, synthetic construct
CSGSSSNIGSNTVN

SEQ ID NO: 1065 - protein, 7 aa, synthetic construct
DNDKRPS

SEQ ID NO: 1066 - protein, 12 aa, synthetic construct
CAAWHDSLNGWV

SEQ ID NO: 1067 - protein, 14 aa, synthetic construct
FSAYGMHWVRQAPG

SEQ ID NO: 1068 - protein, 19 aa, synthetic construct
AVVSYDGREKHYADSVKGR

SEQ ID NO: 1069 - protein, 14 aa, synthetic construct
ARSDGGYDSDSGYY

SEQ ID NO: 1070 - protein, 14 aa, synthetic construct
CSGSTSNIGSNFVY

SEQ ID NO: 1071 - protein, 7 aa, synthetic construct
DNNKRPS

SEQ ID NO: 1072 - protein, 11 aa, synthetic construct
CSSYAYSDNIL

SEQ ID NO: 1073 - protein, 14 aa, synthetic construct
FSNAWMSWVRQAPG

SEQ ID NO: 1074 - protein, 19 aa, synthetic construct
SGISSSGSSAYYADSVKGR

SEQ ID NO: 1075 - protein, 18 aa, synthetic construct
ARHYYYHIAGYYYDTFDI

SEQ ID NO: 1076 - protein, 14 aa, synthetic construct
CSGSSSNIGGNTVN

SEQ ID NO: 1077 - protein, 7 aa, synthetic construct
GNTNRPS

SEQ ID NO: 1078 - protein, 12 aa, synthetic construct
CAAWDDSLSGVV

SEQ ID NO: 1079 - protein, 14 aa, synthetic construct
FSSYAMSWVRQAPG

SEQ ID NO: 1080 - protein, 19 aa, synthetic construct
ATISYHGSDKDYADSVKGR

SEQ ID NO: 1081 - protein, 18 aa, synthetic construct
ARDANYHSSGYYYDVFDI

SEQ ID NO: 1082 - protein, 14 aa, synthetic construct
CSGSSSNIGSNTVN

SEQ ID NO: 1083 - protein, 7 aa, synthetic construct
GNSNRPS

SEQ ID NO: 1084 - protein, 12 aa, synthetic construct
CAAWDDSLSTWV

SEQ ID NO: 1085 - protein, 14 aa, synthetic construct
FSDYYMTWIRQAPG

SEQ ID NO: 1086 - protein, 19 aa, synthetic construct
SGISGSGGYIHYADSVKGR

SEQ ID NO: 1087 - protein, 11 aa, synthetic construct
AREGLLPDAFD

SEQ ID NO: 1088 - protein, 14 aa, synthetic construct
CSGSSSNIGNNYVS

SEQ ID NO: 1089 - protein, 7 aa, synthetic construct
RNNQRPS

SEQ ID NO: 1090 - protein, 12 aa, synthetic construct
CAAWDDSVSGWV

SEQ ID NO: 1091 - protein, 14 aa, synthetic construct
FSSYSMNWVRQAPG

SEQ ID NO: 1092 - protein, 19 aa, synthetic construct
AVMSYDEYNTYYADSVKGR

SEQ ID NO: 1093 - protein, 13 aa, synthetic construct
AKGFYGDYPLWDY

SEQ ID NO: 1094 - protein, 14 aa, synthetic construct
CSGGNSNIGTNTVD

SEQ ID NO: 1095 - protein, 7 aa, synthetic construct
SNNQRPS

SEQ ID NO: 1096 - protein, 12 aa, synthetic construct
CAAWDDSVNGPV

SEQ ID NO: 1097 - protein, 14 aa, synthetic construct
FSSYEMNWVRQAPG

SEQ ID NO: 1098 - protein, 18 aa, synthetic construct
STITGGGSIYDANSVQGR

SEQ ID NO: 1099 - protein, 18 aa, synthetic construct
ARDSTYHSSGYYYDVFDI

SEQ ID NO: 1100 - protein, 14 aa, synthetic construct
CSGSSSNIGSNTVN

SEQ ID NO: 1101 - protein, 7 aa, synthetic construct
GNSNRPS

SEQ ID NO: 1102 - protein, 13 aa, synthetic construct
CAAWDDSLSGHWV

SEQ ID NO: 1103 - protein, 14 aa, synthetic construct
FSSYGMHWVRQAPG

SEQ ID NO: 1104 - protein, 20 aa, synthetic construct
SAVFGSGHGNTFYADAVKGR

SEQ ID NO: 1105 - protein, 14 aa, synthetic construct
AREQLWFGQDAFDI

SEQ ID NO: 1106 - protein, 14 aa, synthetic construct
CSGSSSNIGSNTVN

SEQ ID NO: 1107 - protein, 7 aa, synthetic construct
GNSNRPS

SEQ ID NO: 1108 - protein, 12 aa, synthetic construct
CQSYDSSLSASV

SEQ ID NO: 1109 - protein, 14 aa, synthetic construct
FSDAWMTWVRQAPG

SEQ ID NO: 1110 - protein, 19 aa, synthetic construct
SDLSDSGGSTYYADSVKGR

SEQ ID NO: 1111 - protein, 11 aa, synthetic construct
GRLAAGGPVDY

SEQ ID NO: 1112 - protein, 15 aa, synthetic construct
CTGSSSNIGAGYDVH

SEQ ID NO: 1113 - protein, 7 aa, synthetic construct
SNNQRPS

SEQ ID NO: 1114 - protein, 12 aa, synthetic construct
CSVWDDSLNSWV

SEQ ID NO: 1115 - protein, 5 aa, synthetic construct
VYGVN

SEQ ID NO: 1116 - protein, 16 aa, synthetic construct
MIWGDGSTAYNSALKS

SEQ ID NO: 1117 - protein, 9 aa, synthetic construct
DGRRYALDY

SEQ ID NO: 1118 - protein, 15 aa, synthetic construct
RASESVDSYGDSFLH

SEQ ID NO: 1119 - protein, 7 aa, synthetic construct
RASNLDS

SEQ ID NO: 1120 - protein, 9 aa, synthetic construct
QQSNEDPYT

SEQ ID NO: 1121 - protein, 7 aa, synthetic construct
TLGMGVG

SEQ ID NO: 1122 - protein, 16 aa, synthetic construct
HIWWDADKYYNPALKS

SEQ ID NO: 1123 - protein, 9 aa, synthetic construct
MTGTRYFDV

SEQ ID NO: 1124 - protein, 11 aa, synthetic construct
KASQNINKFIA

SEQ ID NO: 1125 - protein, 7 aa, synthetic construct
YTSTLQP

SEQ ID NO: 1126 - protein, 8 aa, synthetic construct
LQYGVLWT

SEQ ID NO: 1127 - protein, 5 aa, synthetic construct
TYGMS

SEQ ID NO: 1128 - protein, 17 aa, synthetic construct
WIHTYSGVPTYADDFKG

SEQ ID NO: 1129 - protein, 7 aa, synthetic construct
GLYGVDY

SEQ ID NO: 1130 - protein, 11 aa, synthetic construct
KASENVVTYVS

SEQ ID NO: 1131 - protein, 7 aa, synthetic construct
GASNRYT

SEQ ID NO: 1132 - protein, 9 aa, synthetic construct
GQSYTYPYT

SEQ ID NO: 1133 - protein, 5 aa, synthetic construct
TYDLS

SEQ ID NO: 1134 - protein, 17 aa, synthetic construct
YINNGGISTYYSDTVKG

SEQ ID NO: 1135 - protein, 12 aa, synthetic construct
GPFYGSANYFDY

SEQ ID NO: 1136 - protein, 11 aa, synthetic construct
RTSESIYSNLP

SEQ ID NO: 1137 - protein, 7 aa, synthetic construct
DATKLAE

SEQ ID NO: 1138 - protein, 9 aa, synthetic construct
QHFWVTPWT

SEQ ID NO: 1139 - protein, 5 aa, synthetic construct
NYWMN

SEQ ID NO: 1140 - protein, 17 aa, synthetic construct
MIHPSDTETRLNQNFKD

SEQ ID NO: 1141 - protein, 14 aa, synthetic construct
GEGLGAARSVSMDY

SEQ ID NO: 1142 - protein, 11 aa, synthetic construct
RASQDISNYLN

SEQ ID NO: 1143 - protein, 7 aa, synthetic construct
YTAILHS

SEQ ID NO: 1144 - protein, 9 aa, synthetic construct
QQGNTLPWT

SEQ ID NO: 1145 - protein, 5 aa, synthetic construct
DIYMH

SEQ ID NO: 1146 - protein, 17 aa, synthetic construct
RIDPATGNTKHDPKFQD

SEQ ID NO: 1147 - protein, 11 aa, synthetic construct
SPYGDFGAMDY

SEQ ID NO: 1148 - protein, 12 aa, synthetic construct
TASSSVSSNYLH

SEQ ID NO: 1149 - protein, 7 aa, synthetic construct
STSNLPS

SEQ ID NO: 1150 - protein, 9 aa, synthetic construct
HQYHRSPWT

SEQ ID NO: 1151 - protein, 10 aa, synthetic construct
DYSFTNYYIH

SEQ ID NO: 1152 - protein, 17 aa, synthetic construct
WIYPGNVITKYHEKFKG

SEQ ID NO: 1153 - protein, 9 aa, synthetic construct
SRATGYFDY

SEQ ID NO: 1154 - protein, 11 aa, synthetic construct
RASQDIRNYLN

SEQ ID NO: 1155 - protein, 7 aa, synthetic construct
YTSRLHS

SEQ ID NO: 1156 - protein, 9 aa, synthetic construct
QQFNTLPWT

SEQ ID NO: 1157 - protein, 10 aa, synthetic construct
GYTFTSYWMQ

SEQ ID NO: 1158 - protein, 17 aa, synthetic construct
AIYPGDGDTRYTQKFKG

SEQ ID NO: 1159 - protein, 10 aa, synthetic construct
DDGLYYAMDY

SEQ ID NO: 1160 - protein, 12 aa, synthetic construct
TASSSVSSSYLH

SEQ ID NO: 1161 - protein, 7 aa, synthetic construct
STSNLAS

SEQ ID NO: 1162 - protein, 9 aa, synthetic construct
HQYHRSPPT

SEQ ID NO: 1163 - protein, 10 aa, synthetic construct
GYTFTDYSMN

SEQ ID NO: 1164 - protein, 17 aa, synthetic construct
WLNTETGEPIYADDFKG

SEQ ID NO: 1165 - protein, 13 aa, synthetic construct
EAIYYGTSYVLDY

SEQ ID NO: 1166 - protein, 15 aa, synthetic construct
RASESVEYYGTNFMQ

SEQ ID NO: 1167 - protein, 7 aa, synthetic construct
SASNVES

SEQ ID NO: 1168 - protein, 9 aa, synthetic construct
QQSRKDPST

SEQ ID NO: 1169 - protein, 10 aa, synthetic construct
GYSITDYLLH

SEQ ID NO: 1170 - protein, 17 aa, synthetic construct
WIDPEDAKTKYGQKFQS

SEQ ID NO: 1171 - protein, 9 aa, synthetic construct
NSGTGTMDA

SEQ ID NO: 1172 - protein, 11 aa, synthetic construct
KASQNVGSNVD

SEQ ID NO: 1173 - protein, 7 aa, synthetic construct
KASIRYT

SEQ ID NO: 1174 - protein, 9 aa, synthetic construct
MQSKSYPIT

SEQ ID NO: 1175 - protein, 10 aa, synthetic construct
GYTFTDYIIH

SEQ ID NO: 1176 - protein, 17 aa, synthetic construct
WIDPDYGNTDYADKFKK

SEQ ID NO: 1177 - protein, 10 aa, synthetic construct
DDGSPLVMDA

SEQ ID NO: 1178 - protein, 11 aa, synthetic construct
NGSQNIDNYLA

SEQ ID NO: 1179 - protein, 7 aa, synthetic construct
STYSLQT

SEQ ID NO: 1180 - protein, 8 aa, synthetic construct
FQYNNGYM

SEQ ID NO: 1181 - protein, 10 aa, synthetic construct
GFTFSNAWMG

SEQ ID NO: 1182 - protein, 19 aa, synthetic construct
QIKDKSNNYPTYYAESVKG

SEQ ID NO: 1183 - protein, 7 aa, synthetic construct
YGDFFDY

SEQ ID NO: 1184 - protein, 11 aa, synthetic construct
RASEDIYNGLA

SEQ ID NO: 1185 - protein, 7 aa, synthetic construct
NANSLHT

SEQ ID NO: 1186 - protein, 9 aa, synthetic construct
QQYYDHPLT

SEQ ID NO: 1187 - protein, 10 aa, synthetic construct
GYSITDYLLH

SEQ ID NO: 1188 - protein, 17 aa, synthetic construct
WIDPEDGKTKYGQKFQS

SEQ ID NO: 1189 - protein, 9 aa, synthetic construct
NSGTGTMDA

SEQ ID NO: 1190 - protein, 11 aa, synthetic construct
KASQNVGSNVD

SEQ ID NO: 1191 - protein, 7 aa, synthetic construct
KASIRYT

SEQ ID NO: 1192 - protein, 9 aa, synthetic construct
MQSKSYPIT

SEQ ID NO: 1193 - protein, 10 aa, synthetic construct
GFTFSNAWMG

SEQ ID NO: 1194 - protein, 19 aa, synthetic construct
QIKDKSNNYPTYYAESVKG

SEQ ID NO: 1195 - protein, 7 aa, synthetic construct
YGDFFDY

SEQ ID NO: 1196 - protein, 11 aa, synthetic construct
RASEDIYQGLA

SEQ ID NO: 1197 - protein, 7 aa, synthetic construct
NANSLHT

SEQ ID NO: 1198 - protein, 9 aa, synthetic construct
QQYYDHPLT

SEQ ID NO: 1199 - protein, 10 aa, synthetic construct
GYSITDYLLH

SEQ ID NO: 1200 - protein, 17 aa, synthetic construct
WIDPEEGKTKYGQKFQS

SEQ ID NO: 1201 - protein, 9 aa, synthetic construct
NSGTGTMDA

SEQ ID NO: 1202 - protein, 11 aa, synthetic construct
KASQNVGSNVD

SEQ ID NO: 1203 - protein, 7 aa, synthetic construct
KASIRYT

SEQ ID NO: 1204 - protein, 9 aa, synthetic construct
MQSKSYPIT

SEQ ID NO: 1205 - protein, 10 aa, synthetic construct
GYTFTDYIIH

SEQ ID NO: 1206 - protein, 17 aa, synthetic construct
WIDPDYGNTDYADKFKK

SEQ ID NO: 1207 - protein, 10 aa, synthetic construct
DDGSPLVMDA

SEQ ID NO: 1208 - protein, 11 aa, synthetic construct
QGSQNIDNYLA

SEQ ID NO: 1209 - protein, 7 aa, synthetic construct
STYSLQT

SEQ ID NO: 1210 - protein, 8 aa, synthetic construct
FQYNNGYM

SEQ ID NO: 1211 - protein, 10 aa, synthetic construct
GYTFTDYIIH

SEQ ID NO: 1212 - protein, 17 aa, synthetic construct
WIDPDYGNTDYADKFKK

SEQ ID NO: 1213 - protein, 10 aa, synthetic construct
DDGSPLVMDA

SEQ ID NO: 1214 - protein, 11 aa, synthetic construct
NGAQNIDNYLA

SEQ ID NO: 1215 - protein, 7 aa, synthetic construct
STYSLQT

SEQ ID NO: 1216 - protein, 8 aa, synthetic construct
FQYNNGYM

SEQ ID NO: 1217 - protein, 10 aa, synthetic construct
GYTFTDYIIH

SEQ ID NO: 1218 - protein, 17 aa, synthetic construct
WIDPDYGNTDYADKFKK

SEQ ID NO: 1219 - protein, 10 aa, synthetic construct
DDASPLVMDA

SEQ ID NO: 1220 - protein, 11 aa, synthetic construct
NGSQNIDNYLA

SEQ ID NO: 1221 - protein, 7 aa, synthetic construct
STYSLQT

SEQ ID NO: 1222 - protein, 8 aa, synthetic construct
FQYNNGYM

SEQ ID NO: 1223 - protein, 10 aa, synthetic construct
GYSITDYLLH

SEQ ID NO: 1224 - protein, 17 aa, synthetic construct
WIDPEEGKTKYGQKFQS

SEQ ID NO: 1225 - protein, 9 aa, synthetic construct
QSGTGTMDA

SEQ ID NO: 1226 - protein, 11 aa, synthetic construct
KASQNVGSNVD

SEQ ID NO: 1227 - protein, 7 aa, synthetic construct
KASIRYT

SEQ ID NO: 1228 - protein, 9 aa, synthetic construct
MQSKSYPIT

SEQ ID NO: 1229 - protein, 10 aa, synthetic construct
GYSITDYLLH

SEQ ID NO: 1230 - protein, 17 aa, synthetic construct
WIDPEEGKTKYGQKFQS

SEQ ID NO: 1231 - protein, 9 aa, synthetic construct
NSATGTMDA

SEQ ID NO: 1232 - protein, 11 aa, synthetic construct
KASQNVGSNVD

SEQ ID NO: 1233 - protein, 7 aa, synthetic construct
KASIRYT

SEQ ID NO: 1234 - protein, 9 aa, synthetic construct
MQSKSYPIT

SEQ ID NO: 1235 - protein, 10 aa, synthetic construct
GYSITDYLLH

SEQ ID NO: 1236 - protein, 17 aa, synthetic construct
WIDPEDAKTKYGQKFQS

SEQ ID NO: 1237 - protein, 9 aa, synthetic construct
QSGTGTMDA

SEQ ID NO: 1238 - protein, 11 aa, synthetic construct
KASQNVGSNVD

SEQ ID NO: 1239 - protein, 7 aa, synthetic construct
KASIRYT

SEQ ID NO: 1240 - protein, 9 aa, synthetic construct
MQSKSYPIT

SEQ ID NO: 1241 - protein, 10 aa, synthetic construct
GYTFTDYIIH

SEQ ID NO: 1242 - protein, 17 aa, synthetic construct
WIDPDYGNTDYADKFKK

SEQ ID NO: 1243 - protein, 10 aa, synthetic construct
DDASPLVMDA

SEQ ID NO: 1244 - protein, 11 aa, synthetic construct
QGSQNIDNYLA

SEQ ID NO: 1245 - protein, 7 aa, synthetic construct
STYSLQT

SEQ ID NO: 1246 - protein, 8 aa, synthetic construct
FQYNNGYM

SEQ ID NO: 1247 - protein, 10 aa, synthetic construct
GYTFTDYIIH

SEQ ID NO: 1248 - protein, 17 aa, synthetic construct
WIDPDYGNTDYADKFKK

SEQ ID NO: 1249 - protein, 10 aa, synthetic construct
DEGSPLVMDA

SEQ ID NO: 1250 - protein, 11 aa, synthetic construct
NGSQNIDNYLA

SEQ ID NO: 1251 - protein, 7 aa, synthetic construct
STYSLQT

SEQ ID NO: 1252 - protein, 8 aa, synthetic construct
FQYNNGYM

SEQ ID NO: 1253 - protein, 10 aa, synthetic construct
GYTFTDYIIH

SEQ ID NO: 1254 - protein, 17 aa, synthetic construct
WIDPDYGNTDYADKFKK

SEQ ID NO: 1255 - protein, 10 aa, synthetic construct
DEGSPLVMDA

SEQ ID NO: 1256 - protein, 11 aa, synthetic construct
QGSQNIDNYLA

SEQ ID NO: 1257 - protein, 7 aa, synthetic construct
STYSLQT

SEQ ID NO: 1258 - protein, 8 aa, synthetic construct
FQYNQGYM

SEQ ID NO: 1259 - protein, 10 aa, synthetic construct
GYSITDYLLH

SEQ ID NO: 1260 - protein, 17 aa, synthetic construct
WIDPEDAKTKYGQKFQS

SEQ ID NO: 1261 - protein, 9 aa, synthetic construct
NSATGTMDA

SEQ ID NO: 1262 - protein, 11 aa, synthetic construct
KASQNVGSNVD

SEQ ID NO: 1263 - protein, 7 aa, synthetic construct
KASIRYT

SEQ ID NO: 1264 - protein, 9 aa, synthetic construct
MQSKSYPIT

SEQ ID NO: 1265 - protein, 121 aa, synthetic construct
EVQLLESGGGLVQPGGSLRLSCAASGFTFDDYGMSWVRQAPGKGLEWVSVIYSGGSTYYADSVKGRFTISRDNSKNTLYLQMNSLRAEDTAVYYCARDRSSSWYRDGMDVWGQGTLVTVSS

SEQ ID NO: 1266 - protein, 112 aa, synthetic construct
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSLSGWVFGGGTKLTVLG

SEQ ID NO: 1267 - protein, 121 aa, synthetic construct
EVQLLESGGGLVQPGGSLRLSCAASGFTFDDYGMSWVRQAPGKGLEWVSTIYSGDNAYYGASVRGRFTISRDNSKNTLYLQMNSLRAEDTAVYYCARVYSSSWRKRAFDIWGQGTLVTVSS

SEQ ID NO: 1268 - protein, 111 aa, synthetic construct
QSVLTQPPSASGTPGQRVTISCSGTSSNIESNTVNWYQQLPGTAPKLLIYSDNQRPSGVPDRFSGSKSGTSASLAISGLRSEDEADYYCAAWDDSLSGWVFGGGTKLTVLG

SEQ ID NO: 1269 - protein, 118 aa, synthetic construct
EVQLLESGGGLVQPGGSLRLSCAASGFTFSDYYMSWIRQAPGKGLEWVALIWYDGGNEYYADSVKGRFTISRDNSKNTLYLQMNSLRAEDTAVYYCVRETGNYGMDVWGQGTLVTVSS

SEQ ID NO: 1270 - protein, 112 aa, synthetic construct
QSVLTQPPSASGTPGQRVTISCTGSSSNIGAGYDVHWYQQLPGTAPKLLIYRNNQRPSGVPDRFSGSKSGTSASLAISGLRSEDEADYYCATWDDRVNGPVFGGGTKLTVLG

SEQ ID NO: 1271 - protein, 118 aa, synthetic construct
EVQLLESGGGLVQPGGSLRLSCAASGFTFSDYYMSWIRQAPGKGLEWVAIISYDGGGKYFADPVKGRFTISRDNSKNTLYLQMNSLRAEDTAVYYCARYYGDGGFDPWGQGTLVTVSS

SEQ ID NO: 1272 - protein, 112 aa, synthetic construct
QSVLTQPPSASGTPGQRVTISCTGSSSNIGAGYVVHWYQQLPGTAPKLLIYSNNQRPSGVPDRFSGSKSGTSASLAISGLRSEDEADYYCAAWDDSLNGPVFGGGTKLTVLG

SEQ ID NO: 1273 - protein, 115 aa, synthetic construct
EVQLLESGGGLVQPGGSLRLSCAASGFTFSSNYMSWVRQAPGKGLEWVALIWYDGSNKYYADSVKGRFTISRDNSKNTLYLQMNSLRAEDTAVYYCAKDPLFDSWGQGTLVTVSS

SEQ ID NO: 1274 - protein, 112 aa, synthetic construct
QSVLTQPPSASGTPGQRVTISCTGRSSNIGAGYDVHWYQQLPGTAPKLLIYDNNKRPSGVPDRFSGSKSGTSASLAISGLRSEDEADYYCAAWDDSLNGPVFGGGTKLTVLG

SEQ ID NO: 1275 - protein, 123 aa, synthetic construct
EVQLLESGGGLVQPGGSLRLSCAASGFTFNTYSMNWVRQAPGKGLEWVSVLYSDDDTHYADSVKGRFTISRDNSKNTLYLQMNSLRAEDTAVYYCARDCGGDCHSGDDAFDIWGQGTLVTVSS

SEQ ID NO: 1276 - protein, 111 aa, synthetic construct
QSVLTQPPSASGTPGQRVTISCSGSSSNIGSNTVNWYQQLPGTAPKLLIYDNDKRPSGVPDRFSGSKSGTSASLAISGLRSEDEADYYCAAWHDSLNGWVLGGGTKLTVLG

SEQ ID NO: 1277 - protein, 121 aa, synthetic construct
EVQLLESGGGLVQPGGSLRLSCAASGFTFSAYGMHWVRQAPGKGLEWVAVVSYDGREKHYADSVKGRFTISRDNSKNTLYLQMNSLRAEDTAVYYCARSDGGYDSDSGYYWGQGTLVTVSS

SEQ ID NO: 1278 - protein, 110 aa, synthetic construct
QSVLTQPPSASGTPGQRVTISCSGSTSNIGSNFVYWYQQLPGTAPKLLIYDNNKRPSGVPDRFSGSKSGTSASLAISGLRSEDEADYYCSSYAYSDNILFGGGTKLTVLG

SEQ ID NO: 1279 - protein, 125 aa, synthetic construct
EVQLLESGGGLVQPGGSLRLSCAASGFTFSNAWMSWVRQAPGKGLEWVSGISSSGSSAYYADSVKGRFTISRDNSKNTLYLQMNSLRAEDTAVYYCARHYYYHIAGYYYDTFDIWGQGTLVTVSS

SEQ ID NO: 1280 - protein, 111 aa, synthetic construct
QSVLTQPPSASGTPGQRVTISCSGSSSNIGGNTVNWYQQLPGTAPKLLIYGNTNRPSGVPDRFSGSKSGTSASLAISGLRSEDEADYYCAAWDDSLSGVVFGGGTKLTVLG

SEQ ID NO: 1281 - protein, 125 aa, synthetic construct
EVQLLESGGGLVQPGGSLRLSCAASGFTFSSYAMSWVRQAPGKGLEWVATISYHGSDKDYADSVKGRFTISRDNSKNTLYLQMNSLRAEDTAVYYCARDANYHSSGYYYDVFDIWGQGTLVTVSS

SEQ ID NO: 1282 - protein, 111 aa, synthetic construct
QSVLTQPPSASGTPGQRVTISCSGSSSNIGSNTVNWYQQLPGTAPKLLIYGNSNRPSGVPDRFSGSKSGTSASLAISGLRSEDEADYYCAAWDDSLSTWVFGGGTKLTVLG

SEQ ID NO: 1283 - protein, 119 aa, synthetic construct
EVQLLESGGGLVQPGGSLRLSCAASGFTFSDYYMTWIRQAPGKGLEWVSGISGSGGYIHYADSVKGRFTISRDNSKNTLYLQMNSLRAEDTAVYYCAREGLLPDAFDIWGQGTLVTVSS

SEQ ID NO: 1284 - protein, 111 aa, synthetic construct
QSVLTQPPSASGTPGQRVTISCSGSSSNIGNNYVSWYQQLPGTAPKLLIYRNNQRPSGVPDRFSGSKSGTSASLAISGLRSEDEADYYCAAWDDSVSGWVFGGGTKLTVLG

SEQ ID NO: 1285 - protein, 120 aa, synthetic construct
EVQLLESGGGLVQPGGSLRLSCAASGFTFSSYSMNWVRQAPGKGLEWVAVMSYDEYNTYYADSVKGRFTISRDNSKNTLYLQMNSLRAEDTAVYYCAKGFYGDYPLWDYWGQGTLVTVSS

SEQ ID NO: 1286 - protein, 111 aa, synthetic construct
QSVLTQPPSASGTPGQRVTISCSGGNSNIGTNTVDWYQQLPGTAPKLLIYSNNQRPSGVPDRFSGSKSGTSASLAISGLRSEDEADYYCAAWDDSVNGPVFGGGTKLTVLG

SEQ ID NO: 1287 - protein, 124 aa, synthetic construct
EVQLLESGGGLVQPGGSLRLSCAASGFTFSSYEMNWVRQAPGKGLEWVSTITGGGSIYDANSVQGRFTISRDNSKNTLYLQMNSLRAEDTAVYYCARDSTYHSSGYYYDVFDIWGQGTLVTVSS

SEQ ID NO: 1288 - protein, 112 aa, synthetic construct
QSVLTQPPSASGTPGQRVTISCSGSSSNIGSNTVNWYQQLPGTAPKLLIYGNSNRPSGVPDRFSGSKSGTSASLAISGLRSEDEADYYCAAWDDSLSGHWVFGGGTKLTVLG

SEQ ID NO: 1289 - protein, 122 aa, synthetic construct
EVQLLESGGGLVQPGGPLRLSCAASGFTFSSYGMHWVRQAPGKGLEWVSAVFGSGHGNTFYADAVKGRFTISRDNSKNTLYLQMNSLRAEDTAVYYCAREQLWFGQDAFDIWGQGTLVTVSS

SEQ ID NO: 1290 - protein, 111 aa, synthetic construct
QSVLTQPPSASGTPGQRVTISCSGSSSNIGSNTVNWYQQLPGTAPKLLIYGNSNRPSGVPDRFSGSKSGTSASLAISGLRSEDEADYYCQSYDSSLSASVFGGGTKLTVLG

SEQ ID NO: 1291 - protein, 118 aa, synthetic construct
EVQLLESGGGLVQPGGSLRLSCAASGFTFSDAWMTWVRQAPGKGLEWVSDLSDSGGSTYYADSVKGRFTISRDNSKNTLYLQMNSLRAEDTAVYYCGRLAAGGPVDYWGQGTLVTVSS

SEQ ID NO: 1292 - protein, 112 aa, synthetic construct
QSVLTQPPSASGTPGQRVTISCTGSSSNIGAGYDVHWYQQLPGTAPKLLIYSNNQRPSGVPDRFSGSKSGTSASLAISGLRSEDEADYYCSVWDDSLNSWVFGGGTKLTVLG

SEQ ID NO: 1293 - protein, 117 aa, synthetic construct; identical to SEQ ID NO: 975

SEQ ID NO: 1294 - protein, 112 aa, synthetic construct; identical to SEQ ID NO: 976

SEQ ID NO: 1295 - protein, 119 aa, synthetic construct
QVTLKESGPGILQPSQTLSLTCSFSGFSLNTLGMGVGWIRQPSGKGLEWLAHIWWDADKYYNPALKSRLTISKDTSKNHVFLKIANADTADTATYYCARMTGTRYFDVWGTGTTVTVSS

SEQ ID NO: 1296 - protein, 106 aa, synthetic construct
DIQMTQSPSSLSASLGDKVTITCKASQNINKFIAWYQHKPGEGPRLLIHYTSTLQPGIPSRFSGSGSGSDYSFSINNLEPEDIASYYCLQYGVLWTFGGGTKLEIK

SEQ ID NO: 1297 - protein, 118 aa, synthetic construct
QAQIQLVQSGPELKKPGETVKISCKASGYTFTTYGMSWVKQAPGKGLKWMGWIHTYSGVPTYADDFKGRFAFSLETSASTAFLQINNLKNEDTATYFCARGLYGVDYWGQGTTLTVSS

SEQ ID NO: 1298 - protein, 107 aa, synthetic construct
NIVMTQSPKSMSMSVGERVTLSCKASENVVTYVSWYQQKPEQSPKLLIYGASNRYTGVPDRFTGSGSATDFTLTISSVQAEDLADYHCGQSYTYPYTFGGGTTLEIK

SEQ ID NO: 1299 - protein, 119 aa, synthetic construct
QVTLKESGPTLVKPTQTLTLTCTFSGFSLSTLGMGVGWIRQPPGKALEWLAHIWWDADKYYNPALKSRLTITKDTSKNQVVLTMTNMDPVDTATYYCARMTGTRYFDVWGQGTTVTVSS

SEQ ID NO: 1300 - protein, 106 aa, synthetic construct
DIQMTQSPSSLSASVGDRVTITCKASQNINKFIAWYQQKPGKVPKLLIHYTSTLQPGVPSRFSGSGSGSDYTLTISSLQPEDVATYYCLQYGVLWTFGGGTKVEIK

SEQ ID NO: 1301 - protein, 119 aa, synthetic construct; identical to SEQ ID NO: 1299

SEQ ID NO: 1302 - protein, 106 aa, synthetic construct
DIQMTQSPSSLSASVGDRVTITCKASQNINKFIAWYQHKPGKVPKLLIHYTSTLQPGVPSRFSGSGSGSDYTLTISSLQPEDVATYYCLQYGVLWTFGGGTKVEIK

SEQ ID NO: 1303 - protein, 116 aa, synthetic construct
QIQLVQSGAEVKKPGASVKVSCKASGYTFTTYGMSWVRQAPGQGLEWMGWIHTYSGVPTYADDFKGRFAFTLDTSTSTAYMELRSLRSDDTAVYYCARGLYGVDYWGQGTLVTVSS

SEQ ID NO: 1304 - protein, 107 aa, synthetic construct
EIVMTQSPATLSLSPGERATLSCKASENVVTYVSWYQQKPGQAPRLLIYGASNRYTGIPARFSGSGSATDFTLTISSLQPEDFAVYYCGQSYTYPYTFGQGTKLEIK

SEQ ID NO: 1305 - protein, 121 aa, synthetic construct
EVQLVESGGGLVQPGGSLRLSCAASGFAFSTYDLSWVRQAPGKGLEWVAYINNGGISTYYSDTVKGRFTISRDNAKNTLYLQMNSLRAEDTAVYYCVRGPFYGSANYFDYWGQGTLVTVSS

SEQ ID NO: 1306 - protein, 107 aa, synthetic construct
DIQMTQSPSSLSASVGDRVTITCRTSESIYSNLPWYQQKPGKAPKLLLYDATKLAEGVPSRFSGSGSGTDYTLTISSLQPEDFATYYCQHFWVTPWTFGQGTKVEIK

SEQ ID NO: 1307 - protein, 121 aa, synthetic construct
EVQLVESGGGLVQPGGSLRLSCAASGFAFSTYDLSWVRQAPGKGLEWVAYINNGGISTYYSDTVKGRFTISRDNAKNSLYLQMNSLRAEDTAVYYCVRGPFYGSANYFDYWGQGTLVTVSS

SEQ ID NO: 1308 - protein, 107 aa, synthetic construct
DIQMTQSPSSLSASVGDRVTITCRTSESIYSNLPWYQQKPGKSPKLLIYDATKLAEGVPSRFSGSGSGTDFTLTISSLQPEDFATYYCQHFWVTPWTFGQGTKVEIK

SEQ ID NO: 1309 - protein, 121 aa, synthetic construct; identical to SEQ ID NO: 1305

SEQ ID NO: 1310 - protein, 107 aa, synthetic construct
DIQMTQSPSSLSASVGDRVTITCRTSESIYSNLPWYQQKPGKAPKLLIYDATKLAEGVPSRFSGSGSGTDYTLTISSLQPEDFATYYCQHFWVTPWTFGQGTKVEIK

SEQ ID NO: 1311 - protein, 121 aa, synthetic construct; identical to SEQ ID NO: 1307

SEQ ID NO: 1312 - protein, 107 aa, synthetic construct
DIQMTQSPSSLSASVGDRVTITCRTSESIYSNLPWYQQKPGKSPKLLVYDATKLAEGVPSRFSGSGSGTDYTLTISSLQPEDFATYYCQHFWVTPWTFGQGTKVEIK

SEQ ID NO: 1313 - protein, 121 aa, synthetic construct; identical to SEQ ID NO: 1305

SEQ ID NO: 1314 - protein, 107 aa, synthetic construct; identical to SEQ ID NO: 1312

SEQ ID NO: 1315 - protein, 123 aa, synthetic construct
QVQLVQSGAEVKKPGASVKVSCKASGYSFTNYWMNWVRQAPGQRLEWMGMIHPSDTETRLNQNFKDRVTITRDTSASTAYMELSSLRSEDTAVYYCARGEGLGAARSVSMDYWGQGTLVTVSS

SEQ ID NO: 1316 - protein, 107 aa, synthetic construct
DIQMTQSPSSLSASVGDRVTITCRASQDISNYLNWYQQKPDGTVKLLIYYTAILHSGVPSRFSGSGSGTDYSLTISSLQPEDFATYFCQQGNTLPWTFGQGTKVEIK

SEQ ID NO: 1317 - protein, 123 aa, synthetic construct
QVQLVQSGAEVKKPGSSVKVSCKASGYSFTNYWMNWVRQAPGQGLEWMGMIHPSDTETRLNQNFKDKATITADKSTSTAYMELSSLRSEDTAVYYCARGEGLGAARSVSMDYWGQGTLVTVSS

SEQ ID NO: 1318 - protein, 107 aa, synthetic construct; identical to SEQ ID NO: 1316

SEQ ID NO: 1319 - protein, 123 aa, synthetic construct
QVQLVQSGAEVKKPGSSVKVSCKASGYSFTNYWMNWVRQAPGQGLEWMGMIHPSDTETRLNQNFKDRVTITADKSTSTAYMELSSLRSEDTAVYYCARGEGLGAARSVSMDYWGQGTLVTVSS

SEQ ID NO: 1320 - protein, 107 aa, synthetic construct; identical to SEQ ID NO: 1316

SEQ ID NO: 1321 - protein, 123 aa, synthetic construct
QVQLVQSGAEVKKPGSSVKVSCKASGYSFTNYWMNWVKQAPGQGLEWIGMIHPSDTETRLNQNFKDKATLTVDKSTSTAYMELSSLRSEDTAVYYCARGEGLGAARSVSMDYWGQGTLVTVSS

SEQ ID NO: 1322 - protein, 107 aa, synthetic construct; identical to SEQ ID NO: 1316

SEQ ID NO: 1323 - protein, 123 aa, synthetic construct
QVQLVQSGAEVKKPGSSVKVSCKASGYSFTNYWMNWVRQAPGQGLEWMGMIHPSDTETRLNQNFKDRVTITVDKSTSTAYMELSSLRSEDTAVYYCARGEGLGAARSVSMDYWGQGTLVTVSS

SEQ ID NO: 1324 - protein, 107 aa, synthetic construct; identical to SEQ ID NO: 1316

SEQ ID NO: 1325 - protein, 120 aa, synthetic construct
QVQLVQSGAEVKKPGASVKVSCKASGFNNKDIYMHWVRQAPGQGLEWIGRIDPATGNTKHDPKFQDRVTMTSDTSTNTVYMELSSLRSEDTAVYYCARSPYGDFGAMDYWGQGTLVTVSS

SEQ ID NO: 1326 - protein, 108 aa, synthetic construct
DIQMTQSPSSLSASVGDRVTITCTASSSVSSNYLHWYQQKPGKAPKLWIYSTSNLPSGVPSRFSGSGSGTDFTLTISSLQPEDFATYYCHQYHRSPWTFGQGTKVEIK

SEQ ID NO: 1327 - protein, 120 aa, synthetic construct
QVQLVQSGSELKKPGASVKVSCKASGFNNKDIYMHWVRQAPGQGLEWMGRIDPATGNTKHDPKFQDKAVLSSDTSVSTAYLQISSLKAEDTAVYYCARSPYGDFGAMDYWGQGTLVTVSS

SEQ ID NO: 1328 - protein, 108 aa, synthetic construct
DIQMTQSPSSLSASVGDRVTITCTASSSVSSNYLHWYQQKPGSSPKLWIYSTSNLPSGVPSRFSGSGSGTDFTLTISSLQPEDFATYYCHQYHRSPWTFGQGTKVEIK

SEQ ID NO: 1329 - protein, 120 aa, synthetic construct; identical to SEQ ID NO: 1327

SEQ ID NO: 1330 - protein, 108 aa, synthetic construct
DIQMTQSPSSLSASVGDRVTITCTASSSVSSNYLHWYQQKPGKAPKLLIYSTSNLPSGVPSRFSGSGSGTDFTLTISSLQAEDAATYYCHQYHRSPWTFGQGTKVEIK

SEQ ID NO: 1331 - protein, 120 aa, synthetic construct; identical to SEQ ID NO: 1327

SEQ ID NO: 1332 - protein, 108 aa, synthetic construct; identical to SEQ ID NO: 1326

SEQ ID NO: 1333 - protein, 120 aa, synthetic construct; identical to SEQ ID NO: 1327

SEQ ID NO: 1334 - protein, 108 aa, synthetic construct
DIQMTQSPSSLSASVGDRVTITCTASSSVSSNYLHWYQQKPGKAPKLLIYSTSNLPSGVPSRFSGSGSGTDYSLTISSLQPEDFATYYCHQYHRSPWTFGQGTKVEIK

SEQ ID NO: 1335 - protein, 120 aa, synthetic construct; identical to SEQ ID NO: 1325

SEQ ID NO: 1336 - protein, 108 aa, synthetic construct; identical to SEQ ID NO: 1328

SEQ ID NO: 1337 - protein, 120 aa, synthetic construct; identical to SEQ ID NO: 1327

SEQ ID NO: 1338 - protein, 108 aa, synthetic construct
DIQMTQSPSSLSASVGDRVTITCTASSSVSSNYLHWYQQKPGKAPKLLIYSTSNLPSGVPSRFSGSGSGTDFTLTISSLQPEDFATYYCHQYHRSPWTFGQGTKVEIK

SEQ ID NO: 1339 - protein, 118 aa, synthetic construct
QVQLQQSGPELVKPGASVTISCKASDYSFTNYYIHWVKQRPGQGLEWIGWIYPGNVITKYHEKFKGKAILTADTSSTTAYMQLNSLTSEDSAVYFCARSRATGYFDYWGQGTTLTVSS

SEQ ID NO: 1340 - protein, 107 aa, synthetic construct
DIQMTQTTSSLSASLGDRVTISCRASQDIRNYLNWFQQKPDGTIKLLIYYTSRLHSGVPSRFSGSGSGTDYSLTISNLEQEDMATYFCQQFNTLPWTFGGGTKLEIK

SEQ ID NO: 1341 - protein, 118 aa, synthetic construct
QVQLVQSGAEVKKPGASVKVSCKASDYSFTNYYIHWVRQAPGQGLEWMGWIYPGNVITKYHEKFKGRVTMTADTSTSTAYMELSSLRSEDTAVYYCARSRATGYFDYWGQGTLVTVSS

SEQ ID NO: 1342 - protein, 107 aa, synthetic construct
DIQMTQSPSSLSASVGDRVTITCRASQDIRNYLNWYQQKPGKAPKLLIYYTSRLHSGVPSRFSGSGSGTDYTLTISSLQPEDFATYYCQQFNTLPWTFGGGTKVEIK

SEQ ID NO: 1343 - protein, 119 aa, synthetic construct
EVQLQQSGAELARPGASVMLSCKASGYTFTSYWMQWLKQRPGQGLEWIGAIYPGDGDTRYTQKFKGKATLTADKSSSTAYMQLSSLASEDSAVYYCVRDDGLYYAMDYWGQGTSVTVSS

SEQ ID NO: 1344 - protein, 108 aa, synthetic construct
QIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGGGTKLEIK

SEQ ID NO: 1345 - protein, 122 aa, synthetic construct
QIQLVQSGPELKKPGETVKISCKASGYTFTDYSMNWVKQAPGKGLKWMGWLNTETGEPIYADDFKGRFVFSLETSASTAYLEINNLNDEDTATYFCAREAIYYGTSYVLDYWGQGTSVTVSS

SEQ ID NO: 1346 - protein, 111 aa, synthetic construct
DIVLTQSPASLAVSLGQRATISCRASESVEYYGTNFMQWYQQKPGQPPKLLIYSASNVESGVPARFSGSGSGTDFSLNIHPVEEDDIAVYFCQQSRKDPSTFGEGTNLEIK

SEQ ID NO: 1347 - protein, 122 aa, synthetic construct
QIQLVQSGSELKKPGASVKVSCKASGYTFTDYSMNWVRQAPGQGLEWMGWLNTETGEPIYADDFKGRFVFSLDTSVSTAYLQISSLKAEDTAVYFCAREAIYYGTSYVLDYWGQGTLVTVSS

SEQ ID NO: 1348 - protein, 111 aa, synthetic construct
DIVLTQSPASLAVSPGQRATITCRASESVEYYGTNFMQWYQQKPGQPPKLLIYSASNVESGVPARFSGSGSGTDFTLTINPVEANDTANYYCQQSRKDPSTFGQGTKLEIK

SEQ ID NO: 1349 - protein, 122 aa, synthetic construct
QVQLVQSGAEVKKPGASVKVSCKASGYTFTDYSMNWVRQAPGQRLEWMGWLNTETGEPIYADDFKGRVTITRDTSASTAYMELSSLRSEDTAVYYCAREAIYYGTSYVLDYWGQGTLVTVSS

SEQ ID NO: 1350 - protein, 111 aa, synthetic construct
DIVLTQSPASLAVSPGQRATITCRASESVEYYGTNFMQWYQQKPGQPPKLLIYSASNVESGVPARFSGSGSGTDFTLTINPVEANDTANYFCQQSRKDPSTFGQGTKLEIK

SEQ ID NO: 1351 - protein, 122 aa, synthetic construct; identical to SEQ ID NO: 1349

SEQ ID NO: 1352 - protein, 111 aa, synthetic construct
DIVLTQSPASLAVSPGQRATITCRASESVEYYGTNFMQWYQQKPGQPPKLLIYSASNVESGVPARFSGSGSGTDFTLTINPVEAQDTANYFCQQSRKDPSTFGQGTKLEIK

SEQ ID NO: 1353 - protein, 122 aa, synthetic construct; identical to SEQ ID NO: 1347

SEQ ID NO: 1354 - protein, 111 aa, synthetic construct; identical to SEQ ID NO: 1352

SEQ ID NO: 1355 - protein, 448 aa, synthetic construct
QVQLQQSGPELVKPGASVTISCKASDYSFTNYYIHWVKQRPGQGLEWIGWIYPGNVITKYHEKFKGKAILTADTSSTTAYMQLNSLTSEDSAVYFCARSRATGYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1356 - protein, 214 aa, synthetic construct
DIQMTQTTSSLSASLGDRVTISCRASQDIRNYLNWFQQKPDGTIKLLIYYTSRLHSGVPSRFSGSGSGTDYSLTISNLEQEDMATYFCQQFNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1357 - protein, 448 aa, synthetic construct
QVQLVQSGAEVKKPGASVKVSCKASDYSFTNYYIHWVRQAPGQGLEWMGWIYPGNVITKYHEKFKGRVTMTADTSTSTAYMELSSLRSEDTAVYYCARSRATGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1358 - protein, 214 aa, synthetic construct
DIQMTQSPSSLSASVGDRVTITCRASQDIRNYLNWYQQKPGKAPKLLIYYTSRLHSGVPSRFSGSGSGTDYTLTISSLQPEDFATYYCQQFNTLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1359 - protein, 449 aa, synthetic construct
EVQLQQSGAELARPGASVMLSCKASGYTFTSYWMQWLKQRPGQGLEWIGAIYPGDGDTRYTQKFKGKATLTADKSSSTAYMQLSSLASEDSAVYYCVRDDGLYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1360 - protein, 215 aa, synthetic construct
QIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1361 - protein, 452 aa, synthetic construct
QIQLVQSGPELKKPGETVKISCKASGYTFTDYSMNWVKQAPGKGLKWMGWLNTETGEPIYADDFKGRFVFSLETSASTAYLEINNLNDEDTATYFCAREAIYYGTSYVLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1362 - protein, 218 aa, synthetic construct
DIVLTQSPASLAVSLGQRATISCRASESVEYYGTNFMQWYQQKPGQPPKLLIYSASNVESGVPARFSGSGSGTDFSLNIHPVEEDDIAVYFCQQSRKDPSTFGEGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1363 - protein, 452 aa, synthetic construct
QIQLVQSGSELKKPGASVKVSCKASGYTFTDYSMNWVRQAPGQGLEWMGWLNTETGEPIYADDFKGRFVFSLDTSVSTAYLQISSLKAEDTAVYFCAREAIYYGTSYVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1364 - protein, 218 aa, synthetic construct
DIVLTQSPASLAVSPGQRATITCRASESVEYYGTNFMQWYQQKPGQPPKLLIYSASNVESGVPARFSGSGSGTDFTLTINPVEANDTANYYCQQSRKDPSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1365 - protein, 452 aa, synthetic construct
QVQLVQSGAEVKKPGASVKVSCKASGYTFTDYSMNWVRQAPGQRLEWMGWLNTETGEPIYADDFKGRVTITRDTSASTAYMELSSLRSEDTAVYYCAREAIYYGTSYVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1366 - protein, 218 aa, synthetic construct
DIVLTQSPASLAVSPGQRATITCRASESVEYYGTNFMQWYQQKPGQPPKLLIYSASNVESGVPARFSGSGSGTDFTLTINPVEANDTANYFCQQSRKDPSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1367 - protein, 452 aa, synthetic construct; identical to SEQ ID NO: 1365

SEQ ID NO: 1368 - protein, 218 aa, synthetic construct
DIVLTQSPASLAVSPGQRATITCRASESVEYYGTNFMQWYQQKPGQPPKLLIYSASNVESGVPARFSGSGSGTDFTLTINPVEAQDTANYFCQQSRKDPSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1369 - protein, 452 aa, synthetic construct; identical to SEQ ID NO: 1363

SEQ ID NO: 1370 - protein, 218 aa, synthetic construct; identical to SEQ ID NO: 1368

SEQ ID NO: 1371 - protein, 452 aa, synthetic construct; identical to SEQ ID NO: 1363

SEQ ID NO: 1372 - protein, 218 aa, synthetic construct; identical to SEQ ID NO: 1368

SEQ ID NO: 1373 - protein, 118 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSS

SEQ ID NO: 1374 - protein, 107 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIK

SEQ ID NO: 1375 - protein, 119 aa, synthetic construct
EVHLQQSGPEVGRPGSSVKISCKASGYTFTDYIIHWVKQSPGQGLEWMGWIDPDYGNTDYADKFKKKATLTADASSNTAFIHLSRLTSEDTATYFCARDDGSPLVMDAWGQGTSVTVSS

SEQ ID NO: 1376 - protein, 106 aa, synthetic construct
NIQLTQSPSLLSASVGDRVTLSCNGSQNIDNYLAWFQQKLGEAPKLLIYSTYSLQTGIPSRFSGSRSGTDYTLTISSLQPEDVATYFCFQYNNGYMFGAGTKLELK

SEQ ID NO: 1377 - protein, 118 aa, synthetic construct
EVQLVETGGGLVQPGKSLKLTCATSGFTFSNAWMGWVRQSPEKQLEWVAQIKDKSNNYPTYYAESVKGRFTISRDDSESSVYLQMNSLKEEDTAIYYCTGYGDFFDYWGQGVVVTVSS

SEQ ID NO: 1378 - protein, 107 aa, synthetic construct
DIQMTQSPASLSASLGETVTIECRASEDIYNGLAWYQQKPGNSPRLLIFNANSLHTGVPSRFSGSGSGTQYSLKINSLQSEDVASYFCQQYYDHPLTFGSGIKLEIK

SEQ ID NO: 1379 - protein, 118 aa, synthetic construct
EVQLQQSGPELRRPGSSVKLSCKASGYSITDYLLHWVKHSPEHGLEWIGWIDPEDGKTKYGQKFQSKATLTADTSSNAAYMQLSSLTSEDSATYFCAANSGTGTMDAWGQGASVTVSS

SEQ ID NO: 1380 - protein, 107 aa, synthetic construct
DIVMTQSPTSMSISVGDRVTLNCKASQNVGSNVDWYQQKTGQSPKVLLYKASIRYTGVPDRFTGSGSGTEFTFTITNMQAEDLAVYFCMQSKSYPITFGSGTKLEIK

SEQ ID NO: 1381 - protein, 118 aa, synthetic construct
EVQLVESGGGLVKPGGSLRLSCAASGFTFSNAWMGWVRQAPEKGLEWVGQIKDKSNNYPTYYAESVKGRFTISRDDSKNTLYLQMNSLKTEDTAVYYCTTYGDFFDYWGQGTLVTVSS

SEQ ID NO: 1382 - protein, 107 aa, synthetic construct
DIQMTQSPSSLSASVGDRVTITCRASEDIYNALAWYQQKPGKVPKLLLYNANSLHTGVPSRFSGSGSGTDYTLTISSLQPEDFATYFCQQYYDHPLTFGQGTKLEIK

SEQ ID NO: 1383 - protein, 118 aa, synthetic construct; identical to SEQ ID NO: 1381

SEQ ID NO: 1384 - protein, 107 aa, synthetic construct
DIQMTQSPSSLSASVGDRVTITCRASEDIYQGLAWYQQKPGKVPKLLLYNANSLHTGVPSRFSGSGSGTDYTLTISSLQPEDFATYFCQQYYDHPLTFGQGTKLEIK

SEQ ID NO: 1385 - protein, 118 aa, synthetic construct
EVQLVQSGAEVKKPGATVKISCKVSGYSITDYLLHWVQQAPGKGLEWMGWIDPEEGKTKYGQKFQSRVTITADTSTDTAYMELSSLRSEDTAVYYCAANSGTGTMDAWGQGTLVTVSS

SEQ ID NO: 1386 - protein, 107 aa, synthetic construct; identical to SEQ ID NO: 1374

SEQ ID NO: 1387 - protein, 119 aa, synthetic construct
QVQLVQSGAEVKKPGASVKVSCKASGYTFTDYIIHWVRQAPGQGLEWMGWIDPDYGNTDYADKFKKRVTMTADTSISTAFMELSRLRSDDTAVYYCARDDGSPLVMDAWGQGTLVTVSS

SEQ ID NO: 1388 - protein, 106 aa, synthetic construct
DIQMTQSPSSLSASVGDRVTITCQGSQNIDNYLAWFQQKPGKAPKLLIYSTYSLQTGVPSRFSGSRSGTDFTLTISSLQPEDFATYFCFQYNNGYMFGGGTKLELK

SEQ ID NO: 1389 - protein, 119 aa, synthetic construct; identical to SEQ ID NO: 1387

SEQ ID NO: 1390 - protein, 106 aa, synthetic construct
DIQMTQSPSSLSASVGDRVTITCQGSQNIDNYLAWFQQKPGKAPKLLIYSTYSLQTGVPSRFSGSRSGTDFTLTISSLQPEDFATYFCFQYNQGYMFGGGTKLELK

SEQ ID NO: 1391 - protein, 119 aa, synthetic construct; identical to SEQ ID NO: 1387

SEQ ID NO: 1392 - protein, 106 aa, synthetic construct
DIQMTQSPSSLSASVGDRVTITCNGAQNIDNYLAWFQQKPGKAPKLLIYSTYSLQTGVPSRFSGSRSGTDFTLTISSLQPEDFATYFCFQYNNGYMFGGGTKLELK

SEQ ID NO: 1393 - protein, 119 aa, synthetic construct
QVQLVQSGAEVKKPGASVKVSCKASGYTFTDYIIHWVRQAPGQGLEWMGWIDPDYGNTDYADKFKKRVTMTADTSISTAFMELSRLRSDDTAVYYCARDDASPLVMDAWGQGTLVTVSS

SEQ ID NO: 1394 - protein, 106 aa, synthetic construct
DIQMTQSPSSLSASVGDRVTITCNGSQNIDNYLAWFQQKPGKAPKLLIYSTYSLQTGVPSRFSGSRSGTDFTLTISSLQPEDFATYFCFQYNNGYMFGGGTKLELK

SEQ ID NO: 1395 - protein, 118 aa, synthetic construct
EVQLVQSGAEVKKPGATVKISCKVSGYSITDYLLHWVQQAPGKGLEWMGWIDPEEGKTKYGQKFQSRVTITADTSTDTAYMELSSLRSEDTAVYYCAAQSGTGTMDAWGQGTLVTVSS

SEQ ID NO: 1396 - protein, 107 aa, synthetic construct; identical to SEQ ID NO: 1374

SEQ ID NO: 1397 - protein, 118 aa, synthetic construct
EVQLVQSGAEVKKPGATVKISCKVSGYSITDYLLHWVQQAPGKGLEWMGWIDPEEGKTKYGQKFQSRVTITADTSTDTAYMELSSLRSEDTAVYYCAANSATGTMDAWGQGTLVTVSS

SEQ ID NO: 1398 - protein, 107 aa, synthetic construct; identical to SEQ ID NO: 1374

SEQ ID NO: 1399 - protein, 118 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAAQSGTGTMDAWGQGTLVTVSS

SEQ ID NO: 1400 - protein, 107 aa, synthetic construct; identical to SEQ ID NO: 1374

SEQ ID NO: 1401 - protein, 119 aa, synthetic construct; identical to SEQ ID NO: 1393

SEQ ID NO: 1402 - protein, 106 aa, synthetic construct; identical to SEQ ID NO: 1388

SEQ ID NO: 1403 - protein, 119 aa, synthetic construct; identical to SEQ ID NO: 1393

SEQ ID NO: 1404 - protein, 106 aa, synthetic construct; identical to SEQ ID NO: 1390

SEQ ID NO: 1405 - protein, 119 aa, synthetic construct
QVQLVQSGAEVKKPGASVKVSCKASGYTFTDYIIHWVRQAPGQGLEWMGWIDPDYGNTDYADKFKKRVTMTADTSISTAFMELSRLRSDDTAVYYCARDEGSPLVMDAWGQGTLVTVSS

SEQ ID NO: 1406 - protein, 106 aa, synthetic construct; identical to SEQ ID NO: 1394

SEQ ID NO: 1407 - protein, 119 aa, synthetic construct; identical to SEQ ID NO: 1405

SEQ ID NO: 1408 - protein, 106 aa, synthetic construct; identical to SEQ ID NO: 1388

SEQ ID NO: 1409 - protein, 119 aa, synthetic construct; identical to SEQ ID NO: 1405

SEQ ID NO: 1410 - protein, 122 aa, synthetic construct
DIVMTQSPATLSVSPGERATLSCKASQNVGSNVDWYQQKPGQSPRVLIYWYQQKPGQSPRVLIYKASIRYTGIPARFSGSGSGTEFTLTISILQSEDFAVYFCMQSKSYPITFGQGTKLEIK

SEQ ID NO: 1411 - protein, 118 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSATGTMDAWGQGTLVTVSS

SEQ ID NO: 1412 - protein, 122 aa, synthetic construct; identical to SEQ ID NO: 1410

SEQ ID NO: 1413 - protein, 7 aa, synthetic construct
GYTFTDY

SEQ ID NO: 1414 - protein, 6 aa, synthetic construct
NPNYDS

SEQ ID NO: 1415 - protein, 8 aa, synthetic construct
GNSWYFDV

SEQ ID NO: 1416 - protein, 10 aa, synthetic construct
SASSSVRYMY

SEQ ID NO: 1417 - protein, 7 aa, synthetic construct
LTSNLAS

SEQ ID NO: 1418 - protein, 9 aa, synthetic construct
QQWSSNPLT

SEQ ID NO: 1419 - protein, 7 aa, synthetic construct
GYTFTDY

SEQ ID NO: 1420 - protein, 6 aa, synthetic construct
NYDSTS

SEQ ID NO: 1421 - protein, 8 aa, synthetic construct
GNSWYFDV

SEQ ID NO: 1422 - protein, 10 aa, synthetic construct
SASSSVRYMY

SEQ ID NO: 1423 - protein, 7 aa, synthetic construct
LTSNLAS

SEQ ID NO: 1424 - protein, 9 aa, synthetic construct
QQWSSNPLT

SEQ ID NO: 1425 - protein, 10 aa, synthetic construct
GYTFTDYNLD

SEQ ID NO: 1426 - protein, 5 aa, synthetic construct
DYIMH

SEQ ID NO: 1427 - protein, 8 aa, synthetic construct
DINPNYDS

SEQ ID NO: 1428 - protein, 17 aa, synthetic construct
WVDPEYGSTDYAEKFKK

SEQ ID NO: 1429 - protein, 9 aa, synthetic construct
TSYSQKFRG

SEQ ID NO: 1430 - protein, 11 aa, synthetic construct
DDGSYSPFEDY

SEQ ID NO: 1431 - protein, 12 aa, synthetic construct
SASSSVRYMYWY

SEQ ID NO: 1432 - protein, 11 aa, synthetic construct
KASENVVTYVS

SEQ ID NO: 1433 - protein, 16 aa, synthetic construct
RSSQSLVHSNGNTYLH

SEQ ID NO: 1434 - protein, 11 aa, synthetic construct
KASENVVTYVS

SEQ ID NO: 1435 - protein, 11 aa, synthetic construct
QASQNINKYIA

SEQ ID NO: 1436 - protein, 6 aa, synthetic construct
QNINKY

SEQ ID NO: 1437 - protein, 7 aa, synthetic construct
LTSNLAS

SEQ ID NO: 1438 - protein, 7 aa, synthetic construct
VTSNLAS

SEQ ID NO: 1439 - protein, 7 aa, synthetic construct
LTSNLGS

SEQ ID NO: 1440 - protein, 7 aa, synthetic construct
GASNRTY

SEQ ID NO: 1441 - protein, 8 aa, synthetic construct
IKVSNRFS

SEQ ID NO: 1442 - protein, 6 aa, synthetic construct
DPEYGS

SEQ ID NO: 1443 - protein, 7 aa, synthetic construct
YTSTLES

SEQ ID NO: 1444 - skipped

SEQ ID NO: 1445 - protein, 7 aa, synthetic construct
QQRSNWP

SEQ ID NO: 1446 - protein, 9 aa, synthetic construct
GQGYSYPYT

SEQ ID NO: 1447 - protein, 9 aa, synthetic construct
SQTTHVPPT

SEQ ID NO: 1448 - protein, 8 aa, synthetic construct
LQYVNLIT

SEQ ID NO: 1449 - protein, 7 aa, synthetic construct
  VARIANT 5: note = a naturally occcuring amino acid
  VARIANT 6: note = a naturally occcuring amino acid
  VARIANT 2: note = a naturally occcuring amino acid
GXTFXXY

SEQ ID NO: 1450 - protein, 7 aa, synthetic construct
  VARIANT 5: note = a naturally occcuring amino acid
  VARIANT 6: note = a naturally occcuring amino acid
GYTFXXY

SEQ ID NO: 1451 - protein, 7 aa, synthetic construct
  VARIANT 5: note = a naturally occcuring amino acid
  VARIANT 6: note = a naturally occcuring amino acid
GSTFXXY

SEQ ID NO: 1452 - protein, 7 aa, synthetic construct
  VARIANT 5: note = a naturally occcuring amino acid
  VARIANT 6: note = a naturally occcuring amino acid
GTTFXXY

SEQ ID NO: 1453 - protein, 7 aa, synthetic construct
  VARIANT 5: note = a naturally occcuring amino acid
  VARIANT 6: note = a naturally occcuring amino acid
GCTFXXY

SEQ ID NO: 1454 - protein, 7 aa, synthetic construct
  VARIANT 5: note = a naturally occcuring amino acid
  VARIANT 6: note = a naturally occcuring amino acid
GNTFXXY

SEQ ID NO: 1455 - protein, 7 aa, synthetic construct
  VARIANT 5: note = a naturally occcuring amino acid
  VARIANT 6: note = a naturally occcuring amino acid
GQTFXXY

SEQ ID NO: 1456 - protein, 7 aa, synthetic construct
  VARIANT 6: note = a naturally occcuring amino acid
  VARIANT 2: note = a naturally occcuring amino acid
GXTFSXY

SEQ ID NO: 1457 - protein, 7 aa, synthetic construct
  VARIANT 6: note = a naturally occcuring amino acid
  VARIANT 2: note = a naturally occcuring amino acid
GXTFCXY

SEQ ID NO: 1458 - protein, 7 aa, synthetic construct
  VARIANT 6: note = a naturally occcuring amino acid
  VARIANT 2: note = a naturally occcuring amino acid
GXTFYXY

SEQ ID NO: 1459 - protein, 7 aa, synthetic construct
  VARIANT 6: note = a naturally occcuring amino acid
  VARIANT 2: note = a naturally occcuring amino acid
GXTFNXY

SEQ ID NO: 1460 - protein, 7 aa, synthetic construct
  VARIANT 6: note = a naturally occcuring amino acid
  VARIANT 2: note = a naturally occcuring amino acid
GXTFQXY

SEQ ID NO: 1461 - protein, 7 aa, synthetic construct
  VARIANT 2: note = a naturally occcuring amino acid
  VARIANT 5: note = a naturally occcuring amino acid
GXTFXDY

SEQ ID NO: 1462 - protein, 10 aa, synthetic construct
GFSFPSSGVD

SEQ ID NO: 1463 - protein, 16 aa, synthetic construct
VTWGVGSTHYNSALKS

SEQ ID NO: 1464 - protein, 9 aa, synthetic construct
GEWDEGFVY

SEQ ID NO: 1465 - protein, 11 aa, synthetic construct
KASQNVGTAVA

SEQ ID NO: 1466 - protein, 7 aa, synthetic construct
SASNRYT

SEQ ID NO: 1467 - protein, 9 aa, synthetic construct
QQYSSYPLT

SEQ ID NO: 1468 - protein, 10 aa, synthetic construct
GFSFPSSGVD

SEQ ID NO: 1469 - protein, 16 aa, synthetic construct
VTWGVGSTHYNQKFQG

SEQ ID NO: 1470 - protein, 9 aa, synthetic construct
GEWDEGFVY

SEQ ID NO: 1471 - protein, 11 aa, synthetic construct
KASQNVGTAVA

SEQ ID NO: 1472 - protein, 7 aa, synthetic construct
AASTLQS

SEQ ID NO: 1473 - protein, 9 aa, synthetic construct
QQYSSYPLT

SEQ ID NO: 1474 - protein, 10 aa, synthetic construct
GFSFPSSGVD

SEQ ID NO: 1475 - protein, 16 aa, synthetic construct
VTWGVGSTHYNQKFQG

SEQ ID NO: 1476 - protein, 9 aa, synthetic construct
GEWDEGFVY

SEQ ID NO: 1477 - protein, 11 aa, synthetic construct
KASQNVGTAVA

SEQ ID NO: 1478 - protein, 7 aa, synthetic construct
AASTLQS

SEQ ID NO: 1479 - protein, 9 aa, synthetic construct
QQYSSYPLT

SEQ ID NO: 1480 - protein, 10 aa, synthetic construct
  VARIANT 6: note = D or T
  VARIANT 7: note = Y, F, or L
  VARIANT 8: note = a naturally occurring amino acid or is absent
  VARIANT 10: note = a naturally occurring amino acid or is absent
  VARIANT 9: note = a naturally occurring amino acid or is absent
GYTFTXXXXX

SEQ ID NO: 1481 - protein, 117 aa, synthetic construct
QVQLKESGPGLVAPSQSLSITCTVSGFSFPSSGVDWVRQSPGKGLEWLGVTWGVGSTHYNSALKSRLSISKDNSKSQVFLKMNSLQTEDTAIYYCASGEWDEGFVYWGQGTLVTVSA

SEQ ID NO: 1482 - protein, 107 aa, synthetic construct
DIVMTQSQKFMSTTVGDRVSITCKASQNVGTAVAWYQQKPGQSPKLLIYSASNRYTGVPDRFTGSGSGTDFTLTISNMQSEDLADYFCQQYSSYPLTFGGGTKLEIK

SEQ ID NO: 1483 - protein, 117 aa, synthetic construct
QVQLVQSGAEVKKPGASVKVSCKVSGFSFPSSGVDWVRQAPGQGLEWLGVTWGVGSTHYNQKFQGRVTITKDTSKSTVYMELSSLRSEDTAVYYCASGEWDEGFVYWGQGTLVTVSS

SEQ ID NO: 1484 - protein, 107 aa, synthetic construct
DIQLTQSPSFLSASVGDRVTITCKASQNVGTAVAWYQQKPGKAPKLLIYAASTLQSGVPSRFSGSGSGTEFTLTISSLQPEDFATYYCQQYSSYPLTFGGGTKVEIK

SEQ ID NO: 1485 - protein, 117 aa, synthetic construct; identical to SEQ ID NO: 681

SEQ ID NO: 1486 - protein, 117 aa, synthetic construct
EVQLVQFGAELKKPGASVKISCKASGYTFTDYNLDWVKQPHGKGLEWIGDINPNYDSTSYSQKFRGRATLTVDKSTSTAYMELSSLRSEDTAVYYCARGNSWYFDVWGQGTTVTVSS

SEQ ID NO: 1487 - protein, 117 aa, synthetic construct
EVQLVQSGAEVKKPGASVKISCKASGYTFTDYNLDWVKQPPGKGLEWIGDINPNYDSTSYSQKFRGRATLTVDKSTSTAYMELSSLRSEDTAVYYCARGNSWYFDVWGQGTTVTVSS

SEQ ID NO: 1488 - protein, 117 aa, synthetic construct
EVQLVQSGAEVKKPGASVKVSCKASGYTFTDYNLDWVKQPPGKGLEWIGDINPNYDSTSYSQKFRGRVTITVDKSTSTAYMELSSLRSEDTAVYYCARGNSWYFDVWGQGTTVTVSS

SEQ ID NO: 1489 - protein, 117 aa, synthetic construct
EVQLVQSGAEVKKPGASVKVSCKASGYTFTDYNLDWVKQPPGKGLEWIGDINPNYDSTSYSQKFRGRVTITVDESTSTAYMELSSLRSEDTAVYYCARGNSWYFDVWGQGTTVTVSS

SEQ ID NO: 1490 - protein, 117 aa, synthetic construct
EVQLVQSGAEVKKPGASVKVSCKASGYTFTDYNLDWVKQPPGKGLEWIGDINPNYDSTSYSQKFRGRVTITADKSTSTAYMELSSLRSEDTAVYYCARGNSWYFDVWGQGTTVTVSS

SEQ ID NO: 1491 - protein, 106 aa, synthetic construct; identical to SEQ ID NO: 680

SEQ ID NO: 1492 - protein, 106 aa, synthetic construct
QIVLTQSPALMSASPGERATMSCSASSSVRYMYWYQQKPRQAPKPWIYLTSNLASGVPDRFSGSGSGTDYTLTISSLQAEDVATYYCQQWSSNPLTFGQGTKLEIK

SEQ ID NO: 1493 - protein, 106 aa, synthetic construct
QIVLTQSPATLSLSPGERATMSCSASSSVRYMYWYQQKPGQAPKPWIYLTSNLASGVPDRFSGSGSGTDYTLTISSLQAEDVATYYCQQWSSNPLTFGQGTKLEIK

SEQ ID NO: 1494 - protein, 106 aa, synthetic construct
EIVLTQSPATLSLSPGERATMSCSASSSVRYMYWYQQKPGQAPKPWIYLTSNLASGVPDRFSGSGSGTDYTLTISSLQAEDVATYYCQQWSSNPLTFGQGTKLEIK

SEQ ID NO: 1495 - protein, 106 aa, synthetic construct
EIVLTQSPATLSLSPGERATMSCSASSSVRYMYWYQQKPGQAPKPWIYLTSNLASGVPDRFSGSGSGTDFTLTISSLQAEDVATYYCQQWSSNPLTFGQGTKLEIK

SEQ ID NO: 1496 - protein, 106 aa, synthetic construct
EIVLTQSPATLSLSPGERATMSCSASSSVRYMYWYQQKPGQAPKPWIYVTSNLASGVPDRFSGSGSGTDYTLTISSLQAEDVATYYCQQWSSNPLTFGQGTKLEIK

SEQ ID NO: 1497 - protein, 106 aa, synthetic construct
EIVLTQSPATLSLSPGERATMSCSASSSVRYMYWYQQKPGQAPKPWIYLTSNLGSGVPDRFSGSGSGTDYTLTISSLQAEDVATYYCQQWSSNPLTFGQGTKLEIK

SEQ ID NO: 1498 - protein, 444 aa, synthetic construct
EVQLQQFGAELVKPGASVKISCKASGYTFTDYNLDWVKQSHGKSLEWIGDINPNYDSTSYSQKFRGKATLTVDKSSSTAYMELRSLTSEDTAVYYCARGNSWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1499 - protein, 444 aa, synthetic construct
EVQLVQFGAELKKPGASVKISCKASGYTFTDYNLDWVKQPHGKGLEWIGDINPNYDSTSYSQKFRGRATLTVDKSTSTAYMELSSLRSEDTAVYYCARGNSWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1500 - protein, 444 aa, synthetic construct
EVQLVQSGAEVKKPGASVKISCKASGYTFTDYNLDWVKQPPGKGLEWIGDINPNYDSTSYSQKFRGRATLTVDKSTSTAYMELSSLRSEDTAVYYCARGNSWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1501 - protein, 444 aa, synthetic construct
EVQLVQSGAEVKKPGASVKVSCKASGYTFTDYNLDWVKQPPGKGLEWIGDINPNYDSTSYSQKFRGRVTITVDKSTSTAYMELSSLRSEDTAVYYCARGNSWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1502 - protein, 444 aa, synthetic construct
EVQLVQSGAEVKKPGASVKVSCKASGYTFTDYNLDWVKQPPGKGLEWIGDINPNYDSTSYSQKFRGRVTITVDESTSTAYMELSSLRSEDTAVYYCARGNSWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1503 - protein, 444 aa, synthetic construct
EVQLVQSGAEVKKPGASVKVSCKASGYTFTDYNLDWVKQPPGKGLEWIGDINPNYDSTSYSQKFRGRVTITADKSTSTAYMELSSLRSEDTAVYYCARGNSWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1504 - protein, 213 aa, synthetic construct
QIVLTQSPALMSASPGEKVTMTCSASSSVRYMYWYQQKPRSSPKPWIYLTSNLASGVPARFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1505 - protein, 213 aa, synthetic construct
QIVLTQSPALMSASPGERATMSCSASSSVRYMYWYQQKPRQAPKPWIYLTSNLASGVPDRFSGSGSGTDYTLTISSLQAEDVATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1506 - protein, 213 aa, synthetic construct
QIVLTQSPATLSLSPGERATMSCSASSSVRYMYWYQQKPGQAPKPWIYLTSNLASGVPDRFSGSGSGTDYTLTISSLQAEDVATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1507 - protein, 213 aa, synthetic construct
EIVLTQSPATLSLSPGERATMSCSASSSVRYMYWYQQKPGQAPKPWIYLTSNLASGVPDRFSGSGSGTDYTLTISSLQAEDVATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1508 - protein, 213 aa, synthetic construct
EIVLTQSPATLSLSPGERATMSCSASSSVRYMYWYQQKPGQAPKPWIYLTSNLASGVPDRFSGSGSGTDFTLTISSLQAEDVATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1509 - protein, 213 aa, synthetic construct
EIVLTQSPATLSLSPGERATMSCSASSSVRYMYWYQQKPGQAPKPWIYVTSNLASGVPDRFSGSGSGTDYTLTISSLQAEDVATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1510 - protein, 213 aa, synthetic construct
EIVLTQSPATLSLSPGERATMSCSASSSVRYMYWYQQKPGQAPKPWIYLTSNLGSGVPDRFSGSGSGTDYTLTISSLQAEDVATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1511 - protein, 8 aa, synthetic construct
GDTFTSYS

SEQ ID NO: 1512 - protein, 8 aa, synthetic construct
IITILDDT

SEQ ID NO: 1513 - protein, 21 aa, synthetic construct
AGVYEGPNSEGSYDDYGFLKH

SEQ ID NO: 1514 - protein, 6 aa, synthetic construct
RSVAND

SEQ ID NO: 1515 - protein, 11 aa, synthetic construct
QQYAIWPPRET

SEQ ID NO: 1516 - protein, 8 aa, synthetic construct
GFNIKDFF

SEQ ID NO: 1517 - protein, 8 aa, synthetic construct
IDPTNGDT

SEQ ID NO: 1518 - protein, 13 aa, synthetic construct
TREGQDGYNAFDL

SEQ ID NO: 1519 - protein, 6 aa, synthetic construct
QDVSTA

SEQ ID NO: 1520 - protein, 9 aa, synthetic construct
QQHSYTPPT

SEQ ID NO: 1521 - protein, 128 aa, synthetic construct; identical to SEQ ID NO: 801

SEQ ID NO: 1522 - protein, 109 aa, synthetic construct; identical to SEQ ID NO: 802

SEQ ID NO: 1523 - protein, 120 aa, synthetic construct
EVQLVESGGGLVQPGGSLRLSCAASGFNIKDFFIHWVRQSPGKGLEWVADIDPTNGDTRYADSVKGRFTISADTSKNTAYLQMNSLRAEDTAIYYCTREGQDGYNAFDLWGQGTLVTVSS

SEQ ID NO: 1524 - protein, 107 aa, synthetic construct
DIQMTQSPILLSASVGDRVTITCRASQDVSTAVAWYQQRTNGSPRLLIYSASSLYSGVPSRFSGSRSGTDFTLTISSLQPEDIADYYCQQHSYTPPTFGAGTKVEIK

SEQ ID NO: 1525 - protein, 5 aa, synthetic construct
DYAMH

SEQ ID NO: 1526 - protein, 17 aa, synthetic construct
LISIYSDHTNYNQKFQG

SEQ ID NO: 1527 - protein, 12 aa, synthetic construct
NNYGNYGWYFDV

SEQ ID NO: 1528 - protein, 10 aa, synthetic construct
SASSSVSYMH

SEQ ID NO: 1529 - protein, 7 aa, synthetic construct
DTSKLAS

SEQ ID NO: 1530 - protein, 9 aa, synthetic construct
FQGSGYPYT

SEQ ID NO: 1531 - protein, 121 aa, synthetic construct
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 1532 - protein, 106 aa, synthetic construct
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 1533 - protein, 10 aa, synthetic construct
GFTFSDYWMD

SEQ ID NO: 1534 - protein, 17 aa, synthetic construct
NIDEDGSITEYSPFVKG

SEQ ID NO: 1535 - protein, 8 aa, synthetic construct
WGRFGFDS

SEQ ID NO: 1536 - protein, 15 aa, synthetic construct
KSSQSLLSGSFNYLT

SEQ ID NO: 1537 - protein, 7 aa, synthetic construct
YASTRHT

SEQ ID NO: 1538 - protein, 9 aa, synthetic construct
HHHYNAPPT

SEQ ID NO: 1539 - protein, 117 aa, synthetic construct
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 1540 - protein, 111 aa, synthetic construct
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 1541 - protein, 5 aa, synthetic construct
DYNLD

SEQ ID NO: 1542 - protein, 17 aa, synthetic construct
DINPNYDSTSYSQKFRG

SEQ ID NO: 1543 - protein, 8 aa, synthetic construct
GNSWYFDV

SEQ ID NO: 1544 - protein, 10 aa, synthetic construct
SASSSVRYNY

SEQ ID NO: 1545 - protein, 7 aa, synthetic construct
LTSNLAS

SEQ ID NO: 1546 - protein, 10 aa, synthetic construct
PQQWSSNPLT

SEQ ID NO: 1547 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0179203 A1
(43) Pub. Date: Jun. 5, 2025

(54) ANTIBODIES WITH FC MODIFICATIONS AND METHODS OF USING THE SAME

(71) Applicant: The General Hospital Corporation, Boston, MA (US)

(72) Inventor: Denise L. FAUSTMAN, Boston, MA (US)

(21) Appl. No.: 18/818,268

(22) Filed: Aug. 28, 2024

Related U.S. Application Data
(60) Provisional application No. 63/672,340, filed on Jul. 17, 2024.
(60) Provisional application No. 63/605,306, filed on Dec. 1, 2023.

(51) Int. Cl. C07K 16/28

(52) U.S. Cl. CPC C07K 16/2878; C07K 2317/24; C07K 2317/75; C07K 2317/76

(57) ABSTRACT

The disclosure features anti-tumor necrosis factor receptor superfamily (TNFRSF), anti-tumor necrosis factor superfamily (TNFSF), anti-CD28, and anti-ICOS antibodies and antigen-binding fragments thereof with amino acid modifications at the Fc domain, and the use of these antibodies or antigen-binding fragments to modulate immune response. The antibodies and antigen-binding fragments thereof can be used to treat a wide variety of cancers, autoimmune disorders, neurological disorders, infectious diseases, inflammatory diseases, and transplant rejections.

### CROSS REFERENCE TO RELATED APPLICATIONS

This application claims priority to U.S. Provisional Application No. 63/605,306, filed Dec. 1, 2023, and U.S. Provisional Application No. 63/672,340, filed Jul. 17, 2024. Each disclosure is herein incorporated by reference in its entirety.

### SEQUENCE LISTING

The instant application contains a Sequence Listing which has been submitted electronically in XML format and is hereby incorporated by reference in its entirety. Said XML copy, created on Aug. 27, 2024, is named “00786-592004_Sequence_Listing_8_27_24” and is 1,383,906 bytes in size.

### BACKGROUND

Maintaining control of the cell-mediated and humoral immune responses is an important facet of healthy immune system activity. The aberrant regulation of lymphocyte-driven immune responses (e.g., T cell- and B cell-driven immune reactions) has been associated with a wide array of human diseases, as the inappropriate mounting of an immune response against various self and foreign antigens plays a causal role in such pathologies as autoimmune diseases, infectious diseases, inflammatory diseases, neurological diseases or disorders, allergies, graft-versus-host disease, transplantation graft rejections, and a variety of other immunological disorders. On the other hand, the use of immunotherapy is a prominent paradigm for ameliorating various human pathologies where insufficient or excessive immune responses are involved. Targeting signaling pathways within immune cells is a promising direction for the development of therapeutics for immunotherapy.

Proteins belonging to the tumor necrosis factor receptor superfamily (TNFRSF) and their ligands (TNFSF) are intimately involved in the activation, differentiation, and survival of cells of the immune system. TNFRSF members include, but may not be limited to, 4-1BB, CD27, CD30, CD40, DR6, EDAR, Fas, GITR, HVEM, LT-β receptor, NGFR, OPG, OX40, RANK, RELT (TNFRSF19L), TNFR1, TNFR2, TRAIL-R1 (TNFRSF10A), TRAIL-R2 (TNFRSF10B), TRAIL-R4, TRAMP, TROY, XEDAR, and DCR3. TNFSF members include, but may not be limited to, TNF-α, TNF-β, lymphotoxin-β (LT-β), CD40L, FasL, CD30L, 4-1BBL, CD27L, OX40L, TRAIL, LIGHT, RANKL, TWEAK, APRIL, BAFF, VEGI, EDA-A1, EDA-A2, and GITRL. TNFRSF member proteins are oligomeric, type I or type III transmembrane proteins that contain multiple extracellular cysteine-rich domains. Several of these receptors contain intracellular death domains (DDs) that recruit caspase-interacting proteins following ligand binding to initiate the extrinsic pathway of caspase activation. Other TNF superfamily receptors that lack death domains bind TNF receptor-associated factors (TRAFs) and activate intracellular signaling pathways, such as the NF-κB pathway, that can lead to proliferation or differentiation of various cell types. In addition to these functions, several TNF superfamily receptors are also involved in regulating immune cell functions such as B-cell homeostasis and activation, natural killer cell activation, and T-cell co-stimulation. The signaling of TNFRSF member proteins, such as 4-1BB and OX40, and other costimulatory proteins such as CD28 and ICOS, are important in the regulation of immunity during inflammation and cancer.

Various antibodies or antigen-binding fragments that specifically bind TNFRSF member proteins and TNFSF member proteins, as well as CD28 and ICOS, with agonistic or antagonistic activities, have been described in the art. It is beneficial for a therapeutic antibody or antigen-binding fragment (e.g., an antibody or antigen-binding fragment that binds a TNFRSF member protein, a TNFSF member protein, CD28, or ICOS) to have a wide range of tolerable doses and dosing frequencies, so as to allow different dosing regimens for the treatment of specific diseases or disorders in different patient populations. It is also important to ensure that the antibody or antigen-binding fragment (e.g., an antibody or antigen-binding fragment that binds a TNFRSF member protein, a TNFSF member protein, CD28, or ICOS) can be administered at a high dose or dosing frequency without losing efficacy, as some studies have indicated that certain antibodies, when administered at high doses, lose efficacy due to nonproductive binding conformations (see, e.g., Mayes et al. Nat. Rev. Drug Discov. 17:509-527, 2018; incorporated herein by reference). Therefore, there remains a significant need for anti-TNFRSF, anti-TNFSF, anti-CD28, or anti-ICOS antibodies or antigen-binding fragments thereof that can be administered to and tolerated by a subject (e.g., a human subject) at a higher dose or dosing frequency and across a wide dose range that exhibit improved therapeutic profiles.

### SUMMARY OF THE DISCLOSURE

The disclosure features methods of modulating an immune response in a subject by administering to the subject a modified antibody or antigen-binding fragment thereof that specifically binds a human TNFRSF member protein, a human TNFSF member protein, human CD28, or human ICOS. The disclosure also features anti-TNFRSF, anti-TNFSF, anti-CD28, or anti-ICOS antibodies or antigen-binding fragments that contain one or more mutations within the Fc domain and constructs that contain the antibodies or antigen-binding fragments thereof. Additionally, the disclosure features polynucleotides, vectors, and host cells that may be used to produce the antibodies or antigen-binding fragments thereof or the constructs, pharmaceutical compositions and kits containing the antibodies or antigen-binding fragments thereof, constructs, polynucleotides, vectors, or host cells, and methods of modulating an immune response in a subject using the same.

In a first aspect, the disclosure features methods of modulating an immune response in a human subject comprising administering to the subject a modified antibody or antigen-binding fragment thereof that specifically binds a human TNFRSF member protein, in which the modified antibody or antigen-binding fragment thereof comprises an Fc domain with one or more amino acid modifications, and further in which the modified antibody or antigen-binding fragment thereof is administered to the human subject at a higher dose and/or frequency relative to an unmodified antibody or antigen-binding fragment thereof. In some embodiments, the modified antibody or antigen-binding fragment thereof of the disclosure can be administered to the human subject at a wide dose range. The dose range at which a modified antibody or antigen-binding fragment thereof of the disclosure can be administered may be broader than, e.g., a typical (or art-recognized) dose range of a corresponding unmodified antibody or antigen-binding fragment thereof. In some embodiments, the modified antibody or antigen-binding fragment thereof of the disclosure can be administered to the human subject at a dose range of about 10 mg to about 5000 mg, or a dose range of about 1 mg/kg to about 50 mg/kg.

In a second aspect, the disclosure features methods of modulating an immune response in a human subject comprising administering to the subject a modified antibody or antigen-binding fragment thereof that specifically binds a human TNFRSF member protein, in which the modified antibody or antigen-binding fragment thereof comprises an Fc domain with one or more amino acid modifications, and further in which the modified antibody or antigen-binding fragment thereof is administered to the human subject at a dose and/or frequency that produces a greater modulation of the immune response relative to an unmodified antibody or antigen-binding fragment thereof administered at said dose and/or frequency. In some embodiments, the modified antibody or antigen-binding fragment thereof can be administered to the human subject at a wide dose range (e.g., a dose range that may be broader than, e.g., a typical (or art-recognized) dose range of a corresponding unmodified antibody or antigen-binding fragment thereof). The modified antibody or antigen-binding fragment thereof of the disclosure may be administered to the human subject at a frequency of one or more times a month, every three weeks, every two weeks, a week, every six days, every five days, every four days, every three days, every two days, or a day. The modified antibody or antigen-binding fragment thereof of the disclosure may also be administered to the human subject at a dose of about 10 mg to about 5000 mg, or at a dose of about 1 mg/kg to about 50 mg/kg. The modified antibody or antigen-binding fragment thereof of the disclosure can produce a greater (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) modulation (e.g., increase or enhancement; or decrease or suppression) of an immune response (e.g., an immune response against a cancer, such as cellular cytotoxicity and cytokine production; an immune response against an infection, such as antibody production, cytokine production, and cellular cytotoxicity; an immune response related to inflammation, such as cytokine production and activation of eosinophils; or an immune response related to autoimmunity; among others) relative to an unmodified antibody or antigen-binding fragment thereof. An effect of a modified antibody or antigen-binding fragment thereof of the disclosure on the modulation of an immune response can be observed when the modified antibody or antigen-binding fragment thereof is administered to the human subject at a high dose and/or high dosing frequency, which is a dose or dosing frequency that is higher than a typical (or art-recognized) dose or dosing frequency of a corresponding unmodified antibody or antigen-binding fragment thereof. Methods for assessing modulation of an immune response are known in the art. See, e.g., Clay et al. Clin. Cancer Res. 7:1127-1135, 2001; Van der Burg et al. Sci. Transl. Med. 3: 108ps44, 2011; Albert-Vega et al. Front. Immunol. 9:2367, 2018; incorporated herein by reference. In some embodiments, the modified antibody or antigen-binding fragment thereof is administered to the human subject at a higher dose and/or frequency relative to an unmodified antibody or antigen-binding fragment thereof. A higher dose and/or frequency is defined as a dose or dosing frequency that is higher than a typical (or art-recognized) dose or dosing frequency of the unmodified antibody or antigen-binding fragment thereof.

In some embodiments, the TNFRSF member protein is selected from the group consisting of TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, and DCR3.

In some embodiments, the method further comprises determining (e.g., measuring) a pre-treatment level of the soluble TNFRSF member protein in the serum or plasma of the human subject prior to the administration of one or more doses of the modified antibody or antigen-binding fragment thereof. In some embodiments, the method further comprises determining (e.g., measuring) a post-treatment level of the soluble TNFRSF member protein in the serum or plasma of the human subject after the administration of one or more doses of the modified antibody or antigen-binding fragment thereof. In some embodiments, the method further comprises determining (e.g., measuring) a reference level of the soluble TNFRSF member protein in the serum or plasma of a healthy human. In some cases, a reference level is determined in a subject prior to or after the administration of an antibody or antigen-binding fragment thereof disclosed herein, a construct disclosed herein, a polynucleotide disclosed herein, a vector disclosed herein, a host cell disclosed herein, or a pharmaceutical composition disclosed herein. For example, some non-limiting examples of reference levels of the soluble TNFRSF member protein include the level of the soluble TNFRSF member protein in a subject that has not been diagnosed as having a disease, does not present with at least two or more symptoms of a disease, or has not been administered with the anti-TNFRSF antibodies or antigen-binding fragments thereof disclosed herein. An exemplary reference level of soluble TNFR2 (sTNFR2) in the serum of a healthy human is about 2.51 ng/ml (see, e.g., Kartikasari et al. Front. Immunol. 16:13:918254, 2022; incorporated herein by reference).

In some embodiments, the modified antibody or antigen-binding fragment thereof is an agonistic antibody or antigen-binding fragment thereof that specifically binds the TNFRSF member protein, and:
(a) the human subject has a pre-treatment level of the soluble TNFRSF member protein that is lower than or the same as the reference level of the soluble TNFRSF member protein; and/or
(b) the human subject has a post-treatment level of the soluble TNFRSF member protein that is higher than the reference level or the pre-treatment level of the soluble TNFRSF member protein, and in which the method further comprises administering one or more additional doses of the agonistic antibody or antigen-binding fragment thereof to the human subject in one or more additional treatment periods to maintain the higher level.

In some embodiments, the modified antibody or antigen-binding fragment thereof is an antagonistic antibody or antigen-binding fragment thereof that specifically binds the TNFRSF member protein, and:
(a) the human subject has a pre-treatment level of the soluble TNFRSF member protein that is higher relative to the reference level of the soluble TNFRSF member protein; and/or
(b) the human subject has a post-treatment level of the soluble TNFRSF member protein that is lower than the reference level or the pre-treatment level of the soluble TNFRSF member protein, and in which the method further comprises administering one or more additional doses of the antagonistic antibody or antigen-binding fragment thereof to the human subject in one or more additional treatment periods to maintain the lower level.

In some embodiments, the modified antibody or antigen-binding fragment thereof is an agonistic antibody or antigen-binding fragment thereof that specifically binds the TNFRSF member protein.

In some embodiments, the TNFRSF member protein is TNFR2.

In some embodiments, the modified antibody or antigen-binding fragment thereof comprises a heavy chain complementarity determining region (CDR) 1 (CDR-H1) comprising the amino acid sequence of X¹X²X²X¹X²X³X²JJJ, in which each X¹ is independently G, A, V, L, I, M, W, F, or P; each X² is independently Y, S, T, C, N, or Q; X³ is D or E; and each J is independently a naturally occurring amino acid or is absent. In some embodiments, the CDR-H1 comprises the amino acid sequence of GYTFTZ¹Z²JJJ (SEQ ID NO: 1480), in which Z¹ is D or T; Z² is Y, F, or L; and each J is independently a naturally occurring amino acid or is absent. In some embodiments, the CDR-H1 comprises the amino acid sequence of GYTFTDY (SEQ ID NO: 1413). In some embodiments, the antibody or antigen-binding fragment thereof further comprises one or more of a CDR-H2, a CDR-H3, a light chain CDR 1 (CDR-L1), a CDR-L2, and a CDR-L3 set forth in Table 2.

In some embodiments, the modified antibody or antigen-binding fragment thereof comprises a CDR-H1 comprising the amino acid sequence of X¹X²X²X³X¹, in which each X¹ is independently D or E; each X² is independently Y, S, T, C, N, or Q; and X³ is L, A, V, G, I, M, W, F, or P. In some embodiments, the CDR-H1 comprises the amino acid sequence of DYNLD (SEQ ID NO: 1541). In some embodiments, the antibody or antigen-binding fragment thereof further comprises one or more of a CDR-H2, a CDR-H3, a CDR-L1, a CDR-L2, and a CDR-L3 set forth in Table 2.

In some embodiments, the modified antibody or antigen-binding fragment thereof comprises a CDR-H2 comprising the amino acid sequence of X¹X²X³X²X³X³X¹X³X³X³X³X³X³X⁴X²X⁴X², in which each X¹ is independently D or E; each X² is independently I, A, V, L, G, M, W, F, or P; each X³ is independently N, S, T, C, Y, or Q; and each X⁴ is independently K, R, or H. In some embodiments, the CDR-H2 comprises the amino acid sequence of DINPNYDSTSYSQKFRG (SEQ ID NO: 1542). In some embodiments, the antibody or antigen-binding fragment thereof further comprises one or more of a CDR-H1, a CDR-H3, a CDR-L1, a CDR-L2, and a CDR-L3 set forth in Table 2.

In some embodiments, the modified antibody or antigen-binding fragment thereof comprises a CDR-H3 comprising the amino acid sequence of X¹X²X²X¹X²X¹X³X¹, in which each X¹ is independently G, A, V, L, I, M, W, F, or P; each X² is independently N, S, T, C, Y, or Q; and X³ is D or E. In some embodiments, the CDR-H3 comprises the amino acid sequence of GNSWYFDV (SEQ ID NO: 1543). In some embodiments, the antibody or antigen-binding fragment thereof further comprises one or more of a CDR-H1, a CDR-H2, a CDR-L1, a CDR-L2, and a CDR-L3 set forth in Table 2. In some embodiments, the modified antibody or antigen-binding fragment thereof comprises a CDR-L1 comprising the amino acid sequence of X¹X²X¹X¹X¹X²X³X⁴X¹X¹, in which each X¹ is independently S, Y, T, C, N, or Q; each X² is independently A, G, V, L, I, M, W, F, or P; X³ is R, H, or K; and X⁴ is Y, A, V, I, L, M, F, or W. In some embodiments, the CDR-L1 comprises the amino acid sequence of SASSSVRYNY (SEQ ID NO: 1544). In some embodiments, the antibody or antigen-binding fragment thereof further comprises one or more of a CDR-H1, a CDR-H2, a CDR-H3, a CDR-L2, and a CDR-L3 set forth in Table 2.

In some embodiments, the modified antibody or antigen-binding fragment thereof comprises a CDR-L2 comprising the amino acid sequence of X¹X²X²X²X¹X¹X², in which each X¹ is independently L, A, V, G, I, M, W, F, or P; and each X² is independently T, S, C, Y, N, or Q. In some embodiments, the CDR-L2 comprises the amino acid sequence of LTSNLAS (SEQ ID NO: 1545). In some embodiments, the antibody or antigen-binding fragment thereof further comprises one or more of a CDR-H1, a CDR-H2, a CDR-H3, a CDR-L1, and a CDR-L3 set forth in Table 2.

In some embodiments, the modified antibody or antigen-binding fragment thereof comprises a CDR-L3 comprising the amino acid sequence of X¹X²X²X¹X²X²X²X¹X¹X², in which each X¹ is independently P, A, V, L, I, M, W, F, or G; and each X² is independently Q, S, T, C, N, or Y. In some embodiments, the CDR-L3 comprises the amino acid sequence of PQQWSSNPLT (SEQ ID NO: 1546). In some embodiments, the antibody or antigen-binding fragment thereof further comprises one or more of a CDR-H1, a CDR-H2, a CDR-H3, a CDR-L1, and a CDR-L2 set forth in Table 2.

In some embodiments, the modified antibody or antigen-binding fragment thereof comprises a CDR-H1, a CDR-H2, a CDR-H3, a CDR-L1, a CDR-L2, and a CDR-L3 set forth in Table 2.

In some embodiments, the modified antibody or antigen-binding fragment thereof comprises a heavy chain variable region (VH) and a light chain variable region (VL) having amino acid sequences that are at least 80% identical (e.g., 85%, 90%, 95%, 97%, 99%, or 100% identical) to the sequences set forth in Table 3.

In some embodiments, the TNFRSF member protein is selected from the group consisting of CD27, CD40, GITR, OX40, and 4-1BB.

In some embodiments, the modified antibody or antigen-binding fragment thereof comprises the CDR-H1, the CDR-H2, the CDR-H3, the CDR-L1, the CDR-L2, and the CDR-L3 of an antibody selected from the group consisting of varlilumab (CDX-1127), CDX-1140, SEA-CD40, RO7009789, JNJ-64457107 (ADC1013), APX-005M, Chi Lob 7/4, TRX-518, MK-4166, MK-1248, GWN-323, INCAGN01876, BMS-986156, AMG-228, tavolimab (MEDI0562), PF-04518600, BMS-986178, MOXR-0916, GSK-3174998, INCAGN01949, utomilumab (PF-05082566), and urelumab (BMS-663513).

In some embodiments, the modified antibody or antigen-binding fragment thereof comprises a VH and a VL having amino acid sequences that are at least 80% identical (e.g., 85%, 90%, 95%, 97%, 99%, or 100% identical) to the VH and the VL sequences of an antibody selected from the group consisting of varlilumab (CDX-1127), CDX-1140, SEA-CD40, RO7009789, JNJ-64457107 (ADC1013), APX-005M, Chi Lob 7/4, TRX-518, MK-4166, MK-1248, GWN-323, INCAGN01876, BMS-986156, AMG-228, tavolimab (MEDI0562), PF-04518600, BMS-986178, MOXR-0916, GSK-3174998, INCAGN01949, utomilumab (PF-05082566), and urelumab (BMS-663513).

In some embodiments, the human subject has a serum or plasma level of the soluble TNFRSF member protein that is lower than a reference level of the soluble TNFRSF member protein.

In some embodiments, the modified antibody or antigen-binding fragment thereof exerts one or more biological activities selected from the group consisting of:
(a) promoting NFκB activation;
(b) inducing the proliferation of a population of regulatory T (T-reg) cells;
(c) direct killing of, or promoting the death of, a population of CD8+ T cells;
(d) promoting an increase (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) in the level of one or more mRNA molecules encoding a protein selected from the group consisting of CIAP2, TRAF2, Etk, VEGFR2, PI3K, Akt, a protein involved in the angiogenic pathway, an IKK complex, RIP, NIK, MAP3K, a protein involved in the NFκB pathway, NIK, JNK, AP-1, a MEK, MKK3, NEMO, IL2R, Foxp3, IL2, TNF, lymphotoxin α, and lymphotoxin β;
(e) promoting an increase (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) in the level of one or more proteins selected from the group consisting of CIAP2, TRAF2, Etk, VEGFR2, PI3K, Akt, a protein involved in the angiogenic pathway, an IKK complex, RIP, NIK, MAP3K, a protein involved in the NFκB pathway, NIK, JNK, AP-1, a MEK, MKK3, NEMO, IL2R, Foxp3, IL2, TNF, lymphotoxin α, and lymphotoxin β;
(f) promoting an increase (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) in the level of itaconate; and
(g) promoting an increase (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) in the level of the soluble TNFRSF member protein.

In some embodiments, the modified antibody or antigen-binding fragment thereof is an antagonistic antibody or antigen-binding fragment thereof that specifically binds the TNFRSF member protein.

In some embodiments, the TNFRSF member protein is TNFR2.

In some embodiments, the modified antibody or antigen-binding fragment thereof comprises a CDR-H1, a CDR-H2, a CDR-H3, a CDR-L1, a CDR-L2, and a CDR-L3 set forth in Table 4.

In some embodiments, the modified antibody or antigen-binding fragment thereof comprises a VH and a VL having amino acid sequences that are at least 80% identical (e.g., 85%, 90%, 95%, 97%, 99%, or 100% identical) to the sequences set forth in Table 5.

In some embodiments, the human subject has a serum or plasma level of the soluble TNFRSF member protein that is higher than a reference level of the soluble TNFRSF member protein.

In some embodiments, the modified antibody or antigen-binding fragment thereof exerts one or more biological activities selected from the group consisting of:
(a) inhibiting NFκB activation;
(b) reducing or inhibiting the proliferation of a population of T-reg cells;
(c) inducing the proliferation of a population of CD8+ T cells;
(d) reducing or inhibiting the proliferation of a population of cancer cells;
(e) inhibiting the TNFRSF member protein signaling in proliferating cells;
(f) not inhibiting the TNFRSF member protein signaling in resting cells;
(g) reducing or inhibiting the proliferation of a population of myeloid-derived suppressor cells (MDSCs);
(h) promoting a decrease (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) in the level of one or more mRNA molecules encoding a protein selected from the group consisting of cIAP2, TRAF2, Etk, VEGFR2, PI3K, Akt, a protein involved in the angiogenic pathway, an IKK complex, RIP, NIK, MAP3K, a protein involved in the NFκB pathway, NIK, JNK, AP-1, a MEK, MKK3, NEMO, IL2R, Foxp3, IL2, TNF, lymphotoxin α, and lymphotoxin β;
(i) promoting a decrease (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) in the level of one or more proteins selected from the group consisting of CIAP2, TRAF2, Etk, VEGFR2, PI3K, Akt, a protein involved in the angiogenic pathway, an IKK complex, RIP, NIK, MAP3K, a protein involved in the NFκB pathway, NIK, JNK, AP-1, a MEK, MKK3, NEMO, IL2R, Foxp3, IL2, TNF, lymphotoxin α, and lymphotoxin β; and
(j) promoting a decrease (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) in the level of the soluble TNFRSF member protein.

In some embodiments, the modified antibody or antigen-binding fragment thereof is neither an agonist nor an antagonist of the TNFRSF member protein.

In some embodiments, the TNFRSF member protein is TNFR2.

In some embodiments, the modified antibody or antigen-binding fragment thereof comprises a CDR-H1, a CDR-H2, a CDR-H3, a CDR-L1, a CDR-L2, and a CDR-L3 set forth in Table 6.

In some embodiments, the modified antibody or antigen-binding fragment thereof comprises a VH and a VL having amino acid sequences that are at least 80% identical (e.g., 85%, 90%, 95%, 97%, 99%, or 100% identical) to the sequences set forth in Table 7.

In some embodiments, the modified antibody or antigen-binding fragment thereof has less than 90% (e.g., less than 80%, less than 70%, less than 60%, less than 50%, less than 40%, less than 30%, less than 20%, or less than 10%) cross-reactivity with the soluble TNFRSF member protein. In some embodiments, the modified antibody or antigen-binding fragment thereof has less than 50% cross-reactivity with the soluble TNFRSF member protein. For example, the antibody or antigen-binding fragment thereof may bind TNFR2 but exhibits less than 50% cross-reactivity (e.g., less than 40%, 30%, 20%, or 10% cross-reactivity) to soluble TNFR2. The cross-reactivity of an antibody or antigen-binding fragment thereof with an antigen can be determined using methods that are known in the art, such as by assessing the percentage homology of an antigen sequence of a protein bound by an antibody with the sequence of a second protein. A high percentage of homology indicates that the antibody is likely to cross reacts with the second protein. Cross-reactivity of an antibody or antigen-binding fragment thereof with an antigen can be determined by using a biophysical technique, such as a Surface Plasmon Resonance (SPR)-based assay or a Biacore binding assay.

In a third aspect, the disclosure features methods of modulating an immune response in a human subject comprising administering to the subject a modified antibody or antigen-binding fragment thereof that specifically binds a human TNFSF member protein, in which the modified antibody or antigen-binding fragment thereof comprises an Fc domain with one or more amino acid modifications, and further in which the modified antibody or antigen-binding fragment thereof is administered to the human subject at a higher dose and/or frequency relative to an unmodified antibody or antigen-binding fragment thereof. In some embodiments, the modified antibody or antigen-binding fragment thereof can be administered to the human subject at a wide dose range (e.g., a dose range that may be broader than, e.g., a typical (or art-recognized) dose range of a corresponding unmodified antibody or antigen-binding fragment thereof).

In a fourth aspect, the disclosure features methods of modulating an immune response in a human subject comprising administering to the subject a modified antibody or antigen-binding fragment thereof that specifically binds a human TNFSF member protein, in which the modified antibody or antigen-binding fragment thereof comprises an Fc domain with one or more amino acid modifications, and further in which the modified antibody or antigen-binding fragment thereof is administered to the human subject at a dose and/or frequency that produces a greater modulation of the immune response relative to an unmodified antibody or antigen-binding fragment thereof administered at said dose and/or frequency. In some embodiments, the modified antibody or antigen-binding fragment thereof is administered to the human subject at a higher dose and/or frequency relative to an unmodified antibody or antigen-binding fragment thereof. In some embodiments, the modified antibody or antigen-binding fragment thereof can be administered to the human subject at a wide dose range (e.g., a dose range that may be broader than, e.g., a typical (or art-recognized) dose range of a corresponding unmodified antibody or antigen-binding fragment thereof).

In some embodiments, the TNFSF member protein is TRAIL.

In a fifth aspect, the disclosure features methods of modulating an immune response in a human subject comprising administering to the subject a modified antibody or antigen-binding fragment thereof that specifically binds human CD28, in which the modified antibody or antigen-binding fragment thereof comprises an Fc domain with one or more amino acid modifications, and further in which the modified antibody or antigen-binding fragment thereof is administered to the human subject at a higher dose and/or frequency relative to an unmodified antibody or antigen-binding fragment thereof. In some embodiments, the modified antibody or antigen-binding fragment thereof can be administered to the human subject at a wide dose range (e.g., a dose range that may be broader than, e.g., a typical (or art-recognized) dose range of a corresponding unmodified antibody or antigen-binding fragment thereof).

In a sixth aspect, the disclosure features methods of modulating an immune response in a human subject comprising administering to the subject a modified antibody or antigen-binding fragment thereof that specifically binds human CD28, in which the modified antibody or antigen-binding fragment thereof comprises an Fc domain with one or more amino acid modifications, and further in which the modified antibody or antigen-binding fragment thereof is administered to the human subject at a dose and/or frequency that produces a greater modulation of the immune response relative to an unmodified antibody or antigen-binding fragment thereof administered at said dose and/or frequency. In some embodiments, the modified antibody or antigen-binding fragment thereof is administered to the human subject at a higher dose and/or frequency relative to an unmodified antibody or antigen-binding fragment thereof. In some embodiments, the modified antibody or antigen-binding fragment thereof can be administered to the human subject at a wide dose range (e.g., a dose range that may be broader than, e.g., a typical (or art-recognized) dose range of a corresponding unmodified antibody or antigen-binding fragment thereof).

In some embodiments, the modified antibody or antigen-binding fragment thereof is an agonistic antibody or antigen-binding fragment thereof that specifically binds CD28. In some embodiments, the modified antibody or antigen-binding fragment thereof comprises the CDR-H1, the CDR-H2, the CDR-H3, the CDR-L1, the CDR-L2, and the CDR-L3 of theralizumab (TAB-08). In some embodiments, the modified antibody or antigen-binding fragment thereof comprises a VH and a VL having amino acid sequences that are at least 80% identical to the VH and the VL sequences of theralizumab (TAB-08).

In a seventh aspect, the disclosure features methods of modulating an immune response in a human subject comprising administering to the subject a modified antibody or antigen-binding fragment thereof that specifically binds human ICOS, in which the modified antibody or antigen-binding fragment thereof comprises an Fc domain with one or more amino acid modifications, and further in which the modified antibody or antigen-binding fragment thereof is administered to the human subject at a higher dose and/or frequency relative to an unmodified antibody or antigen-binding fragment thereof. In some embodiments, the modified antibody or antigen-binding fragment thereof can be administered to the human subject at a wide dose range (e.g., a dose range that may be broader than, e.g., a typical (or art-recognized) dose range of a corresponding unmodified antibody or antigen-binding fragment thereof).

In an eighth aspect, the disclosure features methods of modulating an immune response in a human subject comprising administering to the subject a modified antibody or antigen-binding fragment thereof that specifically binds human ICOS, in which the modified antibody or antigen-binding fragment thereof comprises an Fc domain with one or more amino acid modifications, and further in which the modified antibody or antigen-binding fragment thereof is administered to the human subject at a dose and/or frequency that produces a greater modulation of the immune response relative to an unmodified antibody or antigen-binding fragment thereof administered at said dose and/or frequency. In some embodiments, the modified antibody or antigen-binding fragment thereof is administered to the human subject at a higher dose and/or frequency relative to an unmodified antibody or antigen-binding fragment thereof. In some embodiments, the modified antibody or antigen-binding fragment thereof can be administered to the human subject at a wide dose range (e.g., a dose range that may be broader than, e.g., a typical (or art-recognized) dose range of a corresponding unmodified antibody or antigen-binding fragment thereof).

In some embodiments, the modified antibody or antigen-binding fragment thereof is an agonistic antibody or antigen-binding fragment thereof that specifically binds ICOS. In some embodiments, the modified antibody or antigen-binding fragment thereof comprises the CDR-H1, the CDR-H2, the CDR-H3, the CDR-L1, the CDR-L2, and the CDR-L3 of GSK-3359609 or JTX-2011. In some embodiments, the modified antibody or antigen-binding fragment thereof comprises a VH and a VL having amino acid sequences that are at least 80% identical to the VH and the VL sequences of GSK-3359609 or JTX-2011.

In some embodiments, one or more doses of the modified antibody or antigen-binding fragment thereof is administered to the human subject in one or more treatment periods, in which each dose comprises from about 0.1 mg to about 5000 mg of the modified antibody or antigen-binding fragment thereof. In some embodiments, each dose comprises from about 0.001 mg/kg to about 50 mg/kg of the modified antibody or antigen-binding fragment thereof. In some embodiments, each treatment period lasts one week, two weeks, three weeks, four weeks, five weeks, six weeks, seven weeks, eight weeks, or more.

In some embodiments, the modified antibody or antigen-binding fragment thereof is administered to the human subject at a frequency of one or more times a month, every three weeks, every two weeks, a week, every six days, every five days, every four days, every three days, every two days, or a day.

In some embodiments, the modified antibody or antigen-binding fragment thereof is administered to the human subject at a dose of about 10 mg to about 5000 mg. In some embodiments, the antibody or antigen-binding fragment thereof is administered to the human subject at a dose of about 1 mg/kg to about 50 mg/kg.

In some embodiments, the one or more amino acid modifications comprise one or more substitutions, deletions, insertions, or chemical modifications of the Fc domain.

In some embodiments, the modified antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, and a chimeric antibody or antigen-binding fragment thereof. In some embodiments, the modified antibody or antigen-binding fragment thereof is a human, humanized, or chimeric antibody or antigen-binding fragment thereof.

In some embodiments, the one or more amino acid modifications at the Fc domain decrease (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) the binding affinity of the modified antibody or antigen-binding fragment thereof for an Fc receptor relative to the corresponding unmodified antibody or antigen-binding fragment thereof. A decrease (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) in the binding affinity of the antibody or antigen-binding fragment thereof of the disclosure for an Fc receptor may manifest as an equilibrium dissociation constant (K_{D}) between the antibody or antigen-binding fragment thereof and its antigen (e.g., a TNFRSF member protein) that is higher (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) than the K_{D} between an unmodified antibody or antigen-binding fragment thereof and the antigen. The K_{D} between an antibody or antigen-binding fragment thereof and an antigen can be measured by methods such as surface plasmon resonance (SPR) (e.g., using BIACORE™ systems). In some embodiments, the effector function of the modified antibody or antigen-binding fragment thereof is not dependent on antibody-dependent cellular cytotoxicity (ADCC) or antibody-dependent cellular phagocytosis (ADCP). The effector function (e.g., ADCC or ADCP) of the antibody or antigen-binding fragment thereof of the disclosure may be determined by methods that are known in the art (see, e.g., Parekh et al. MAbs 4:310-318, 2012; and Kamen et al. J. Immunol. Methods 468:55-60, 2019). In some embodiments, the one or more amino acid modifications at the Fc domain improve a biological activity of the modified antibody or antigen-binding fragment thereof relative to the corresponding unmodified antibody or antigen-binding fragment thereof. In some embodiments, the modified antibody or antigen-binding fragment thereof exhibits dimeric binding with its antigen (e.g., a TNFRSF member protein, a TNFSF member protein, CD28, or ICOS) at a high dose, in which the modified antibody or antigen-binding fragment thereof comprises a first arm and a second arm, further in which the first arm binds to the antigen, and the second arm binds to an adjacent protein (e.g., an adjacent antigen of the antibody or antigen-binding fragment thereof). In some embodiments, the modified antibody or antigen-binding fragment thereof forms a hexagonal network with its antigen (e.g., a TNFRSF member protein, a TNFSF member protein, CD28, or ICOS) at a high dose.

In some embodiments, the Fc receptor is selected from the group consisting of FcγRI, FcγRII, and FcγRIII. In some embodiments, the biological activity of the modified antibody or antigen-binding fragment thereof is independent of ADCC or ADCP. In some embodiments, the adjacent protein is an antigen of the antibody or antigen-binding fragment thereof (e.g., a TNFRSF member protein, a TNFSF member protein, CD28, or ICOS).

In some embodiments, the Fc domain is selected from the group consisting of an IgG Fc domain, an IgA Fc domain, an IgD Fc domain, an IgE Fc domain, and an IgM Fc domain.

In some embodiments, the Fc domain is an IgG Fc domain.

In some embodiments, the Fc domain is a human IgG1 Fc domain. In some embodiments, the one or more amino acid modifications are one or more amino acid substitutions selected from the group consisting of:
(i) L235E;
(ii) L234A and L235A;
(iii) L234A, L235A, and P329G
(iv) L234E, L235F, and P331S;
(v) D265A;
(vi) G237A;
(vii) E318A;
(viii E233P
(ix) G236R and L328R;
(x) L234A, L235A, G237A, P238S, H268A, A330S, and P331S; and
(xi) N297G or N297Q,
in which the amino acid positions are numbered according to the EU index.

In some embodiments, the modified antibody or antigen-binding fragment thereof is an IgG1 antibody or antigen-binding fragment thereof.

In some embodiments, the Fc domain is a human IgG2 Fc domain. In some embodiments, the one or more amino acid modifications are one or more amino acid substitutions selected from the group consisting of:
(i) D265A;
(ii) G237A;
(iii) E318A;
(iv) H268Q, V309L, A330S, and P331S;
(v) V234A, G237A, P238S, H268A, V309L, A330S, and P331S; and
(vi) N297G or N297Q,
in which the amino acid positions are numbered according to the EU index.

In some embodiments, the modified antibody or antigen-binding fragment thereof is an IgG2 antibody or antigen-binding fragment thereof.

In some embodiments, the modified antibody or antigen-binding fragment thereof is an IgG3 antibody or antigen-binding fragment thereof.

In some embodiments, the Fc domain is a human IgG3 Fc domain. In some embodiments, the one or more amino acid modifications are one or more amino acid substitutions selected from the group consisting of:
(i) L235E;
(ii) L234A and L235A;
(iii) L234A, L235A, and P329G
(iv) L234E, L235F, and P331S;
(v) D265A;
(vi) G237A;
(vii) E318A;
(viii) E233P
(ix) G236R and L328R;
(x) L234A, L235A, G237A, P238S, H268A, A330S, and P331S; and
(xi) N297G or N297Q,
in which the amino acid positions are numbered according to the EU index.

In some embodiments, the Fc domain is a human IgG4 Fc domain. In some embodiments, the one or more amino acid modifications are one or more amino acid substitutions selected from the group consisting of:
(i) L235E;
(ii) D265A;
(iii) G237A;
(iv) E318A;
(v) E233P
(vi) G236R and L328R; and
(vii) N297G or N297Q,
in which the amino acid positions are numbered according to the EU index.

In some embodiments, the modified antibody or antigen-binding fragment thereof comprises a human IgG4 hinge region having the amino acid substitution S228P (S241P according to Kabat). In some embodiments, the one or more amino acid modifications are one or more amino acid substitutions selected from the group consisting of:
(i) L235E; and
(ii) F234A and L235A,
in which the amino acid positions are numbered according to the EU index.

In some embodiments, the modified antibody or antigen-binding fragment thereof is an IgG4 antibody or antigen-binding fragment thereof. In some embodiments, the modified antibody or antigen-binding fragment thereof comprises the amino acid substitution S228P.

In some embodiments, the modified antibody or antigen-binding fragment thereof does not comprise any one of the amino acid substitutions set forth in Table 18, in which the amino acid positions are numbered according to the EU index.

In some embodiments, the one or more amino acid modifications are one or more amino acid deletions. In some embodiments, the one or more amino acid deletions are deletions of all amino acid residues of the Fc domain. In some embodiments, the modified antibody or antigen-binding fragment thereof lacks an Fc domain. In some embodiments, the modified antibody or antigen-binding fragment thereof is selected from the group consisting of a single-chain Fv molecule (scFv), a diabody, a triabody, an antibody-like protein scaffold, a Fv fragment, a Fab fragment, a F(ab′)₂ molecule, and a tandem scFv (taFv).

In some embodiments, the modified antibody or antigen-binding fragment thereof is conjugated to a therapeutic agent. In some embodiments, the therapeutic agent is selected from the group consisting of a chemotherapy agent, an immunotherapy agent, an agonist of a TNFRSF member protein, an antagonist of a TNFRSF member protein, an agonist of a TNFSF member protein, an antagonist of a TNFSF member protein, an agonist of CD28, an antagonist of CD28, an agonist of ICOS, and an antagonist of ICOS.

In some embodiments, the method comprises administering an additional therapeutic agent. In some embodiments, the additional therapeutic agent is selected from the group consisting of a chemotherapy agent, an immunotherapy agent, an agonist of a TNFRSF member protein, an antagonist of a TNFRSF member protein, an agonist of a TNFSF member protein, an antagonist of a TNFSF member protein, an agonist of CD28, an antagonist of CD28, an agonist of ICOS, and an antagonist of ICOS.

In some embodiments, the modified antibody or antigen-binding fragment thereof is administered to the human subject intravenously or subcutaneously. In some embodiments, the modified antibody or antigen-binding fragment thereof is administered to the human subject subcutaneously.

In some embodiments, the method inhibits an immune response mediated by a B cell, a CD8+ T cell, or a T-reg cell in the human subject.

In some embodiments, the method promotes an immune response mediated by a B cell, a CD8+ T cell, or a T-reg cell in the human subject.

In some embodiments, the human subject is in need of a tissue or organ regeneration. In some embodiments, the tissue or organ is selected from the group consisting of a pancreas, salivary gland, pituitary gland, kidney, heart, lung, hematopoietic system, cranial nerves, heart, aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes.

In some embodiments, the method is for treating a cell proliferation disorder in the human subject. In some embodiments, the cell proliferation disorder is a cancer selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer. In some embodiments, the cell proliferation disorder is cancer selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary central nervous system (CNS) lymphoma, anal cancer, appendix cancer, astrocytoma, atypical teratoid/rhabdoid tumor, basal cell carcinoma, bile duct cancer, extrahepatic cancer, Ewing sarcoma family, osteosarcoma and malignant fibrous histiocytoma, central nervous system embryonal tumors, central nervous system germ cell tumors, craniopharyngioma, ependymoma, bronchial tumors, Burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, Langerhans cell histiocytosis, Hodgkin lymphoma, hypopharyngeal cancer, islet cell tumors, pancreatic neuroendocrine tumors, Wilms tumor and other childhood kidney tumors, Langerhans cell histiocytosis, small cell lung cancer, cutaneous T-cell lymphoma, intraocular melanoma, Merkel cell carcinoma, mesothelioma, metastatic squamous neck cancer, midline tract carcinoma, multiple endocrine neoplasia syndromes, multiple myeloma/plasma cell neoplasm (e.g., multiple myeloma or refractory multiple myeloma), myelodysplastic syndromes, nasal cavity and paranasal sinus cancer, nasopharyngeal cancer, neuroblastoma, non-Hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi sarcoma, rhabdomyosarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia. In some embodiments, the modified antibody or antigen-binding fragment thereof is an agonistic antibody or antigen-binding fragment thereof that specifically binds a TNFRSF member protein selected from the group consisting of TRAIL-R1 (TNFRSF10A), TRAIL-R2 (TNFRSF10B), TNFR1, Fas, CD40, CD27, 4-1BB, OX40, GITR, and XEDAR. In some embodiments, the modified antibody or antigen-binding fragment thereof is an antagonistic antibody or antigen-binding fragment thereof that specifically binds a TNFRSF member protein selected from the group consisting of TRAMP, NGFR, TRAIL-R4, TNFR2, HVEM, CD30, TROY, and RELT. In some embodiments, the modified antibody or antigen-binding fragment thereof is an antagonistic antibody or antigen-binding fragment thereof that specifically binds TNFR2 comprising a CDR-H1, a CDR-H2, a CDR-H3, a CDR-L1, a CDR-L2, and a CDR-L3 set forth in Table 4. In some embodiments, the modified antibody or antigen-binding fragment thereof is an antagonistic antibody or antigen-binding fragment thereof that specifically binds TNFR2 comprising a VH and a VL having amino acid sequences that are at least 80% identical to the sequences set forth in Table 5.

In some embodiments, the method is for treating an autoimmune disease or an inflammatory disease in the human subject. In some embodiments, the autoimmune disease is selected from the group consisting of Type I diabetes, alopecia areata, ankylosing spondylitis, antiphospholipid syndrome, autoimmune Addison's disease, autoimmune hemolytic anemia, autoimmune hepatitis, Behçet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, limited scleroderma (CREST syndrome), cold agglutinin disease, Crohn's disease, discoid lupus, essential mixed cryoglobulinemia, fibromyalgia-fibromyositis, Graves' disease, Guillain-Barre syndrome, Hashimoto's thyroiditis, hypothyroidism, inflammatory bowel disease, autoimmune lymphoproliferative syndrome (ALPS), idiopathic pulmonary fibrosis, idiopathic thrombocytopenia purpura (ITP), IgA nephropathy, juvenile arthritis, lichen planus, lupus, lupus nephritis, Ménière's disease, mixed connective tissue disease, multiple sclerosis, myasthenia gravis, Pemphigus vulgaris, Pemphigus foliaceus, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Takayasu's arteritis, temporal arteritis/giant cell arteritis, ulcerative colitis, uveitis, vasculitis, vitiligo, Wegener's granulomatosis, Goodpasture syndrome, transplant rejection, celiac disease, esophagitis, and inflammatory central nervous system disorders. In some embodiments, the inflammatory central nervous system disorder is Alzheimer's disease.

In some embodiments, the method is for treating an infectious disease in the human subject. In some embodiments, the infectious disease is caused by one or more agents selected from the group consisting of a virus, a bacterium, a fungus, and a parasite. In some embodiments, the infectious disease is caused by a virus selected from the group consisting of hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi's sarcoma associated-herpesvirus (KSHV), influenza virus, severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, mumps virus, poliovirus, human enterovirus (e.g., A, B, C, and D), hepatitis A virus, coxsackievirus, hepatitis B virus, human papilloma virus, adeno-associated virus, astrovirus, JC virus, BK virus, SV40 virus, Norwalk virus, rotavirus, human immunodeficiency virus (HIV), and human T-lymphotropic virus Types I and II. In some embodiments, the infectious disease is caused by a bacterium belonging to a genus selected from the group consisting of Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus. In some embodiments, the infectious disease is caused by a fungus belonging to a genus selected from the group consisting of Aspergillus, Candida, Malassezia, Trichosporon, Fusarium, Acremonium, Rhizopus, Mucor, Pneumocystis, and Absidia. In some embodiments, the infectious disease is caused by a parasite selected from the group consisting of Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. Exemplary helminthic parasites include Richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus.

In some embodiments, the method is for treating an inflammatory disease in the human subject. In some embodiments, the inflammatory disease is selected from the group consisting of acute or chronic inflammation, cardiac fibrosis, lung fibrosis, osteoarthritis, rheumatoid arthritis, atherosclerosis, type I diabetes, type II diabetes, graft-versus-host disease, multiple sclerosis, osteomyelitis, psoriasis, Crohn's disease, Sjögren's syndrome, lupus erythematosus, and ulcerative colitis.

In some embodiments, the method is for treating a neurological disease or disorder in the human subject. In some embodiments, the neurological disease or disorder is selected from the group consisting of a brain tumor, a brain metastasis, a brain injury, a spinal cord injury, a nerve injury, schizophrenia, epilepsy, Parkinson's disease, autism, Huntington's disease, stroke, Alzheimer's disease, multiple sclerosis, amyotrophic lateral sclerosis (ALS), and myasthenia gravis.

In some embodiments, the method is for treating an allergy in the human subject. In some embodiments, the allergy is selected from the group consisting of food allergy, seasonal allergy, pet allergy, hives, hay fever, allergic conjunctivitis, poison ivy allergy oak allergy, mold allergy, drug allergy, dust allergy, cosmetic allergy, and chemical allergy.

In some embodiments, the method is for treating a transplant rejection in the human subject. In some embodiments, the transplant rejection is an allograft rejection. In some embodiments, the transplant rejection is selected from the group consisting of skin graft rejection, bone graft rejection, vascular tissue graft rejection, ligament graft rejection, and organ graft rejection. In some embodiments, the ligament graft rejection is selected from the group consisting of cricothyroid ligament graft rejection, periodontal ligament graft rejection, suspensory ligament of the lens graft rejection, palmar radiocarpal ligament graft rejection, dorsal radiocarpal ligament graft rejection, ulnar collateral ligament graft rejection, radial collateral ligament graft rejection, suspensory ligament of the breast graft rejection, anterior sacroiliac ligament graft rejection, posterior sacroiliac ligament graft rejection, sacrotuberous ligament graft rejection, sacrospinous ligament graft rejection, inferior pubic ligament graft rejection, superior pubic ligament graft rejection, anterior cruciate ligament graft rejection, lateral collateral ligament graft rejection, posterior cruciate ligament graft rejection, medial collateral ligament graft rejection, cranial cruciate ligament graft rejection, caudal cruciate ligament graft rejection, and patellar ligament graft rejection. In some embodiments, the organ graft rejection is selected from the group consisting of heart graft rejection, lung graft rejection, kidney graft rejection, liver graft rejection, pancreas graft rejection, intestine graft rejection, and thymus graft rejection.

In some embodiments, the method is for treating a graft-versus-host disease in the human subject. In some embodiments, the graft-versus-host disease arises from a bone marrow transplant, or one or more blood cells selected from the group consisting of hematopoietic stem cells, common myeloid progenitor cells, common lymphoid progenitor cells, megakaryocytes, monocytes, basophils, eosinophils, neutrophils, macrophages, T cells, B cells, natural killer cells, and dendritic cells.

In some embodiments, the method is for regenerating a tissue or organ in the human subject. In some embodiments, the tissue or organ is selected from the group consisting of pancreas, salivary gland, pituitary gland, kidney, heart, lung, hematopoietic system, cranial nerves, heart, aorta, olfactory gland, ear, nerve, eye, thymus, tongue, bone, liver, small intestine, large intestine, gastrointestinal, lung, brain, skin, peripheral nervous system, central nervous system, spinal cord, breast, embryonic structures, embryo, and testes. In some embodiments, the tissue or organ expresses the TNFRSF member protein, the TNFSF member protein, CD28, or ICOS.

In a ninth aspect, the disclosure features an antibody or antigen-binding fragment thereof that specifically binds to a human TNFRSF member protein, in which the antibody or antigen-binding fragment thereof comprises at least 50 amino acid residues of an Fc domain and one or more amino acid substitutions within the Fc domain, further in which:
(a) the Fc domain is a human IgG1 Fc domain, and the one or more amino acid substitutions are selected from the group consisting of:
   (i) L235E;
   (ii) L234A and L235A;
   (iii) L234A, L235A, and P329G
   (iv) L234E, L235F, and P331S;
   (v) D265A;
   (vi) G237A;
   (vii) E318A;
   (viii) E233P
   (ix) G236R and L328R;
   (x) L234A, L235A, G237A, P238S, H268A, A330S, and P331S; and
   (xi) N297G or N297Q;
(b) the Fc domain is a human IgG2 Fc domain, and the one or more amino acid substitutions are selected from the group consisting of:
   (i) D265A;
   (ii) G237A;
   (iii) E318A;
   (iv) H268Q, V309L, A330S, and P331S;
   (v) V234A, G237A, P238S, H268A, V309L, A330S, and P331S; and
   (vi) N297G or N297Q;
(c) the Fc domain is a human IgG3 Fc domain, and the one or more amino acid substitutions are selected from the group consisting of:
   (i) L235E;
   (ii) L234A and L235A;
   (iii) L234A, L235A, and P329G
   (iv) L234E, L235F, and P331S;
   (v) D265A;
   (vi) G237A;
   (vii) E318A;
   (viii) E233P
   (ix) G236R and L328R;
   (x) L234A, L235A, G237A, P238S, H268A, A330S, and P331S; and
   (xi) N297G or N297Q;
(d) the Fc domain is a human IgG4 Fc domain, and the one or more amino acid substitutions are selected from the group consisting of:
   (i) L235E;
   (ii) D265A;
   (iii) G237A;
   (iv) E318A;
   (v) E233P
   (vi) G236R and L328R; and
   (vii) N297G or N297Q; or
(e) the antibody or antigen-binding fragment thereof comprises a human IgG4 hinge region having the amino acid substitution S228P (S241P according to Kabat), the Fc domain is a human IgG4 Fc domain, and, optionally, the one or more amino acid substitutions are selected from the group consisting of:
   (i) L235E; and
   (ii) F234A and L235A,
and in which the amino acid positions are numbered according to the EU index.

In some embodiments, the antibody or antigen-binding fragment thereof is an IgG4 antibody or antigen-binding fragment thereof. In some embodiments, the modified antibody or antigen-binding fragment thereof comprises the amino acid substitution S228P.

In some embodiments, the TNFRSF member protein is selected from the group consisting of TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, and DCR3. In some embodiments, the TNFRSF member protein is TNFR2.

In some embodiments, the antibody or antigen-binding fragment thereof is an agonistic antibody or antigen-binding fragment thereof that specifically binds the TNFRSF member protein.

In some embodiments, the TNFRSF member protein is TNFR2. In some embodiments, the antibody or antigen-binding fragment thereof comprises a CDR-H1 comprising the amino acid sequence of X¹X²X²X¹X²X³X²JJJ, in which each X¹ is independently G, A, V, L, I, M, W, F, or P; each X² is independently Y, S, T, C, N, or Q; X³ is D or E; and each J is independently a naturally occurring amino acid or is absent. In some embodiments, the CDR-H1 comprises the amino acid sequence of GYTFTZ¹Z²JJJ (SEQ ID NO: 1480), in which Z¹ is D or T; Z² is Y, F, or L; and each J is independently a naturally occurring amino acid or is absent. In some embodiments, the CDR-H1 comprises the amino acid sequence of GYTFTDY (SEQ ID NO: 1413). In some embodiments, the antibody or antigen-binding fragment thereof further comprises one or more of a CDR-H2, a CDR-H3, a CDR-L1, a CDR-L2, and a CDR-L3 set forth in Table 2.

In some embodiments, the antibody or antigen-binding fragment thereof comprises a CDR-H1 comprising the amino acid sequence of X¹X²X²X³X¹, in which each X¹ is independently D or E; each X² is independently Y, S, T, C, N, or Q; and X³ is L, A, V, G, I, M, W, F, or P. In some embodiments, the CDR-H1 comprises the amino acid sequence of DYNLD (SEQ ID NO: 1541). In some embodiments, the antibody or antigen-binding fragment thereof further comprises one or more of a CDR-H2, a CDR-H3, a CDR-L1, a CDR-L2, and a CDR-L3 set forth in Table 2.

In some embodiments, the modified antibody or antigen-binding fragment thereof comprises a CDR-H2 comprising the amino acid sequence of X¹X²X³X²X³X³X¹X³X³X³X³X³X³X⁴X²X⁴X², in which each X¹ is independently D or E; each X² is independently I, A, V, L, G, M, W, F, or P; each X³ is independently N, S, T, C, Y, or Q; and each X⁴ is independently K, R, or H. In some embodiments, the CDR-H2 comprises the amino acid sequence of DINPNYDSTSYSQKFRG (SEQ ID NO: 1542). In some embodiments, the antibody or antigen-binding fragment thereof further comprises one or more of a CDR-H1, a CDR-H3, a CDR-L1, a CDR-L2, and a CDR-L3 set forth in Table 2.

In some embodiments, the modified antibody or antigen-binding fragment thereof comprises a CDR-H3 comprising the amino acid sequence of X¹X²X²X¹X²X¹X³X¹, in which each X¹ is independently G, A, V, L, I, M, W, F, or P; each X² is independently N, S, T, C, Y, or Q; and X³ is D or E. In some embodiments, the CDR-H3 comprises the amino acid sequence of GNSWYFDV (SEQ ID NO: 1543). In some embodiments, the antibody or antigen-binding fragment thereof further comprises one or more of a CDR-H1, a CDR-H2, a CDR-L1, a CDR-L2, and a CDR-L3 set forth in Table 2.

In some embodiments, the modified antibody or antigen-binding fragment thereof comprises a CDR-L1 comprising the amino acid sequence of X¹X²X¹X¹X¹X²X³X⁴X¹X¹, in which each X¹ is independently S, Y, T, C, N, or Q; each X² is independently A, G, V, L, I, M, W, F, or P; X³ is R, H, or K; and X⁴ is Y, A, V, I, L, M, F, or W. In some embodiments, the CDR-L1 comprises the amino acid sequence of SASSSVRYNY (SEQ ID NO: 1544). In some embodiments, the antibody or antigen-binding fragment thereof further comprises one or more of a CDR-H1, a CDR-H2, a CDR-H3, a CDR-L2, and a CDR-L3 set forth in Table 2.

In some embodiments, the modified antibody or antigen-binding fragment thereof comprises a CDR-L2 comprising the amino acid sequence of X¹X²X²X²X¹X¹X², in which each X¹ is independently L, A, V, G, I, M, W, F, or P; and each X² is independently T, S, C, Y, N, or Q. In some embodiments, the CDR-L2 comprises the amino acid sequence of LTSNLAS (SEQ ID NO: 1545). In some embodiments, the antibody or antigen-binding fragment thereof further comprises one or more of a CDR-H1, a CDR-H2, a CDR-H3, a CDR-L1, and a CDR-L3 set forth in Table 2.

In some embodiments, the modified antibody or antigen-binding fragment thereof comprises a CDR-L3 comprising the amino acid sequence of X¹X²X²X¹X²X²X²X¹X¹X², in which each X¹ is independently P, A, V, L, I, M, W, F, or G; and each X² is independently Q, S, T, C, N, or Y. In some embodiments, the CDR-L3 comprises the amino acid sequence of PQQWSSNPLT (SEQ ID NO: 1546). In some embodiments, the antibody or antigen-binding fragment thereof further comprises one or more of a CDR-H1, a CDR-H2, a CDR-H3, a CDR-L1, and a CDR-L2 set forth in Table 2.

In some embodiments, the antibody or antigen-binding fragment thereof comprises a CDR-H1, a CDR-H2, a CDR-H3, a CDR-L1, a CDR-L2, and a CDR-L3 set forth in Table 2. In some embodiments, the antibody or antigen-binding fragment thereof comprises a VH and a VL having amino acid sequences that are at least 80% identical (e.g., 85%, 90%, 95%, 97%, 99%, or 100% identical) to the sequences set forth in Table 3.

In some embodiments, the TNFRSF member protein is selected from the group consisting of CD27, CD40, GITR, OX40, and 4-1BB. In some embodiments, the antibody or antigen-binding fragment thereof comprises the CDR-H1, the CDR-H2, the CDR-H3, the CDR-L1, the CDR-L2, and the CDR-L3 of an antibody selected from the group consisting of varlilumab (CDX-1127), CDX-1140, SEA-CD40, RO7009789, JNJ-64457107 (ADC1013), APX-005M, Chi Lob 7/4, TRX-518, MK-4166, MK-1248, GWN-323, INCAGN01876, BMS-986156, AMG-228, tavolimab (MEDI0562), PF-04518600, BMS-986178, MOXR-0916, GSK-3174998, INCAGN01949, utomilumab (PF-05082566), and urelumab (BMS-663513). In some embodiments, the antibody or antigen-binding fragment thereof comprises a VH and a VL having amino acid sequences that are at least 80% identical to the VH and the VL sequences of an antibody selected from the group consisting of varlilumab (CDX-1127), CDX-1140, SEA-CD40, RO7009789, JNJ-64457107 (ADC1013), APX-005M, Chi Lob 7/4, TRX-518, MK-4166, MK-1248, GWN-323, INCAGN01876, BMS-986156, AMG-228, tavolimab (MEDI0562), PF-04518600, BMS-986178, MOXR-0916, GSK-3174998, INCAGN01949, utomilumab (PF-05082566), and urelumab (BMS-663513).

In some embodiments, the antibody or antigen-binding fragment thereof is an antagonistic antibody or antigen-binding fragment thereof that specifically binds the TNFRSF member protein.

In some embodiments, the TNFRSF member protein is TNFR2. In some embodiments, the antibody or antigen-binding fragment thereof comprises a CDR-H1, a CDR-H2, a CDR-H3, a CDR-L1, a CDR-L2, and a CDR-L3 set forth in Table 4. In some embodiments, the antibody or antigen-binding fragment thereof comprises a VH and a VL having amino acid sequences that are at least 80% identical (e.g., 85%, 90%, 95%, 97%, 99%, or 100% identical) to the sequences set forth in Table 5.

In some embodiments, the antibody or antigen-binding fragment thereof is neither an agonist nor an antagonist of the TNFRSF member protein. In some embodiments, the TNFRSF member protein is TNFR2. In some embodiments, the antibody or antigen-binding fragment thereof comprises a CDR-H1, a CDR-H2, a CDR-H3, a CDR-L1, a CDR-L2, and a CDR-L3 set forth in Table 6. In some embodiments, the antibody or antigen-binding fragment thereof comprises a VH and a VL having amino acid sequences that are at least 80% identical (e.g., 85%, 90%, 95%, 97%, 99%, or 100% identical) to the sequences set forth in Table 7.

In some embodiments, the antibody or antigen-binding fragment thereof has less than 90% (e.g., less than 80%, less than 70%, less than 60%, less than 50%, less than 40%, less than 30%, less than 20%, or less than 10%) cross-reactivity with the soluble TNFRSF member protein. In some embodiments, the antibody or antigen-binding fragment thereof has less than 50% cross-reactivity with the soluble TNFRSF member protein.

In a tenth aspect, the disclosure features an agonistic antibody or antigen-binding fragment thereof that specifically binds human TNFR2, comprising:
(A) means for binding an epitope within human TNFR2, comprising:
   (a) a CDR-H1 comprising the amino acid sequence of:
      (i) X¹X²X²X¹X²X³X²JJJ, in which each X¹ is independently G, A, V, L, I, M, W, F, or P; each X² is independently Y, S, T, C, N, or Q; X³ is D or E; and each J is independently a naturally occurring amino acid or is absent; or
      (ii) X¹X²X²X³X¹, in which each X¹ is independently D or E; each X² is independently Y, S, T, C, N, or Q; and X³ is L, A, V, G, I, M, W, F, or P;
   (b) a CDR-H2 comprising the amino acid sequence of X¹X²X³X²X³X³X¹X³X³X³X³X³X³X⁴X²X⁴X², in which each X¹ is independently D or E; each X² is independently I, A, V, L, G, M, W, F, or P; each X³ is independently N, S, T, C, Y, or Q; and each X⁴ is independently K, R, or H;
   (c) a CDR-H3 comprising the amino acid sequence of X¹X²X²X¹X²X¹X³X¹, in which each X¹ is independently G, A, V, L, I, M, W, F, or P; each X² is independently N, S, T, C, Y, or Q; and X³ is D or E;
   (d) a CDR-L1 comprising the amino acid sequence of X¹X²X¹X¹X¹X²X³X⁴X¹X¹, in which each X¹ is independently S, Y, T, C, N, or Q; each X² is independently A, G, V, L, I, M, W, F, or P; X³ is R, H, or K; and X⁴ is Y, A, V, I, L, M, F, or W;
   (e) a CDR-L2 comprising the amino acid sequence of X¹X²X²X²X¹X¹X², in which each X¹ is independently L, A, V, G, I, M, W, F, or P; and each X² is independently T, S, C, Y, N, or Q; and/or
   (f) a CDR-L3 comprising the amino acid sequence of X¹X²X²X¹X²X²X²X¹X¹X², in which each X¹ is independently P, A, V, L, I, M, W, F, or G; and each X² is independently Q, S, T, C, N, or Y; and
(B) an Fc domain comprising one or more amino acid substitutions, in which:
   (a) the Fc domain is a human IgG1 Fc domain, and the one or more amino acid substitutions are selected from the group consisting of:
      (i) L235E;
      (ii) L234A and L235A;
      (iii) L234A, L235A, and P329G
      (iv) L234E, L235F, and P331S;
      (v) D265A;
      (vi) G237A;
      (vii) E318A;
      (viii) E233P
      (ix) G236R and L328R;
      (x) L234A, L235A, G237A, P238S, H268A, A330S, and P331S; and
      (xi) N297G or N297Q;
   (b) the Fc domain is a human IgG2 Fc domain, and the one or more amino acid substitutions are selected from the group consisting of:
      (i) D265A;
      (ii) G237A;
      (iii) E318A;
      (iv) H268Q, V309L, A330S, and P331S;
      (v) V234A, G237A, P238S, H268A, V309L, A330S, and P331S; and
      (vi) N297G or N297Q;
   (c) the Fc domain is a human IgG3 Fc domain, and the one or more amino acid substitutions are selected from the group consisting of:
      (i) L235E;
      (ii) L234A and L235A;
      (iii) L234A, L235A, and P329G
      (iv) L234E, L235F, and P331S;
      (v) D265A;
      (vi) G237A;
      (vii) E318A;
      (viii) E233P
      (ix) G236R and L328R;
      (x) L234A, L235A, G237A, P238S, H268A, A330S, and P331S; and
      (xi) N297G or N297Q;
   (d) the Fc domain is a human IgG4 Fc domain, and the one or more amino acid substitutions are selected from the group consisting of:
      (i) L235E;
      (ii) D265A;
      (iii) G237A;
      (iv) E318A;
      (v) E233P
      (vi) G236R and L328R; and
      (vii) N297G or N297Q; or
   (e) the antibody or antigen-binding fragment thereof comprises a human IgG4 hinge region having the amino acid substitution S228P (S241P according to Kabat), the Fc domain is a human IgG4 Fc domain, and, optionally, the one or more amino acid substitutions are selected from the group consisting of:
      (i) L235E; and
      (ii) F234A and L235A,
   and in which the amino acid positions are numbered according to the EU index.

In some embodiments, the antibody or antigen-binding fragment thereof is an IgG4 antibody or antigen-binding fragment thereof. In some embodiments, the modified antibody or antigen-binding fragment thereof comprises the amino acid substitution S228P.

In some embodiments, the CDR-H1 comprises the amino acid sequence of GYTFTZ¹Z²JJJ (SEQ ID NO: 1480), in which Z¹ is D or T; Z² is Y, F, or L; and each J is independently a naturally occurring amino acid or is absent. In some embodiments, the CDR-H1 comprises the amino acid sequence of GYTFTDY (SEQ ID NO: 1413). In some embodiments, the antibody or antigen-binding fragment thereof further comprises one or more of a CDR-H2, a CDR-H3, a CDR-L1, a CDR-L2, and a CDR-L3 set forth in Table 2.

In an eleventh aspect, the disclosure features an antibody or antigen-binding fragment thereof that specifically binds to a human TNFSF member protein, in which the antibody or antigen-binding fragment thereof comprises at least 50 amino acid residues of an Fc domain and one or more amino acid substitutions within the Fc domain, further in which:
(a) the Fc domain is a human IgG1 Fc domain, and the one or more amino acid substitutions are selected from the group consisting of:
   (i) L235E;
   (ii) L234A and L235A;
   (iii) L234A, L235A, and P329G
   (iv) L234E, L235F, and P331S;
   (v) D265A;
   (vi) G237A;
   (vii) E318A;
   (viii) E233P
   (ix) G236R and L328R;
   (x) L234A, L235A, G237A, P238S, H268A, A330S, and P331S; and
   (xi) N297G or N297Q;
(b) the Fc domain is a human IgG2 Fc domain, and the one or more amino acid substitutions are selected from the group consisting of:
   (i) D265A;
   (ii) G237A;
   (iii) E318A;
   (iv) H268Q, V309L, A330S, and P331S;
   (v) V234A, G237A, P238S, H268A, V309L, A330S, and P331S; and
   (vi) N297G or N297Q;
(c) the Fc domain is a human IgG3 Fc domain, and the one or more amino acid substitutions are selected from the group consisting of:
   (i) L235E;
   (ii) L234A and L235A;
   (iii) L234A, L235A, and P329G
   (iv) L234E, L235F, and P331S;
   (v) D265A;
   (vi) G237A;
   (vii) E318A;
   (viii E233P
   (ix) G236R and L328R;
   (x) L234A, L235A, G237A, P238S, H268A, A330S, and P331S; and
   (xi) N297G or N297Q;
(d) the Fc domain is a human IgG4 Fc domain, and the one or more amino acid substitutions are selected from the group consisting of:
   (i) L235E;
   (ii) D265A;
   (iii) G237A;
   (iv) E318A;
   (v) E233P
   (vi) G236R and L328R; and
   (vii) N297G or N297Q; or
(e) the antibody or antigen-binding fragment thereof comprises a human IgG4 hinge region having the amino acid substitution S228P (S241P according to Kabat), the Fc domain is a human IgG4 Fc domain, and, optionally, the one or more amino acid substitutions are selected from the group consisting of:
   (i) L235E; and
   (ii) F234A and L235A,
and in which the amino acid positions are numbered according to the EU index.

In some embodiments, the antibody or antigen-binding fragment thereof is an IgG4 antibody or antigen-binding fragment thereof. In some embodiments, the modified antibody or antigen-binding fragment thereof comprises the amino acid substitution S228P.

In some embodiments, the TNFSF member protein is TRAIL.

In a twelfth aspect, the disclosure features an antibody or antigen-binding fragment thereof that specifically binds to human CD28, in which the antibody or antigen-binding fragment thereof comprises at least 50 amino acid residues of an Fc domain and one or more amino acid substitutions within the Fc domain, further in which:
(a) the Fc domain is a human IgG1 Fc domain, and the one or more amino acid substitutions are selected from the group consisting of:
   (i) L235E;
   (ii) L234A and L235A;
   (iii) L234A, L235A, and P329G
   (iv) L234E, L235F, and P331S;
   (v) D265A;
   (vi) G237A;
   (vii) E318A;
   (viii) E233P
   (ix) G236R and L328R;
   (x) L234A, L235A, G237A, P238S, H268A, A330S, and P331S; and
   (xi) N297G or N297Q;
(b) the Fc domain is a human IgG2 Fc domain, and the one or more amino acid substitutions are selected from the group consisting of:
   (i) D265A;
   (ii) G237A;
   (iii) E318A;
   (iv) H268Q, V309L, A330S, and P331S;
   (v) V234A, G237A, P238S, H268A, V309L, A330S, and P331S; and
   (vi) N297G or N297Q;
(c) the Fc domain is a human IgG3 Fc domain, and the one or more amino acid substitutions are selected from the group consisting of:
   (i) L235E;
   (ii) L234A and L235A;
   (iii) L234A, L235A, and P329G
   (iv) L234E, L235F, and P331S;
   (v) D265A;
   (vi) G237A;
   (vii) E318A;
   (viii E233P
   (ix) G236R and L328R;
   (x) L234A, L235A, G237A, P238S, H268A, A330S, and P331S; and
   (xi) N297G or N297Q;
(d) the Fc domain is a human IgG4 Fc domain, and the one or more amino acid substitutions are selected from the group consisting of:
   (i) L235E;
   (ii) D265A;
   (iii) G237A;
   (iv) E318A;
   (v) E233P
   (vi) G236R and L328R; and
   (vii) N297G or N297Q; or
(e) the antibody or antigen-binding fragment thereof comprises a human IgG4 hinge region having the amino acid substitution S228P (S241P according to Kabat), the Fc domain is a human IgG4 Fc domain, and, optionally, the one or more amino acid substitutions are selected from the group consisting of:
   (i) L235E; and
   (ii) F234A and L235A,
and in which the amino acid positions are numbered according to the EU index.

In some embodiments, the antibody or antigen-binding fragment thereof is an IgG4 antibody or antigen-binding fragment thereof. In some embodiments, the modified antibody or antigen-binding fragment thereof comprises the amino acid substitution S228P.

In some embodiments, the antibody or antigen-binding fragment thereof is an agonistic antibody or antigen-binding fragment thereof that specifically binds CD28. In some embodiments, the antibody or antigen-binding fragment thereof comprises the CDR-H1, the CDR-H2, the CDR-H3, the CDR-L1, the CDR-L2, and the CDR-L3 of theralizumab (TAB-08). In some embodiments, the antibody or antigen-binding fragment thereof comprises a VH and a VL having amino acid sequences that are at least 80% identical (e.g., 85%, 90%, 95%, 97%, 99%, or 100% identical) to the VH and the VL sequences of theralizumab (TAB-08).

In a thirteenth aspect, the disclosure features an antibody or antigen-binding fragment thereof that specifically binds to human ICOS, in which the antibody or antigen-binding fragment thereof comprises at least 50 amino acid residues of an Fc domain and one or more amino acid substitutions within the Fc domain, further in which:
(a) the Fc domain is a human IgG1 Fc domain, and the one or more amino acid substitutions are selected from the group consisting of:
   (i) L235E;
   (ii) L234A and L235A;
   (iii) L234A, L235A, and P329G
   (iv) L234E, L235F, and P331S;
   (v) D265A;
   (vi) G237A;
   (vii) E318A;
   (viii) E233P
   (ix) G236R and L328R;
   (x) L234A, L235A, G237A, P238S, H268A, A330S, and P331S; and
   (xi) N297G or N297Q;
(b) the Fc domain is a human IgG2 Fc domain, and the one or more amino acid substitutions are selected from the group consisting of:
   (i) D265A;
   (ii) G237A;
   (iii) E318A;
   (iv) H268Q, V309L, A330S, and P331S;
   (v) V234A, G237A, P238S, H268A, V309L, A330S, and P331S; and
   (vi) N297G or N297Q;
(c) the Fc domain is a human IgG3 Fc domain, and the one or more amino acid substitutions are selected from the group consisting of:
   (i) L235E;
   (ii) L234A and L235A;
   (iii) L234A, L235A, and P329G
   (iv) L234E, L235F, and P331S;
   (v) D265A;
   (vi) G237A;
   (vii) E318A;
   (viii) E233P
   (ix) G236R and L328R;
   (x) L234A, L235A, G237A, P238S, H268A, A330S, and P331S; and
   (xi) N297G or N297Q;
(d) the Fc domain is a human IgG4 Fc domain, and the one or more amino acid substitutions are selected from the group consisting of:
   (i) L235E;
   (ii) D265A;
   (iii) G237A;
   (iv) E318A;
   (v) E233P
   (vi) G236R and L328R; and
   (vii) N297G or N297Q; or
(e) the antibody or antigen-binding fragment thereof comprises a human IgG4 hinge region having the amino acid substitution S228P (S241P according to Kabat), the Fc domain is a human IgG4 Fc domain, and, optionally, the one or more amino acid substitutions are selected from the group consisting of:
   (i) L235E; and
   (ii) F234A and L235A,
and in which the amino acid positions are numbered according to the EU index.

In some embodiments, the antibody or antigen-binding fragment thereof is an IgG4 antibody or antigen-binding fragment thereof. In some embodiments, the modified antibody or antigen-binding fragment thereof comprises the amino acid substitution S228P.

In some embodiments, the antibody or antigen-binding fragment thereof is an agonistic antibody or antigen-binding fragment thereof that specifically binds ICOS. In some embodiments, the antibody or antigen-binding fragment thereof comprises the CDR-H1, the CDR-H2, the CDR-H3, the CDR-L1, the CDR-L2, and the CDR-L3 of GSK-3359609 or JTX-2011. In some embodiments, the antibody or antigen-binding fragment thereof comprises a VH and a VL having amino acid sequences that are at least 80% identical (e.g., 85%, 90%, 95%, 97%, 99%, or 100% identical) to the VH and the VL sequences of GSK-3359609 or JTX-2011.

In some embodiments, the antibody or antigen-binding fragment thereof does not comprise any one of the amino acid substitutions set forth in Table 18, and in which the amino acid positions are numbered according to the EU index.

In some embodiments, the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, and a chimeric antibody or antigen-binding fragment thereof. In some embodiments, the antibody or antigen-binding fragment thereof is a human, humanized, or chimeric antibody or antigen-binding fragment thereof.

In a fourteenth aspect, the disclosure features an antibody or antigen-binding fragment thereof that specifically binds to a human TNFRSF member protein, a human TNFSF member protein, human CD28, or human ICOS, in which the antibody or antigen-binding fragment thereof does not comprise an Fc domain.

In some embodiments, the TNFRSF member protein is selected from the group consisting of TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, and DCR3. In some embodiments, the TNFRSF member protein is TNFR2.

In some embodiments, the antibody or antigen-binding fragment thereof is an agonistic antibody or antigen-binding fragment thereof that specifically binds TNFR2. In some embodiments, the antibody or antigen-binding fragment thereof comprises a CDR-H1, a CDR-H2, a CDR-H3, a CDR-L1, a CDR-L2, and a CDR-L3 set forth in Table 2. In some embodiments, the antibody or antigen-binding fragment thereof comprises a VH and a VL having amino acid sequences that are at least 80% identical (e.g., 85%, 90%, 95%, 97%, 99%, or 100% identical) to the sequences set forth in Table 3.

In some embodiments, the antibody or antigen-binding fragment thereof is an antagonistic antibody or antigen-binding fragment thereof that specifically binds TNFR2. In some embodiments, the antibody or antigen-binding fragment thereof comprises a CDR-H1, a CDR-H2, a CDR-H3, a CDR-L1, a CDR-L2, and a CDR-L3 set forth in Table 4. In some embodiments, the antibody or antigen-binding fragment thereof comprises a VH and a VL having amino acid sequences that are at least 80% identical (e.g., 85%, 90%, 95%, 97%, 99%, or 100% identical) to the sequences set forth in Table 5.

In some embodiments, the TNFSF member protein is TRAIL.

In some embodiments, the antibody or antigen-binding fragment thereof is an agonistic antibody or antigen-binding fragment thereof that specifically binds CD28. In some embodiments, the antibody or antigen-binding fragment thereof comprises the CDR-H1, the CDR-H2, the CDR-H3, the CDR-L1, the CDR-L2, and the CDR-L3 of theralizumab (TAB-08). In some embodiments, the antibody or antigen-binding fragment thereof comprises a VH and a VL having amino acid sequences that are at least 80% identical (e.g., 85%, 90%, 95%, 97%, 99%, or 100% identical) to the VH and the VL sequences of theralizumab (TAB-08).

In some embodiments, the antibody or antigen-binding fragment thereof is an agonistic antibody or antigen-binding fragment thereof that specifically binds ICOS. In some embodiments, the antibody or antigen-binding fragment thereof comprises the CDR-H1, the CDR-H2, the CDR-H3, the CDR-L1, the CDR-L2, and the CDR-L3 of GSK-3359609 or JTX-2011. In some embodiments, the antibody or antigen-binding fragment thereof comprises a VH and a VL having amino acid sequences that are at least 80% identical (e.g., 85%, 90%, 95%, 97%, 99%, or 100% identical) to the VH and the VL sequences of GSK-3359609 or JTX-2011.

In some embodiments, the antibody or antigen-binding fragment thereof is selected from the group consisting of a single-chain Fv molecule (scFv), a diabody, a triabody, an antibody-like protein scaffold, a Fv fragment, a Fab fragment, a F(ab′)₂ molecule, and a tandem scFv (taFv).

In a fifteenth aspect, the disclosure features methods of producing the antibody or antigen-binding fragment thereof disclosed in the ninth, tenth, eleventh, twelfth, thirteenth, or fourteenth aspect of the disclosure, the method comprising expressing a polynucleotide encoding the antibody or antigen-binding fragment thereof in a host cell and recovering the antibody or antigen-binding fragment thereof from host cell medium.

In a sixteenth aspect, the disclosure features constructs comprising a first polypeptide domain and a second polypeptide domain, in which the first polypeptide domain and the second polypeptide domain are each, independently, an antigen-binding fragment disclosed in the ninth, tenth, eleventh, twelfth, thirteenth, or fourteenth aspect of the disclosure.

In some embodiments, the first polypeptide domain and the second polypeptide domain are bound by a covalent linker. In some embodiments, the covalent linker comprises an amide bond or a disulfide bond.

In a seventeenth aspect, the disclosure features polynucleotides encoding the antibody or antigen-binding fragment thereof disclosed in the ninth, tenth, eleventh, twelfth, thirteenth, or fourteenth aspect of the disclosure or the construct disclosed in the sixteenth aspect of the disclosure.

In an eighteenth aspect, the disclosure features vectors comprising the polynucleotide disclosed in the seventeenth aspect of the disclosure.

In some embodiments, the vector is an expression vector. In some embodiments, the expression vector is a eukaryotic expression vector.

In some embodiments, the vector is a viral vector. In some embodiments, the viral vector is selected from the group consisting of an adenovirus, retrovirus, poxvirus, adeno-associated virus, baculovirus, herpes simplex virus, and vaccinia virus. In some embodiments, the adenovirus is a serotype 1-60 adenovirus. In some embodiments, the adenovirus is a serotype 5, 26, 35, or 48 adenovirus. In some embodiments, the retrovirus is a γ-retrovirus or a lentivirus. In some embodiments, the vaccinia virus is a modified vaccinia Ankara (MVA).

In an nineteenth aspect, the disclosure features isolated host cells comprising the vector disclosed in the eighteenth aspect of the disclosure.

In some embodiments, the host cell is a prokaryotic cell or a eukaryotic cell. In some embodiments, the eukaryotic cell is a mammalian cell. In some embodiments, the mammalian cell is a CHO cell.

In a twentieth aspect, the disclosure features pharmaceutical compositions comprising the antibody or antigen-binding fragment thereof disclosed in the ninth, tenth, eleventh, twelfth, thirteenth, or fourteenth aspect of the disclosure, the construct disclosed in the sixteenth aspect of the disclosure, the polynucleotide disclosed in the seventeenth aspect of the disclosure, the vector disclosed in the eighteenth aspect of the disclosure, or the host cell disclosed in the nineteenth aspect of the disclosure, and a pharmaceutically acceptable carrier or excipient.

In a twenty-first aspect, the disclosure features methods of modulating an immune response in a human subject, the method comprising administering to the subject the antibody or antigen-binding fragment thereof disclosed in the ninth, tenth, eleventh, twelfth, thirteenth, or fourteenth aspect of the disclosure, the construct disclosed in the sixteenth aspect of the disclosure, the polynucleotide disclosed in the seventeenth aspect of the disclosure, the vector disclosed in the eighteenth aspect of the disclosure, the host cell disclosed in the nineteenth aspect of the disclosure, or the pharmaceutical composition disclosed in the twentieth aspect of the disclosure.

In some embodiments, the method is for treating a cell proliferation disorder, an autoimmune disease, an infectious disease, an inflammatory disease, a neurological disease, an allergy, a transplant rejection (e.g., an allograft rejection), or a graft-versus-host disease in the human subject. In some embodiments, the method is for regenerating a tissue or organ in the human subject.

In some embodiments, the cell proliferation disorder is a cancer. In some embodiments, the antibody or antigen-binding fragment thereof is an agonistic antibody or antigen-binding fragment thereof that specifically binds a TNFRSF member protein selected from the group consisting of TRAIL-R1 (TNFRSF10A), TRAIL-R2 (TNFRSF10B), TNFR1, Fas, CD40, CD27, 4-1BB, OX40, GITR, and XEDAR. In some embodiments, the antibody or antigen-binding fragment thereof is an antagonistic antibody or antigen-binding fragment thereof that specifically binds a TNFRSF member protein selected from the group consisting of TRAMP, NGFR, TRAIL-R4, TNFR2, HVEM, CD30, TROY, and RELT.

In some embodiments, the human subject has a serum or plasma level of a soluble TNFRSF member protein that is lower than or the same as a reference level of the soluble TNFRSF member protein, and the method comprising administering to the subject an agonistic antibody or antigen-binding fragment thereof that specifically binds the TNFRSF member protein. In some embodiments, the human subject has a serum or plasma level of a soluble TNFRSF member protein that is higher than a reference level of the soluble TNFRSF member protein, and the method comprising administering to the subject an antagonistic antibody or antigen-binding fragment thereof that specifically binds the TNFRSF member protein.

In some embodiments, the antibody or antigen-binding fragment thereof that specifically binds a TNFRSF member protein of the disclosure is administered to human subject at a low dose. In some instances, the low dose is a dose level that is lower than, e.g., a typical (or art-recognized) dose level of a corresponding antibody or antigen-binding fragment thereof. In some embodiments, the antibody or antigen-binding fragment thereof is administered once every week at a dose of from about 0.8 mg/kg to about 1.2 mg/kg (e.g., about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, or about 1.2 mg/kg). In some embodiments, the antibody or antigen-binding fragment thereof is administered once every two weeks at a dose of from about 1.65 mg/kg to about 2.5 mg/kg (e.g., about 1.65 mg/kg, about 1.7 mg/kg, about 1.75 mg/kg, about 1.8 mg/kg, about 1.85 mg/kg, about 1.9 mg/kg, about 1.95 mg/kg, about 2.0 mg/kg, about 2.05 mg/kg, about 2.1 mg/kg, about 2.15 mg/kg, about 2.2 mg/kg, about 2.25 mg/kg, about 2.3 mg/kg, about 2.35 mg/kg, about 2.4 mg/kg, about 2.45 mg/kg, or about 2.5 mg/kg). In some embodiments, the antibody or antigen-binding fragment thereof is administered once every three weeks at a dose of from about 2.2 mg/kg to about 3.0 mg/kg (e.g., about 2.2 mg/kg, about 2.25 mg/kg, about 2.3 mg/kg, about 2.35 mg/kg, about 2.4 mg/kg, about 2.45 mg/kg, about 2.5 mg/kg, about 2.55 mg/kg, about 2.6 mg/kg, about 2.65 mg/kg, about 2.7 mg/kg, about 2.75 mg/kg, about 2.8 mg/kg, about 2.85 mg/kg, about 2.9 mg/kg, about 2.95 mg/kg, or about 3.0 mg/kg). In some embodiments, the antibody or antigen-binding fragment thereof is administered once every four weeks at a dose of from about 2.5 mg/kg to about 4.2 mg/kg (e.g., about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, or about 4.2 mg/kg).

In a twenty-second aspect, the disclosure features kits comprising an agent selected from the group consisting of the antibody or antigen-binding fragment thereof disclosed in the ninth, tenth, eleventh, twelfth, thirteenth, or fourteenth aspect of the disclosure, the construct disclosed in the sixteenth aspect of the disclosure, the polynucleotide disclosed in the seventeenth aspect of the disclosure, the vector disclosed in the eighteenth aspect of the disclosure, the host cell disclosed in the nineteenth aspect of the disclosure, or the pharmaceutical composition disclosed in the twentieth aspect of the disclosure.

In some embodiments, the kit further comprises instructions for transfecting the vector into a host cell. In some embodiments, the kit further comprises instructions for expressing the antibody or antigen-binding fragment thereof or the construct in a host cell. In some embodiments, the kit further comprises a reagent that can be used to express the antibody or antigen-binding fragment thereof or the construct in a host cell. In some embodiments, the kit further comprises instructions for administering the agent to a human subject. In some embodiments, the kit further comprises instructions for making or using the agent. In some embodiments, the kit further comprises instructions for determining a level of the soluble TNFRSF member protein.

In a twenty-third aspect, the disclosure features methods comprising:
(a)
   (i) detecting a level of a soluble TNFRSF member protein in a biological fluid from a subject, in which the level of the soluble TNFRSF member protein in the biological fluid is higher (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) relative to a reference level of the soluble TNFRSF member protein; and
   (ii) administering to the subject one or more doses of an antagonistic antibody or antigen-binding fragment thereof that specifically binds the TNFRSF member protein at a dosing frequency, wherein the method is for decreasing the level of the soluble TNFRSF member protein; or
(b)
   (i) detecting a level of a soluble TNFRSF member protein in a biological fluid from a subject, in which the level of the soluble TNFRSF member protein in the biological fluid is normal or lower (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) relative to a reference level of the soluble TNFRSF member protein; and
   (ii) administering to the subject one or more doses of an agonistic antibody or antigen-binding fragment thereof that specifically binds the TNFRSF member protein at a dosing frequency, wherein the method is for augmenting the level of the soluble TNFRSF member protein.

In some embodiments, the normal level of the soluble TNFRSF member protein in a biological fluid is the level of the soluble TNFRSF member protein in the biological fluid (e.g., serum or plasma) of an otherwise healthy human. In some embodiments, the normal level of soluble TNFR2 in the serum of a human subject is from about 1.53 ng/ml to about 3.00 ng/ml (e.g., from about 1.80 ng/ml to about 3.00 ng/ml, from about 2.10 ng/ml to about 3.00 ng/ml, from about 2.40 ng/ml to about 3.00 ng/ml, from about 2.70 ng/ml to about 3.00 ng/ml, from about 1.53 ng/ml to about 2.70 ng/ml, from about 1.80 ng/ml to about 2.70 ng/ml, from about 2.10 ng/ml to about 2.70 ng/ml, from about 2.40 ng/ml to about 2.70 ng/ml, from about 1.53 ng/ml to about 2.40 ng/ml, from about 1.80 ng/ml to about 2.40 ng/ml, from about 2.10 ng/ml to about 2.40 ng/ml, from about 1.53 ng/ml to about 2.10 ng/ml, from about 1.80 ng/ml about 2.10 ng/ml, from about 1.53 ng/ml to about 1.80 ng/ml, about 1.53 ng/ml, about 1.80 ng/ml, about 2.10 ng/ml, about 2.40 ng/ml, about 2.70 ng/ml, or about 3.00 ng/ml). In some embodiments, the augmented level of the soluble TNFRSF member protein in a biological fluid is higher (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) than the normal level of the soluble TNFRSF member protein in the biological fluid. In some embodiments, the augmented level of soluble TNFR2 in the serum of a human subject is from about 2.53 ng/ml to about 10 ng/ml (e.g., from about 2.53 ng/ml to about 3 ng/ml, from about 2.53 ng to about 4 ng/ml, from about 2.53 ng to about 6 ng/ml, from about 2.53 ng to about 8 ng/ml, from about 3 ng/ml to about 4 ng/ml, from about 3 ng/ml to about 6 ng/ml, from about 3 ng/ml to about 8 ng/ml, from about 3 ng/ml to about 10 ng/ml, from about 4 ng/ml to about 6 ng/ml, from about 4 ng/ml to about 8 ng/ml, from about 4 ng/ml to about 10 ng/ml, from about 6 ng/ml to about 8 ng/ml, from about 6 ng/ml to about 10 ng/ml, from about 8 ng/ml to about 10 ng/ml, about 2.53 ng/ml, about 3 ng/ml, about 4 ng/ml, about 6 ng/ml, about 8 ng/ml, or about 10 ng/ml). When the level of the soluble TNFRSF member protein in the human subject is higher (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) than the normal level of soluble TNFRSF member protein, an antagonistic antibody or antigen-binding fragment thereof that specifically binds the corresponding TNFRSF member protein may be used to lower the level of the soluble TNFRSF member protein in the human subject (e.g., to the normal level of the soluble TNFRSF member protein).

In some embodiments, the decreased (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) level of the soluble TNFRSF member protein in the human subject indicates the presence of inadequate T cell suppression and the need for more TNFR2 function such as a cell proliferation disorder, an autoimmune disease, an infectious disease, an inflammatory disease, a neurological disease, an allergy, a transplant rejection (e.g., an allograft rejection), or a graft-versus-host disease in the human subject. In some embodiments, the neurological disease is Alzheimer's disease. In this case, a corresponding TNFRSF agonist (an agonistic antibody or antigen-binding fragment thereof that binds the TNFRSF member protein corresponding to the soluble TNFRSF member protein, such as the agonistic antibodies or antigen-binding fragments disclosed herein) can be used to increase the level of the soluble TNFRSF member protein. This strategy can be used in the screening for antibodies, in clinical trials to determine optimal dosing levels of TNFRSF agonists, or in clinical practice to make sure the dosing levels of TNFRSF agonists administered to a subject are therapeutically effective. For example, lowered levels of soluble TNFR2 in a human subject may indicate a flare of autoimmunity or impending transplant rejection. Increased levels of soluble TNFR2 in a human subject may indicate cancer with a Treg- and/or TNFR2-driven mechanism in need of a therapy to lower soluble TNFR2 levels (e.g., by eliminating suppressor cells such as Treg cells or MDSCs). In organ or tissue transplantation, the host may have a normal level of soluble TNFR2, but the desire would be to elevate the level of soluble TNFR2 (e.g., by administering to the host a TNFR2 agonist, such as an agonistic antibody that binds TNFR2 as described herein) to maintain the viability of the transplanted organ or tissue by promoting Treg cellular activity.

In some embodiments, the method comprises detecting a normal level of the soluble TNFRSF member protein or an increased (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) level of the soluble TNFRSF member protein in a biological fluid of the subject relative to a reference level of the soluble TNFRSF member protein, in which the method further comprises changing (e.g., increasing or decreasing) the dosage and/or dosing frequency of the antagonistic antibody or antigen-binding fragment thereof that specifically binds the TNFRSF member protein. In some embodiments, the antagonistic antibody or antigen-binding fragment thereof that specifically binds the TNFRSF member protein decreases or increases (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) the level of the TNFRSF member protein. In a preferred embodiment, the antagonistic antibody or antigen-binding fragment thereof that specifically binds the TNFRSF member protein decreases (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) the level of the TNFRSF member protein.

In some embodiments, the method comprises detecting a normal level of the soluble TNFRSF member protein or an decreased (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) level of the soluble TNFRSF member protein in a biological fluid of the subject relative to a reference level of the soluble TNFRSF member protein, in which the method further comprises changing (e.g., increasing or decreasing) the dosage and/or dosing frequency of the agonistic antibody or antigen-binding fragment thereof that specifically binds the TNFRSF member protein. In some embodiments, the agonistic antibody or antigen-binding fragment thereof that specifically binds the TNFRSF member protein decreases or increases (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) the level of the TNFRSF member protein. In a preferred embodiment, the agonistic antibody or antigen-binding fragment thereof that specifically binds the TNFRSF member protein increases (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) the level of the TNFRSF member protein.

In some embodiments, the method comprises detecting an increased (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) level of the soluble TNFRSF member protein in a biological fluid of the subject relative to a reference level of the soluble TNFRSF member protein, in which the method further comprises:
(a) increasing the dosage and/or dosing frequency of the antagonistic antibody or antigen-binding fragment thereof that specifically binds the TNFRSF member protein; or
(b) decreasing the dosage and/or dosing frequency of the agonistic antibody or antigen-binding fragment thereof that specifically binds the TNFRSF member protein.

In some embodiments, the method comprises detecting a normal or decreased (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) level of the soluble TNFRSF member protein in a biological fluid of the subject relative to a reference level of the soluble TNFRSF member protein, in which the method further comprises:
(a) increasing the dosage and/or dosing frequency of the agonistic antibody or antigen-binding fragment thereof that specifically binds the TNFRSF member protein; or
(b) decreasing the dosage and/or dosing frequency of the antagonistic antibody or antigen-binding fragment thereof that specifically binds the TNFRSF member protein.

In some embodiments, the TNFRSF member protein is selected from the group consisting of TNFR2, 4-1BB, CD27, CD30, CD40, DR6, EDAR, Fas, GITR, HVEM, LT-β receptor, NGFR, OPG, OX40, RANK, RELT (TNFRSF19L), TNFR1, TRAIL-R1 (TNFRSF10A), TRAIL-R2 (TNFRSF10B), TRAIL-R4, TRAMP, TROY, XEDAR, and DCR3. In some embodiments, the TNFRSF member protein is TNFR2.

In some embodiments, the antagonistic or agonistic antibody or antigen-binding fragment thereof that specifically binds the TNFRSF member protein is administered to the human subject at a dosing frequency of once every week, once every two weeks, once every three weeks, or once every four weeks.

In some embodiments, the antagonistic or agonistic antibody or antigen-binding fragment thereof that specifically binds the TNFRSF member protein is administered to the human subject at a dose of from about 0.8 mg/kg to about 1.2 mg/kg (e.g., about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, or about 1.2 mg/kg), from about 1.65 mg/kg to about 2.5 mg/kg (e.g., about 1.65 mg/kg, about 1.7 mg/kg, about 1.75 mg/kg, about 1.8 mg/kg, about 1.85 mg/kg, about 1.9 mg/kg, about 1.95 mg/kg, about 2.0 mg/kg, about 2.05 mg/kg, about 2.1 mg/kg, about 2.15 mg/kg, about 2.2 mg/kg, about 2.25 mg/kg, about 2.3 mg/kg, about 2.35 mg/kg, about 2.4 mg/kg, about 2.45 mg/kg, or about 2.5 mg/kg), from about 2.2 mg/kg to about 3.0 mg/kg (e.g., about 2.2 mg/kg, about 2.25 mg/kg, about 2.3 mg/kg, about 2.35 mg/kg, about 2.4 mg/kg, about 2.45 mg/kg, about 2.5 mg/kg, about 2.55 mg/kg, about 2.6 mg/kg, about 2.65 mg/kg, about 2.7 mg/kg, about 2.75 mg/kg, about 2.8 mg/kg, about 2.85 mg/kg, about 2.9 mg/kg, about 2.95 mg/kg, or about 3.0 mg/kg), or from about 2.5 mg/kg to about 4.2 mg/kg (e.g., about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, or about 4.2 mg/kg).

In some embodiments, the antagonistic or agonistic antibody or antigen-binding fragment thereof that specifically binds the TNFRSF member protein is administered to the human subject:
(a) once every week at a dose of from about 0.8 mg/kg to about 1.2 mg/kg (e.g., about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, or about 1.2 mg/kg);
(b) once every two weeks at a dose of from about 1.65 mg/kg to about 2.5 mg/kg (e.g., about 1.65 mg/kg, about 1.7 mg/kg, about 1.75 mg/kg, about 1.8 mg/kg, about 1.85 mg/kg, about 1.9 mg/kg, about 1.95 mg/kg, about 2.0 mg/kg, about 2.05 mg/kg, about 2.1 mg/kg, about 2.15 mg/kg, about 2.2 mg/kg, about 2.25 mg/kg, about 2.3 mg/kg, about 2.35 mg/kg, about 2.4 mg/kg, about 2.45 mg/kg, or about 2.5 mg/kg);
(c) once every three weeks at a dose of from about 2.2 mg/kg to about 3.0 mg/kg (e.g., about 2.2 mg/kg, about 2.25 mg/kg, about 2.3 mg/kg, about 2.35 mg/kg, about 2.4 mg/kg, about 2.45 mg/kg, about 2.5 mg/kg, about 2.55 mg/kg, about 2.6 mg/kg, about 2.65 mg/kg, about 2.7 mg/kg, about 2.75 mg/kg, about 2.8 mg/kg, about 2.85 mg/kg, about 2.9 mg/kg, about 2.95 mg/kg, or about 3.0 mg/kg); or
(d) once every four weeks at a dose of from about 2.5 mg/kg to about 4.2 mg/kg (e.g., about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, or about 4.2 mg/kg).

In some embodiments, the method further comprises decreasing the dose range of the antagonistic or the agonistic antibody or antigen-binding fragment thereof that specifically binds the TNFRSF member protein.

In some embodiments, the antagonistic or agonistic antibody or antigen-binding fragment thereof that specifically binds the TNFRSF member protein is administered to the human subject subcutaneously. In some embodiments, the level of soluble TNFR2 in a human subject is the serum or plasma level of soluble TNFR2 in the human subject. In some embodiments, the burden (e.g., cell number) of Treg cells or MDSCs (e.g., TNFR2-expressing Treg cells or MDSCs) is measured directly in the tumor microenvironment of a human subject having cancer.

In some embodiments, the method is for treating a cell proliferation disorder, an autoimmune disease, an infectious disease, an inflammatory disease, a neurological disease, an allergy, a transplant rejection (e.g., an allograft rejection), or a graft-versus-host disease in the human subject. In some embodiments, the method is for regenerating a tissue or organ in the human subject. In some embodiments, the method is for treating a cell proliferation disorder, and the antibody or antigen-binding fragment thereof is an antagonist antibody or antigen-binding fragment thereof that specifically binds TRAMP, DR6, NGFR, RANK, FN14, TNFR2, LT-β receptor, HVEM, CD30, BCMA, TACI, BAFF, TROY, or RELT. In some embodiments, the method is for treating a cell proliferation disorder, and the antibody or antigen-binding fragment thereof is an agonistic antibody or antigen-binding fragment thereof that specifically binds TRAIL-R1, TRAIL-R2, TRAIL-R3, TRAIL-R4, TNFR1, Fas, CD40, CD27, 4-1BB, OX40, GITR, or XEDAR. In some embodiments, the method is for treating an autoimmune disease, and the antibody or antigen-binding fragment thereof is an antagonist antibody or antigen-binding fragment thereof that specifically binds TRAIL-R1, TRAIL-R2, TRAIL-R3, TRAIL-R4, TNFR1, CD40, CD27, 4-1BB, OX40, GITR, or XEDAR. In some embodiments, the method is for treating an autoimmune disease, and the antibody or antigen-binding fragment thereof is an agonistic antibody or antigen-binding fragment thereof that specifically binds TRAMP, DR6, NGFR, FN14, TNFR2, LT-β receptor, HVEM, CD30, or RELT. In some embodiments, the method is for treating an infectious disease, and the antibody or antigen-binding fragment thereof is an antagonistic antibody or antigen-binding fragment thereof that specifically binds TRAMP, DR6, TNFR2, HVEM, or RELT. In some embodiments, the method is for treating an infectious disease, and the antibody or antigen-binding fragment thereof is an agonistic antibody or antigen-binding fragment thereof that specifically binds TRAIL-R1, TRAIL-R2, TRAIL-R3, TRAIL-R4, CD40, CD27, 4-1BB, OX40, BCMA, TACI, or BAFF. In some embodiments, the method is for treating an inflammatory disease, and the antibody or antigen-binding fragment thereof is an agonistic antibody or antigen-binding fragment thereof that specifically binds DR6, FN14, TNFR2, HVEM, or RELT. In some embodiments, the method is for treating a neurological disease, and the antibody or antigen-binding fragment thereof is an antagonistic antibody or antigen-binding fragment thereof that specifically binds DR6. In some embodiments, the method is for treating a neurological disease (e.g., a CNS disease), and the antibody or antigen-binding fragment thereof is an agonistic antibody or antigen-binding fragment thereof that specifically binds TNFR2. In some embodiments, the method is for treating an allergy, and the antibody or antigen-binding fragment thereof is an antagonistic antibody or antigen-binding fragment thereof that specifically binds TNFR1, CD40, CD27, 4-1BB, OX40, GITR, or XEDAR. In some embodiments, the method is for treating a transplant rejection (e.g., an allograft rejection) or a graft-versus-host disease, and the antibody or antigen-binding fragment thereof is an antagonistic antibody or antigen-binding fragment thereof that specifically binds TNFR1, 4-1BB, OX40, GITR, or XEDAR. In some embodiments, the method is for treating a transplant rejection (e.g., an allograft rejection) or a graft-versus-host disease, and the antibody or antigen-binding fragment thereof is an agonistic antibody or antigen-binding fragment thereof that specifically binds CD30 or TNFR2.

In some embodiments, the biological fluid is serum or plasma.

In some embodiments, the reference level of the soluble TNFRSF member protein is the level of the soluble TNFRSF in the biological fluid of a healthy human.

In some embodiments, the one or more doses of the antibody or antigen-binding fragment thereof is administered to the human subject in one or more treatment periods, in which:
(a) each dose comprises:
   (i) from about 0.1 mg to about 5000 mg of the antibody or antigen-binding fragment thereof; or
   (ii) from about 0.001 mg/kg to about 50 mg/kg of the antibody or antigen-binding fragment thereof; and/or
(b) each treatment period lasts one week, two weeks, three weeks, four weeks, five weeks, six weeks, seven weeks, eight weeks, or more.

In some embodiments, the dosing frequency is one or more times a month, every three weeks, every two weeks, a week, every six days, every five days, every four days, every three days, every two days, or a day. In some embodiments, the dosing frequency is once every week (Q1W), once every two weeks (Q2W), once every three weeks (Q3W), or once every four weeks (Q4W).

In some embodiments, the antagonistic or agonistic antibody or antigen-binding fragment thereof that specifically binds the TNFRSF member protein is administered to the human subject subcutaneously.

In a twenty-fourth aspect, the disclosure features an antibody or antigen-binding fragment thereof that specifically binds human TNFR2, in which the antibody or antigen-binding fragment thereof comprises a CDR-H1, a CDR-H2, a CDR-H3, a CDR-L1, a CDR-L2, and a CDR-L3, and further in which:
(a) the CDR-H1 comprises the amino acid sequence of GXTFXXY (SEQ ID NO: 1449), wherein each X is independently a naturally occurring amino acid;
(b) the CDR-H2 comprises the amino acid sequence of NPNYDS (SEQ ID NO: 1414), NYDSTS (SEQ ID NO: 1420), or a variant thereof with up to two conservative amino acid substitutions;
(c) the CDR-H3 comprises the amino acid sequence of GNSWYFDV (SEQ ID NO: 1415) or a variant thereof with up to two conservative amino acid substitutions;
(d) the CDR-L1 comprises the amino acid sequence of SASSSVRYMY (SEQ ID NO: 1416) or a variant thereof with up to two conservative amino acid substitutions;
(e) the CDR-L2 comprises the amino acid sequence of LTSNLAS (SEQ ID NO: 1417) or a variant thereof with up to two conservative amino acid substitutions; and
(f) the CDR-L3 comprises the amino acid sequence of QQWSSNPLT (SEQ ID NO: 1418) or a variant thereof with up to two conservative amino acid substitutions.

In some embodiments, the CDR-H1 comprises an amino acid sequence selected from the group consisting of GYTFXXY (SEQ ID NO: 1450), GSTFXXY (SEQ ID NO: 1451), GTTFXXY (SEQ ID NO: 1452), GCTFXXY (SEQ ID NO: 1453), GNTFXXY (SEQ ID NO: 1454), GQTFXXY (SEQ ID NO: 1455), GXTFSXY (SEQ ID NO: 1456), GXTFCXY (SEQ ID NO: 1457), GXTFYXY (SEQ ID NO: 1458), GXTFNXY (SEQ ID NO: 1459), GXTFOXY (SEQ ID NO: 1460), and GXTFXDY (SEQ ID NO: 1461), wherein each X is independently a naturally occurring amino acid

In some embodiments, the CDR-H1 comprises the amino acid sequence of GYTFTDY (SEQ ID NO: 1413) or a variant thereof with up to two conservative amino acid substitutions.

In some embodiments, the CDR-H1 comprises the amino acid sequence of GYTFTDY (SEQ ID NO: 1413), the CDR-H2 comprises the amino acid sequence of NPNYDS (SEQ ID NO: 1414), the CDR-H3 comprises the amino acid sequence of GNSWYFDV (SEQ ID NO: 1415), the CDR-L1 comprises the amino acid sequence of SASSSVRYMY (SEQ ID NO: 1416), the CDR-L2 comprises the amino acid sequence of LTSNLAS (SEQ ID NO: 1417), and the CDR-L3 comprises the amino acid sequence of QQWSSNPLT (SEQ ID NO: 1418).

In some embodiments, the CDR-H1 comprises the amino acid sequence of GYTFTDY (SEQ ID NO: 1413), the CDR-H2 comprises the amino acid sequence of NYDSTS (SEQ ID NO: 1420), the CDR-H3 comprises the amino acid sequence of GNSWYFDV (SEQ ID NO: 1415), the CDR-L1 comprises the amino acid sequence of SASSSVRYMY (SEQ ID NO: 1416), the CDR-L2 comprises the amino acid sequence of LTSNLAS (SEQ ID NO: 1417), and the CDR-L3 comprises the amino acid sequence of QQWSSNPLT (SEQ ID NO: 1418).

In a twenty-fifth aspect, the disclosure features an antibody or antigen-binding fragment thereof that specifically binds human TNFR2, in which the antibody or antigen-binding fragment thereof comprises a CDR-H1, a CDR-H2, a CDR-H3, a CDR-L1, a CDR-L2, and a CDR-L3, and further in which:
(a) the CDR-H1 comprises the amino acid sequence of GYTFTDYNLD (SEQ ID NO: 1425), DYIMH (SEQ ID NO: 1426), or a variant thereof with up to two conservative amino acid substitutions;
(b) the CDR-H2 comprises the amino acid sequence of DINPNYDS (SEQ ID NO: 1427), WVDPEYGSTDYAEKFKK (SEQ ID NO: 1428), or a variant thereof with up to two conservative amino acid substitutions;
(c) the CDR-H3 comprises the amino acid sequence of TSYSQKFRG (SEQ ID NO: 1429), DDGSYSPFEDY (SEQ ID NO: 1430), or a variant thereof with up to two conservative amino acid substitutions;
(d) the CDR-L1 comprises an amino acid sequence selected from the group consisting of SASSSVRYMYWY (SEQ ID NO: 1431), KASENVVTYVS (SEQ ID NO: 1432), RSSQSLVHSNGNTYLH (SEQ ID NO: 1433), KASENVVTYVS (SEQ ID NO: 1434), QASQNINKYIA (SEQ ID NO: 1435), QNINKY (SEQ ID NO: 1436), and a variant thereof with up to two conservative amino acid substitutions;
(e) the CDR-L2 comprises an amino acid sequence selected from the group consisting of LTSNLAS (SEQ ID NO: 1437), VTSNLAS (SEQ ID NO: 1438), LTSNLGS (SEQ ID NO: 1439), GASNRTY (SEQ ID NO: 1440), IKVSNRFS (SEQ ID NO: 1441), DPEYGS (SEQ ID NO: 1442), YTSTLES (SEQ ID NO: 1443), YTS, and a variant thereof with up to two conservative amino acid substitutions; and
(f) the CDR-L3 comprises an amino acid sequence selected from the group consisting of QQRSNWP (SEQ ID NO: 1445), GQGYSYPYT (SEQ ID NO: 1446), SQTTHVPPT (SEQ ID NO: 1447), LQYVNLIT (SEQ ID NO: 1448), and a variant thereof with up to two conservative amino acid substitutions.

In some embodiments, the CDR-H1 comprises the amino acid sequence of GYTFTDYNLD (SEQ ID NO: 1425) or DYIMH (SEQ ID NO: 1426); the CDR-H2 comprises the amino acid sequence of DINPNYDS (SEQ ID NO: 1427) or WVDPEYGSTDYAEKFKK (SEQ ID NO: 1428); the CDR-H3 comprises the amino acid sequence of TSYSQKFRG (SEQ ID NO: 1429) or DDGSYSPFEDY (SEQ ID NO: 1430); the CDR-L1 comprises an amino acid sequence selected from the group consisting of SASSSVRYMYWY (SEQ ID NO: 1431), KASENVVTYVS (SEQ ID NO: 1432), RSSQSLVHSNGNTYLH (SEQ ID NO: 1433), KASENVVTYVS (SEQ ID NO: 1434), QASQNINKYIA (SEQ ID NO: 1435), and QNINKY (SEQ ID NO: 1436); the CDR-L2 comprises an amino acid sequence selected from the group consisting of LTSNLAS (SEQ ID NO: 1437), VTSNLAS (SEQ ID NO: 1438), LTSNLGS (SEQ ID NO: 1439), GASNRTY (SEQ ID NO: 1440), IKVSNRFS (SEQ ID NO: 1441), DPEYGS (SEQ ID NO: 1442), YTSTLES (SEQ ID NO: 1443), and YTS; and the CDR-L3 comprises an amino acid sequence selected from the group consisting of QQRSNWP (SEQ ID NO: 1445), GQGYSYPYT (SEQ ID NO: 1446), SQTTHVPPT (SEQ ID NO: 1447), and LQYVNLIT (SEQ ID NO: 1448).

In a twenty-sixth aspect, the disclosure features an antibody or antigen-binding fragment thereof that specifically binds human TNFR2, in which the antibody or antigen-binding fragment thereof comprises:
(a) a VH having an amino acid sequence that is at least 80% identical (e.g., 85%, 90%, 95%, 97%, 99%, or 100% identical) to any one of SEQ ID NOs: 1486-1490; and
(b) a VL having an amino acid sequence that is at least 80% identical (e.g., 85%, 90%, 95%, 97%, 99%, or 100% identical) to any one of SEQ ID NOs: 1492-1497.

In some embodiments, the antibody or antigen-binding fragment thereof comprises:
(a) a VH having the amino acid sequence of any one of SEQ ID NOs: 1486-1490; and
(b) a VL having the amino acid sequence of any one of SEQ ID NOs: 1492-1497.

In some embodiments, the antibody or antigen binding fragment thereof comprises:
(a) a VH having an amino acid sequence that is at least 80% identical (e.g., 85%, 90%, 95%, 97%, 99%, or 100% identical) to SEQ ID NO: 1488 and a VL having an amino acid sequence that is at least 80% identical (e.g., 85%, 90%, 95%, 97%, 99%, or 100% identical) to SEQ ID NO: 1495;
(b) a VH having an amino acid sequence that is at least 80% identical (e.g., 85%, 90%, 95%, 97%, 99%, or 100% identical) to SEQ ID NO: 1488 and a VL having an amino acid sequence that is at least 80% identical (e.g., 85%, 90%, 95%, 97%, 99%, or 100% identical) to SEQ ID NO: 1497;
(c) a VH having an amino acid sequence that is at least 80% identical (e.g., 85%, 90%, 95%, 97%, 99%, or 100% identical) to SEQ ID NO: 1489 and a VL having an amino acid sequence that is at least 80% identical (e.g., 85%, 90%, 95%, 97%, 99%, or 100% identical) to SEQ ID NO: 1496;
(d) a VH having an amino acid sequence that is at least 80% identical (e.g., 85%, 90%, 95%, 97%, 99%, or 100% identical) to SEQ ID NO: 1490 and a VL having an amino acid sequence that is at least 80% identical (e.g., 85%, 90%, 95%, 97%, 99%, or 100% identical) to SEQ ID NO: 1496; or
(e) a VH having an amino acid sequence that is at least 80% identical (e.g., 85%, 90%, 95%, 97%, 99%, or 100% identical) to SEQ ID NO: 1490 and a VL having an amino acid sequence that is at least 80% identical (e.g., 85%, 90%, 95%, 97%, 99%, or 100% identical) to SEQ ID NO: 1497.

In some embodiments, the antibody or antigen binding fragment thereof comprises:
(a) a VH having the amino acid sequence of SEQ ID NO: 1488 and a VL having the amino acid sequence of SEQ ID NO: 1495;
(b) a VH having the amino acid sequence of SEQ ID NO: 1488 and a VL having the amino acid sequence of SEQ ID NO: 1497;
(c) a VH having the amino acid sequence of SEQ ID NO: 1489 and a VL having the amino acid sequence of SEQ ID NO: 1496;
(d) a VH having the amino acid sequence of SEQ ID NO: 1490 and a VL having the amino acid sequence of SEQ ID NO: 1496; or
(e) a VH having the amino acid sequence of SEQ ID NO: 1490 and a VL having the amino acid sequence of SEQ ID NO: 1497.

In some embodiments, the antibody or antigen-binding fragment thereof comprises a VH having an amino acid sequence that is at least 80% identical (e.g., 85%, 90%, 95%, 97%, 99%, or 100% identical) to SEQ ID NO: 1488 and a VL having an amino acid sequence that is at least 80% identical (e.g., 85%, 90%, 95%, 97%, 99%, or 100% identical) to SEQ ID NO: 1495.

In some embodiments, the antibody or antigen-binding fragment thereof comprises a VH having the amino acid sequence of SEQ ID NO: 1488 and a VL having the amino acid sequence of SEQ ID NO: 1495.

In some embodiments, the antibody or antigen-binding fragment thereof is a human antibody or antigen-binding fragment thereof. In some embodiments, the antibody or antigen-binding fragment thereof is an IgG antibody or antigen-binding fragment thereof. In some embodiments, the antibody or antigen-binding fragment thereof is a human IgG1, human IgG2, human IgG3, or human IgG4 antibody or antigen-binding fragment thereof. In some embodiments, the antibody or antigen-binding fragment thereof is a human IgG4 antibody or antigen-binding fragment thereof.

In some embodiments, the antibody or antigen-binding fragment thereof further comprises a human IgG4 Fc domain. In some embodiments, the human IgG4 Fc domain comprises a human IgG4 hinge region having the amino acid substitution S228P numbered according to the EU index (S241P according to Kabat). In some embodiments, the modified antibody or antigen-binding fragment thereof comprises the amino acid substitution S228P.

In a twenty-seventh aspect, the disclosure features an antibody or antigen-binding fragment thereof that specifically binds human TNFR2, in which the antibody or antigen-binding fragment thereof comprises:
(a) a heavy chain having an amino acid sequence that is at least 80% identical (e.g., 85%, 90%, 95%, 97%, 99%, or 100% identical) to any one of SEQ ID NOs: 1498-1503; and
(b) a light chain having an amino acid sequence that is at least 80% identical (e.g., 85%, 90%, 95%, 97%, 99%, or 100% identical) to any one of SEQ ID NOs: 1504-1510.

In some embodiments, the antibody or antigen-binding fragment thereof comprises:
(a) a heavy chain having the amino acid sequence of any one of SEQ ID NOs: 1498-1503; and
(b) a light chain having the amino acid sequence of any one of SEQ ID NOs: 1504-1510.

In some embodiments, the antibody or antigen binding fragment thereof comprises:
(a) a heavy chain having an amino acid sequence that is at least 80% identical (e.g., 85%, 90%, 95%, 97%, 99%, or 100% identical) to SEQ ID NO: 1498 and a light chain having an amino acid sequence that is at least 80% identical (e.g., 85%, 90%, 95%, 97%, 99%, or 100% identical) to SEQ ID NO: 1504;
(b) a heavy chain having an amino acid sequence that is at least 80% identical (e.g., 85%, 90%, 95%, 97%, 99%, or 100% identical) to SEQ ID NO: 1501 and a light chain having an amino acid sequence that is at least 80% identical (e.g., 85%, 90%, 95%, 97%, 99%, or 100% identical) to SEQ ID NO: 1508;
(c) a heavy chain having an amino acid sequence that is at least 80% identical (e.g., 85%, 90%, 95%, 97%, 99%, or 100% identical) to SEQ ID NO: 1501 and a light chain having an amino acid sequence that is at least 80% identical (e.g., 85%, 90%, 95%, 97%, 99%, or 100% identical) to SEQ ID NO: 1510;
(d) a heavy chain having an amino acid sequence that is at least 80% identical (e.g., 85%, 90%, 95%, 97%, 99%, or 100% identical) to SEQ ID NO: 1502 and a light chain having an amino acid sequence that is at least 80% identical (e.g., 85%, 90%, 95%, 97%, 99%, or 100% identical) to SEQ ID NO: 1509;
(e) a heavy chain having an amino acid sequence that is at least 80% identical (e.g., 85%, 90%, 95%, 97%, 99%, or 100% identical) to SEQ ID NO: 1503 and a light chain having an amino acid sequence that is at least 80% identical (e.g., 85%, 90%, 95%, 97%, 99%, or 100% identical) to SEQ ID NO: 1509; or
(f) a heavy chain having an amino acid sequence that is at least 80% identical (e.g., 85%, 90%, 95%, 97%, 99%, or 100% identical) to SEQ ID NO: 1503 and a light chain having an amino acid sequence that is at least 80% identical (e.g., 85%, 90%, 95%, 97%, 99%, or 100% identical) to SEQ ID NO: 1510.

In some embodiments, the antibody or antigen binding fragment thereof comprises:
(a) a heavy chain having the amino acid sequence of SEQ ID NO: 1498 and a light chain having the amino acid sequence of SEQ ID NO: 1504;
(b) a heavy chain having the amino acid sequence of SEQ ID NO: 1501 and a light chain having the amino acid sequence of SEQ ID NO: 1508;
(c) a heavy chain having the amino acid sequence of SEQ ID NO: 1501 and a light chain having the amino acid sequence of SEQ ID NO: 1510;
(d) a heavy chain having the amino acid sequence of SEQ ID NO: 1502 and a light chain having the amino acid sequence of SEQ ID NO: 1509;
(e) a heavy chain having the amino acid sequence of SEQ ID NO: 1503 and a light chain having the amino acid sequence of SEQ ID NO: 1509; or
(f) a heavy chain having the amino acid sequence of SEQ ID NO: 1503 and a light chain having the amino acid sequence of SEQ ID NO: 1510.

In some embodiments, the antibody or antigen-binding fragment thereof comprises a heavy chain having an amino acid sequence that is at least 80% identical (e.g., 85%, 90%, 95%, 97%, 99%, or 100% identical) to SEQ ID NO: 1501 and a light chain having an amino acid sequence that is at least 80% identical (e.g., 85%, 90%, 95%, 97%, 99%, or 100% identical) to SEQ ID NO: 1508.

In some embodiments, the antibody or antigen-binding fragment thereof comprises a heavy chain having the amino acid sequence of SEQ ID NO: 1501 and a light chain having the amino acid sequence of SEQ ID NO: 1508.

In some embodiments, the antibody or antigen-binding fragment thereof is a human IgG4 antibody or antigen-binding fragment thereof.

In some embodiments of the twenty-fourth, twenty-fifth, twenty-sixth, or twenty-seventh aspect, the antibody or antigen-binding fragment thereof is an agonistic antibody or antigen-binding fragment thereof that specifically binds human TNFR2.

In some embodiments, the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, an antibody-like protein scaffold, a Fv fragment, a Fab fragment, a F(ab′)₂ molecule, and a tandem scFv (taFv). In some embodiments, the antibody or antigen-binding fragment thereof is a human, humanized, or chimeric antibody or antigen-binding fragment thereof.

In a twenty-eighth aspect, the disclosure features methods of producing the antibody or antigen-binding fragment thereof disclosed in the twenty-fourth, twenty-fifth, twenty-sixth, or twenty-seventh aspect, the methods including expressing a polynucleotide encoding the antibody or antigen-binding fragment thereof in a host cell and recovering the antibody or antigen-binding fragment thereof from host cell medium.

In a twenty-ninth aspect, the disclosure features constructs comprising a first polypeptide domain and a second polypeptide domain, in which the first polypeptide domain and the second polypeptide domain are each, independently, an antigen-binding fragment disclosed in the twenty-fourth, twenty-fifth, twenty-sixth, or twenty-seventh aspect of the disclosure.

In some embodiments, the first polypeptide domain and the second polypeptide domain are bound by a covalent linker. In some embodiments, the covalent linker comprises an amide bond or a disulfide bond.

In a thirtieth aspect, the disclosure features polynucleotides encoding the antibody or antigen-binding fragment thereof disclosed in the twenty-fourth, twenty-fifth, twenty-sixth, or twenty-seventh aspect of the disclosure or the construct disclosed in the twenty-seventh aspect of the disclosure.

In a thirty-first aspect, the disclosure features vectors comprising the polynucleotide disclosed in the thirtieth aspect of the disclosure.

In some embodiments, the vector is an expression vector. In some embodiments, the expression vector is a eukaryotic expression vector.

In some embodiments, the vector is a viral vector. In some embodiments, the viral vector is selected from the group consisting of an adenovirus, retrovirus, poxvirus, adeno-associated virus, baculovirus, herpes simplex virus, and vaccinia virus. In some embodiments, the adenovirus is a serotype 1-60 adenovirus. In some embodiments, the adenovirus is a serotype 5, 26, 35, or 48 adenovirus. In some embodiments, the retrovirus is a γ-retrovirus or a lentivirus. In some embodiments, the vaccinia virus is a modified vaccinia Ankara (MVA).

In a thirty-second aspect, the disclosure features isolated host cells comprising the vector disclosed in the thirty-first aspect of the disclosure.

In some embodiments, the host cell is a prokaryotic cell or a eukaryotic cell. In some embodiments, the eukaryotic cell is a mammalian cell. In some embodiments, the mammalian cell is a CHO cell.

In a thirty-third aspect, the disclosure features pharmaceutical compositions comprising the antibody or antigen-binding fragment thereof disclosed in the twenty-fourth, twenty-fifth, twenty-sixth, or twenty-seventh aspect of the disclosure, the construct disclosed in the twenty-ninth aspect of the disclosure, the polynucleotide disclosed in the thirtieth aspect of the disclosure, the vector disclosed in the thirty-first aspect of the disclosure, or the host cell disclosed in the thirty-second aspect of the disclosure, and a pharmaceutically acceptable carrier or excipient.

In a thirty-fourth aspect, the disclosure features methods of modulating an immune response in a human subject, the method comprising administering to the subject the antibody or antigen-binding fragment thereof disclosed in the twenty-fourth, twenty-fifth, twenty-sixth, or twenty-seventh aspect of the disclosure, the construct disclosed in the twenty-ninth aspect of the disclosure, the polynucleotide disclosed in the thirtieth aspect of the disclosure, the vector disclosed in the thirty-first aspect of the disclosure, the host cell disclosed in the thirty-second aspect of the disclosure, or the pharmaceutical composition disclosed in the thirty-third aspect of the disclosure.

In some embodiments, the method is for treating a cell proliferation disorder, an autoimmune disease, an infectious disease, an inflammatory disease, a neurological disease, an allergy, a transplant rejection (e.g., an allograft rejection), or a graft-versus-host disease in the human subject. In some embodiments, the method is for regenerating a tissue or organ in the human subject.

In some embodiments, the method is for treating a cancer. In some embodiments, the human subject has a blood (e.g., serum or plasma) level of soluble TNFR2 that is normal or lower than a reference level of soluble TNFR2. In some embodiments, the method is for increasing the serum or plasma level of soluble TNFR2 in the human subject

In some embodiments, the method further comprises:
(a) obtaining a biological fluid (e.g., blood, plasma, or serum) from the human subject;
(b) detecting a decreased (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) level of soluble TNFR2 in the biological fluid of the subject relative to a reference level of soluble TNFR2; and
(c) administering to the subject one or more doses of the antibody or antigen-binding fragment thereof at a dosing frequency.

In some embodiments, the method further comprises:
(a) detecting a normal or decreased (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) level of soluble TNFR2 in the subject relative to a reference level of soluble TNFR2, and increasing the level of soluble TNFR2 by administering an increased dosage and/or dosing frequency of the antibody or antigen-binding fragment thereof to increase the level of soluble TNFR2; or
(b) detecting an increased (e.g., by 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more) level of soluble TNFR2 in the subject relative to a reference level of soluble TNFR2, and increasing the dosage and/or dosing frequency of the antibody or antigen-binding fragment thereof.

In some embodiments, the antibody or antigen-binding fragment thereof is administered to the human subject at a dosing frequency of once every week, once every two weeks, once every three weeks, or once every four weeks. In some embodiments, the antibody or antigen-binding fragment thereof is administered to the human subject at a dose of from about 0.8 mg/kg to about 1.2 mg/kg (e.g., about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, or about 1.2 mg/kg), from about 1.65 mg/kg to about 2.5 mg/kg (e.g., about 1.65 mg/kg, about 1.7 mg/kg, about 1.75 mg/kg, about 1.8 mg/kg, about 1.85 mg/kg, about 1.9 mg/kg, about 1.95 mg/kg, about 2.0 mg/kg, about 2.05 mg/kg, about 2.1 mg/kg, about 2.15 mg/kg, about 2.2 mg/kg, about 2.25 mg/kg, about 2.3 mg/kg, about 2.35 mg/kg, about 2.4 mg/kg, about 2.45 mg/kg, or about 2.5 mg/kg), from about 2.2 mg/kg to about 3.0 mg/kg (e.g., about 2.2 mg/kg, about 2.25 mg/kg, about 2.3 mg/kg, about 2.35 mg/kg, about 2.4 mg/kg, about 2.45 mg/kg, about 2.5 mg/kg, about 2.55 mg/kg, about 2.6 mg/kg, about 2.65 mg/kg, about 2.7 mg/kg, about 2.75 mg/kg, about 2.8 mg/kg, about 2.85 mg/kg, about 2.9 mg/kg, about 2.95 mg/kg, or about 3.0 mg/kg), or from about 2.5 mg/kg to about 4.2 mg/kg (e.g., about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, or about 4.2 mg/kg). In some embodiments, the antibody or antigen-binding fragment thereof is administered to the human subject:
(a) once every week at a dose of from about 0.8 mg/kg to about 1.2 mg/kg (e.g., about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, or about 1.2 mg/kg);
(b) once every two weeks at a dose of from about 1.65 mg/kg to about 2.5 mg/kg (e.g., about 1.65 mg/kg, about 1.7 mg/kg, about 1.75 mg/kg, about 1.8 mg/kg, about 1.85 mg/kg, about 1.9 mg/kg, about 1.95 mg/kg, about 2.0 mg/kg, about 2.05 mg/kg, about 2.1 mg/kg, about 2.15 mg/kg, about 2.2 mg/kg, about 2.25 mg/kg, about 2.3 mg/kg, about 2.35 mg/kg, about 2.4 mg/kg, about 2.45 mg/kg, or about 2.5 mg/kg);
(c) once every three weeks at a dose of from about 2.2 mg/kg to about 3.0 mg/kg (e.g., about 2.2 mg/kg, about 2.25 mg/kg, about 2.3 mg/kg, about 2.35 mg/kg, about 2.4 mg/kg, about 2.45 mg/kg, about 2.5 mg/kg, about 2.55 mg/kg, about 2.6 mg/kg, about 2.65 mg/kg, about 2.7 mg/kg, about 2.75 mg/kg, about 2.8 mg/kg, about 2.85 mg/kg, about 2.9 mg/kg, about 2.95 mg/kg, or about 3.0 mg/kg); or
(d) once every four weeks at a dose of from about 2.5 mg/kg to about 4.2 mg/kg (e.g., about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, or about 4.2 mg/kg).

In some embodiments, the method further comprises decreasing the dose range of the antibody or antigen-binding fragment thereof.

### Definitions

As used herein, the term “about” refers to a value that is no more than 10% above or below the value being described. For example, the term “about 5 nM” indicates a range of from 4.5 nM to 5.5 nM.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, primatized antibodies, heteroconjugate antibodies (e.g., bi-tri- and quad-specific antibodies, diabodies, triabodies, and tetrabodies), and antigen-binding fragments of antibodies, including e.g., Fab′, F(ab′)₂, Fab, Fv, IgG, and scFv fragments. Moreover, unless otherwise indicated, the term “monoclonal antibody” (mAb) is meant to include both intact molecules, as well as antibody fragments (such as, for example, Fab and F(ab′)₂ fragments) that are capable of specifically binding to a target protein.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and VL domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. An “antigen-binding fragment” may also refer to a linear or non-linear peptide, which may be cyclic, bicylic, and/or conformationally biased. It will be appreciated by one of skill in the art that a conformationally biased antigen-binding fragment will entail a structure-based design such that the peptide includes an antigen-binding site mimic based on the 3D structure of the peptide-antigen complex. Such structural information enables the design and generation of mimics of continuous, as well as of sequentially discontinuous antigen-binding sites, which are composed of two or more protein segments that are distant in protein sequence but brought into spatial proximity through protein folding. Mimicking such discontinuous antigen-binding sites by synthetic peptides often involves splicing and/or molecular scaffolds (e.g., disulfide bonds) to enable conformation bias. See e.g., Groß et al., Front. Bioeng. Biotechnol. 4(39), 2016. Antigen-binding fragments can be produced by recombinant DNA techniques, enzymatic or chemical cleavage of intact immunoglobulins, or, in some embodiments, by chemical peptide synthesis procedures known in the art.

As used herein, the terms “agonistic TNFRSF antibody,” “agonistic anti-TNFRSF antibody,” and “TNFRSF agonist antibody” refer to TNFRSF antibodies that are capable of inducing or promoting the activation of a TNFRSF member protein (e.g., TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, or DCR3), augmenting one or more signal transduction pathways mediated by the TNFRSF member protein, and/or inducing or promoting at least one activity mediated by the activation of the TNFRSF member protein. For example, agonistic TNFRSF antibodies may induce or promote the growth and proliferation of T-reg cells. Agonistic TNFRSF antibodies may induce or promote TNFRSF activation by allosterically binding TNFRSF. In this way, agonistic TNFRSF antibodies may promote or stabilize the trimerization of the TNFRSF member protein that is induced by interacting with one or more ligands in the TNF superfamily (e.g., CD40L for CD40, TNFα for TNFR1 and TNFR2, 4-1BBL for 4-1BB, CD70 for CD27, CD153 for CD30, N-APP for DR6, EDA-A1 for EDAR, FasL for Fas, GITRL for GITR, LTα for HVEM, LT-β (TNF-C) for LT-β receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, EDA-A2 for XEDAR, or FasL, LIGHT, and TL1A for DCR3, among others), thus resulting in induction of the TNFRSF member protein activity.

As used herein, the terms “antagonistic TNFRSF antibody,” “antagonistic anti-TNFRSF antibody,” and “TNFRSF antagonist antibody” refer to TNFRSF antibodies that are capable of inhibiting or reducing the activation of a TNFRSF member protein (e.g., TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, or DCR3), attenuating one or more signal transduction pathways mediated by the TNFRSF member protein, and/or reducing or inhibiting at least one activity mediated by the activation of the TNFRSF member protein. For example, antagonistic TNFRSF antibodies may inhibit or reduce the growth and proliferation of T-reg cells. Antagonistic TNFRSF antibodies may inhibit or reduce TNFRSF activation by blocking TNFRSF from binding one or more ligands in the TNF superfamily, e.g., CD40L for CD40, TNFα for TNFR1 and TNFR2, 4-1BBL for 4-1BB, CD70 for CD27, CD153 for CD30, N-APP for DR6, EDA-A1 for EDAR, FasL for Fas, GITRL for GITR, LTα for HVEM, LT-β (TNF-C) for LT-β receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, EDA-A2 for XEDAR, or FasL, LIGHT, and TL1A for DCR3, among others. In this way, antagonistic TNFRSF antibodies may block the trimerization of the TNFRSF member protein that would otherwise be induced by interacting with one or more ligands in the TNF superfamily (e.g., CD40L, TNFα, 4-1BBL, CD70, CD153, or RANKL, among others), thus resulting in suppression of the TNFRSF member protein activity.

As used herein, the term “bispecific antibody” refers to an antibody (e.g., a monoclonal, often a human or humanized, antibody) that has binding specificities for at least two different antigens. For example, one of the binding specificities can be directed towards a TNFRSF member protein (e.g., TNFR2, TNFR1, CD40, 4-1BB, CD27, CD30, DR6, EDAR, Fas, GITR, HVEM, LT-β receptor, NGFR, OPG, OX40, RANK, RELT (TNFRSF19L), TRAIL-R2 (TNFRSF10B), TRAIL-R1 (TNFRSF10A), TRAIL-R4, TRAMP, TROY, XEDAR, or DCR3), and the other can be for any other antigen, e.g., for a cell-surface protein, receptor, receptor subunit, tissue-specific antigen, virally derived protein, virally encoded envelope protein, bacterially derived protein, or bacterial surface protein, etc.

As used herein, the term “chimeric antibody” refers to an antibody having variable domain sequences (e.g., CDR sequences) derived from an immunoglobulin of one source organism, such as rat or mouse, and constant regions derived from an immunoglobulin of a different organism (e.g., a human, a non-human primate, pig, goat, rabbit, hamster, cat, dog, guinea pig, a member of the Bovidae family (such as cattle, cow, bison, buffalo, elk, and yaks, among others), sheep, or horse, among others). Methods for producing chimeric antibodies are known in the art. See, e.g., Morrison, Science 229:1202-7, 1985; Oi et al., BioTechniques 4:214-221, 1986; Gillies et al., J. Immunol. Methods 125:191-202, 1985; U.S. Pat. Nos. 5,807,715; 4,816,567; and 4,816,397; incorporated herein by reference.

As used herein, the term “diabodies” refers to bivalent antibodies comprising two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain to form a homodimeric structure. Accordingly, the term “triabodies” refers to trivalent antibodies comprising three peptide chains, each of which contains one VH domain and one VL domain joined by a linker that is exceedingly short (e.g., a linker composed of 1-2 amino acids) to permit intramolecular association of VH and VL domains within the same peptide chain. In order to fold into their native structure, peptides configured in this way typically trimerize to position the VH and VL domains of neighboring peptide chains spatially proximal to one another to permit proper folding (see Holliger et al., Proc. Natl. Acad. Sci. USA 90:6444-6448, 1993; incorporated herein by reference).

As used herein, the term “monoclonal antibody” refers to an antibody that is derived from a single clone, including any eukaryotic, prokaryotic, or phage clone, and not the method by which it is produced.

As used herein, the term “multispecific antibodies” refers to antibodies that exhibit affinity for more than one target antigen. Multispecific antibodies can have structures similar to full immunoglobulin molecules and include Fc regions, for example, IgG Fc regions. Such structures can include, but are not limited to, IgG-Fv, IgG-(scFv)₂, DVD-Ig, (scFv)₂-(scFv)₂-Fc, and (scFv)₂-Fc-(scFv)₂. In case of IgG-(scFv)₂, the scFv can be attached to either the N-terminal or the C-terminal end of either the heavy chain or the light chain. Exemplary multi-specific molecules that include Fc regions and into which anti-TNFRSF antibodies or antigen-binding fragments thereof can be incorporated have been reviewed by Kontermann, mAbs 4:182-197, 2012; Yazaki et al., Protein Engineering, Design & Selection 26:187-193, 2013; and Grote et al., 2012, in Proetzel & Ebersbach (eds.), Antibody Methods and Protocols, Methods in Molecular Biology vol. 901, chapter 16:247-263; incorporated herein by reference. In some embodiments, antibody fragments can be components of multi-specific molecules without Fc regions, based on fragments of IgG or DVD or scFv. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, Nature Medicine 9:129-134, 2003; incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, V_{L}, and V_{H}) is substantially non-immunogenic in humans, with only minor sequence changes or variations. A human antibody can be produced in a human cell (e.g., by recombinant expression), or by a non-human animal or a prokaryotic or eukaryotic cell that can express functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibody refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All, or substantially all, of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,225,539; 5,530,101; 5,585,089; 5,693,761; 5,693,762; and 6,180,370; EP239400; PCT publication WO 91/09967; EP592106; and EP519596; incorporated herein by reference.

As used herein, the term “primatized antibody” refers to an antibody comprising framework regions from primate-derived antibodies and other regions, such as CDRs and/or constant regions, from antibodies of a non-primate source. Methods for producing primatized antibodies are known in the art. See, e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), to enhance the solubility of the scFv fragment (e.g., hydrophilic linkers, such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019; Flo et al., Gene 77:51, 1989; Bird et al., Science 242:423, 1988; Pantoliano et al., Biochemistry 30:10117, 1991; Milenic et al., Cancer Research 51:6363, 1991; and Takkinen et al., Protein Engineering 4:837, 1991. The VL and VH domains of a scFv molecule can be derived from one or more antibody molecules. It is also understood by one of ordinary skill in the art that the variable regions of the scFv molecules described herein can be modified, such that they vary in amino acid sequence from the antibody molecule from which they were derived. For example, in some embodiments, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

As used herein, a “disulfide-bonded isoform” of an antibody or antigen-binding fragment thereof is a form of the antibody or antigen-binding fragment thereof having a particular internal disulfide bonding pattern. Disulfide-bonded isoforms are structural isomers of a given antibody or antigen-binding fragment thereof that do not differ from one another in amino acid sequence but exhibit different disulfide bond connectivity. For example, in the context of a human IgG2 antibody or variant thereof, the antibody may exist in one of four possible disulfide-bonded isoforms, represented herein as isoforms IgG2-A, IgG2-B, IgG2-A/B1, and IgG2-A/B2.

As used herein, the term “complementarity determining region” (CDR) refers to a hypervariable region found both in the light chain and the heavy chain variable domains of an antibody. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may comprise modifications in these hybrid hypervariable positions. The variable domains of native heavy and light chains each comprise four framework regions that primarily adopt a β-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, Bethesda, Md. 1987); incorporated herein by reference). As used herein, numbering of immunoglobulin amino acid residues is done according to the EU numbering system (see, e.g., Edelman et al. Proc. Natl. Acad. Sci. USA 63:78-85, 1969; incorporated herein by reference), unless otherwise indicated. The CDRs can be identified using sequence or structure-based methods that have been described by Kabat et al., Chothia et al. (J. Mol. Biol. 196:901-917, 1987), and MacCallum et al. (J. Mol. Biol. 262:732-745, 1996), where the definitions include overlapping or subsets of amino acid residues when compared against each other. The term “CDR” may be, for example, a CDR as defined by Kabat or Chothia based on sequence comparisons.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and/or steric volume. These properties are summarized for each of the twenty naturally occurring amino acids in Table 1 below.

TABLE 1
Representative physicochemical properties
of naturally occurring amino acids.

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

From this table, it is appreciated that the conservative amino acid families include, but are not limited to: (i) G, A, V, L, I, P, and M; (ii) D and E; (iii) C, S, and T; (iv) H, K, and R; (v) N and Q; and (vi) F, Y, and W. A conservative mutation or substitution is therefore one that substitutes one amino acid for a member of the same amino acid family (e.g., a substitution of Ser for Thr, or Lys for Arg). As is appreciated in the art, a polypeptide may retain its biological properties or functions after the introduction of one or more conservative substitutions.

Amino acid substitutions may be represented herein using the convention (AA1)(N)(AA2), where “AA1” represents the amino acid normally present at particular site within an amino acid sequence, “N” represents the residue number within the amino acid sequence at which the substitution occurs, and “AA2” represents the amino acid present in the amino acid sequence after the substitution is effectuated. For example, the notation “C232S” in the context of an antibody hinge region, such as an IgG2 antibody hinge region, refers to a substitution of the naturally occurring cysteine residue for a serine residue at amino acid residue 232 of the indicated hinge amino acid sequence. Likewise, the notation “C233S” in the context of an antibody hinge region, such as an IgG2 antibody hinge region, refers to a substitution of the naturally occurring cysteine residue for a serine residue at amino acid residue 233 of the indicated hinge amino acid sequence.

As used herein, the term “hydrophobic side chain” refers to an amino acid side chain that exhibits relatively low solubility in water due to, e.g., the steric or electronic properties of the chemical moieties present within the side chain. Examples of amino acids containing hydrophobic side chains include those containing aliphatic hydrocarbons, such as alanine, valine, leucine, isoleucine, proline, and methionine, as well as amino acids containing aromatic ring systems that are electrostatically neutral at physiological pH, such as tryptophan, phenylalanine, and tyrosine.

As used herein, the term “conjugate” refers to a compound formed by the chemical bonding of a reactive functional group of one molecule with an appropriately reactive functional group of another molecule.

As used herein, the term “chemotherapeutic agent” refers to any chemical agent with therapeutic usefulness in the treatment of cancer, such as a cancer described herein. Chemotherapeutic agents encompass both chemical and biological agents. These agents can function to inhibit a cellular activity upon which a cancer cell depends for continued survival. Categories of chemotherapeutic agents include alkylating/alkaloid agents, antimetabolites, hormones, hormone analogs, and antineoplastic drugs. Exemplary chemotherapeutic agents suitable for use in conjunction with the compositions and methods described herein include, without limitation, those set forth in Slapak and Kufe, Principles of Cancer Therapy, Chapter 86 in Harrison's Principles of Internal medicine, 14^{th} edition; Perry et al., Chemotherapeutic, Chapter 17 in Abeloff, Clinical Oncology 2^{nd} ed., 2000; Baltzer L. and Berkery R. (eds): Oncology Pocket Guide to Chemotherapeutic, 2^{nd} ed. St. Louis, Mosby-Year Book, 1995; Fischer D. S., Knobf M. F., Durivage H. J. (eds): The Cancer Chemotherapeutic Handbook, 4^{th} ed. St. Louis, Mosby-Year Handbook, the disclosures of each of which are incorporated herein by reference as they pertain to chemotherapeutic agents.

As used herein, the term “immunotherapy agent” refers to a compound, such as an antibody, antigen-binding fragment thereof, single-chain polypeptide, or construct as described herein, that specifically binds an immune checkpoint protein (e.g., immune checkpoint receptor or ligand) and exerts an antagonistic effect on the receptor or ligand, thereby reducing or inhibiting the signal transduction of the receptor or ligand that would otherwise lead to a downregulation of the immune response. Immunotherapy agents include compounds, such as antibodies, antigen-binding fragments, single-chain polypeptides, and constructs, capable of specifically binding receptors expressed on the surfaces of hematopoietic cells, such as lymphocytes (e.g., T cells), and suppressing the signaling induced by the receptor or ligand that would otherwise lead to tolerance towards an endogenous (“self”) antigen, such as a tumor-associated antigen. Immunotherapy agents may reduce the signaling induced by the receptor or ligand by, for example, 1%, 2%, 3%, 4%, 5%, 10%, 20%, 30%, 40%, 50%, 60%, 70%, 80%, 90%, 95%, 96%, 97%, 98%, 99%, 99.9%, or 100% relative to the signaling induced by the receptor or ligand exhibited in the absence of the immunotherapy agent. Exemplary assays that can be used to measure the extent of receptor or ligand signaling include, for example, enzyme-linked immunosorbent assay (ELISA) techniques to measure protein expression alterations that are associated with a particular signal transduction pathway, as well as polymerase chain reaction (PCR)-based techniques, such as quantitative PCR, reverse-transcription PCR, and real-time PCR experiments useful for determining changes in gene expression associated with a particular signal transduction pathway, among others. Exemplary methods that can be used to determine whether an agent is an “immunotherapy agent” include the assays described in Mahoney et al., Cancer Immunotherapy 14:561-584, 2015, the disclosure of which is incorporated herein by reference in its entirety. Examples of immunotherapy agents include, e.g., antibodies or antigen-binding fragments thereof that specifically bind one or more of TL1A, CD40L, LIGHT, BTLA, LAG3, TIM3, Singlecs, ICOS, B7-H3, B7-H4, VISTA, TMIGD2, BTNL2, CD48, KIR, LIR, LIR antibody, ILT, NKG2D, NKG2A, MICA, MICB, CD244, CSF1R, IDO, TGFβ, CD39, CD73, CXCR4, CXCL12, SIRPA, CD47, VEGF, and neuropilin. Additional examples of immunotherapy agents include Targretin, Interferon-alpha, clobetasol, Peg Interferon (e.g., PEGASYS®), prednisone, Romidepsin, Bexarotene, methotrexate, Triamcinolone cream, anti-chemokines, Vorinostat, gabapentin, antibodies to lymphoid cell surface receptors and/or lymphokines, antibodies to surface cancer proteins, and/or small molecular therapies such as Vorinostat. Particular examples of immunotherapy agents that may be used in or in conjunction with the compositions and methods described herein include anti-PD-1 antibodies and antigen-binding fragments thereof, such as nivolumab, pembrolizumab, avelumab, durvalumab, and atezolizumab, as well as anti-PD-L1 antibodies and antigen-binding fragments thereof, such as atezolizumab and avelumab, and anti-CTLA-4 antibodies and antigen-binding fragments thereof, such as ipilimumab or tremelimumab.

As used herein in the context of a TNFRSF agonist or antagonist, the term “construct” refers to a fusion protein containing a first polypeptide domain bound to a second polypeptide domain. The polypeptide domains may each independently be agonistic or antagonistic antigen-binding fragments that bind TNFRSF member proteins (e.g., single chain polypeptides as described herein). The first polypeptide domain may be covalently bound to the second polypeptide domain, for instance, by way of a linker, such as a peptide linker or a disulfide bridge, among others. Exemplary linkers that may be used to join the polypeptide domains of a TNFRSF agonist or antagonist construct include, without limitation, those that are described in Leriche et al., Bioorg. Med. Chem. 20:571-582, 2012, the disclosure of which is incorporated herein by reference in its entirety.

As used herein, the term “endogenous” describes a molecule (e.g., a polypeptide, nucleic acid, or cofactor) that is found naturally in a particular organism (e.g., a human) or in a particular location within an organism (e.g., an organ, tissue, or cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or cultured matter.

As used herein, the term “epitope” refers to a portion of an antigen that is recognized and bound by a polypeptide, such as an antibody, antigen-binding fragment thereof, single-chain polypeptide, or construct as described herein. In the context of a protein antigen (such as a human TNFRSF member protein), an epitope may be a continuous epitope, which is a single, uninterrupted segment of one or more amino acids covalently linked to one another by peptide bonds in which all of the component amino acids bind the polypeptide (e.g., antibody, antigen-binding fragment thereof, single-chain polypeptide, or construct thereof). Continuous epitopes may be composed, for instance, of 1, 5, 10, 15, 20, or more amino acids within an antigen, such as a human TNFRSF member protein. In some embodiments, an epitope may be a discontinuous epitope, which contains two or more amino acids each separated from one another in the amino acid sequence of an antigen by one or more intervening amino acid residues. Despite this separation by intervening amino acids, the segments that compose a discontinuous epitope may be, for instance, spatially proximal to one another in the three-dimensional conformation of the antigen.

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem. 20:571-582, 2012).

As used herein, the term “hinge region” refers to the domain of an antibody or antigen-binding fragment thereof (e.g., an IgG2 antibody or antigen-binding fragment thereof) located between the antigen-binding portion(s) of the antibody or antigen-binding fragment thereof, such as the Fab region of the antibody or antigen-binding fragment thereof, and the portion of the antibody or antigen-binding fragment thereof that dictates the isotype of the antibody or antigen-binding fragment thereof, such as the Fc region of the antibody or antigen-binding fragment thereof. For example, in the context of a monoclonal antibody, the hinge region is the polypeptide situated approximately in the center of each heavy chain, connecting the CH1 domain to the CH2 and CH3 domains. The hinge region of an antibody or antigen-binding fragment thereof may provide a chemical linkage between chains of the antibody or antigen-binding fragment thereof. For instance, in a monoclonal antibody, the cysteine residues within the hinge region form inter-chain disulfide bonds, thereby providing explicit covalent bonds between heavy chains. The amino acid sequence of wild-type human IgG2 is ERKCCVECPPCP (SEQ ID NO: 51). As used herein, antibody hinge regions are numbered according to the numbering system of Kabat et al, Sequences of Proteins of Immunological Interest (National Institute of Health, Bethesda, Md. 1987), the disclosure of which is incorporated herein by reference.

As used herein, the term “myeloid-derived suppressor cell” or “MDSC” refers to a cell of the immune system that modulates the activity of a variety of effector cells and antigen-presenting cells, such as T cells, NK cells, dendritic cells, and macrophages, among others. Myeloid derived suppressor cells are distinguished by their gene expression profile, and express all or a subset of proteins and small molecules selected from the group consisting of B7-1 (CD80), B7-H1 (PD-L1), CCR2, CD1d, CD1d1, CD2, CD31 (PECAM-1), CD43, CD44, complement component C5a R1, F4/80 (EMR1), Fcγ RIII (CD16), Fcγ RII (CD32), Fcγ RIIA (CD32a), Fcγ RIIB (CD32b), Fcγ RIIB/C (CD32b/c), Fcγ RIIC (CD32c), Fcγ RIIIA (CD16A), Fcγ RIIIB (CD16b), galectin-3, GP130, Gr-1 (Ly-6G), ICAM-1 (CD54), IL-1RI, IL-4Rα, IL-6Ra, integrin α4 (CD49d), integrin αL (CD11a), integrin αM (CD11b), M-CSFR, MGL1 (CD301a), MGL1/2 (CD301a/b), MGL2 (CD301b), nitric oxide, PSGL-1 (CD162), L-selectin (CD62L), siglec-3 (CD33), transferrin receptor (TfR), VEGFR1 (Flt-1), and VEGFR2 (KDR or Flk-1). Particularly, MDSCs do not express proteins selected from the group consisting of B7-2 (CD86), B7-H4, CD11c, CD14, CD21, CD23 (FcεRII), CD34, CD35, CD40 (TNFRSF5), CD117 (c-kit), HLA-DR, and Sca-1 (Ly6).

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, the molecules are identical at that position.

As used herein, the term “proliferation” in the context of a population of cells, such as a population of TNFRSF-expressing cells (e.g., T-reg cells, MDSCs, or TNFRSF-expressing cancer cells) refers to mitotic and cytokinetic division of a cell to produce a plurality of cells. Cell proliferation may be evidenced, for example, by a finding that the quantity of cells (e.g., TNFRSF-expressing cells) in a subject or sample of cells has increased over a given time period, such as over the course of one or more hours, days, or weeks. One of skill in the art may monitor cell proliferation using a variety of known techniques, such as by way of visual microscopy, hemocytometry, flow cytometry, fluorescence activated cell sorting, and other assays known in the art. In the present disclosure, cell proliferation is considered changed (e.g., increased or decreased) when the rate of proliferation of a population of cells, such as a population of TNFRSF-expressing cells contacted with an agonistic or antagonistic TNFRSF antibody or antigen-binding fragment thereof described herein, is changed relative to the rate of proliferation of a population of control cells, such as a population of TNFRSF-expressing cells not contacted with the agonistic or antagonistic TNFRSF antibody or antigen-binding fragment thereof. A change in the rate of proliferation may manifest, for example, as a change (e.g., an increase or decrease) in the quantity of cells of interest in a subject or sample over a given time period, such as a change in the quantity of cells of interest in a subject or sample of 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more, over a given time period. Additionally or alternatively, change of cell proliferation may be evidenced by a finding that the rate at which cells of interest (e.g., TNFRSF-expressing cells contacted with an agonistic or antagonistic TNFRSF antibody or antigen-binding fragment thereof described herein) are dividing is changed (e.g., increased or decreased), e.g., by %, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more, relative to the rate at which control cells (e.g., TNFRSF-expressing cells not contacted with the agonistic or antagonistic TNFRSF antibody or antigen-binding fragment thereof) are dividing.

As used herein, the term “pharmacokinetic profile” refers to the absorption, distribution, metabolism, and clearance of a drug over time following administration of the drug to a subject.

As used herein, the terms “TNFRSF natural ligand,” “endogenous TNFRSF ligand,” and “TNFRSF endogenous ligand” refer to an endogenous ligand of a TNFRSF member protein that may form a TNFRSF ligand-TNFRSF member protein complex and induce the formation of the active, homotrimeric conformation of the TNFRSF member protein. Exemplary TNFRSF ligand-TNFRSF member protein complexes may include, but are not limited to, the CD40L-CD40 complex, the 4-1BBL-4-1BB complex, the CD70-CD27 complex, the CD153-CD30 complex, the N-APP-DR6 complex, the EDA-A1-EDAR complex, the FasL-Fas complex, the GITRL-GITR complex, the LT-α-HVEM complex, the LT-β (TNF-C)-LT-β receptor complex, the NGF-NGFR complex, the TRAIL-OPG complex, the OX40L-OX40 complex, the RANKL-RANK complex, the TNFα-TNFR1 complex, the TRAIL-TRAIL-R2 (TNFRSF10B) complex, the TRAIL-TRAIL-R1 (TNFRSF10A) complex, the TRAIL-TRAIL-R4 complex, the TL1A-TRAMP complex, the EDA-A2-XEDAR complex, the FasL-DCR3 complex, the LIGHT-DCR3 complex, and the TL1A-DCR3 complex.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a K_{d} of less than 100 nM. For example, an antibody or antigen-binding fragment thereof that specifically binds to an antigen will bind to the antigen with a K_{d} of up to 100 nM (e.g., between 1 pM and 100 nM). An antibody or antigen-binding fragment thereof that does not exhibit specific binding to a particular antigen or epitope thereof will exhibit a K_{d} of greater than 100 nM (e.g., greater than 500 nm, 1 μM, 100 μM, 500 μM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “subject” and “patient” refer to an organism that receives treatment for a particular disease or condition as described herein (such as cancer or an infectious disease). Examples of subjects and patients include mammals, such as humans, primates, pigs, goats, rabbits, hamsters, cats, dogs, guinea pigs, members of the Bovidae family (such as cattle, cow, bison, buffalo, elk, and yaks, among others), sheep, and horses, among others, receiving treatment for diseases or conditions, for example, cell proliferation disorders, such as cancer or infectious diseases.

As used herein, the term “transfection” refers to any of a wide variety of techniques commonly used for the introduction of an exogenous polynucleotide into a prokaryotic or eukaryotic host cell, e.g., electroporation, lipofection, calcium phosphate precipitation, diethylaminoethyl (DEAE)-dextran transfection, and the like.

As used herein, the terms “treat” or “treatment” refer to therapeutic treatment, in which the objective is to prevent or slow down (lessen) an undesired physiological change or disorder, such as the progression of a cell proliferation disorder, such as cancer, an autoimmune disease, or an infectious disease. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable (e.g., in particular in a subject that is not treated with a composition described herein). Those in need of treatment include those already with the condition or disorder, as well as those prone to have the condition or disorder, or those in which the condition or disorder is to be prevented.

As used herein, the terms “tumor necrosis factor receptor superfamily,” “TNFR superfamily,” “TNFRS,” “TNFRSF,” or “TNFRSF members” refer to a group of type I transmembrane proteins with a carboxy-terminal intracellular domain and an amino-terminal extracellular domain characterized by a common cysteine-rich domain (CRD). The TNFR superfamily includes receptors that mediate cellular signaling due to binding by one or more ligands in the TNF superfamily. The TNFR superfamily can be divided into two subgroups: receptors containing the intracellular death domain and those lacking this domain. The death domain is an 80 amino acid motif that propagates apoptotic signal transduction cascades following receptor activation. Exemplary TNFR superfamily members that contain the intracellular death domain include TNFR1, while TNFR2 represents a TNFR superfamily protein that does not contain this domain. Members of the TNFR superfamily include CD40, TNFR1, TNFR2, RANK, CD30, lymphotoxin beta receptor (LT-β receptor or LT-βR), OX40, Fas receptor, decoy receptor 3 (DCR3), CD27, 4-1BB, death receptor 4 (DR4), death receptor 5 (DR5), decoy receptor 1 (DCR1), decoy receptor 2 (DCR2), osteoprotegerin, TWEAK receptor, TACI, BAFF receptor, Herpesvirus entry mediator, nerve growth factor receptor, B cell maturation antigen, glucocorticoid-induced TNFR-related protein, TROY, death receptor 6 (DR6), death receptor 3 (DR3), and ectodysplasin A2 receptor.

As used herein, the terms “TNFRSF signaling,” “TNFRSF member protein signaling,” “TNFRSF signal transduction,” “TNFRSF member protein signal transduction,” and the like, are used interchangeably and refer to the cellular events that normally occur upon activation of a TNFRSF member (e.g., TNFR2, TNFR1, CD40, 4-1BB, CD27, CD30, DR6, EDAR, Fas, GITR, HVEM, LT-β receptor, NGFR, OPG, OX40, RANK, RELT (TNFRSF19L), TRAIL-R2 (TNFRSF10B), TRAIL-R1 (TNFRSF10A), TRAIL-R4, TRAMP, TROY, XEDAR, or DCR3) on the surface of a TNFRSF member expressing cell, such as T-reg cell, MDSC, or TNFRSF member expressing cancer cell, by an endogenous TNFRSF member ligand, such as CD40L for CD40, TNFα for TNFR1 and TNFR2, 4-1BBL for 4-1BB, CD70 for CD27, CD153 for CD30, N-APP for DR6, EDA-A1 for EDAR, FasL for Fas, GITRL for GITR, LTα for HVEM, LT-β (TNF-C) for LT-β receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, EDA-A2 for XEDAR, or FasL, LIGHT, and TL1A for DCR3, among others. TNFRSF member signaling may be evidenced by a finding that expression is increased for one or more genes selected from the group consisting of CHUK, NFKBIE, NFKBIA, MAP3K11, TRAF2, TRAF3, relB, and CIAP2/BIRC3. TNFRSF member signaling is considered to be “inhibited” as used herein when the expression (and/or post-translational modification in the event that such a modification is required for activity of the encoded protein) of one or more, or all, of the foregoing genes is decreased in a TNFRSF member expressing cell upon contacting the cell with an agent, such as an agonistic or antagonistic TNFRSF antibody or antigen-binding fragment thereof described herein, relative to a TNFRSF member expressing cell that is not contacted with the agent (e.g., an agonistic or antagonistic TNFRSF antibody or antigen-binding fragment thereof). TNFRSF member signaling is considered to be “inhibited,” for example, when the expression or post-translational modification (e.g., phosphorylation) of one or more of CHUK, NFKBIE, NFKBIA, MAP3K11, TRAF2, TRAF3, relB, or CIAP2/BIRC3, in a TNFRSF member expressing cell contacted with an antagonistic TNFRSF member polypeptide is decreased by about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% relative to the expression or post-translational modification (e.g., phosphorylation) of one or more of these genes in a TNFRSF member expressing cell not contacted with the antagonistic TNFRSF member polypeptide. Exemplary assays that can be used to determine expression level and phosphorylation state are known in the art and include, e.g., Western blot assays to determine protein content and quantitative reverse transcription polymerase chain reaction (RT-PCR) experiments to determine mRNA content.

As used herein, the term “vector” includes a nucleic acid vector, e.g., a DNA vector, such as a plasmid, an RNA vector, and a virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of antibodies and antibody fragments described herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, internal ribosomal entry site (IRES), and polyadenylation signal site to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “VH” refers to the variable region of an immunoglobulin heavy chain of an antibody, including the heavy chain of an Fv, scFv, or Fab. The term “VL” refers to the variable region of an immunoglobulin light chain, including the light chain of an Fv, scFv, dsFv, or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

As used herein, the term “reference level” refers to a threshold level or a level in a control subject or control patient population. A reference level depends on the assay performed and can be determined by one of ordinary skill in the art. A reference level can be a baseline level or a level in the same subject measured at an earlier or later point in time. In some cases, a reference level is determined in a subject prior to or after the administration of an antibody or antigen-binding fragment thereof disclosed herein, a construct disclosed herein, a polynucleotide disclosed herein, a vector disclosed herein, a host cell disclosed herein, or a pharmaceutical composition disclosed herein. For example, some non-limiting examples of reference levels of human soluble TNFR2 include the level of human soluble TNFR2 in a subject that: has not been diagnosed as having a disease; does not present with at least two or more symptoms of a disease; or has not been administered with the anti-TNFRSF antibodies or antigen-binding fragments thereof disclosed herein.

As used herein, the term “modified antibody or antigen-binding fragment thereof” refers to an antibody or antigen-binding fragment thereof (e.g., an antibody or antigen-binding fragment thereof that specifically binds a TNFRSF member protein, such as the antibodies or antigen-binding fragments thereof of the disclosure) that comprises one or more amino acid modifications (e.g., substitutions, deletions, insertions, or chemical modifications, among others). For example, the antibody or antigen-binding fragment thereof may have one or more amino acid modifications at the Fc domain. The term “unmodified antibody or antigen-binding fragment thereof” refers to an antibody or antigen-binding fragment thereof corresponding to a modified antibody or antigen-binding fragment thereof but that does not comprise the one or more amino acid modifications.

As used herein, the term “biological fluid” refers to the source of the fluid, and includes (but is not limited to) amniotic fluid, aqueous humor, blood and blood plasma (and herein blood refers to the plasma component, unless otherwise expressly stated or indicated in context), cerumen (ear wax), Cowper's fluid, chime, interstitial fluid, lymph fluids, mammalian milk, mucus, pleural fluid, pus, saliva, sebum, semen, serum, sweat tears, urine, vaginal secretion, vomit, and exudates (from wounds or lesions). In some embodiments, the biological fluid of the disclosure is serum. In some embodiments, the biological fluid of the disclosure is plasma.

As used herein, the term “immune response” includes, but is not limited to, T cell mediated and/or B cell mediated immune responses. Exemplary immune responses include T cell responses, e.g., cytokine production and cellular cytotoxicity. T-cell responses include Th1 and/or Th2 responses. In addition, the term immune response includes responses that are indirectly promoted by T cell activation, e.g., antibody production (humoral responses) and activation of cytokine responsive cells, e.g., eosinophils, macrophages. In addition, the term immune response includes immune responses that are indirectly affected by T cell activation, e.g., antibody production (humoral responses) and activation of cytokine responsive cells, e.g., macrophages. Immune cells involved in the immune response include lymphocytes, such as T cells (CD4+, CD8+, Th1 and Th2 cells, memory T cells) and B cells; antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer (NK) cells; myeloid cells, such as macrophages, eosinophils, mast cells, basophils, and granulocytes. An immune response can also refer to any of innate immunity, humoral immunity, cellular immunity, autoimmunity, inflammatory response, and acquired (adaptive) immunity.

As used herein, the term “dose range” refers to an upper and a lower limit of an acceptable variation of the amount (e.g., dose) of an agent specified (e.g., an antibody or antigen-binding fragment thereof of the disclosure that specifically binds a TNFRSF member protein, a TNFSF member protein, CD28, or ICOS). Typically, a dose of an agent in any amount within the specified range can be administered to patients undergoing treatment.

### BRIEF DESCRIPTION OF THE DRAWINGS

FIG. 1A is a graph showing the live cell count of Jeko-1 tumor cells at varying concentrations of a fully humanized TNFR2 antagonist antibody. The experiment was conducted as described in Example 1 below.

FIG. 1B is a graph showing the live cell count of Jeko-1 tumor cells at varying concentrations of a fully humanized TNFR2 antagonist antibody with reduced interaction with Fc receptor.

FIG. 2A is a graph showing the percentage of Treg cells in human T cells treated with a fully humanized TNFR2 agonist antibody. The experiment was conducted as described in Example 2 below.

FIG. 2B is a graph showing the percentage of Treg cells in human T cells treated with a fully humanized TNFR2 agonist antibody with LALA mutations in the Fc region.

FIG. 3A is a graph showing the percentage of Treg cells in human T cells treated with a human anti-human TNFR2 agonist. The experiment was conducted as described in Example 3 below.

FIG. 3B is a graph showing the percentage of Treg cells in human T cells treated with a human anti-human TNFR2 agonist with LALA mutations in the Fc region.

FIG. 3C is a graph showing the percentage of Treg cells in human T cells treated with a mouse anti-human TNFR2 agonist.

FIG. 4A is a graph showing Treg cell proliferation in response to increasing concentrations of humanized TNFR2 agonists with IgG1 framework (black squares) or modified IgG4 framework (black circles).

FIG. 4B is a graph showing the change of the percentage of effector T cells (Teff) in total T cells in response to increasing concentrations of humanized TNFR2 agonists with IgG1 framework (black squares) or modified IgG4 framework (black circles).

### DETAILED DESCRIPTION

Antibodies or antigen-binding fragments thereof of this disclosure that specifically bind human TNFRSF member proteins (e.g., TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, or DCR3, among others), TNFSF member proteins (e.g., TRAIL), CD28, or ICOS comprise one or more amino acid modifications (e.g., substitutions, deletions, insertions, or chemical modifications) at the Fc domain. Such modifications decrease an effector function (e.g., ADCC or ADCP) of the antibody or antigen-binding fragment thereof mediated by an Fc receptor (e.g., FcγRI, FcγRII, or FcγRIII) and enhance a biological activity (e.g., agonistic or antagonistic activity) of the antibody or antigen-binding fragment thereof.

### Anti-TNFRSF Antibodies or Antigen-Binding Fragments Thereof

Antibodies or antigen-binding fragments that specifically bind to TNFRSF member proteins (e.g., TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, or DCR3, among others) are well known in the art. The antibody or antigen-binding fragment thereof can be an agonistic antibody or antigen-binding fragment thereof, which, upon binding with a TNFRSF member protein, promotes or augments the activation of the TNFRSF member protein. Alternatively, the antibody or antigen-binding fragment thereof can be an antagonistic antibody or antigen-binding fragment thereof, which, upon binding with a TNFRSF member protein, inhibits or reduces the activation of the TNFRSF member protein.

### Agonistic Anti-TNFR2 Antibodies or Antigen-Binding Fragments Thereof

In some embodiments, the antibody or antigen-binding fragment thereof is an agonistic antibody or antigen-binding fragment thereof that specifically binds TNFR2 comprising one or more CDRs with amino acid sequences selected from Table 2 below, or variants thereof with up to two conservative amino acid substitutions. For example, the antibody or the antigen-binding fragment thereof may include a CDR-H1, a CDR-H2, a CDR-H3, a CDR-L1, a CDR-L2, and a CDR-L3 selected from Table 2.

TABLE 2
CDR sequences for agonistic anti-TNFR2 antibodies
or antigen-binding fragments thereof.

| Publication | | SEQ ID |
|---|---|---|
| Number | CDR sequences | NO: |
| WO 2020/102739 | CDR-H1: GYTFTDYNI | 1 |
| | CDR-H2: INPNYDST | 2 |
| | CDR-H3: CARGNSWYFDV | 3 |
| | CDR-L1: SSVRY | 4 |
| | CDR-L2: LTS | N/A |
| | CDR-L3: CQQWSSNPLT | 6 |
| WO 2020/102739 | CDR-H1: GYTFTDYNL | 7 |
| | CDR-H2: INPNYDST | 8 |
| | CDR-H3: CARGNSWYFDV | 9 |
| | CDR-L1: SSVRY | 10 |
| | CDR-L2: LTS | N/A |
| | CDR-L3: CQQWSSNPLT | 12 |
| WO 2021/231922 | CDR-H1: DYIMH | 13 |
| | CDR-H2: WVDPEYGST | 14 |
| | CDR-H3: DDGSYSPFD | 15 |
| | CDR-L1: KASENVVTYVS | 16 |
| | CDR-L2: GASNRYT | 17 |
| | CDR-L3: GQGYSYPY | 18 |
| WO 2021/231922 | CDR-H1: DYIMH | 19 |
| | CDR-H2: WVDPEYGST | 20 |
| | CDR-H3: DDGSYSPFD | 21 |
| | CDR-L1: RASENVVTYVS | 22 |
| | CDR-L2: GASNRYT | 23 |
| | CDR-L3: GQGYSYPY | 24 |
| WO 2020/061210 | CDR-H1: TFGMS | 25 |
| | CDR-H2: WINTYSGVPTYADDFKG | 26 |
| | CDR-H3: RSNFAY | 27 |
| | CDR-L1: RASESVDSSGNSFMH | 28 |
| | CDR-L2: RASNLES | 29 |
| | CDR-L3: QQSNEDPWT | 30 |
| WO 2020/061210 | CDR-H1: GYTFTTF | 31 |
| | CDR-H2: NTYSGV | 32 |
| | CDR-H3: RSNFAY | 33 |
| | CDR-L1: RASESVDSSGNSFMH | 34 |
| | CDR-L2: RASNLES | 35 |
| | CDR-L3: QQSNEDPWT | 36 |
| WO 2020/061210 | CDR-H1: GYTFTTFGMS | 37 |
| | CDR-H2: WINTYSGVPT | 38 |
| | CDR-H3: RSNFAY | 39 |
| | CDR-L1: RASESVDSSGNSFMH | 40 |
| | CDR-L2: RASNLES | 41 |
| | CDR-L3: QQSNEDPWT | 42 |
| WO 2020/061210 | CDR-H1: GYTFTTF | 43 |
| | CDR-H2: INTYSGVP | 44 |
| | CDR-H3: ARRSNFAY | 45 |
| | CDR-L1: ESVDSSGNSF | 46 |
| | CDR-L2: RAS | N/A |
| | CDR-L3: CQQSNEDPWT | 48 |
| WO 2020/061210 | CDR-H1: GYTFTTF | 49 |
| | CDR-H2: NTYSGV | 50 |
| | CDR-H3: RSNFAY | 51 |
| | CDR-L1: RASESLTASGNSFMH | 52 |
| | CDR-L2: RASNLES | 53 |
| | CDR-L3: QQSRHVNWT | 54 |
| WO 2020/061210 | CDR-H1: GYTFTTF | 55 |
| | CDR-H2: NTYSGV | 56 |
| | CDR-H3: RSNFAY | 57 |
| | CDR-L1: RASQTVDSSGNSFMH | 58 |
| | CDR-L2: LGNRLES | 59 |
| | CDR-L3: QQSNEDPWT | 60 |
| WO 2020/061210 | CDR-H1: GYTFTTF | 61 |
| | CDR-H2: NTYSGV | 62 |
| | CDR-H3: RSNFAY | 63 |
| | CDR-L1: RASESLTASGNSFMH | 64 |
| | CDR-L2: RASNLES | 65 |
| | CDR-L3: QQSRHVNWT | 66 |
| WO 2020/061210 | CDR-H1: TFGMS | 67 |
| | CDR-H2: WINTYSGVPHYAQGFTG | 68 |
| | CDR-H3: RSNFAY | 69 |
| | CDR-L1: RASESLTASGNSFMH | 70 |
| | CDR-L2: RASNLES | 71 |
| | CDR-L3: QQSRHVNWT | 72 |
| WO 2020/061210 | CDR-H1: GYTFTTFG | 73 |
| | CDR-H2: INTYSGVP | 74 |
| | CDR-H3: ARRSNFAY | 75 |
| | CDR-L1: ESLTASGNSF | 76 |
| | CDR-L2: RAS | N/A |
| | CDR-L3: QQSRHVNWT | 78 |
| WO 2020/180712 | CDR-H1: TYAMS | 79 |
| | CDR-H2: IISDGGDATFYADSVKG | 80 |
| | CDR-H3: DGTSAAAFDS | 81 |
| | CDR-L1: QASQDITNFLN | 82 |
| | CDR-L2: DASTLQT | 83 |
| | CDR-L3: QQSDSYPIT | 84 |
| WO 2020/180712 | CDR-H1: TYAMS | 85 |
| | CDR-H2: IISDGGDATVYADSVKG | 86 |
| | CDR-H3: DGTSAAAFDS | 87 |
| | CDR-L1: QASQDITNFLN | 88 |
| | CDR-L2: DASTLQT | 89 |
| | CDR-L3: QQSDSYPIT | 90 |
| WO 2020/180712 | CDR-H1: TYAMS | 91 |
| | CDR-H2: IISDGGDATVYADSVKG | 92 |
| | CDR-H3: DGTSAAAFDS | 93 |
| | CDR-L1: QASQDITNFLN | 94 |
| | CDR-L2: DASTLQT | 95 |
| | CDR-L3: QQSDSYPIT | 96 |
| WO 2020/180712 | CDR-H1: TYAMS | 97 |
| | CDR-H2: IISDGGDATFYADSVKG | 98 |
| | CDR-H3: DGTSAAAFDS | 99 |
| | CDR-L1: QASQDITNFLN | 100 |
| | CDR-L2: DASRRRT | 101 |
| | CDR-L3: QQSDSYPIT | 102 |
| WO 2020/180712 | CDR-H1: TYAMS | 103 |
| | CDR-H2: IISDGGDATVYADSVKG | 104 |
| | CDR-H3: DGTSAAAFDS | 105 |
| | CDR-L1: QASQDITNFLN | 106 |
| | CDR-L2: DASRRRT | 107 |
| | CDR-L3: QQSDSYPIT | 108 |
| WO 2020/180712 | CDR-H1: TYAMS | 109 |
| | CDR-H2: IISDGGDATVYADSVKG | 110 |
| | CDR-H3: DGTSAAAFDS | 111 |
| | CDR-L1: HASQDITNFLN | 112 |
| | CDR-L2: DASTLQT | 113 |
| | CDR-L3: QQSDSYPIT | 114 |
| WO 2020/180712 | CDR-H1: TYAMS | 115 |
| | CDR-H2: IISDGGDATVYADSVKG | 116 |
| | CDR-H3: DRTSAAAFDS | 117 |
| | CDR-L1: QASQDITNFLN | 118 |
| | CDR-L2: DASTLQT | 119 |
| | CDR-L3: QQSDSYPIT | 120 |
| WO 2020/180712 | CDR-H1: TYAMS | 121 |
| | CDR-H2: ISDGGDATVYADSVKG | 122 |
| | CDR-H3: DGTSAAAFDS | 123 |
| | CDR-L1: QASQDITNFLN | 124 |
| | CDR-L2: DASTLQT | 125 |
| | CDR-L3: QQSDSYPIT | 126 |
| WO 2020/180712 | CDR-H1: TYAMS | 127 |
| | CDR-H2: IISDGGDATVYADSVKG | 128 |
| | CDR-H3: DGTSAAAFDS | 129 |
| | CDR-L1: QASQDITNFLN | 130 |
| | CDR-L2: DASTLQT | 131 |
| | CDR-L3: QQSDSYPIT | 132 |
| WO 2020/180712 | CDR-H1: TYAMS | 133 |
| | CDR-H2: IISDGGDVTVYADSVKG | 134 |
| | CDR-H3: DRTSAAAFDS | 135 |
| | CDR-L1: QASQDITNFLN | 136 |
| | CDR-L2: DASTLQT | 137 |
| | CDR-L3: QQSDSYPIT | 138 |
| WO 2020/180712 | CDR-H1: TYAMS | 139 |
| | CDR-H2: IISDGGDATVYADSVKG | 140 |
| | CDR-H3: DRTSAAAFDS | 141 |
| | CDR-L1: QASQDITNFLN | 142 |
| | CDR-L2: DASTLQT | 143 |
| | CDR-L3: QQSDSYPIT | 144 |
| WO 2020/180712 | CDR-H1: TYAMS | 145 |
| | CDR-H2: IISDGGDATVYADSVKG | 146 |
| | CDR-H3: DGTSAAAFDS | 147 |
| | CDR-L1: QASQDITNFLN | 148 |
| | CDR-L2: DASTLQT | 149 |
| | CDR-L3: QQSDSYPIT | 150 |
| WO 2020/180712 | CDR-H1: SYAMT | 151 |
| | CDR-H2: GISGGGAATFYADSVKG | 152 |
| | CDR-H3: QSQYVGGFDY | 153 |
| | CDR-L1: TGSSSNIGAGYGVH | 154 |
| | CDR-L2: GNTNRPS | 155 |
| | CDR-L3: QSYDSSLSGWV | 156 |
| WO 2020/180712 | CDR-H1: SYAMT | 157 |
| | CDR-H2: GISGGGAATFYADSVKG | 158 |
| | CDR-H3: QSQYVGGFDY | 159 |
| | CDR-L1: TGSSSNIGAGYGVH | 160 |
| | CDR-L2: GNTNRPS | 161 |
| | CDR-L3: QSYDSSLSGWV | 162 |
| WO 2020/180712 | CDR-H1: SYAMT | 163 |
| | CDR-H2: GISGGGAATFYADSVKG | 164 |
| | CDR-H3: QSQYVGGFDY | 165 |
| | CDR-L1: TGSSSNFGAGYGVH | 166 |
| | CDR-L2: GNTNRPS | 167 |
| | CDR-L3: QSYDSSLSGWV | 168 |
| WO 2020/180712 | CDR-H1: SYAMT | 169 |
| | CDR-H2: GISGDGAATFYADSVKG | 170 |
| | CDR-H3: QSQYVGGFDY | 171 |
| | CDR-L1: TGSSSNFGAGYGVH | 172 |
| | CDR-L2: GNTNRPS | 173 |
| | CDR-L3: QSYDSSLSGWV | 174 |
| WO 2020/180712 | CDR-H1: SYAMS | 175 |
| | CDR-H2: GISAGGGETFYADSVKG | 176 |
| | CDR-H3: VHPISYGFDI | 177 |
| | CDR-L1: QASQDIKKYLN | 178 |
| | CDR-L2: DASTLQT | 179 |
| | CDR-L3: QQSDNTPVT | 180 |
| WO 2020/180712 | CDR-H1: SYAIH | 181 |
| | CDR-H2: VISSDGGYKNYADSVKG | 182 |
| | CDR-H3: DRQIGD | 183 |
| | CDR-L1: RASHGISNYLA | 184 |
| | CDR-L2: AASTLQS | 185 |
| | CDR-L3: QQYNTYRT | 186 |
| WO 2020/180712 | CDR-H1: TYAMT | 187 |
| | CDR-H2: AISDSGGDTFYADSVKG | 188 |
| | CDR-H3: DGTYAARFDY | 189 |
| | CDR-L1: QASQDISNYLN | 190 |
| | CDR-L2: DASTLET | 191 |
| | CDR-L3: QQSDSFPIT | 192 |
| WO 2020/180712 | CDR-H1: NFGIS | 193 |
| | CDR-H2: GIVPVLGIATYAQKFQG | 194 |
| | CDR-H3: GSAWYDGSFQY | 195 |
| | CDR-L1: RASQSVSSTFLA | 196 |
| | CDR-L2: DASTRAT | 197 |
| | CDR-L3: QQYDSWPFT | 198 |
| WO 2020/180712 | CDR-H1: SYGVS | 199 |
| | CDR-H2: RIVPVFGIANYAQKFQG | 200 |
| | CDR-H3: QSPYVTYSSYYFDY | 201 |
| | CDR-L1: RASQSVSNNFLA | 202 |
| | CDR-L2: DASTRAT | 203 |
| | CDR-L3: QQYGSWPIT | 204 |
| WO 2020/180712 | CDR-H1: NYGFS | 205 |
| | CDR-H2: GIVPVFGIATYAQKFQG | 206 |
| | CDR-H3: DYSYYPDDPRYFEY | 207 |
| | CDR-L1: RASQSVSSTFLA | 208 |
| | CDR-L2: AASSRAT | 209 |
| | CDR-L3: QQYGSWPLT | 210 |
| WO 2021/055253 | CDR-H1: SYTMG | 211 |
| | CDR-H2: FISSSGHTYYANWAKG | 212 |
| | CDR-H3: EGGYGGYDYTGIFNL | 213 |
| | CDR-L1: QATESISSWLA | 214 |
| | CDR-L2: GASTLES | 215 |
| | CDR-L3: QQGYIYTNVDNT | 216 |
| WO 2021/055253 | CDR-H1: SYTMG | 217 |
| | CDR-H2: FISSSGHTYYANWAKG | 218 |
| | CDR-H3: DGGYGGYDYTGIFNL | 219 |
| | CDR-L1: QATESISSWLA | 220 |
| | CDR-L2: GASTLES | 221 |
| | CDR-L3: QQGYIYTNVDNT | 222 |
| WO 2021/055253 | CDR-H1: SYGVN | 223 |
| | CDR-H2: GINTGGSTYYANWAKG | 224 |
| | CDR-H3: TSGNNVYNYFTL | 225 |
| | CDR-L1: QASQSIPSLLA | 226 |
| | CDR-L2: APSTLAS | 227 |
| | CDR-L3: QSYYYGDNTYNNI | 228 |
| WO 2021/055253 | CDR-H1: TYDIN | 229 |
| | CDR-H2: IIYTGGITNFANWAKG | 230 |
| | CDR-H3: GGYDSEGYVYPDAFDP | 231 |
| | CDR-L1: QASESISNLLA | 232 |
| | CDR-L2: RASILTS | 233 |
| | CDR-L3: QHGYTGTNVQNV | 234 |
| WO 2021/055253 | CDR-H1: NYAMG | 235 |
| | CDR-H2: SRRTDGITYYANWAEG | 236 |
| | CDR-H3: DVGGEGGWYFNL | 237 |
| | CDR-L1: QASQSINIYLA | 238 |
| | CDR-L2: DASKLAS | 239 |
| | CDR-L3: QQGINNIG | 240 |
| WO 2021/055253 | CDR-H1: NYAMG | 241 |
| | CDR-H2: SRRTDGITYYANWAEG | 242 |
| | CDR-H3: DVGGDGGWYFNL | 243 |
| | CDR-L1: QASQSINIYLA | 244 |
| | CDR-L2: DASKLAS | 245 |
| | CDR-L3: QQGINNIG | 246 |
| WO 2021/055253 | CDR-H1: SYAMG | 247 |
| | CDR-H2: DISTSGNAYYATWVKG | 248 |
| | CDR-H3: ADYGGETYAFDP | 249 |
| | CDR-L1: QASQSISSYLN | 250 |
| | CDR-L2: SASTLAS | 251 |
| | CDR-L3: QQGYSDSNIDNV | 252 |
| WO 2021/055253 | CDR-H1: SHHMI | 253 |
| | CDR-H2: IIDAGSGSTYYASWAKG | 254 |
| | CDR-H3: GGLTESLGTYFDL | 255 |
| | CDR-L1: QASESIDSGLA | 256 |
| | CDR-L2: DSSTLAS | 257 |
| | CDR-L3: QSNYDTGSSVYDWGS | 258 |
| WO 2021/055253 | CDR-H1: DYFMT | 259 |
| | CDR-H2: IINTGGDSYYATWAKG | 260 |
| | CDR-H3: DTGYGGYDYAGSFDP | 261 |
| | CDR-L1: QASENINSWLA | 262 |
| | CDR-L2: EASKLAS | 263 |
| | CDR-L3: QQGYIYIDVGNI | 264 |
| WO 2021/055253 | CDR-H1: VSYWIC | 265 |
| | CDR-H2: CTDGGDGSSYYASWVNG | 266 |
| | CDR-H3: DRSDVFNL | 267 |
| | CDR-L1: QAGQSIDSNLA | 268 |
| | CDR-L2: RASTLAS | 269 |
| | CDR-L3: QSFYVTISAMVDYP | 270 |
| WO 2021/055253 | CDR-H1: RYAMA | 271 |
| | CDR-H2: YIDTGDSTYYATWAKG | 272 |
| | CDR-H3: VGVRMYL | 273 |
| | CDR-L1: QASQSISSYLS | 274 |
| | CDR-L2: RASTLES | 275 |
| | CDR-L3: QCGYYGGSYIGA | 276 |
| WO 2021/055253 | CDR-H1: SYGIS | 277 |
| | CDR-H2: YIYPDYGSTDYATWVNG | 278 |
| | CDR-H3: GYASSSGYYDPKYFGL | 279 |
| | CDR-L1: RASEDIESYLA | 280 |
| | CDR-L2: DASDLAS | 281 |
| | CDR-L3: QHGFYTSRSDSV | 282 |
| WO 2021/055253 | CDR-H1: SYDMS | 283 |
| | CDR-H2: YIWSSGSAYYATWAEG | 284 |
| | CDR-H3: RYVGSSYDT | 285 |
| | CDR-L1: QSSQSVSSNNYLS | 286 |
| | CDR-L2: AASYLAS | 287 |
| | CDR-L3: LGDYDNDIDHA | 288 |
| WO 2021/055253 | CDR-H1: SYAMG | 289 |
| | CDR-H2: FIDTGGSTYYANWAKG | 290 |
| | CDR-H3: VGARMYL | 291 |
| | CDR-L1: QASQSISNLLA | 292 |
| | CDR-L2: RASTLES | 293 |
| | CDR-L3: QCSYYGGSYIGA | 294 |
| WO 2021/055253 | CDR-H1: RYYMS | 295 |
| | CDR-H2: YIDPIFGNTYYASWVNG | 296 |
| | CDR-H3: DGDAGYDGYGYGTDL | 297 |
| | CDR-L1: QASENIYSGLA | 298 |
| | CDR-L2:SAFTLAS | 299 |
| | CDR-L3: QTYYYGSVTYFNA | 300 |
| WO 2021/055253 | CDR-H1: SHYMI | 301 |
| | CDR-H2: IITSSDYIYYARWAKGR | 302 |
| | CDR-H3: YNYDDDGELFNL | 303 |
| | CDR-L1: QSSQSIDANNDLA | 304 |
| | CDR-L2: LASKLAS | 305 |
| | CDR-L3: LGGYDDDADNT | 306 |
| WO 2021/055253 | CDR-H1: NNYYMC | 307 |
| | CDR-H2: CIYPSIVGPTYYANWAKG | 308 |
| | CDR-H3: DRYDDYGDYFNL | 309 |
| | CDR-L1: QASQSIYNYLS | 310 |
| | CDR-L2: YASTLAS | 311 |
| | CDR-L3: QSNSGVNGNRYGNA | 312 |
| WO 2021/141907 | CDR-H1: SYSFTDYN | 313 |
| | CDR-H2: IFPKYGTTSYNQKFKG | 314 |
| | CDR-H3: ATDGGTWYFDV | 315 |
| | CDR-L1: SSVTY | 316 |
| | CDR-L2: LTSNLASGVPA | 317 |
| | CDR-L3: QQWSSNPPT | 318 |
| WO 2021/141907 | CDR-H1: SYSFTDYN | 319 |
| | CDR-H2: IFPKYGTTSYAQKLQG | 320 |
| | CDR-H3: ATDGGTWYFDV | 321 |
| | CDR-L1: SSVTY | 322 |
| | CDR-L2: LTSNLASGVPS | 323 |
| | CDR-L3: QQWSSNPPT | 324 |
| WO 2021/141907 | CDR-H1: GYTFTDYY | 325 |
| | CDR-H2: INPNDGGTTYSQKFKG | 326 |
| | CDR-H3: AREGNYYAYDVRVWYFDV | 327 |
| | CDR-L1: QDIITY | 328 |
| | CDR-L2: STSSLNSGVPS | 329 |
| | CDR-L3: QQYSELPYT | 330 |
| WO 2021/141907 | CDR-H1: GYTFTDYY | 331 |
| | CDR-H2: INPNDGGTTYAQKFQG | 332 |
| | CDR-H3: AREGNYYAYDVRVWYFDV | 333 |
| | CDR-L1: QDIITY | 334 |
| | CDR-L2: STSSLNSGVPS | 335 |
| | CDR-L3: QQYSELPYT | 336 |
| WO 2021/141907 | CDR-H1: GFTFSDAW | 337 |
| | CDR-H2: VRNKANNHATYYAESVKG | 338 |
| | CDR-H3: TRSVGGYGTTYWYFDV | 339 |
| | CDR-L1: QNLLNSGNQKNY | 340 |
| | CDR-L2: GASTRESGVPD | 341 |
| | CDR-L3: QSEHSYPYT | 342 |
| WO 2021/141907 | CDR-H1: GFTFSDAW | 343 |
| | CDR-H2: VRNKANNHATYYAASVKG | 344 |
| | CDR-H3: TRSVGGYGTTYWYFDV | 345 |
| | CDR-L1: QNLLNSGNQKNY | 346 |
| | CDR-L2: GASTRESGVPD | 347 |
| | CDR-L3: QSEHSYPYT | 348 |
| WO 2022/003690 | CDR-H1: GDTFTSYS | 349 |
| | CDR-H2: IITILDDT | 350 |
| | CDR-H3: AGVYEGPNSEGSYDDYGFLKH | 351 |
| | CDR-L1: ESVAND | 352 |
| | CDR-L2: GAS | N/A |
| | CDR-L3: QQYANWPPRET | 354 |
| WO 2022/003690 | CDR-H1: GDTFTSYS | 355 |
| | CDR-H2: IITILDDT | 356 |
| | CDR-H3: AGVYEGPNSEGSYDDYGFLKH | 357 |
| | CDR-L1: ESVAND | 358 |
| | CDR-L2: GAS | N/A |
| | CDR-L3: QQYANWPPRET | 360 |
| WO 2022/003690 | CDR-H1: GDTFTSYS | 361 |
| | CDR-H2: IITDLDDT | 362 |
| | CDR-H3: AGVYEGPNSEGSYDDYGFLKH | 363 |
| | CDR-L1: ESGAND | 364 |
| | CDR-L2: GAS | N/A |
| | CDR-L3: QQYANWPPRGT | 366 |
| WO 2022/003690 | CDR-H1: GDTFTSYS | 367 |
| | CDR-H2: IITILDDT | 368 |
| | CDR-H3: AGVYEGPNSEGSYDDYGFLKH | 369 |
| | CDR-L1: ESVAND | 370 |
| | CDR-L2: GAS | N/A |
| | CDR-L3: QQYTNWPPRET | 372 |
| WO 2022/003690 | CDR-H1: GDTFTSYS | 373 |
| | CDR-H2: IITILDDT | 374 |
| | CDR-H3: AGVYEGPNSEGSYDDYGFLKH | 375 |
| | CDR-L1: ESVAND | 376 |
| | CDR-L2: GAS | N/A |
| | CDR-L3: QQYAIWPPRET | 378 |
| WO 2022/003690 | CDR-H1: GDTFTSYS | 379 |
| | CDR-H2: IITILDDT | 380 |
| | CDR-H3: AGVYEGPNSEGSYDDYGFLKH | 381 |
| | CDR-L1: RSVAND | 382 |
| | CDR-L2: GAS | N/A |
| | CDR-L3: QQYAIWPPRET | 384 |
| WO 2022/003690 | CDR-H1: GDTFTSYS | 385 |
| | CDR-H2: IITILDDT | 386 |
| | CDR-H3: AGVYEGPNSEGSYDDYGFLKH | 387 |
| | CDR-L1: ESVANN | 388 |
| | CDR-L2: GAS | N/A |
| | CDR-L3: QQYARWPPTET | 390 |
| WO 2022/003690 | CDR-H1: GDTFTSYS | 391 |
| | CDR-H2: IITIPDDT | 392 |
| | CDR-H3: AGVYEGPNSEGSYDDYGFLKH | 393 |
| | CDR-L1: ESVATD | 394 |
| | CDR-L2: GAS | N/A |
| | CDR-L3: QQYDNWPPRET | 396 |
| WO 2022/003690 | CDR-H1: GDTFTSYS | 397 |
| | CDR-H2: IITILDDT | 398 |
| | CDR-H3: AGVYEGPNSEGSYDDYGFLKH | 399 |
| | CDR-L1: ESVRND | 400 |
| | CDR-L2: GAS | N/A |
| | CDR-L3: QQYANWPPHET | 402 |
| WO 2022/003690 | CDR-H1: GDTFTSYS | 403 |
| | CDR-H2: IITIDDDT | 404 |
| | CDR-H3: AGVYEGPNSEGSYDDYGFYKH | 405 |
| | CDR-L1: ESVGND | 406 |
| | CDR-L2: GAS | N/A |
| | CDR-L3: QQYANWPPRET | 408 |
| WO 2022/003690 | CDR-H1: GDTFTSYS | 409 |
| | CDR-H2: IITILDDS | 410 |
| | CDR-H3: AGVYEGPNSEGSYDDYGFLKH | 411 |
| | CDR-L1: ESVAND | 412 |
| | CDR-L2: DAS | N/A |
| | CDR-L3: QQYANWPWRET | 414 |
| WO 2022/003690 | CDR-H1: GDTFTSYS | 415 |
| | CDR-H2: IITIDDDT | 416 |
| | CDR-H3: AGVYEGPNSEGSYDDYGFYKH | 417 |
| | CDR-L1: ESVAND | 418 |
| | CDR-L2: GAS | N/A |
| | CDR-L3: QQGANWPPRET | 420 |
| WO 2022/003690 | CDR-H1: GDTFTSYS | 421 |
| | CDR-H2: IITIYDDT | 422 |
| | CDR-H3: AGVYEGPNSEGSYDDYGFFKH | 423 |
| | CDR-L1: ESVAND | 424 |
| | CDR-L2: GAS | N/A |
| | CDR-L3: QQYANWPPRET | 426 |
| WO 2022/003690 | CDR-H1: GDTFTSYS | 427 |
| | CDR-H2: IITILDDT | 428 |
| | CDR-H3: AGVYEGPNSEGSYDDYGFLKY | 429 |
| | CDR-L1: ESVAVD | 430 |
| | CDR-L2: GAS | N/A |
| | CDR-L3: QQYANKPPRET | 432 |
| WO 2022/003690 | CDR-H1: GDTFTSYS | 433 |
| | CDR-H2: IITILDDT | 434 |
| | CDR-H3: AGVYEGPNSEGSYDDYGALKH | 435 |
| | CDR-L1: ESVAND | 436 |
| | CDR-L2: GAS | N/A |
| | CDR-L3: QQYANWPPRET | 438 |
| WO 2022/003690 | CDR-H1: GDTFTSYS | 439 |
| | CDR-H2: IITDLDDT | 440 |
| | CDR-H3: AGVYEGPNSEGSYDDYGFLKH | 441 |
| | CDR-L1: ESGAND | 442 |
| | CDR-L2: GAS | N/A |
| | CDR-L3: QQYANWPPRGT | 444 |
| WO 2022/003690 | CDR-H1: GDTFTSYS | 445 |
| | CDR-H2: IITDLDDT | 446 |
| | CDR-H3: AGVYEGPNSEGSYDDYGQLKH | 447 |
| | CDR-L1: ESVAND | 448 |
| | CDR-L2: GAS | N/A |
| | CDR-L3: QQYANWPPRET | 450 |
| WO 2022/003690 | CDR-H1: GDTFTSYS | 451 |
| | CDR-H2: IITILDDT | 452 |
| | CDR-H3: AGVYEGPNSEGSYDDYGQLKH | 453 |
| | CDR-L1: ESVAND | 454 |
| | CDR-L2: GAS | N/A |
| | CDR-L3: QQYANTPPRET | 456 |
| WO 2022/003690 | CDR-H1: GDTFTSYS | 457 |
| | CDR-H2: IITDLDDT | 458 |
| | CDR-H3: AGVYEGPNSEGSYDDYGFLKH | 459 |
| | CDR-L1: ESVAND | 460 |
| | CDR-L2: GAS | N/A |
| | CDR-L3: QQYANWPPRET | 462 |
| WO 2022/003690 | CDR-H1: GDTFTSYS | 463 |
| | CDR-H2: IITILDDT | 464 |
| | CDR-H3: AGVYEGPNSEGSYDDYGFLKH | 465 |
| | CDR-L1: ESVGND | 466 |
| | CDR-L2: GAS | N/A |
| | CDR-L3: QQYANWPPRET | 468 |
| WO 2022/003690 | CDR-H1: GDTFTSYS | 469 |
| | CDR-H2: IITIDDDT | 470 |
| | CDR-H3: AGVYEGPNSEGSYDDYGQLKH | 471 |
| | CDR-L1: ESVGND | 472 |
| | CDR-L2: GAS | N/A |
| | CDR-L3: QQYANVPPRET | 474 |
| WO 2022/003690 | CDR-H1: GDTFTSYS | 475 |
| | CDR-H2: IITIDDDT | 476 |
| | CDR-H3: AGVYEGPNSEGSYDDYGQLKH | 477 |
| | CDR-L1: ESVGND | 478 |
| | CDR-L2: GIS | N/A |
| | CDR-L3: QQYANDPPRET | 480 |
| WO 2022/003690 | CDR-H1: GYTFTDLG | 481 |
| | CDR-H2: INTHTGMP | 482 |
| | CDR-H3: ARDHPLGLDY | 483 |
| | CDR-L1: QSVDVNGVSY | 484 |
| | CDR-L2: KAS | N/A |
| | CDR-L3: QQSREDPYT | 486 |
| WO 2022/003690 | CDR-H1: GYTFTDLG | 487 |
| | CDR-H2: INTHTGEP | 488 |
| | CDR-H3: TRDHPLGLDY | 489 |
| | CDR-L1: QSVDVNGVSY | 490 |
| | CDR-L2: KAS | N/A |
| | CDR-L3: QQSREDPYT | 492 |
| WO 2022/003690 | CDR-H1: GYTFTDLG | 493 |
| | CDR-H2: INTHTGEP | 494 |
| | CDR-H3: TRDHPLGLDY | 495 |
| | CDR-L1: QSVDVNGLSY | 496 |
| | CDR-L2: KAS | N/A |
| | CDR-L3: QQSREDPYT | 498 |
| WO 2022/003690 | CDR-H1: GYTFTDLG | 499 |
| | CDR-H2: INTHTGKP | 500 |
| | CDR-H3: TRDHPLGLDY | 501 |
| | CDR-L1: QSVDVNGVSY | 502 |
| | CDR-L2: KAS | N/A |
| | CDR-L3: QQSREDPYT | 504 |
| WO 2022/003690 | CDR-H1: GYTFTDLG | 505 |
| | CDR-H2: INTQTGEP | 506 |
| | CDR-H3: TRDHPLGLDY | 507 |
| | CDR-L1: QSVDVAGVSY | 508 |
| | CDR-L2: KAS | N/A |
| | CDR-L3: QQAREDPYT | 510 |
| WO 2022/003690 | CDR-H1: GYTFTDLG | 511 |
| | CDR-H2: INTQTGEP | 512 |
| | CDR-H3: ARDHPLGLDY | 513 |
| | CDR-L1: QSVDVNGVSY | 514 |
| | CDR-L2: KAS | N/A |
| | CDR-L3: QQSREDPYT | 516 |
| WO 2022/003690 | CDR-H1: GYTFTDLG | 517 |
| | CDR-H2: INTHTGEP | 518 |
| | CDR-H3: TRDHPLGLDY | 519 |
| | CDR-L1: QSVDVNGVSY | 520 |
| | CDR-L2: KAS | N/A |
| | CDR-L3: QQSREDPYT | 522 |
| WO 2022/003690 | CDR-H1: GYTFTDLG | 523 |
| | CDR-H2: INTHTGEP | 524 |
| | CDR-H3: ARDHPLGLDY | 525 |
| | CDR-L1: QSVDVNGVSY | 526 |
| | CDR-L2: KAV | N/A |
| | CDR-L3: QQSREDPYT | 528 |
| WO 2022/003690 | CDR-H1: GYTFTDLG | 529 |
| | CDR-H2: INTITGEP | 530 |
| | CDR-H3: TRDHPLGLDY | 531 |
| | CDR-L1: QSLDVNGVSY | 532 |
| | CDR-L2: KAS | N/A |
| | CDR-L3: QQSREDPYT | 534 |
| WO 2022/003690 | CDR-H1: GYTFTDLG | 535 |
| | CDR-H2: INTHTGEP | 536 |
| | CDR-H3: TRDHPLGLDY | 537 |
| | CDR-L1: QSVDVNGVSY | 538 |
| | CDR-L2: KAS | N/A |
| | CDR-L3: QQNREDPYT | 540 |
| WO 2022/003690 | CDR-H1: GYTFTDLG | 541 |
| | CDR-H2: INTITGEP | 542 |
| | CDR-H3: TRDHPLGLDY | 543 |
| | CDR-L1: QSVDVNGVSY | 544 |
| | CDR-L2: KAS | N/A |
| | CDR-L3: QQSREDPYT | 546 |
| WO 2022/003690 | CDR-H1: GYTFTDLG | 547 |
| | CDR-H2: INTHTGEP | 548 |
| | CDR-H3: TRDHPLGLDY | 549 |
| | CDR-L1: QSVDVNGVSY | 550 |
| | CDR-L2: KAS | N/A |
| | CDR-L3: QQAREDPYT | 552 |
| WO 2022/003690 | CDR-H1: GFNIRDTY | 553 |
| | CDR-H2: IDPDNGDT | 554 |
| | CDR-H3: SRDLADGYNAFDR | 555 |
| | CDR-L1: QDVSTA | 556 |
| | CDR-L2: SAS | N/A |
| | CDR-L3: QQPTYTPPT | 558 |
| WO 2022/003690 | CDR-H1: GFNIRDTF | 559 |
| | CDR-H2: IDPDNGDT | 560 |
| | CDR-H3: SREGEDGYNAWDL | 561 |
| | CDR-L1: QDVSTA | 562 |
| | CDR-L2: SAS | N/A |
| | CDR-L3: QQPPYTPPT | 564 |
| WO 2022/003690 | CDR-H1: GFNIRATY | 565 |
| | CDR-H2: IDPTNGDT | 566 |
| | CDR-H3: TREGDDGYNAWDK | 567 |
| | CDR-L1: QDVSTA | 568 |
| | CDR-L2: SAS | N/A |
| | CDR-L3: QQPSYTPPT | 570 |
| WO 2020/089473 | CDR-H1: FSDYYMSWVRQAPG | 571 |
| | CDR-H2: ANINTDGSEKYYLDSVKGR | 572 |
| | CDR-H3: AREEYGAFDI | 573 |
| | CDR-L1: CSGSSSNIGSNTVN | 574 |
| | CDR-L2: DNNKRPS | 575 |
| | CDR-L3: CQSFDRGLSGSIV | 576 |
| WO 2020/089473 | CDR-H1: FSSYAMHWVRQAPG | 577 |
| | CDR-H2: SAISGGATTTYYADSVKGR | 578 |
| | CDR-H3: AKGGTGDPYYFDY | 579 |
| | CDR-L1: CTGSSSNIGAGYDVH | 580 |
| | CDR-L2: RNNQRPS | 58 |
| | CDR-L3: CAARDDGLSGPV | 582 |
| WO 2020/089473 | CDR-H1: FSNAWMSWVRQAPG | 583 |
| | CDR-H2: SSISSASGYIYYGDSVKGR | 584 |
| | CDR-H3: ARGTLYGDFDEF | 585 |
| | CDR-L1: CSGSSSNIGNNAVN | 586 |
| | CDR-L2: GNTNRPS | 587 |
| | CDR-L3: CQSYDSSLSGYVV | 588 |
| WO 2020/089473 | CDR-H1: FSSNEMSWIRQAPG | 589 |
| | CDR-H2: SVIYSGGSTYYADSVKGR | 590 |
| | CDR-H3: ARREGWLVPFDY | 591 |
| | CDR-L1: CSGSSSNIGSNTVN | 592 |
| | CDR-L2: GNIIRPS | 593 |
| | CDR-L3: CQSFDTTLSGSIV | 594 |
| WO 2020/089473 | CDR-H1: FSRYWMHWVRQVPG | 595 |
| | CDR-H2: SGISDSGVVTYYADSVKGR | 596 |
| | CDR-H3: ARAQSVAFDI | 597 |
| | CDR-L1: CSGSSSNIGAGHDVH | 598 |
| | CDR-L2: YDDLLPS | 599 |
| | CDR-L3: CAAWDDSLSGWV | 600 |
| WO 2020/089473 | CDR-H1: FSSYAMSWVRQAPG | 601 |
| | CDR-H2: SVISGSGGSTYYADAVKGR | 602 |
| | CDR-H3: TTDSGSGSYL | 603 |
| | CDR-L1: CTGSSSNIGAGYDVH | 604 |
| | CDR-L2: SNNQRPS | 605 |
| | CDR-L3: CAAWDDSLNGPV | 606 |
| WO 2020/089473 | CDR-H1: FSSNYMSWVRQAPG | 607 |
| | CDR-H2: SVISGSGGSTYYADSVKGR | 608 |
| | CDR-H3: ARDRGWFDP | 609 |
| | CDR-L1: CSGSRSNIDNSYVS | 610 |
| | CDR-L2: RNNQRPS | 611 |
| | CDR-L3: CATWDDSLSGPV | 612 |
| WO 2020/089473 | CDR-H1: FSRHAMNWVRQAPG | 613 |
| | CDR-H2: SSISTGSSYIDYADSVKGR | 614 |
| | CDR-H3: AREKGHYYYGMDV | 615 |
| | CDR-L1: CTGSSSNIGAGYDVH | 616 |
| | CDR-L2: GNSYRPS | 617 |
| | CDR-L3: CQSYDTSLSAYVV | 618 |
| WO 2020/089473 | CDR-H1: FSNAWMSWVRQAPG | 619 |
| | CDR-H2: SAISVSGINTYYADSVKGR | 620 |
| | CDR-H3: ARDTGSLGVDY | 621 |
| | CDR-L1: CSGSSSNIGSNTVN | 622 |
| | CDR-L2: RNNQRPS | 623 |
| | CDR-L3: CQSYDSSLSISV | 624 |
| WO 2020/089473 | CDR-H1: SSSYWMSWVRQAPG | 625 |
| | CDR-H2: SAISGSGGSTYYADSVKGR | 626 |
| | CDR-H3: AREYSGYEFDF | 627 |
| | CDR-L1: CTGSSSNIGARSDVH | 628 |
| | CDR-L2: GNRNRPS | 629 |
| | CDR-L3: CQSFDRGLSGSIV | 630 |
| WO 2020/089473 | CDR-H1: SSSYWMSWVRQAPG | 631 |
| | CDR-H2: SAISGSGGSTYYADSVKGR | 632 |
| | CDR-H3: AREYSGYEFDF | 633 |
| | CDR-L1: CTGSSSNIGARSDVH | 634 |
| | CDR-L2: GNRNRPS | 635 |
| | CDR-L3: CQSFDRGLSGSIV | 636 |
| WO 2020/089473 | CDR-H1: FSSNYMSWVRQAPG | 637 |
| | CDR-H2: SSISSSSSYIYYADSVKGR | 638 |
| | CDR-H3: ARDRGRTGTDY | 639 |
| | CDR-L1: CSGTTSNIGSYAVN | 640 |
| | CDR-L2: GNINRPS | 641 |
| | CDR-L3: CQSYDSSLSASL | 642 |
| WO 2020/089473 | CDR-H1: FSSYAMSWVRQAPG | 643 |
| | CDR-H2: STIIGSGANTWYADSVKGR | 644 |
| | CDR-H3: ARHEGYYYYGMDV | 645 |
| | CDR-L1: CTGSSSNIGAGYVVH | 646 |
| | CDR-L2: GNSNRPS | 647 |
| | CDR-L3: CAAWDDSLNGRV | 648 |
| WO 2021/200840 | CDR-H1: GYNMN | 649 |
| | CDR-H2: KIDPYYGGTDYNLRFKD | 650 |
| | CDR-H3: EPNYGSNDWYFDV | 651 |
| | CDR-L1: RASQDIDNYLN | 652 |
| | CDR-L2: YTSILHS | 653 |
| | CDR-L3: QQGNTLPWT | 654 |
| WO 2021/200840 | CDR-H1: ESIIH | 655 |
| | CDR-H2: WFYPGSDNINYNEKFKD | 656 |
| | CDR-H3: HEGPYVYFDY | 657 |
| | CDR-L1: KASQDVSTAVA | 658 |
| | CDR-L2: YWTSTRHT | 659 |
| | CDR-L3: QHHYSTPYT | 660 |
| WO 2021/200840 | CDR-H1: SYWMQ | 661 |
| | CDR-H2: AIYPGDGDSRYIQKFKG | 662 |
| | CDR-H3: DGNYYAMDY | 663 |
| | CDR-L1: TASSSVSSTYLH | 664 |
| | CDR-L2: STSNLAS | 665 |
| | CDR-L3: HQYHRSPPT | 666 |
| WO 2021/200840 | CDR-H1: DTYMH | 667 |
| | CDR-H2: RIDPANGYTEYDPKFQD | 668 |
| | CDR-H3: TQLYY | 669 |
| | CDR-L1: TASSSVSSTYLH | 670 |
| | CDR-L2: STSNLAS | 671 |
| | CDR-L3: HQYHRSPLT | 672 |
| WO 2021/200840 | CDR-H1: VYGVN | 673 |
| | CDR-H2: MIWGDGSTAYNSALKS | 674 |
| | CDR-H3: DGRRYALDY | 675 |
| | CDR-L1: RASESVDSYGDSFLH | 676 |
| | CDR-L2: RASNLDS | 677 |
| | CDR-L3: QQSNEDPYT | 678 |
| WO 2023/228082 | CDR-H1: GFSFPSSGVD | 1462 |
| | CDR-H2: VTWGVGSTHYNSALKS | 1463 |
| | CDR-H3: GEWDEGFVY | 1464 |
| | CDR-L1: KASQNVGTAVA | 1465 |
| | CDR-L2: SASNRYT | 1466 |
| | CDR-L3: QQYSSYPLT | 1467 |
| WO 2023/228082 | CDR-H1: GFSFPSSGVD | 1468 |
| | CDR-H2: VTWGVGSTHYNQKFQG | 1469 |
| | CDR-H3: GEWDEGFVY | 1470 |
| | CDR-L1: KASQNVGTAVA | 1471 |
| | CDR-L2: AASTLQS | 1472 |
| | CDR-L3: QQYSSYPLT | 1473 |
| WO 2023/228082 | CDR-H1: GFSFPSSGVD | 1474 |
| | CDR-H2: VTWGVGSTHYNQKFQG | 1475 |
| | CDR-H3: GEWDEGFVY | 1476 |
| | CDR-L1: KASQNVGTAVA | 1477 |
| | CDR-L2: AASTLQS | 1478 |
| | CDR-L3: QQYSSYPLT | 1479 |
| | CDR-H1: GYTFTDY | 1413 |
| | CDR-H2: NPNYDS | 1414 |
| | CDR-H3: GNSWYFDV | 1415 |
| | CDR-L1: SASSSVRYMY | 1416 |
| | CDR-L2: LTSNLAS | 1417 |
| | CDR-L3: QQWSSNPLT | 1418 |
| | CDR-H1: GYTFTDY | 1419 |
| | CDR-H2: NYDSTS | 1420 |
| | CDR-H3: GNSWYFDV | 1421 |
| | CDR-L1: SASSSVRYMY | 1422 |
| | CDR-L2: LTSNLAS | 1423 |
| | CDR-L3: QQWSSNPLT | 1424 |
| | CDR-H1: Any one of: | |
| | GYTFTDYNLD | 1425 |
| | DYIMH | 1426 |
| | CDR-H2: Any one of: | 1427 |
| | DINPNYDS | |
| | WVDPEYGSTDYAEKFKK | 1428 |
| | CDR-H3: Any one of: | |
| | TSYSQKFRG | 1429 |
| | DDGSYSPFEDY | 1430 |
| | CDR-L1: Any one of: | |
| | SASSSVRYMYWY | 1431 |
| | KASENVVTYVS | 1432 |
| | RSSQSLVHSNGNTYLH | 1433 |
| | KASENVVTYVS | 1434 |
| | QASQNINKYIA | 1435 |
| | QNINKY | 1436 |
| | CDR-L2: Any one of: | |
| | LTSNLAS | 1437 |
| | VTSNLAS | 1438 |
| | LTSNLGS | 1439 |
| | GASNRTY | 1440 |
| | IKVSNRFS | 1441 |
| | DPEYGS | 1442 |
| | YTSTLES | 1443 |
| | YTS | |
| | CDR-L3: Any one of: | |
| | QQRSNWP | 1445 |
| | GQGYSYPYT | 1446 |
| | SQTTHVPPT | 1447 |
| | LQYVNLIT | 1448 |
| | CDR-H1: X¹X²X²X¹X²X³X²JJJ, | |
| | wherein: | |
| | each X¹ is independently G, A, V, L, I, M, W, F, or | |
| | P: | N/A |
| | each X² is independently Y, S, T, C, N, or Q; | |
| | X³ is D or E; and | |
| | each J is independently a naturally occurring | |
| | amino acid or is absent | |
| | CDR-H2: Any sequence | |
| | CDR-H3: Any sequence | |
| | CDR-L1: Any sequence | |
| | CDR-L2: Any sequence | |
| | CDR-L3: Any sequence | |
| | CDR-H1: GYTFTZ¹Z²JJJ, | 1480 |
| | wherein: | |
| | Z¹ is D or T; | |
| | Z² is Y, F, or L; and | |
| | each J is independently a naturally occurring | |
| | amino acid or is absent | |
| | CDR-H2: Any sequence | |
| | CDR-H3: Any sequence | |
| | CDR-L1: Any sequence | |
| | CDR-L2: Any sequence | |
| | CDR-L3: Any sequence | |
| | CDR-H1: DYNLD | 1541 |
| | CDR-H2: DINPNYDSTSYSQKFRG | 1542 |
| | CDR-H3: GNSWYFDV | 1543 |
| | CDR-L1: SASSSVRYNY | 1544 |
| | CDR-L2: LTSNLAS | 1545 |
| | CDR-L3: PQQWSSNPLT | 1546 |
| | CDR-H1: X¹X²X²X³X¹, | N/A |
| | wherein: | |
| | each X¹ is independently D or E; | |
| | each X² is independently Y, S, T, C, N, or Q; and | |
| | X³ is L, A, V, G, I, M, W, F, or P | |
| | CDR-H2: Any sequence | |
| | CDR-H3: Any sequence | |
| | CDR-L1: Any sequence | |
| | CDR-L2: Any sequence | |
| | CDR-L3: Any sequence | |
| | CDR-H1: Any sequence | |
| | CDR-H2: X¹X²X³X²X³X³X¹X³X³X³X³X³X³X⁴X²X⁴X², | N/A |
| | wherein: | |
| | each X¹ is independently D or E; | |
| | each X² is independently I, A, V, L, G, M, W, F, or | |
| | P; | |
| | each X³ is independently N, S, T, C, Y, or Q; and | |
| | each X⁴ is independently K, R, or H | |
| | CDR-H3: Any sequence | |
| | CDR-L1: Any sequence | |
| | CDR-L2: Any sequence | |
| | CDR-L3: Any sequence | |
| | CDR-H1: Any sequence | |
| | CDR-H2: Any sequence | |
| | CDR-H3: X¹X²X²X¹X²X¹X³X¹, | N/A |
| | wherein: | |
| | each X¹ is independently G, A, V, L, I, M, W, F, or | |
| | P; | |
| | each X² is independently N, S, T, C, Y, or Q; and | |
| | X³ is D or E | |
| | CDR-L1: Any sequence | |
| | CDR-L2: Any sequence | |
| | CDR-L3: Any sequence | |
| | CDR-H1: Any sequence | |
| | CDR-H2: Any sequence | |
| | CDR-H3: Any sequence | |
| | CDR-L1: X¹X²X¹X¹X¹X²X³X⁴X¹X¹, | N/A |
| | wherein: | |
| | each X¹ is independently S, Y, T, C, N, or Q; | |
| | each X² is independently A, G, V, L, I, M, W, F, or | |
| | P; | |
| | X³ is R, H, or K; and | |
| | X⁴ is Y, A, V, I, L, M, F, or W | |
| | CDR-L2: Any sequence | |
| | CDR-L3: Any sequence | |
| | CDR-H1: Any sequence | |
| | CDR-H2: Any sequence | |
| | CDR-H3: Any sequence | |
| | CDR-L1: Any sequence | |
| | CDR-L2: X¹X²X²X²X¹X¹X², | N/A |
| | wherein: | |
| | each X¹ is independently L, A, V, G, I, M, W, F, or | |
| | P; and | |
| | each X² is independently T, S, C, Y, N, or Q | |
| | CDR-L3: Any sequence | |
| | CDR-H1: Any sequence | |
| | CDR-H2: Any sequence | |
| | CDR-H3: Any sequence | |
| | CDR-L1: Any sequence | |
| | CDR-L2: Any sequence | |
| | CDR-L3: X¹X²X²X¹X²X²X²X¹X¹X², | N/A |
| | wherein: | |
| | each X¹ is independently P, A, V, L, I, M, W, F, or | |
| | G; and | |
| | each X² is independently Q, S, T, C, N, or Y | |

The CDR-H1 of agonistic antibodies or antigen-binding fragments thereof that specifically bind TNFR2 can tolerate a wide range of amino acid modifications. See, e.g., International Patent Application Publication No. WO 2021/231922, which is incorporated herein by reference. In some embodiments, the agonistic antibody or antigen-binding fragment thereof that specifically binds TNFR2 comprises a CDR-H1 having the amino acid sequence GXTFXXY (SEQ ID NO: 1449), in which each X is independently a naturally occurring amino acid. In some embodiments, the CDR-H1 has an amino acid sequence selected from the group consisting of GYTFXXY (SEQ ID NO: 1450), GSTFXXY (SEQ ID NO: 1451), GTTFXXY (SEQ ID NO: 1452), GCTFXXY (SEQ ID NO: 1453), GNTFXXY (SEQ ID NO: 1454), GQTFXXY (SEQ ID NO: 1455), GXTFSXY (SEQ ID NO: 1456), GXTFCXY (SEQ ID NO: 1457), GXTFYXY (SEQ ID NO: 1458), GXTFNXY (SEQ ID NO: 1459), GXTFOXY (SEQ ID NO: 1460), and GXTFXDY (SEQ ID NO: 1461), in which each X is independently a naturally occurring amino acid. In some embodiments, the CDR-H1 has the amino acid sequence of GYTFTDY (SEQ ID NO: 1413) or a variant thereof with up to two conservative amino acid substitutions. In some embodiments, the agonistic antibody or antigen-binding fragment thereof that specifically binds TNFR2 further comprises one or more of a CDR-H2 having the amino acid sequence NPNYDS (SEQ ID NO: 1414), NYDSTS (SEQ ID NO: 1420), or a variant thereof with up to two conservative amino acid substitutions; a CDR-H3 having the amino acid sequence GNSWYFDV (SEQ ID NO: 1415) or a variant thereof with up to two conservative amino acid substitutions; a CDR-L1 having the amino acid sequence SASSSVRYMY (SEQ ID NO: 1416) or a variant thereof with up to two conservative amino acid substitutions; a CDR-L2 having the amino acid sequence LTSNLAS (SEQ ID NO: 1417) or a variant thereof with up to two conservative amino acid substitutions; and a CDR-L3 having the amino acid sequence QQWSSNPLT (SEQ ID NO: 1418) or a variant thereof with up to two conservative amino acid substitutions.

Additional agonistic antibodies or antigen-binding fragments thereof that specifically bind TNFR2 may comprise one or more of:
(a) a CDR-H1 comprising the amino acid sequence of GYTFTDYNLD (SEQ ID NO: 1425), DYIMH (SEQ ID NO: 1426), or a variant thereof with up to two conservative amino acid substitutions;
(b) a CDR-H2 comprising the amino acid sequence of DINPNYDS (SEQ ID NO: 1427), WVDPEYGSTDYAEKFKK (SEQ ID NO: 1428), or a variant thereof with up to two conservative amino acid substitutions;
(c) a CDR-H3 comprising the amino acid sequence of TSYSQKFRG (SEQ ID NO: 1429), DDGSYSPFEDY (SEQ ID NO: 1430), or a variant thereof with up to two conservative amino acid substitutions;
(d) a CDR-L1 comprising an amino acid sequence selected from the group consisting of SASSSVRYMYWY (SEQ ID NO: 1431), KASENVVTYVS (SEQ ID NO: 1432), RSSQSLVHSNGNTYLH (SEQ ID NO: 1433), KASENVVTYVS (SEQ ID NO: 1434), QASQNINKYIA (SEQ ID NO: 1435), QNINKY (SEQ ID NO: 1436), and a variant thereof with up to two conservative amino acid substitutions;
(e) a CDR-L2 comprising an amino acid sequence selected from the group consisting of LTSNLAS (SEQ ID NO: 1437), VTSNLAS (SEQ ID NO: 1438), LTSNLGS (SEQ ID NO: 1439), GASNRTY (SEQ ID NO: 1440), IKVSNRFS (SEQ ID NO: 1441), DPEYGS (SEQ ID NO: 1442), YTSTLES (SEQ ID NO: 1443), YTS, and a variant thereof with up to two conservative amino acid substitutions; and
(f) a CDR-L3 comprising an amino acid sequence selected from the group consisting of QQRSNWP (SEQ ID NO: 1445), GQGYSYPYT (SEQ ID NO: 1446), SQTTHVPPT (SEQ ID NO: 1447), LQYVNLIT (SEQ ID NO: 1448), and a variant thereof with up to two conservative amino acid substitutions.

In some embodiments, the antibody or antigen-binding fragment thereof is an agonistic antibody or antigen-binding fragment thereof that specifically binds TNFR2 comprising a VH and/or a VL having amino acid sequences that are at least 80% identical (e.g., 85%, 90%, 95%, 97%, 99%, or 100% identical) to a VH and/or a VL sequence selected from Table 3 below. In some embodiments, the antibody or antigen-binding fragment may be any antibody or antigen-binding fragment thereof disclosed in International Patent Application Publication Nos. WO 2020/102739, WO 2021/231922, WO 2020/061210, WO 2020/180712, WO 2021/055253, WO 2022/003690, WO 2020/089473, WO 2021/200840, or WO 2023/228082; incorporated herein by reference.

In some embodiments, the antibody or antigen-binding fragment thereof may be any antibody or antigen-binding fragment thereof disclosed in Examples 1, 3, 4, 10, 13, and 16, and Tables 1-3, 8, 11, and 20, of International Patent Application Publication No. WO 2023/228082; incorporated herein by reference. The antibodies or antigen-binding fragment thereof of International Patent Application Publication No. WO 2023/228082 can be administered to a human subject subcutaneously and/or at a low dose, low dose range, or low dosing frequency.

TABLE 3
VH and VL sequences for agonistic anti-TNFR2 antibodies
or antigen-binding fragments thereof.

| Publication Number | VH SEQ ID NO: | VL SEQ ID NO: |
|---|---|---|
| WO 2020/102739 | 679 | 680 |
| | 681 | 682 |
| WO 2021/231922 | 683 | 684 |
| | 685 | 686 |
| | 687 | 688 |
| | 689 | 690 |
| | 691 | 692 |
| | 693 | 694 |
| WO 2020/061210 | 695 | 696 |
| | 697 | 698 |
| | 699 | 700 |
| | 701 | 702 |
| | 703 | 704 |
| | 705 | 706 |
| | 707 | 708 |
| | 709 | 710 |
| | 711 | 712 |
| WO 2020/180712 | 713 | 714 |
| | 715 | 716 |
| | 717 | 718 |
| | 719 | 720 |
| | 721 | 722 |
| | 723 | 724 |
| | 725 | 726 |
| | 727 | 728 |
| | 729 | 730 |
| | 731 | 732 |
| | 733 | 734 |
| | 735 | 736 |
| | 737 | 738 |
| | 739 | 740 |
| | 741 | 742 |
| | 743 | 744 |
| | 745 | 746 |
| | 747 | 748 |
| | 749 | 750 |
| | 751 | 752 |
| | 753 | 754 |
| | 755 | 756 |
| WO 2021/055253 | 757 | 758 |
| | 759 | 760 |
| | 761 | 762 |
| | 763 | 764 |
| | 765 | 766 |
| | 767 | 768 |
| | 769 | 770 |
| | 771 | 772 |
| | 773 | 774 |
| | 775 | 776 |
| | 777 | 778 |
| | 779 | 780 |
| | 781 | 782 |
| | 783 | 784 |
| | 785 | 786 |
| | 787 | 788 |
| | 789 | 790 |
| WO 2022/003690 | 791 | 792 |
| | 793 | 794 |
| | 795 | 796 |
| | 797 | 798 |
| | 799 | 800 |
| | 801 | 802 |
| | 803 | 804 |
| | 805 | 806 |
| | 807 | 808 |
| | 809 | 810 |
| | 811 | 812 |
| | 813 | 814 |
| | 815 | 816 |
| | 817 | 818 |
| | 819 | 820 |
| | 821 | 822 |
| | 823 | 824 |
| | 825 | 826 |
| | 827 | 828 |
| | 829 | 830 |
| | 831 | 832 |
| | 833 | 834 |
| | 835 | 836 |
| | 837 | 838 |
| | 839 | 840 |
| | 841 | 842 |
| | 843 | 844 |
| | 845 | 846 |
| | 847 | 848 |
| | 849 | 850 |
| | 851 | 852 |
| | 853 | 854 |
| | 855 | 856 |
| | 857 | 858 |
| | 859 | 860 |
| | 861 | 862 |
| | 863 | 864 |
| | 865 | 866 |
| | 867 | 868 |
| | 869 | 870 |
| | 871 | 872 |
| | 873 | 874 |
| | 875 | 876 |
| | 877 | 878 |
| | 879 | 880 |
| | 881 | 882 |
| | 883 | 884 |
| | 885 | 886 |
| | 887 | 888 |
| | 889 | 890 |
| | 891 | 892 |
| | 893 | 894 |
| | 895 | 896 |
| | 897 | 898 |
| | 899 | 900 |
| | 901 | 902 |
| | 903 | 904 |
| | 905 | 906 |
| | 907 | 908 |
| | 909 | 910 |
| | 911 | 912 |
| | 913 | 914 |
| | 915 | 916 |
| | 917 | 918 |
| | 919 | 920 |
| | 921 | 922 |
| | 923 | 924 |
| | 925 | 926 |
| | 927 | 928 |
| | 929 | 930 |
| | 931 | 932 |
| | 933 | 934 |
| | 935 | 936 |
| | 937 | 938 |
| | 939 | 940 |
| WO 2020/089473 | 941 | 942 |
| | 943 | 944 |
| | 945 | 946 |
| | 947 | 948 |
| | 949 | 950 |
| | 951 | 952 |
| | 953 | 954 |
| | 955 | 956 |
| | 957 | 958 |
| | 959 | 960 |
| | 961 | 962 |
| | 963 | 964 |
| | 965 | 966 |
| WO 2021/200840 | 967 | 968 |
| | 969 | 970 |
| | 971 | 972 |
| | 973 | 974 |
| | 975 | 976 |
| WO 2023/228082 | 1481 | 1482 |
| | 1483 | 1484 |
| | 1485 | 1491 |
| | 1486 | 1492 |
| | 1487 | 1493 |
| | 1488 | 1494 |
| | 1489 | 1495 |
| | 1490 | 1496 |
| | | 1497 |

Additional agonistic antibodies or antigen-binding fragments thereof that specifically bind TNFR2 may comprise one or more of:
(a) a CDR-H1 comprising the amino acid sequence of GYTFTDYNL (SEQ ID NO: 7), or a variant thereof with up to two conservative amino acid substitutions;
(b) a CDR-H2 comprising the amino acid sequence of INPNYDST (SEQ ID NO: 8), or a variant thereof with up to two conservative amino acid substitutions;
(c) a CDR-H3 comprising the amino acid sequence of CARGNSWYFDV (SEQ ID NO: 9), or a variant thereof with up to two conservative amino acid substitutions;
(d) a CDR-L1 comprising an amino acid sequence of SSVRY (SEQ ID NO: 10), or a variant thereof with up to two conservative amino acid substitutions;
(e) a CDR-L2 comprising an amino acid sequence of LTS, or a variant thereof with up to two conservative amino acid substitutions; and
(f) a CDR-L3 comprising an amino acid sequence of CQQWSSNPLT (SEQ ID NO: 12), or a variant thereof with up to two conservative amino acid substitutions.

In some embodiments, the antibody or antigen-binding fragment thereof comprises:
(a) a VH having an amino acid sequence that is at least 80% identical (e.g., 85%, 90%, 95%, 97%, 99%, or 100% identical) to any one of SEQ ID NOs: 1486-1490; and
(b) a VL having an amino acid sequence that is at least 80% identical (e.g., 85%, 90%, 95%, 97%, 99%, or 100% identical) to any one of SEQ ID NOs: 1492-1497.

In some embodiments, the antibody or antigen-binding fragment thereof comprises:
(a) a VH having the amino acid sequence of any one of SEQ ID NOs: 1486-1490; and
(b) a VL having the amino acid sequence of any one of SEQ ID NOs: 1492-1497.

In some embodiments, the antibody or antigen binding fragment thereof comprises:
(a) a VH having an amino acid sequence that is at least 80% identical (e.g., 85%, 90%, 95%, 97%, 99%, or 100% identical) to SEQ ID NO: 1488 and a VL having an amino acid sequence that is at least 80% identical (e.g., 85%, 90%, 95%, 97%, 99%, or 100% identical) to SEQ ID NO: 1495;
(b) a VH having an amino acid sequence that is at least 80% identical (e.g., 85%, 90%, 95%, 97%, 99%, or 100% identical) to SEQ ID NO: 1488 and a VL having an amino acid sequence that is at least 80% identical (e.g., 85%, 90%, 95%, 97%, 99%, or 100% identical) to SEQ ID NO: 1497;
(c) a VH having an amino acid sequence that is at least 80% identical (e.g., 85%, 90%, 95%, 97%, 99%, or 100% identical) to SEQ ID NO: 1489 and a VL having an amino acid sequence that is at least 80% identical (e.g., 85%, 90%, 95%, 97%, 99%, or 100% identical) to SEQ ID NO: 1496;
(d) a VH having an amino acid sequence that is at least 80% identical (e.g., 85%, 90%, 95%, 97%, 99%, or 100% identical) to SEQ ID NO: 1490 and a VL having an amino acid sequence that is at least 80% identical (e.g., 85%, 90%, 95%, 97%, 99%, or 100% identical) to SEQ ID NO: 1496; or
(e) a VH having an amino acid sequence that is at least 80% identical (e.g., 85%, 90%, 95%, 97%, 99%, or 100% identical) to SEQ ID NO: 1490 and a VL having an amino acid sequence that is at least 80% identical (e.g., 85%, 90%, 95%, 97%, 99%, or 100% identical) to SEQ ID NO: 1497.

In some embodiments, the antibody or antigen binding fragment thereof comprises:
(a) a VH having the amino acid sequence of SEQ ID NO: 1488 and a VL having the amino acid sequence of SEQ ID NO: 1495;
(b) a VH having the amino acid sequence of SEQ ID NO: 1488 and a VL having the amino acid sequence of SEQ ID NO: 1497;
(c) a VH having the amino acid sequence of SEQ ID NO: 1489 and a VL having the amino acid sequence of SEQ ID NO: 1496;
(d) a VH having the amino acid sequence of SEQ ID NO: 1490 and a VL having the amino acid sequence of SEQ ID NO: 1496; or
(e) a VH having the amino acid sequence of SEQ ID NO: 1490 and a VL having the amino acid sequence of SEQ ID NO: 1497.

In some embodiments, the antibody or antigen-binding fragment thereof comprises a VH having an amino acid sequence that is at least 80% identical (e.g., 85%, 90%, 95%, 97%, 99%, or 100% identical) to SEQ ID NO: 1488 and a VL having an amino acid sequence that is at least 80% identical (e.g., 85%, 90%, 95%, 97%, 99%, or 100% identical) to SEQ ID NO: 1495.

In some embodiments, the antibody or antigen-binding fragment thereof comprises a VH having the amino acid sequence of SEQ ID NO: 1488 and a VL having the amino acid sequence of SEQ ID NO: 1495.

In some embodiments, the antibody or antigen-binding fragment thereof is a human antibody or antigen-binding fragment thereof. In some embodiments, the antibody or antigen-binding fragment thereof is an IgG antibody or antigen-binding fragment thereof. In some embodiments, the antibody or antigen-binding fragment thereof is a human IgG1, human IgG2, human IgG3, or human IgG4 antibody or antigen-binding fragment thereof. In some embodiments, the antibody or antigen-binding fragment thereof is a human IgG4 antibody or antigen-binding fragment thereof.

In some embodiments, the antibody or antigen-binding fragment thereof comprises:
(a) a heavy chain having an amino acid sequence that is at least 80% identical (e.g., 85%, 90%, 95%, 97%, 99%, or 100% identical) to any one of SEQ ID NOs: 1498-1503; and
(b) a light chain having an amino acid sequence that is at least 80% identical (e.g., 85%, 90%, 95%, 97%, 99%, or 100% identical) to any one of SEQ ID NOs: 1504-1510.

In some embodiments, the antibody or antigen-binding fragment thereof comprises:
(a) a heavy chain having the amino acid sequence of any one of SEQ ID NOs: 1498-1503; and
(b) a light chain having the amino acid sequence of any one of SEQ ID NOs: 1504-1510.

In some embodiments, the antibody or antigen binding fragment thereof comprises:
(a) a heavy chain having an amino acid sequence that is at least 80% identical (e.g., 85%, 90%, 95%, 97%, 99%, or 100% identical) to SEQ ID NO: 1498 and a light chain having an amino acid sequence that is at least 80% identical (e.g., 85%, 90%, 95%, 97%, 99%, or 100% identical) to SEQ ID NO: 1504;
(b) a heavy chain having an amino acid sequence that is at least 80% identical (e.g., 85%, 90%, 95%, 97%, 99%, or 100% identical) to SEQ ID NO: 1501 and a light chain having an amino acid sequence that is at least 80% identical (e.g., 85%, 90%, 95%, 97%, 99%, or 100% identical) to SEQ ID NO: 1508;
(c) a heavy chain having an amino acid sequence that is at least 80% identical (e.g., 85%, 90%, 95%, 97%, 99%, or 100% identical) to SEQ ID NO: 1501 and a light chain having an amino acid sequence that is at least 80% identical (e.g., 85%, 90%, 95%, 97%, 99%, or 100% identical) to SEQ ID NO: 1510;
(d) a heavy chain having an amino acid sequence that is at least 80% identical (e.g., 85%, 90%, 95%, 97%, 99%, or 100% identical) to SEQ ID NO: 1502 and a light chain having an amino acid sequence that is at least 80% identical (e.g., 85%, 90%, 95%, 97%, 99%, or 100% identical) to SEQ ID NO: 1509;
(e) a heavy chain having an amino acid sequence that is at least 80% identical (e.g., 85%, 90%, 95%, 97%, 99%, or 100% identical) to SEQ ID NO: 1503 and a light chain having an amino acid sequence that is at least 80% identical (e.g., 85%, 90%, 95%, 97%, 99%, or 100% identical) to SEQ ID NO: 1509; or
(f) a heavy chain having an amino acid sequence that is at least 80% identical (e.g., 85%, 90%, 95%, 97%, 99%, or 100% identical) to SEQ ID NO: 1503 and a light chain having an amino acid sequence that is at least 80% identical (e.g., 85%, 90%, 95%, 97%, 99%, or 100% identical) to SEQ ID NO: 1510.

In some embodiments, the antibody or antigen binding fragment thereof comprises:
(a) a heavy chain having the amino acid sequence of SEQ ID NO: 1498 and a light chain having the amino acid sequence of SEQ ID NO: 1504;
(b) a heavy chain having the amino acid sequence of SEQ ID NO: 1501 and a light chain having the amino acid sequence of SEQ ID NO: 1508;
(c) a heavy chain having the amino acid sequence of SEQ ID NO: 1501 and a light chain having the amino acid sequence of SEQ ID NO: 1510;
(d) a heavy chain having the amino acid sequence of SEQ ID NO: 1502 and a light chain having the amino acid sequence of SEQ ID NO: 1509;
(e) a heavy chain having the amino acid sequence of SEQ ID NO: 1503 and a light chain having the amino acid sequence of SEQ ID NO: 1509; or
(f) a heavy chain having the amino acid sequence of SEQ ID NO: 1503 and a light chain having the amino acid sequence of SEQ ID NO: 1510.

In some embodiments, the antibody or antigen-binding fragment thereof comprises a heavy chain having an amino acid sequence that is at least 80% identical (e.g., 85%, 90%, 95%, 97%, 99%, or 100% identical) to SEQ ID NO: 1501 and a light chain having an amino acid sequence that is at least 80% identical (e.g., 85%, 90%, 95%, 97%, 99%, or 100% identical) to SEQ ID NO: 1508.

In some embodiments, the antibody or antigen-binding fragment thereof comprises a heavy chain having the amino acid sequence of SEQ ID NO: 1501 and a light chain having the amino acid sequence of SEQ ID NO: 1508.

In some embodiments, the antibody or antigen-binding fragment thereof is a human IgG4 antibody or antigen-binding fragment thereof.

### Antagonistic Anti-TNFR2 Antibodies or Antigen-Binding Fragments Thereof

In some embodiments, the antibody or antigen-binding fragment thereof is an antagonistic antibody or antigen-binding fragment thereof that specifically binds TNFR2 comprising one or more CDRs with amino acid sequences selected from Table 4 below, or variants thereof with up to two conservative amino acid substitutions. For example, the antibody or the antigen-binding fragment thereof may include a CDR-H1, a CDR-H2, a CDR-H3, a CDR-L1, a CDR-L2, and a CDR-L3 selected from Table 4.

TABLE 4
CDR sequences for antagonistic anti-TNFR2
antibodies or antigen-binding
fragments thereof.

| | | SEQ |
|---|---|---|
| Publication | | ID |
| Number | CDR sequences | NO: |
| WO 2016/187068 | CDR-H1: GYTFTDYL | 977 |
| | CDR-H2: VDPEYGST | 978 |
| | CDR-H3: ARDDGSYSPFDYWG | 979 |
| | CDR-L1: QNINKY | 980 |
| | CDR-L2: TYS | N/A |
| | CDR-L3: CLQYVNLLT | 982 |
| WO 2016/187068 | CDR-H1: GYTFTDYL | 983 |
| | CDR-H2: VDPEYGST | 984 |
| | CDR-H3: ARDDGSYSPFDYWG | 985 |
| | CDR-L1: QNINKY | 986 |
| | CDR-L2: TYS | N/A |
| | CDR-L3: CLQYVNLIT | 988 |
| WO 2016/187068 | CDR-H1: GYTFTDYI | 989 |
| | CDR-H2: VDPEYGST | 990 |
| | CDR-H3: ARDDGSYSPFDYWG | 991 |
| | CDR-L1: QNINKY | 992 |
| | CDR-L2: TYS | N/A |
| | CDR-L3: CLQYVNLLT | 994 |
| WO 2016/187068 | CDR-H1: GYTFTDYI | 995 |
| | CDR-H2: VDPEYGST | 996 |
| | CDR-H3: ARDDGSYSPFDYWG | 997 |
| | CDR-L1: QNINKY | 998 |
| | CDR-L2: TYS | N/A |
| | CDR-L3: CLQYVNLIT | 1000 |
| WO 2016/187068 | CDR-H1: GYTFTDYL | 1001 |
| | CDR-H2: VDPEYGST | 1002 |
| | CDR-H3: ARDDGSYSPFDYWG | 1003 |
| | CDR-L1: QNINKY | 1004 |
| | CDR-L2: YTS | N/A |
| | CDR-L3: CLQYVNLLT | 1006 |
| WO 2016/187068 | CDR-H1: GYTFTDYL | 1007 |
| | CDR-H2: VDPEYGST | 1008 |
| | CDR-H3: ARDDGSYSPFDYWG | 1009 |
| | CDR-L1: QNINKY | 1010 |
| | CDR-L2: YTS | N/A |
| | CDR-L3: CLQYVNLIT | 1012 |
| WO 2016/187068 | CDR-H1: GYTFTDYI | 1013 |
| | CDR-H2: VDPEYGST | 1014 |
| | CDR-H3: ARDDGSYSPFDYWG | 1015 |
| | CDR-L1: QNINKY | 1016 |
| | CDR-L2: YTS | |
| | CDR-L3: CLQYVNLLT | 1018 |
| WO 2016/187068 | CDR-H1: GYTFTDYI | 1019 |
| | CDR-H2: VDPEYGST | 1020 |
| | CDR-H3: ARDDGSYSPFDYWG | 1021 |
| | CDR-L1: QNINKY | 1022 |
| | CDR-L2: YTS | N/A |
| | CDR-L3: CLQYVNLIT | 1024 |
| WO 2016/187068 | CDR-H1: GFTFSSY | 1025 |
| | CDR-H2: SSGGSY | 1026 |
| | CDR-H3: QRVDGYSSYWYFDV | 1027 |
| | CDR-L1: SASSSVYYMY | 1028 |
| | CDR-L2: STSNLAS | 1029 |
| | CDR-L3: QQRRNYPYT | 1030 |
| WO 2020/089474 | CDR-H1: FDDYGMSWVRQAPG | 1031 |
| | CDR-H2: SVIYSGGSTYYADSVKGR | 1032 |
| | CDR-H3: CARDRSSSWYRDGMDV | 1033 |
| | CDR-L1: CTGSSSNIGAGYDVH | 1034 |
| | CDR-L2: GNSNRPS | 1035 |
| | CDR-L3: CAAWDDSLSGWV | 1036 |
| WO 2020/089474 | CDR-H1: FDDYGMSWVRQAPG | 1037 |
| | CDR-H2: STIYSGDNAYYGASVRGR | 1038 |
| | CDR-H3: ARVYSSSWRKRAFDI | 1039 |
| | CDR-L1: CSGTSSNIESNTVN | 1040 |
| | CDR-L2: SDNQRPS | 1041 |
| | CDR-L3: CAAWDDSLSGWV | 1042 |
| WO 2020/089474 | CDR-H1: FSDYYMSWIRQAPG | 1043 |
| | CDR-H2: ALIWYDGGNEYYADSVKGR | 1044 |
| | CDR-H3: VRETGNYGMDV | 1045 |
| | CDR-L1: CTGSSSNIGAGYDVH | 1046 |
| | CDR-L2: RNNQRPS | 1047 |
| | CDR-L3: CATWDDRVNGPV | 1048 |
| WO 2020/089474 | CDR-H1: FSDYYMSWIRQAPG | 1049 |
| | CDR-H2: ALIWYDGGNEYYADSVKGR | 1050 |
| | CDR-H3: ARYYGDGGFDP | 1051 |
| | CDR-L1: CTGSSSNIGAGYVVH | 1052 |
| | CDR-L2: SNNQRPS | 1053 |
| | CDR-L3: CAAWDDSLNGPV | 1054 |
| WO 2020/089474 | CDR-H1: FSSNYMSWVRQAPG | 1055 |
| | CDR-H2: ALIWYDGSNKYYADSVKGR | 1056 |
| | CDR-H3: AKDPLFDS | 1057 |
| | CDR-L1: CTGRSSNIGAGYDVH | 1058 |
| | CDR-L2: DNNKRPS | 1059 |
| | CDR-L3: CAAWDDSLNGPV | 1060 |
| WO 2020/089474 | CDR-H1: FNTYSMNWVRQAPG | 1061 |
| | CDR-H2: SVLYSDDDTHYADSVKGR | 1062 |
| | CDR-H3: ARDCGGDCHSGDDAFDI | 1063 |
| | CDR-L1: CSGSSSNIGSNTVN | 1064 |
| | CDR-L2: DNDKRPS | 1065 |
| | CDR-L3: CAAWHDSLNGWV | 1066 |
| WO 2020/089474 | CDR-H1: FSAYGMHWVRQAPG | 1067 |
| | CDR-H2: AVVSYDGREKHYADSVKGR | 1068 |
| | CDR-H3: ARSDGGYDSDSGYY | 1069 |
| | CDR-L1: CSGSTSNIGSNFVY | 1070 |
| | CDR-L2: DNNKRPS | 1071 |
| | CDR-L3: CSSYAYSDNIL | 1072 |
| WO 2020/089474 | CDR-H1: FSNAWMSWVRQAPG | 1073 |
| | CDR-H2: SGISSSGSSAYYADSVKGR | 1074 |
| | CDR-H3: ARHYYYHIAGYYYDTFDI | 1075 |
| | CDR-L1: CSGSSSNIGGNTVN | 1076 |
| | CDR-L2: GNTNRPS | 1077 |
| | CDR-L3: CAAWDDSLSGVV | 1078 |
| WO 2020/089474 | CDR-H1: FSSYAMSWVRQAPG | 1079 |
| | CDR-H2: ATISYHGSDKDYADSVKGR | 1080 |
| | CDR-H3: ARDANYHSSGYYYDVFDI | 1081 |
| | CDR-L1: CSGSSSNIGSNTVN | 1082 |
| | CDR-L2: GNSNRPS | 1083 |
| | CDR-L3: CAAWDDSLSTWV | 1084 |
| WO 2020/089474 | CDR-H1: FSDYYMTWIRQAPG | 1085 |
| | CDR-H2: SGISGSGGYIHYADSVKGR | 1086 |
| | CDR-H3: AREGLLPDAFD | 1087 |
| | CDR-L1: CSGSSSNIGNNYVS | 1088 |
| | CDR-L2: RNNQRPS | 1089 |
| | CDR-L3: CAAWDDSVSGWV | 1090 |
| WO 2020/089474 | CDR-H1: FSSYSMNWVRQAPG | 1091 |
| | CDR-H2: AVMSYDEYNTYYADSVKGR | 1092 |
| | CDR-H3: AKGFYGDYPLWDY | 1093 |
| | CDR-L1: CSGGNSNIGTNTVD | 1094 |
| | CDR-L2: SNNQRPS | 1095 |
| | CDR-L3: CAAWDDSVNGPV | 1096 |
| WO 2020/089474 | CDR-H1: FSSYEMNWVRQAPG | 1097 |
| | CDR-H2: STITGGGSIYDANSVQGR | 1098 |
| | CDR-H3: ARDSTYHSSGYYYDVFDI | 1099 |
| | CDR-L1: CSGSSSNIGSNTVN | 1100 |
| | CDR-L2: GNSNRPS | 1101 |
| | CDR-L3: CAAWDDSLSGHWV | 1102 |
| WO 2020/089474 | CDR-H1: FSSYGMHWVRQAPG | 1103 |
| | CDR-H2: SAVFGSGHGNTFYADAVKGR | 1104 |
| | CDR-H3: AREQLWFGQDAFDI | 1105 |
| | CDR-L1: CSGSSSNIGSNTVN | 1106 |
| | CDR-L2: GNSNRPS | 1107 |
| | CDR-L3: CQSYDSSLSASV | 1108 |
| WO 2020/089474 | CDR-H1: FSDAWMTWVRQAPG | 1109 |
| | CDR-H2: SDLSDSGGSTYYADSVKGR | 1110 |
| | CDR-H3: GRLAAGGPVDY | 1111 |
| | CDR-L1: CTGSSSNIGAGYDVH | 1112 |
| | CDR-L2: SNNQRPS | 1113 |
| | CDR-L3: CSVWDDSLNSWV | 1114 |
| WO 2021/200840 | CDR-H1: VYGVN | 1115 |
| | CDR-H2: MIWGDGSTAYNSALKS | 1116 |
| | CDR-H3: DGRRYALDY | 1117 |
| | CDR-L1: RASESVDSYGDSFLH | 1118 |
| | CDR-L2: RASNLDS | 1119 |
| | CDR-L3: QQSNEDPYT | 1120 |
| WO 2021/249542 | CDR-H1: TLGMGVG | 1121 |
| | CDR-H2: HIWWDADKYYNPALKS | 1122 |
| | CDR-H3: MTGTRYFDV | 1123 |
| | CDR-L1: KASQNINKFIA | 1124 |
| | CDR-L2: YTSTLQP | 1125 |
| | CDR-L3: LQYGVLWT | 1126 |
| WO 2021/249542 | CDR-H1: TYGMS | 1127 |
| | CDR-H2: WIHTYSGVPTYADDFKG | 1128 |
| | CDR-H3: GLYGVDY | 1129 |
| | CDR-L1: KASENVVTYVS | 1130 |
| | CDR-L2: GASNRYT | 1131 |
| | CDR-L3: GQSYTYPYT | 1132 |
| WO 2022/161425 | CDR-H1: TYDLS | 1133 |
| | CDR-H2: YINNGGISTYYSDTVKG | 1134 |
| | CDR-H3: GPFYGSANYFDY | 1135 |
| | CDR-L1: RTSESIYSNLP | 1136 |
| | CDR-L2: DATKLAE | 1137 |
| | CDR-L3: QHFWVTPWT | 1138 |
| WO 2022/161425 | CDR-H1: NYWMN | 1139 |
| | CDR-H2: MIHPSDTETRLNQNFKD | 1140 |
| | CDR-H3: GEGLGAARSVSMDY | 1141 |
| | CDR-L1: RASQDISNYLN | 1142 |
| | CDR-L2: YTAILHS | 1143 |
| | CDR-L3: QQGNTLPWT | 1144 |
| WO 2022/161425 | CDR-H1: DIYMH | 1145 |
| | CDR-H2: RIDPATGNTKHDPKFQD | 1146 |
| | CDR-H3: SPYGDFGAMDY | 1147 |
| | CDR-L1: TASSSVSSNYLH | 1148 |
| | CDR-L2: STSNLPS | 1149 |
| | CDR-L3: HQYHRSPWT | 1150 |
| WO 2022/166846 | CDR-H1: DYSFTNYYIH | 1151 |
| | CDR-H2: WIYPGNVITKYHEKFKG | 1152 |
| | CDR-H3: SRATGYFDY | 1153 |
| | CDR-L1: RASQDIRNYLN | 1154 |
| | CDR-L2: YTSRLHS | 1155 |
| | CDR-L3: QQFNTLPWT | 1156 |
| WO 2022/166846 | CDR-H1: GYTFTSYWMQ | 1157 |
| | CDR-H2: AIYPGDGDTRYTQKFKG | 1158 |
| | CDR-H3: DDGLYYAMDY | 1159 |
| | CDR-L1: TASSSVSSSYLH | 1160 |
| | CDR-L2: STSNLAS | 1161 |
| | CDR-L3: HQYHRSPPT | 1162 |
| WO 2022/166846 | CDR-H1: GYTFTDYSMN | 1163 |
| | CDR-H2: WLNTETGEPIYADDFKG | 1164 |
| | CDR-H3: EAIYYGTSYVLDY | 1165 |
| | CDR-L1: RASESVEYYGTNFMQ | 1166 |
| | CDR-L2: SASNVES | 1167 |
| | CDR-L3: QQSRKDPST | 1168 |
| WO 2022/267926 | CDR-H1: GYSITDYLLH | 1169 |
| | CDR-H2: WIDPEDAKTKYGQKFQS | 1170 |
| | CDR-H3: NSGTGTMDA | 1171 |
| | CDR-L1: KASQNVGSNVD | 1172 |
| | CDR-L2: KASIRYT | 1173 |
| | CDR-L3: MQSKSYPIT | 1174 |
| WO 2022/267926 | CDR-H1: GYTFTDYIIH | 1175 |
| | CDR-H2: WIDPDYGNTDYADKFKK | 1176 |
| | CDR-H3: DDGSPLVMDA | 1177 |
| | CDR-L1: NGSQNIDNYLA | 1178 |
| | CDR-L2: STYSLQT | 1179 |
| | CDR-L3: FQYNNGYM | 1180 |
| WO 2022/267926 | CDR-H1: GFTFSNAWMG | 1181 |
| | CDR-H2: QIKDKSNNYPTYYAESVKG | 1182 |
| | CDR-H3: YGDFFDY | 1183 |
| | CDR-L1: RASEDIYNGLA | 1184 |
| | CDR-L2: NANSLHT | 1185 |
| | CDR-L3: QQYYDHPLT | 1186 |
| WO 2022/267926 | CDR-H1: GYSITDYLLH | 1187 |
| | CDR-H2: WIDPEDGKTKYGQKFQS | 1188 |
| | CDR-H3: NSGTGTMDA | 1189 |
| | CDR-L1: KASQNVGSNVD | 1190 |
| | CDR-L2: KASIRYT | 1191 |
| | CDR-L3: MQSKSYPIT | 1192 |
| WO 2022/267926 | CDR-H1: GFTFSNAWMG | 1193 |
| | CDR-H2: QIKDKSNNYPTYYAESVKG | 1194 |
| | CDR-H3: YGDFFDY | 1195 |
| | CDR-L1: RASEDIYQGLA | 1196 |
| | CDR-L2: NANSLHT | 1197 |
| | CDR-L3: QQYYDHPLT | 1198 |
| WO 2022/267926 | CDR-H1: GYSITDYLLH | 1199 |
| | CDR-H2: WIDPEEGKTKYGQKFQS | 1200 |
| | CDR-H3: NSGTGTMDA | 1201 |
| | CDR-L1: KASQNVGSNVD | 1202 |
| | CDR-L2: KASIRYT | 1203 |
| | CDR-L3: MQSKSYPIT | 1204 |
| WO 2022/267926 | CDR-H1: GYTFTDYIIH | 1205 |
| | CDR-H2: WIDPDYGNTDYADKFKK | 1206 |
| | CDR-H3: DDGSPLVMDA | 1207 |
| | CDR-L1: QGSQNIDNYLA | 1208 |
| | CDR-L2: STYSLQT | 1209 |
| | CDR-L3: FQYNNGYM | 1210 |
| WO 2022/267926 | CDR-H1: GYTFTDYIIH | 1211 |
| | CDR-H2: WIDPDYGNTDYADKFKK | 1212 |
| | CDR-H3: DDGSPLVMDA | 1213 |
| | CDR-L1: NGAQNIDNYLA | 1214 |
| | CDR-L2: STYSLQT | 1215 |
| | CDR-L3: FQYNNGYM | 1216 |
| WO 2022/267926 | CDR-H1: GYTFTDYIIH | 1217 |
| | CDR-H2: WIDPDYGNTDYADKFKK | 1218 |
| | CDR-H3: DDASPLVMDA | 1219 |
| | CDR-L1: NGSQNIDNYLA | 1220 |
| | CDR-L2: STYSLQT | 1221 |
| | CDR-L3: FQYNNGYM | 1222 |
| WO 2022/267926 | CDR-H1: GYSITDYLLH | 1223 |
| | CDR-H2: WIDPEEGKTKYGQKFQS | 1224 |
| | CDR-H3: QSGTGTMDA | 1225 |
| | CDR-L1: KASQNVGSNVD | 1226 |
| | CDR-L2: KASIRYT | 1227 |
| | CDR-L3: MQSKSYPIT | 1228 |
| WO 2022/267926 | CDR-H1: GYSITDYLLH | 1229 |
| | CDR-H2: WIDPEEGKTKYGQKFQS | 1230 |
| | CDR-H3: NSATGTMDA | 1231 |
| | CDR-L1: KASQNVGSNVD | 1232 |
| | CDR-L2: KASIRYT | 1233 |
| | CDR-L3: MQSKSYPIT | 1234 |
| WO 2022/267926 | CDR-H1: GYSITDYLLH | 1235 |
| | CDR-H2: WIDPEDAKTKYGQKFQS | 1236 |
| | CDR-H3: QSGTGTMDA | 1237 |
| | CDR-L1: KASQNVGSNVD | 1238 |
| | CDR-L2: KASIRYT | 1239 |
| | CDR-L3: MQSKSYPIT | 1240 |
| WO 2022/267926 | CDR-H1: GYTFTDYIIH | 1241 |
| | CDR-H2: WIDPDYGNTDYADKFKK | 1242 |
| | CDR-H3: DDASPLVMDA | 1243 |
| | CDR-L1: QGSQNIDNYLA | 1244 |
| | CDR-L2: STYSLQT | 1245 |
| | CDR-L3: FQYNNGYM | 1246 |
| WO 2022/267926 | CDR-H1: GYTFTDYIIH | 1247 |
| | CDR-H2: WIDPDYGNTDYADKFKK | 1248 |
| | CDR-H3: DEGSPLVMDA | 1249 |
| | CDR-L1: NGSQNIDNYLA | 1250 |
| | CDR-L2: STYSLQT | 1251 |
| | CDR-L3: FQYNNGYM | 1252 |
| WO 2022/267926 | CDR-H1: GYTFTDYIIH | 1253 |
| | CDR-H2: WIDPDYGNTDYADKFKK | 1254 |
| | CDR-H3: DEGSPLVMDA | 1255 |
| | CDR-L1: QGSQNIDNYLA | 1256 |
| | CDR-L2: STYSLQT | 1257 |
| | CDR-L3: FQYNQGYM | 1258 |
| WO 2022/267926 | CDR-H1: GYSITDYLLH | 1259 |
| | CDR-H2: WIDPEDAKTKYGQKFQS | 1260 |
| | CDR-H3: NSATGTMDA | 1261 |
| | CDR-L1: KASQNVGSNVD | 1262 |
| | CDR-L2: KASIRYT | 1263 |
| | CDR-L3: MQSKSYPIT | 1264 |

In some embodiments, the antibody or antigen-binding fragment thereof is an antagonistic antibody or antigen-binding fragment thereof that specifically binds TNFR2 comprising a VH and/or a VL having amino acid sequences that are at least 80% identical (e.g., 85%, 90%, 95%, 97%, 99%, or 100% identical) to a VH and/or a VL sequence selected from Table 5 below. In some embodiments, the antibody or antigen-binding fragment may be any antibody or antigen-binding fragment thereof disclosed in International Patent Application Publication Nos. WO 2020/089474, WO 2021/200840, WO 2021/249542, WO 2022/161425, WO 2022/166846, or WO 2022/267926; incorporated herein by reference.

TABLE 5
VH and VL sequences for antagonistic anti-TNFR2
antibodies or antigen-binding fragments thereof.

| Publication Number | VH SEQ ID NO: | VL SEQ ID NO: |
|---|---|---|
| WO 2020/089474 | 1265 | 1266 |
| | 1267 | 1268 |
| | 1269 | 1270 |
| | 1271 | 1272 |
| | 1273 | 1274 |
| | 1275 | 1276 |
| | 1277 | 1278 |
| | 1279 | 1280 |
| | 1281 | 1282 |
| | 1283 | 1284 |
| | 1285 | 1286 |
| | 1287 | 1288 |
| | 1289 | 1290 |
| | 1291 | 1292 |
| WO 2021/200840 | 1293 | 1294 |
| WO 2021/249542 | 1295 | 1296 |
| | 1297 | 1298 |
| | 1299 | 1300 |
| | 1301 | 1302 |
| | 1303 | 1304 |
| WO 2022/161425 | 1305 | 1306 |
| | 1307 | 1308 |
| | 1309 | 1310 |
| | 1311 | 1312 |
| | 1313 | 1314 |
| | 1315 | 1316 |
| | 1317 | 1318 |
| | 1319 | 1320 |
| | 1321 | 1322 |
| | 1323 | 1324 |
| | 1325 | 1326 |
| | 1327 | 1328 |
| | 1329 | 1330 |
| | 1331 | 1332 |
| | 1333 | 1334 |
| | 1335 | 1336 |
| | 1337 | 1338 |
| WO 2022/166846 | 1339 | 1340 |
| | 1341 | 1342 |
| | 1343 | 1344 |
| | 1345 | 1346 |
| | 1347 | 1348 |
| | 1349 | 1350 |
| | 1351 | 1352 |
| | 1353 | 1354 |
| | 1355 | 1356 |
| | 1357 | 1358 |
| | 1359 | 1360 |
| | 1361 | 1362 |
| | 1363 | 1364 |
| | 1365 | 1366 |
| | 1367 | 1368 |
| | 1369 | 1370 |
| | 1371 | 1372 |
| WO 2022/267926 | 1373 | 1374 |
| | 1375 | 1376 |
| | 1377 | 1378 |
| | 1379 | 1380 |
| | 1381 | 1382 |
| | 1383 | 1384 |
| | 1385 | 1386 |
| | 1387 | 1388 |
| | 1389 | 1390 |
| | 1391 | 1392 |
| | 1393 | 1394 |
| | 1395 | 1396 |
| | 1397 | 1398 |
| | 1399 | 1400 |
| | 1401 | 1402 |
| | 1403 | 1404 |
| | 1405 | 1406 |
| | 1407 | 1408 |
| | 1409 | 1410 |
| | 1411 | 1412 |

Anti-TNFR2 Antibodies or Antigen-Binding Fragments Thereof that are Neither Agonistic Nor Antagonistic

In some embodiments, the antibody or antigen-binding fragment thereof specifically binds TNFR2 but is neither an agonist nor an antagonist. Such an antibody or antigen-binding fragment thereof is one that comprises one or more CDRs with amino acid sequences selected from Table 6 below, or variants thereof with up to two conservative amino acid substitutions. For example, the antibody or the antigen-binding fragment thereof may include a CDR-H1, a CDR-H2, a CDR-H3, a CDR-L1, a CDR-L2, and a CDR-L3 selected from Table 6.

TABLE 6
CDR sequences for anti-TNFR2 antibodies
or antigen-binding fragments thereof
that are neither agonists nor
antagonists.

| Publication | | SEQ ID |
|---|---|---|
| Number | CDR sequences | NO: |
| WO 2022/003693 | CDR-H1: GDTFTSYS | 1511 |
| | CDR-H2: IITILDDT | 1512 |
| | CDR-H3: AGVYEGPNS | 1513 |
| | EGSYDDYGFLKH | |
| | CDR-L1: RSVAND | 1514 |
| | CDR-L2: GAS | N/A |
| | CDR-L3: QQYAIWPPRET | 1515 |
| WO 2022/003693 | CDR-H1: GFNIKDFF | 1516 |
| | CDR-H2: IDPTNGDT | 1517 |
| | CDR-H3: TREGQDGYNAFDL | 1518 |
| | CDR-L1: QDVSTA | 1519 |
| | CDR-L2: SAS | N/A |
| | CDR-L3: QQHSYTPPT | 1520 |

In some embodiments, the antibody or antigen-binding fragment thereof is neither an agonist nor an antagonist that specifically binds TNFR2 comprising a VH and/or a VL having amino acid sequences that are at least 80% identical (e.g., 85%, 90%, 95%, 97%, 99%, or 100% identical) to a VH and/or a VL sequence selected from Table 7 below. In some embodiments, the antibody or antigen-binding fragment may be any antibody or antigen-binding fragment thereof disclosed in International Patent Application Publication No. WO 2022/003693; incorporated herein by reference.

TABLE 7
VH and VL sequences for antagonistic anti-TNFR2
antibodies or antigen-binding fragments thereof.

| Publication Number | VH SEQ ID NO: | VL SEQ ID NO: |
|---|---|---|
| WO 2022/003693 | 1521 | 1522 |
| | 1523 | 1524 |

### Antibodies or Antigen-Binding Fragments Thereof Binding Other TNFRSF Member Proteins

Antibodies or antigen-binding fragments thereof that specifically bind other TNFRSF member proteins (e.g., 4-1BB, CD27, CD30, CD40, DR6, EDAR, Fas, GITR, HVEM, LT-β receptor, NGFR, OPG, OX40, RANK, RELT (TNFRSF19L), TNFR1, TRAIL-R1 (TNFRSF10A), TRAIL-R2 (TNFRSF10B), TRAIL-R4, TRAMP, TROY, XEDAR, and DCR3, among others) are also well known in the art. Each of the antibodies or antigen binding fragments described below can be made into a modified antibody or antigen-binding fragment thereof that includes a modified Fc domain, as described herein.

In some embodiments, the antibody or antigen-binding fragment may be any antibody or antigen-binding fragment that specifically binds 4-1BB disclosed in International Patent Application Publication Nos. WO 2005/035584, WO 2012/032433, WO 2017/205745, WO 2018/017761, WO 2018/098370, WO 2018/114748, WO 2018/114754, WO 2018/127473, WO 2019/091436, WO 2019/140425, WO 2019/175125, WO 2019/196868, WO 2020/007817, WO 2020/073131, WO 2020/111913, WO 2021/030488, WO 2021/089588, WO 2021/093753, WO 2021/101346, WO 2021/118246, WO 2021/132746, WO 2021/167915, WO 2021/207827, WO 2022/039490, WO 2022/148413, WO 2022/178114, WO 2022/200478, WO 2023/073225, WO 2023/110788, WO 2023/134657, or WO 2023/174140; incorporated herein by reference.

In some embodiments, the anti-4-1BB antibody or antigen-binding fragment thereof comprises one or more CDR sequences of utomilumab (PF-05082566) with up to two conservative amino acid substitutions. In some embodiments, the anti-4-1BB antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of utomilumab (PF-05082566). Utomilumab (PF-05082566) and other anti-4-1BB antibodies are disclosed, e.g., in WO 2012/032433; incorporated herein by reference.

In some embodiments, the anti-4-1BB antibody or antigen-binding fragment thereof comprises one or more CDR sequences of urelumab (BMS-663513) with up to two conservative amino acid substitutions. In some embodiments, the anti-4-1BB antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of urelumab (BMS-663513). Urelumab (BMS-663513) and other anti-4-1BB antibodies are disclosed, e.g., in WO 2005/035584 and WO 2016/029073; incorporated herein by reference.

In some embodiments, the antibody or antigen-binding fragment may be any antibody or antigen-binding fragment that specifically binds CD27 disclosed in International Patent Application Publication Nos. WO 2011/130434, WO 2010/001908, WO 2012/004367, WO 2015/016718, WO 2019/196117, or WO 2021/087016; incorporated herein by reference.

In some embodiments, the anti-CD27 antibody or antigen-binding fragment thereof comprises one or more CDR sequences of varlilumab (CDX-1127) with up to two conservative amino acid substitutions. In some embodiments, the anti-CD27 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of varlilumab (CDX-1127). Varlilumab (CDX-1127) and other anti-CD27 antibodies are disclosed, e.g., in WO 2011/130434; incorporated herein by reference.

In some embodiments, the antibody or antigen-binding fragment may be any antibody or antigen-binding fragment that specifically binds CD30 disclosed in International Patent Application Publication Nos. WO 2008/025020, WO 2017/066122, WO 2020/135559, WO 2007/040653, WO 2023/057571, WO 2021/231568, WO 2021/091815, WO 2022/120084, WO 2021/155129, WO 2023/076989, WO 03/104432, WO 97/17374, or WO 96/22384; incorporated herein by reference.

In some embodiments, the antibody or antigen-binding fragment may be any antibody or antigen-binding fragment that specifically binds CD40 disclosed in International Patent Application Publication Nos. WO 2017/004016, WO 2018/185045, WO 2016/196314, WO 2017/040932, WO 2017/184619, WO 2017/004006, WO 2016/023960, WO 2018/189220, WO 2014/207064, WO 2016/069919, WO 2009/062054, WO 2012/075111, WO 2020/070035, WO 2007/075326, WO 2006/128103, WO 2017/205742, WO 2018/011421, WO 2012/149356, WO 2014/070934, WO 2014/065402, WO 2014/065403, WO 2005/063289, WO 2010/104761, WO 2021/081303, WO 2020/253722, WO 2020/154335, WO 2019/093342, WO 2018/017763, WO 2013/034904, WO 2019/106608, WO 2020/207470, WO 2018/088850, WO 2018/085533, WO 2021/183428, WO 2021/140222, WO 2019/057792, WO 2012/041635, WO 2018/178046, WO 2020/014974, WO 2020/065409, WO 2021/222188, WO 2022/078357, WO 2020/230899, WO 2020/230901, WO 2019/204756, WO 2021/239968, WO 2022/133123, WO 2022/101458, WO 2022/251311, WO 2023/274007, WO 2022/243261, WO 2023/284714, WO 2023/046037, WO 03/040170, WO 2023/274201, WO 2023/198194, WO 2023/020475, WO 2023/125652, WO 2022/037662, WO 2021/243422, or WO 2023/052581; incorporated herein by reference.

In some embodiments, the anti-CD40 antibody or antigen-binding fragment thereof comprises one or more CDR sequences of CDX-1140 with up to two conservative amino acid substitutions. In some embodiments, the anti-CD40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of CDX-1140. CDX-1140 and other anti-CD40 antibodies are disclosed, e.g., in WO 2017/184619 and Vitale et al. Cancer Immunol. Immunother. 68:233-245, 2019; incorporated herein by reference.

In some embodiments, the anti-CD40 antibody or antigen-binding fragment thereof comprises one or more CDR sequences of SEA-CD40 with up to two conservative amino acid substitutions. In some embodiments, the anti-CD40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of SEA-CD40. SEA-CD40 and other anti-CD40 antibodies are disclosed, e.g., in WO 2016/069919; incorporated herein by reference.

In some embodiments, the anti-CD40 antibody or antigen-binding fragment thereof comprises one or more CDR sequences of RO7009789 with up to two conservative amino acid substitutions. In some embodiments, the anti-CD40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of RO7009789. RO7009789 and other anti-CD40 antibodies are disclosed, e.g., in U.S. Pat. No. 7,338,660; WO 03/040170; and Vonderheide et al. J. Clin. Oncol. 25:876, 2007; incorporated herein by reference.

In some embodiments, the anti-CD40 antibody or antigen-binding fragment thereof comprises one or more CDR sequences of JNJ-64457107 (ADC1013) with up to two conservative amino acid substitutions. In some embodiments, the anti-CD40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of JNJ-64457107 (ADC1013). JNJ-64457107 (ADC1013) and other anti-CD40 antibodies are disclosed, e.g., in WO 2016/023960 and Mangsbo et al. Clin. Cancer Res. 21:1115-1126, 2015; incorporated herein by reference.

In some embodiments, the anti-CD40 antibody or antigen-binding fragment thereof comprises one or more CDR sequences of APX-005M with up to two conservative amino acid substitutions. In some embodiments, the anti-CD40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of APX-005M. APX-005M and other anti-CD40 antibodies are disclosed, e.g., in WO 2014/070934 and WO 2018/085533; incorporated herein by reference.

In some embodiments, the anti-CD40 antibody or antigen-binding fragment thereof comprises one or more CDR sequences of Chi Lob 7/4 with up to two conservative amino acid substitutions. In some embodiments, the anti-CD40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of Chi Lob 7/4. Chi Lob 7/4 and other anti-CD40 antibodies are disclosed, e.g., in US 2009/0074711 and Johnson et al. Clin. Cancer Res. 21:1321-1328, 2015; incorporated herein by reference.

In some embodiments, the antibody or antigen-binding fragment may be any antibody or antigen-binding fragment that specifically binds DR6 disclosed in International Patent Application Publication No. WO 2010/062904; incorporated herein by reference.

In some embodiments, the antibody or antigen-binding fragment may be any antibody or antigen-binding fragment that specifically binds EDAR disclosed in International Patent Application Publication No. WO 2010/113117; incorporated herein by reference.

In some embodiments, the antibody or antigen-binding fragment may be any antibody or antigen-binding fragment that specifically binds HVEM disclosed in International Patent Application Publication Nos. WO 2017/096189, WO 2016/054638, WO 2017/096276, WO 2017/087678, WO 2015/187835, WO 2015/184099, WO 2020/218951, WO 2018/094300, WO 2006/105021, WO 2018/185618, WO 2018/017889, WO 2020/163646, WO 2011/028683, WO 2018/158658, WO 2015/031667, WO 2009/009116, WO 2017/214548, WO 2018/091739, WO 2018/018039, WO 2020/108636, WO 2019/201301, WO 2019/184898, WO 2022/240161, WO 2023/175614, or WO 2021/178814; incorporated herein by reference.

In some embodiments, the antibody or antigen-binding fragment may be any antibody or antigen-binding fragment that specifically binds GITR disclosed in International Patent Application Publication Nos. WO 2020/222235, WO 2014/184360, WO 2015/031667, WO 2022/208505, WO 2022/197866, or WO 2021/160266, WO 2006/105021, WO 2011/028683, WO 2015/026684, WO 2015/184099, WO 2016/196792; incorporated herein by reference.

In some embodiments, the anti-GITR antibody or antigen-binding fragment thereof comprises one or more CDR sequences of TRX-518 with up to two conservative amino acid substitutions. In some embodiments, the anti-GITR antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of TRX-518. TRX-518 and other anti-GITR antibodies are disclosed, e.g., in U.S. Pat. Nos. 7,812,135; 8,388,967; 9,028,823; WO 2006/105021; and Ponte J et al. Clinical Immunology 135: S96, 2010; incorporated herein by reference.

In some embodiments, the anti-GITR antibody or antigen-binding fragment thereof comprises one or more CDR sequences of MK-4166 or MK-1248 with up to two conservative amino acid substitutions. In some embodiments, the anti-GITR antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of MK-4166 or MK-1248. MK-4166, MK-1248, and other anti-GITR antibodies are disclosed, e.g., in U.S. Pat. No. 8,709,424; WO 2011/028683; WO 2015/026684; and Mahne et al. Cancer Res. 77:1108-1118, 2017; incorporated herein by reference.

In some embodiments, the anti-GITR antibody or antigen-binding fragment thereof comprises one or more CDR sequences of GWN-323 with up to two conservative amino acid substitutions. In some embodiments, the anti-GITR antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of GWN-323. GWN-323 and other anti-GITR antibodies are disclosed, e.g., in Piha-Paul et al. J. Immunother. Cancer 9:e002863, 2021; incorporated herein by reference.

In some embodiments, the anti-GITR antibody or antigen-binding fragment thereof comprises one or more CDR sequences of INCAGN01876 with up to two conservative amino acid substitutions. In some embodiments, the anti-GITR antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of INCAGN01876. INCAGN01876 and other anti-GITR antibodies are disclosed, e.g., in U.S. Pat. No. 10,155,818 and WO 2015/184099; incorporated herein by reference.

In some embodiments, the anti-GITR antibody or antigen-binding fragment thereof comprises one or more CDR sequences of BMS-986156 with up to two conservative amino acid substitutions. In some embodiments, the anti-GITR antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of BMS-986156. BMS-986156 and other anti-GITR antibodies are disclosed, e.g., in U.S. Pat. No. 9,228,016 and WO 2016/196792; incorporated herein by reference.

In some embodiments, the anti-GITR antibody or antigen-binding fragment thereof comprises one or more CDR sequences of AMG-228 with up to two conservative amino acid substitutions. In some embodiments, the anti-GITR antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of AMG-228. AMG-228 and other anti-GITR antibodies are disclosed, e.g., in U.S. Pat. No. 9,464,139 and WO 2015/031667; incorporated herein by reference.

In some embodiments, the antibody or antigen-binding fragment may be any antibody or antigen-binding fragment that specifically binds OX40 disclosed in International Patent Application Publication Nos. WO 2018/177220, WO 2017/063162, WO 2018/112346, WO 2017/096281, WO 2017/096182, WO 2016/179517, WO 2018/202649, WO 2010/096418, WO 2013/008171, WO 2013/068563, WO 2015/095423, WO 2015/153514, WO 2016/073380, WO 2016/081384, WO 2016/200836, WO 2017/130076, WO 2019/086497, WO 2018/031490, WO 2018/017888, WO 2018/002339, WO 2020/103836, WO 2007/062245, WO 2021/098851, WO 2019/214624, WO 2013/028231, WO 2019/089921, WO 2012/027328, WO 2020/151761, WO 2019/100320, WO 2020/259667, WO 2021/030488, WO 2023/109900, WO 2022/258015, WO 2022/178114, WO 2023/089079, WO 2020/063668, WO 2023/143597, WO 2020/119789, WO 2020/119793, WO 2020/119792, WO 2022/002009, or WO 2022/262749; incorporated herein by reference.

In some embodiments, the anti-OX40 antibody or antigen-binding fragment thereof comprises one or more CDR sequences of tavolimab (MEDI0562) with up to two conservative amino acid substitutions. In some embodiments, the anti-OX40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of tavolimab (MEDI0562). tavolimab (MEDI0562) and other anti-OX40 antibodies are disclosed, e.g., in WO 2015/095423; WO 2015/153514; WO 2016/073380; and WO 2016/081384; incorporated herein by reference.

In some embodiments, the anti-OX40 antibody or antigen-binding fragment thereof comprises one or more CDR sequences of PF-04518600 with up to two conservative amino acid substitutions. In some embodiments, the anti-OX40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of PF-04518600. PF-04518600 and other anti-OX40 antibodies are disclosed, e.g., in WO 2017/130076; incorporated herein by reference.

In some embodiments, the anti-OX40 antibody or antigen-binding fragment thereof comprises one or more CDR sequences of BMS-986178 with up to two conservative amino acid substitutions. In some embodiments, the anti-OX40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of BMS-986178. BMS-986178 and other anti-OX40 antibodies are disclosed, e.g., in WO 2019/089921; incorporated herein by reference.

In some embodiments, the anti-OX40 antibody or antigen-binding fragment thereof comprises one or more CDR sequences of MOXR-0916 with up to two conservative amino acid substitutions. In some embodiments, the anti-OX40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of MOXR-0916. MOXR-0916 and other anti-OX40 antibodies are disclosed, e.g., in WO 2016/200836; incorporated herein by reference.

In some embodiments, the anti-OX40 antibody or antigen-binding fragment thereof comprises one or more CDR sequences of GSK-3174998 with up to two conservative amino acid substitutions. In some embodiments, the anti-OX40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of GSK-3174998.

In some embodiments, the anti-OX40 antibody or antigen-binding fragment thereof comprises one or more CDR sequences of INCAGN01949 with up to two conservative amino acid substitutions. In some embodiments, the anti-OX40 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of INCAGN01949. INCAGN01949 and other anti-OX40 antibodies are disclosed, e.g., in WO 2016/179517; incorporated herein by reference.

In some embodiments, the antibody or antigen-binding fragment may be any antibody or antigen-binding fragment that specifically binds RANK disclosed in International Patent Application Publication No. WO 2020/113274; incorporated herein by reference.

In some embodiments, the antibody or antigen-binding fragment may be any antibody or antigen-binding fragment that specifically binds a TRAIL receptor (e.g., TRAIL-R1 (TNFRSF10A), TRAIL-R2 (TNFRSF10B), or TRAIL-R4) disclosed in International Patent Application Publication Nos. WO 2016/111344, WO 2017/028279, WO 2007/027713, or WO 2016/074245; incorporated herein by reference.

### Anti-TNFSF Antibodies or Antigen-Binding Fragments Thereof

Antibodies or antigen-binding fragments thereof that specifically bind TNFSF member proteins (e.g., TNF-α, TNF-β, lymphotoxin-β (LT-β), CD40L, FasL, CD30L, 4-1BBL, CD27L, OX40L, TRAIL, LIGHT, RANKL, TWEAK, APRIL, BAFF, VEGI, EDA-A1, EDA-A2, and GITRL, among others) are well known in the art. Each of the antibodies or antigen binding fragments described below can be made into a modified antibody or antigen-binding fragment thereof that includes a modified Fc domain, as described herein. In some embodiments, the antibody or antigen-binding fragment may be any antibody or antigen-binding fragment that specifically binds TRAIL.

### CDRs of Agonisitc Antibodies or Antigen-Binding Fragments Thereof Binding TNFRSF Member Proteins

The CDR-H1 of agonistic antibodies or antigen-binding fragments thereof that bind TNFRSF member proteins (e.g., TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, and DCR3) is of particular importance in the determination of the binding specificity and agonistic activity of such antibodies or antigen-binding fragments thereof. Efficient signaling of TNFRSF member proteins requires the formation of a homotrimeric receptor structure. Antibodies or antigen-binding fragments thereof that contain a particular CDR-H1 sequence that specifically binds one or more amino acid residues within the trimer interface of a TNFRSF member protein may stabilize the trimeric structure, thereby acting as an agonist of said TNFRSF member protein.

In some embodiments, the CDR-H1 of an agonistic TNFR2 antibody or antigen-binding fragment thereof of the disclosure comprises the amino acid sequence of X¹X²X²X¹X²X³X²JJJ, in which each X¹ is independently G, A, V, L, I, M, W, F, or P; each X² is independently Y, S, T, C, N, or Q; X³ is D or E; and each J is independently a naturally occurring amino acid or is absent. For example, each amino acid residue of a particular CDR-H1 sequence of an agonistic TNFR2 antibody or antigen-binding fragment thereof, namely GYTFTDY (SEQ ID NO: 1413), can be substituted by amino acids with a similar physiochemical property (e.g., polarity, electric charge, acidity, or alkalinity), as shown in Table 8 below.

TABLE 8
Acceptable amino acid substitutions of
an exemplary CDR-H1 sequence of an
agonistic TNFR2 antibody or antigen-
binding fragment thereof.
CDR-H1 Sequence: GYTFTDY (SEQ ID NO: 1413)

| Amino Acid | Wild-type | Acceptable Amino |
|---|---|---|
| Position | Residue | Acid Substitutions |
| 1 | G | A, V, L, I, M, W, F, or P |
| 2 | Y | S, T, C, N, or Q |
| 3 | T | S, C, Y, N, or Q |
| 4 | F | G, A, V, L, I, M, W, or P |
| 5 | T | S, C, Y, N, or Q |
| 6 | D | E |
| 7 | Y | S, T, C, N, or Q |

In some embodiments, the CDR-H1 of an agonistic TNFR2 antibody or antigen-binding fragment thereof of the disclosure comprises the amino acid sequence of GYTFTZ¹Z²JJJ (SEQ ID NO: 1480), in which Z¹ is D or T; Z² is Y, F, or L; and each J is independently a naturally occurring amino acid or is absent. It is known in the art that an antibody comprising a CDR-H1 comprising the amino acid sequence of SEQ ID NO: 1480 binds human TNFR2 and exerts agonistic activity. For example, International Patent Application Publication No. WO 2020/061210 teaches that an antibody comprising a CDR-H1 having the amino acid sequence of GYTFTTF (SEQ ID NO: 31), GYTFTTFGMS (SEQ ID NO: 37), or GYTFTTFG (SEQ ID NO: 73) is an agonistic antibody that binds human TNFR2. International Patent Application Publication No. WO 2021/141907 teaches that an antibody comprising a CDR-H1 having the amino acid sequence of GYTFTDYY (SEQ ID NO: 325) is an agonistic antibody that binds human TNFR2. International Patent Application Publication No. WO 2022/003690 teaches that an antibody comprising a CDR-H1 having the amino acid sequence of GYTFTDLG (SEQ ID NO: 481) is an agonistic antibody that binds human TNFR2. In some embodiments, the agonistic TNFR2 antibody or antigen-binding fragment thereof of the disclosure further comprises one or more of a CDR-H2, a CDR-H3, a CDR-L1, a CDR-L2, and a CDR-L3 set forth in Table 2.

In some embodiments, the CDR-H1 of an agonistic TNFR2 antibody or antigen-binding fragment thereof of the disclosure comprises the amino acid sequence of GYTFTDY (SEQ ID NO: 1413) or a variant thereof with one or more conservative amino acid substitutions. In some embodiments, the CDR-H1 of an agonistic TNFR2 antibody or antigen-binding fragment thereof of the disclosure comprises the amino acid sequence of GYTFTDY (SEQ ID NO: 1413) or a variant thereof with up to two conservative amino acid substitutions. In some embodiments, the CDR-H1 of an agonistic TNFR2 antibody or antigen-binding fragment thereof of the disclosure comprises the amino acid sequence of GYTFTDY (SEQ ID NO: 1413). In some embodiments, the agonistic TNFR2 antibody or antigen-binding fragment thereof of the disclosure further comprises one or more of a CDR-H2, a CDR-H3, a CDR-L1, a CDR-L2, and a CDR-L3 set forth in Table 2.

In some embodiments, the CDR-H1 of an agonistic TNFR2 antibody or antigen-binding fragment thereof of the disclosure comprises the amino acid sequence of X¹X²X²X³X¹, in which each X¹ is independently D or E; each X² is independently Y, S, T, C, N, or Q; and X³ is L, A, V, G, I, M, W, F, or P. For example, each amino acid residue of a particular CDR-H1 sequence of an agonistic TNFR2 antibody or antigen-binding fragment thereof, namely DYNLD (SEQ ID NO: 1541), can be substituted by amino acids with a similar physiochemical property (e.g., polarity, electric charge, acidity, or alkalinity), as shown in Table 9 below.

TABLE 9
Acceptable amino acid substitutions of an exemplary CDR-H1 sequence
of an agonistic TNFR2 antibody or antigen-binding fragment thereof.
CDR-H1 Sequence: DYNLD (SEQ ID NO: 1541)

| Amino Acid | Wild-type | |
|---|---|---|
| Position | Residue | Acceptable Amino Acid Substitutions |
| 1 | D | E |
| 2 | Y | S, T, C, N, or Q |
| 3 | N | S, T, C, Y, or Q |
| 4 | L | A, V, G, I, M, W, F, or P |
| 5 | D | E |

In some embodiments, the CDR-H1 of an agonistic TNFR2 antibody or antigen-binding fragment thereof of the disclosure comprises the amino acid sequence of DYNLD (SEQ ID NO: 1541) or a variant thereof with one or more conservative amino acid substitutions. In some embodiments, the CDR-H1 of an agonistic TNFR2 antibody or antigen-binding fragment thereof of the disclosure comprises the amino acid sequence of DYNLD (SEQ ID NO: 1541) or a variant thereof with up to two conservative amino acid substitutions. In some embodiments, the CDR-H1 of an agonistic TNFR2 antibody or antigen-binding fragment thereof of the disclosure comprises the amino acid sequence of DYNLD (SEQ ID NO: 1541). In some embodiments, the agonistic TNFR2 antibody or antigen-binding fragment thereof of the disclosure further comprises one or more of a CDR-H2, a CDR-H3, a CDR-L1, a CDR-L2, and a CDR-L3 set forth in Table 2.

The CDR-H2, CDR-H3, CDR-L1, CDR-L2, and CDR-L3 of agonistic antibodies or antigen-binding fragments thereof that bind TNFRSF member proteins (e.g., TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, and DCR3) can tolerate significant degree of variation. Amino acid substitutions (e.g., conservative amino acid substitutions) at the CDR-H2, CDR-H3, CDR-L1, CDR-L2, or CDR-L3 of agonistic antibodies or antigen-binding fragments thereof that bind TNFRSF member proteins do not affect the binding specificity and agonist activity of these antibodies or antigen-binding fragments thereof.

In some embodiments, the CDR-H2 of an agonistic TNFR2 antibody or antigen-binding fragment thereof of the disclosure comprises the amino acid sequence of X¹X²X³X²X³X³X¹X³X³X³X³X³X³X⁴X²X⁴X², in which each X¹ is independently D or E; each X² is independently I, A, V, L, G, M, W, F, or P; each X³ is independently N, S, T, C, Y, or Q; and each X⁴ is independently K, R, or H. For example, each amino acid residue of a particular CDR-H2 sequence of an agonistic TNFR2 antibody or antigen-binding fragment thereof, namely DINPNYDSTSYSQKFRG (SEQ ID NO: 1542), can be substituted by amino acids with a similar physiochemical property (e.g., polarity, electric charge, acidity, or alkalinity), as shown in Table 10 below.

TABLE 10
Acceptable amino acid substitutions of an
exemplary CDR-H2 sequence of an agonistic
TNFR2 antibody or antigen-binding
fragment thereof.
CDR-H2 Sequence:
DINPNYDSTSYSQKFRG (SEQ ID NO: 1542)

| Amino | Wild- | |
|---|---|---|
| Acid | type | Acceptable Amino |
| Position | Residue | Acid Substitutions |
| 1 | D | E |
| 2 | | A, V, L, G, M, W, F, or P |
| 3 | N | S, T, C, Y, or Q |
| 4 | P | A, V, L, I, M, W, F, or G |
| 5 | N | S, T, C, Y, or Q |
| 6 | Y | S, T, C, N, or Q |
| 7 | D | E |
| 8 | S | Y, T, C, N, or Q |
| 9 | T | S, C, Y, N, or Q |
| 10 | S | Y, T, C, N, or Q |
| 11 | Y | S, T, C, N, or Q |
| 12 | S | Y, T, C, N, or Q |
| 13 | Q | S, T, C, N, or Y |
| 14 | K | R or H |
| 15 | F | G, A, V, L, I, M, W, or P |
| 16 | R | H or K |
| 17 | G | A, V, L, I, M, W, F, or P |

In some embodiments, the CDR-H2 of an agonistic TNFR2 antibody or antigen-binding fragment thereof of the disclosure comprises the amino acid sequence of DINPNYDSTSYSQKFRG (SEQ ID NO: 1542) or a variant thereof with one or more conservative amino acid substitutions. In some embodiments, the CDR-H2 of an agonistic TNFR2 antibody or antigen-binding fragment thereof of the disclosure comprises the amino acid sequence of DINPNYDSTSYSQKFRG (SEQ ID NO: 1542) or a variant thereof with up to two conservative amino acid substitutions. In some embodiments, the CDR-H2 of an agonistic TNFR2 antibody or antigen-binding fragment thereof of the disclosure comprises the amino acid sequence of DINPNYDSTSYSQKFRG (SEQ ID NO: 1542). In some embodiments, the agonistic TNFR2 antibody or antigen-binding fragment thereof of the disclosure further comprises one or more of a CDR-H1, a CDR-H3, a CDR-L1, a CDR-L2, and a CDR-L3 set forth in Table 2.

In some embodiments, the CDR-H3 of an agonistic TNFR2 antibody or antigen-binding fragment thereof of the disclosure comprises the amino acid sequence of X¹X²X²X¹X²X¹X³X¹, in which each X¹ is independently G, A, V, L, I, M, W, F, or P; each X² is independently N, S, T, C, Y, or Q; and X³ is D or E. For example, each amino acid residue of a particular CDR-H3 sequence of an agonistic TNFR2 antibody or antigen-binding fragment thereof, namely GNSWYFDV (SEQ ID NO: 1543), can be substituted by amino acids with a similar physiochemical property (e.g., polarity, electric charge, acidity, or alkalinity), as shown in Table 11 below.

TABLE 11
Acceptable amino acid substitutions of
an exemplary CDR-H3 sequence of an
agonistic TNFR2 antibody or antigen-
binding fragment thereof.
CDR-H3 Sequence: GNSWYFDV (SEQ ID NO: 1543)

| Amino Acid | Wild-type | Acceptable Amino |
|---|---|---|
| Position | Residue | Acid Substitutions |
| 1 | G | A, V, L, I, M, W, F, or P |
| 2 | N | S, T, C, Y, or Q |
| 3 | S | Y, T, C, N, or Q |
| 4 | W | A, V, L, I, M, F, P, or G |
| 5 | Y | S, T, C, N, or Q |
| 6 | F | G, A, V, L, I, M, W, or P |
| 7 | D | E |
| 8 | V | G, A, F, L, I, M, W, or P |

In some embodiments, the CDR-H3 of an agonistic TNFR2 antibody or antigen-binding fragment thereof of the disclosure comprises the amino acid sequence of GNSWYFDV (SEQ ID NO: 1543) or a variant thereof with one or more conservative amino acid substitutions. In some embodiments, the CDR-H3 of an agonistic TNFR2 antibody or antigen-binding fragment thereof of the disclosure comprises the amino acid sequence of GNSWYFDV (SEQ ID NO: 1543) or a variant thereof with up to two conservative amino acid substitutions. In some embodiments, the CDR-H3 of an agonistic TNFR2 antibody or antigen-binding fragment thereof of the disclosure comprises the amino acid sequence of GNSWYFDV (SEQ ID NO: 1543). In some embodiments, the agonistic TNFR2 antibody or antigen-binding fragment thereof of the disclosure further comprises one or more of a CDR-H1, a CDR-H2, a CDR-L1, a CDR-L2, and a CDR-L3 set forth in Table 2.

In some embodiments, the CDR-L1 of an agonistic TNFR2 antibody or antigen-binding fragment thereof of the disclosure comprises the amino acid sequence of X¹X²X¹X¹X¹X²X³X⁴X¹X¹, in which each X¹ is independently S, Y, T, C, N, or Q; each X² is independently A, G, V, L, I, M, W, F, or P; X³ is R, H, or K; and X⁴ is Y, A, V, I, L, M, F, or W. For example, each amino acid residue of a particular CDR-L1 sequence of an agonistic TNFR2 antibody or antigen-binding fragment thereof, namely SASSSVRYNY (SEQ ID NO: 1544), can be substituted by amino acids with a similar physiochemical property (e.g., polarity, electric charge, acidity, or alkalinity), as shown in Table 12 below.

TABLE 12
Acceptable amino acid substitutions of an
exemplary CDR-L1 sequence of an agonistic
TNFR2 antibody or antigen-binding
fragment thereof.
CDR-L1 Sequence:
SASSSVRYNY (SEQ ID NO: 1544)

| Amino Acid | Wild-type | Acceptable Amino |
|---|---|---|
| Position | Residue | Acid Substitutions |
| 1 | S | Y, T, C, N, or Q |
| 2 | A | G, V, L, I, M, W, F, or P |
| 3 | S | Y, T, C, N, or Q |
| 4 | S | Y, T, C, N, or Q |
| 5 | S | Y, T, C, N, or Q |
| 6 | V | G, A, F, L, I, M, W, or P |
| 7 | R | H or K |
| 8 | Y | A, V, I, L, M, F, or W |
| 9 | N | S, T, C, Y, or Q |
| 10 | Y | S, T, C, N, or Q |

In some embodiments, the CDR-L1 of an agonistic TNFR2 antibody or antigen-binding fragment thereof of the disclosure comprises the amino acid sequence of SASSSVRYNY (SEQ ID NO: 1544) or a variant thereof with one or more conservative amino acid substitutions. In some embodiments, the CDR-L1 of an agonistic TNFR2 antibody or antigen-binding fragment thereof of the disclosure comprises the amino acid sequence of SASSSVRYNY (SEQ ID NO: 1544) or a variant thereof with up to two conservative amino acid substitutions. In some embodiments, the CDR-L1 of an agonistic TNFR2 antibody or antigen-binding fragment thereof of the disclosure comprises the amino acid sequence of SASSSVRYNY (SEQ ID NO: 1544). In some embodiments, the agonistic TNFR2 antibody or antigen-binding fragment thereof of the disclosure further comprises one or more of a CDR-H1, a CDR-H2, a CDR-H3, a CDR-L2, and a CDR-L3 set forth in Table 2.

In some embodiments, the CDR-L2 of an agonistic TNFR2 antibody or antigen-binding fragment thereof of the disclosure comprises the amino acid sequence of X¹X²X²X²X¹X¹X², in which each X¹ is independently L, A, V, G, I, M, W, F, or P; and each X² is independently T, S, C, Y, N, or Q. For example, each amino acid residue of a particular CDR-L2 sequence of an agonistic TNFR2 antibody or antigen-binding fragment thereof, namely LTSNLAS (SEQ ID NO: 1545), can be substituted by amino acids with a similar physiochemical property (e.g., polarity, electric charge, acidity, or alkalinity), as shown in Table 13 below.

TABLE 13
Acceptable amino acid substitutions of
an exemplary CDR-L2 sequence of an
agonistic TNFR2 antibody or antigen-
binding fragment thereof.
CDR-L2 Sequence: LTSNLAS (SEQ ID NO: 1545)

| Amino Acid | Wild-type | Acceptable Amino |
|---|---|---|
| Position | Residue | Acid Substitutions |
| 1 | L | A, V, G, I, M, W, F, or P |
| 2 | T | S, C, Y, N, or Q |
| 3 | S | Y, T, C, N, or Q |
| 4 | N | S, T, C, Y, or Q |
| 5 | L | A, V, G, I, M, W, F, or P |
| 6 | A | G, V, L, I, M, W, F, or P |
| 7 | S | Y, T, C, N, or Q |

In some embodiments, the CDR-L2 of an agonistic TNFR2 antibody or antigen-binding fragment thereof of the disclosure comprises the amino acid sequence of LTSNLAS (SEQ ID NO: 1545) or a variant thereof with one or more conservative amino acid substitutions. In some embodiments, the CDR-L2 of an agonistic TNFR2 antibody or antigen-binding fragment thereof of the disclosure comprises the amino acid sequence of LTSNLAS (SEQ ID NO: 1545) or a variant thereof with up to two conservative amino acid substitutions. In some embodiments, the CDR-L2 of an agonistic TNFR2 antibody or antigen-binding fragment thereof of the disclosure comprises the amino acid sequence of LTSNLAS (SEQ ID NO: 1545). In some embodiments, the agonistic TNFR2 antibody or antigen-binding fragment thereof of the disclosure further comprises one or more of a CDR-H1, a CDR-H2, a CDR-H3, a CDR-L1, and a CDR-L3 set forth in Table 2.

In some embodiments, the CDR-L3 of an agonistic TNFR2 antibody or antigen-binding fragment thereof of the disclosure comprises the amino acid sequence of X¹X²X²X¹X²X²X²X¹X¹X², in which each X¹ is independently P, A, V, L, I, M, W, F, or G; and each X² is independently Q, S, T, C, N, or Y. For example, each amino acid residue of a particular CDR-L3 sequence of an agonistic TNFR2 antibody or antigen-binding fragment thereof, namely PQQWSSNPLT (SEQ ID NO: 1546), can be substituted by amino acids with a similar physiochemical property (e.g., polarity, electric charge, acidity, or alkalinity), as shown in Table 14 below.

TABLE 14
Acceptable amino acid substitutions of an
exemplary CDR-L3 sequence of an agonistic
TNFR2 antibody or antigen-
binding fragment thereof.
CDR-L3 Sequence:
PQQWSSNPLT (SEQ ID NO: 1546)

| Amino Acid | Wild-type | Acceptable Amino |
|---|---|---|
| Position | Residue | Acid Substitutions |
| 1 | P | A, V, L, I, M, W, F, or G |
| 2 | Q | S, T, C, N, or Y |
| 3 | Q | S, T, C, N, or Y |
| 4 | W | A, V, L, I, M, W, F, or G |
| 5 | S | Y, T, C, N, or Q |
| 6 | S | Y, T, C, N, or Q |
| 7 | N | S, T, C, Y, or Q |
| 8 | P | A, V, L, I, M, W, F, or G |
| 9 | L | A, V, G, I, M, W, F, or P |
| 10 | T | S, Y, C, N, or Q |

In some embodiments, the CDR-L3 of an agonistic TNFR2 antibody or antigen-binding fragment thereof of the disclosure comprises the amino acid sequence of PQQWSSNPLT (SEQ ID NO: 1546) or a variant thereof with one or more conservative amino acid substitutions. In some embodiments, the CDR-L3 of an agonistic TNFR2 antibody or antigen-binding fragment thereof of the disclosure comprises the amino acid sequence of PQQWSSNPLT (SEQ ID NO: 1546) or a variant thereof with up to two conservative amino acid substitutions. In some embodiments, the CDR-L3 of an agonistic TNFR2 antibody or antigen-binding fragment thereof of the disclosure comprises the amino acid sequence of PQQWSSNPLT (SEQ ID NO: 1546). In some embodiments, the agonistic TNFR2 antibody or antigen-binding fragment thereof of the disclosure further comprises one or more of a CDR-H1, a CDR-H2, a CDR-H3, a CDR-L1, and a CDR-L2 set forth in Table 2.

In some embodiments, any of the agonistic TNFR2 antibody or antigen-binding fragment thereof disclosed herein can be administered subcutaneously to a human subject. In some embodiments, any of the agonistic TNFR2 antibody or antigen-binding fragment thereof disclosed herein can be administered to a human subject at a low dose.

Cross-Reactivity of Anti-TNFSF Antibodies or Antigen-Binding Fragments Thereof with Soluble TNFRSF Member Proteins

Anti-TNFRSF antibodies or antigen-binding fragments thereof of the disclosure (e.g., antibodies or antigen-binding fragments thereof that bind a member of the TNFRSF family of proteins, such as TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, and DCR3) may have low cross-reactivity with the soluble form of the TNFRSF member protein. For example, a TNFR2 antibody or antigen-binding fragment thereof of the disclosure may have low cross-reactivity with soluble TNFR2. Such anti-TNFRSF antibodies or antigen-binding fragments thereof may have superior biological activity as compared with antibodies or antigen-binding fragments thereof that cross-react with the soluble form of the TNFRSF member protein.

It is known in the art that an agonistic antibody or antigen-binding fragment thereof that binds a TNFRSF member protein can activate the expression of the soluble form of the TNFRSF member protein. If such an agonistic antibody or antigen-binding fragment thereof cross-reacts with the soluble form of the TNFRSF member protein, the soluble form of the TNFRSF member protein can act as a decoy to neutralize the agonistic antibody or antigen-binding fragment thereof, leading to diminished agonist activity of the antibody or antigen-binding fragment thereof, and may require increased dosing amount and/or frequency of the antibody or antigen-binding fragment thereof to achieve a therapeutic effect in a subject.

In some embodiments, the modified antibody or antigen-binding fragment thereof has less than 90% (e.g., less than 80%, less than 70%, less than 60%, less than 50%, less than 40%, less than 30%, less than 20%, or less than 10%) cross-reactivity with the soluble TNFRSF member protein. In some embodiments, the modified antibody or antigen-binding fragment thereof has less than 50% cross-reactivity with the soluble TNFRSF member protein. The cross-reactivity of an antibody or antigen-binding fragment thereof with an antigen can be determined using methods that are known in the art, such as by assessing the percentage homology of an antigen sequence of a protein bound by an antibody with that the sequence of a second protein. A high percentage of homology indicates that it is highly likely that the antibody cross reacts with the second protein.

Due to the low cross-reactivity of the anti-TNFRSF antibodies or antigen-binding fragments thereof of the disclosure with soluble forms of TNFRSF member proteins, the anti-TNFRSF antibodies or antigen-binding fragments thereof of the disclosure may be used (e.g., administered to a human subject) at a low dose, e.g., a dose that is lower than a typical (or art recognized) dose level of a corresponding antibody or antigen-binding fragment thereof. In some embodiments, the antibody or antigen-binding fragment thereof is administered once every week at a dose of from about 0.8 mg/kg to about 1.2 mg/kg (e.g., about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, or about 1.2 mg/kg). In some embodiments, the antibody or antigen-binding fragment thereof is administered once every two weeks at a dose of from about 1.65 mg/kg to about 2.5 mg/kg (e.g., about 1.65 mg/kg, about 1.7 mg/kg, about 1.75 mg/kg, about 1.8 mg/kg, about 1.85 mg/kg, about 1.9 mg/kg, about 1.95 mg/kg, about 2.0 mg/kg, about 2.05 mg/kg, about 2.1 mg/kg, about 2.15 mg/kg, about 2.2 mg/kg, about 2.25 mg/kg, about 2.3 mg/kg, about 2.35 mg/kg, about 2.4 mg/kg, about 2.45 mg/kg, or about 2.5 mg/kg). In some embodiments, the antibody or antigen-binding fragment thereof is administered once every three weeks at a dose of from about 2.2 mg/kg to about 3.0 mg/kg (e.g., about 2.2 mg/kg, about 2.25 mg/kg, about 2.3 mg/kg, about 2.35 mg/kg, about 2.4 mg/kg, about 2.45 mg/kg, about 2.5 mg/kg, about 2.55 mg/kg, about 2.6 mg/kg, about 2.65 mg/kg, about 2.7 mg/kg, about 2.75 mg/kg, about 2.8 mg/kg, about 2.85 mg/kg, about 2.9 mg/kg, about 2.95 mg/kg, or about 3.0 mg/kg). In some embodiments, the antibody or antigen-binding fragment thereof is administered once every four weeks at a dose of from about 2.5 mg/kg to about 4.2 mg/kg (e.g., about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, or about 4.2 mg/kg).

### Anti-CD28 and Anti-ICOS Antibodies or Antigen-Binding Fragments Thereof

Antibodies or antigen-binding fragments thereof that specifically bind CD28 or ICOS are well known in the art. Each of the antibodies or antigen binding fragments described below can be made into a modified antibody or antigen-binding fragment thereof that includes a modified Fc domain, as described herein.

In some embodiments, the antibody or antigen-binding fragment may be any antibody or antigen-binding fragment that specifically binds CD28 disclosed in International Patent Application Publication Nos. WO 2020/127618, WO 2020/076853, WO 2016/185016, WO 2020/132066, WO 2018/177966, WO 2019/246514, WO 2010/007376, WO 2020/198009, WO 2021/259890, WO 2021/155380, WO 2020/127628, WO 2021/260064, WO 2015/198147, WO 2022/061098, WO 2011/101791, WO 2020/153605, WO 2006/050949, WO 2020/132024, WO 2016/146702, WO 2012/080351, WO 2019/240934, WO 2011/042891, WO 2022/253867, WO 2022/269019, WO 2023/143547, WO 01/88159, WO 2023/068382, WO 2020/210392, WO 2023/126445, WO 2023/215498, WO 2023/193239, WO 2022/170033, WO 2022/094299, WO 2023/215799, WO 2022/232392, WO 2023/180303, WO 02/051871, or WO 2023/031943; incorporated herein by reference.

In some embodiments, the anti-CD28 antibody or antigen-binding fragment thereof comprises one or more CDR sequences of theralizumab (TAB-08) with up to two conservative amino acid substitutions. In some embodiments, the anti-CD28 antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of theralizumab (TAB-08). Theralizumab (TAB-08) and other anti-CD28 antibodies are disclosed, e.g., in US 2006/0009382 and Beyersdorf et al. Ann. Rheum. Dis. 64 Suppl 4:iv91-95, 2005; incorporated herein by reference.

In some embodiments, the antibody or antigen-binding fragment may be any antibody or antigen-binding fragment that specifically binds ICOS disclosed in International Patent Application Publication Nos. WO 2016/120789, WO 2016/154177, WO 2018/029474, WO 2018/025221, WO 2019/122049, WO 2012/131004, WO 2020/102233, WO 2021/043961, WO 2019/229614, WO 2019/229613, WO 2019/171294, WO 2020/260326, WO 2020/031087, WO 2021/046293, WO 2022/254227, WO 2020/086476, WO 2022/098910, WO 2021/229032, or WO 2021/216417; incorporated herein by reference.

In some embodiments, the anti-ICOS antibody or antigen-binding fragment thereof comprises one or more CDR sequences of GSK-3359609 with up to two conservative amino acid substitutions. In some embodiments, the anti-ICOS antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of GSK-3359609. GSK-3359609 and other anti-ICOS antibodies are disclosed, e.g., in WO 2016/120789; incorporated herein by reference.

In some embodiments, GSK-3359609 comprises a CDR-H1 comprising the amino acid sequence of DYAMH (SEQ ID NO: 1525), a CDR-H2 comprising the amino acid sequence of LISIYSDHTNYNQKFQG (SEQ ID NO: 1526), a CDR-H3 comprising the amino acid sequence of NNYGNYGWYFDV (SEQ ID NO: 1527), a CDR-L1 comprising the amino acid sequence of SASSSVSYMH (SEQ ID NO: 1528), a CDR-L2 comprising the amino acid sequence of DTSKLAS (SEQ ID NO: 1529), and a CDR-L3 comprising the amino acid sequence of FQGSGYPYT (SEQ ID NO: 1530). In some embodiments, GSK-3359609 comprises a VH comprising the amino acid sequence of SEQ ID NO: 1531, a VL comprising the amino acid sequence of SEQ ID NO: 1532.

In some embodiments, the anti-ICOS antibody or antigen-binding fragment thereof comprises one or more CDR sequences of JTX-2011 with up to two conservative amino acid substitutions. In some embodiments, the anti-ICOS antibody or antigen-binding fragment thereof comprises a VH and/or a VL having amino an acid sequence that is at least 80% identical (e.g., 85%, 90%, 95%, 97%, 99%, or 100% identical) to the VH and/or VL sequence of JTX-2011. JTX-2011 and other anti-ICOS antibodies are disclosed, e.g., in WO 2016/154177; incorporated herein by reference.

In some embodiments, JTX-2011 comprises a CDR-H1 comprising the amino acid sequence of GFTFSDYWMD (SEQ ID NO: 1533), a CDR-H2 comprising the amino acid sequence of NIDEDGSITEYSPFVKG (SEQ ID NO: 1534), a CDR-H3 comprising the amino acid sequence of WGRFGFDS (SEQ ID NO: 1535), a CDR-L1 comprising the amino acid sequence of KSSQSLLSGSFNYLT (SEQ ID NO: 1536), a CDR-L2 comprising the amino acid sequence of YASTRHT (SEQ ID NO: 1537), and a CDR-L3 comprising the amino acid sequence of HHHYNAPPT (SEQ ID NO: 1538). In some embodiments, JTX-2011 comprises a VH comprising the amino acid sequence of SEQ ID NO: 1539, a VL comprising the amino acid sequence of SEQ ID NO: 1540.

### Amino Acid Modifications at the Fc Domain

Anti-TNFRSF, anti-TNFSF, anti-CD28, or anti-ICOS antibodies or antigen-binding fragments thereof of the disclosure contain at least 50 amino acid residues of an Fc domain. Anti-TNFRSF, anti-TNFSF, anti-CD28, or anti-ICOS antibodies or antigen-binding fragments thereof of the disclosure further contain one or more amino acid modifications (e.g., substitutions, deletions, insertions, or chemical modifications) at the Fc domain. Methods for introducing such amino acid modifications to a polypeptide (e.g., an anti-TNFRSF, anti-TNFSF, anti-CD28, or anti-ICOS antibody or antigen-binding fragment thereof of the disclosure, or the Fc domain thereof) are known in the art. It is also known to a person of ordinary skill in the art that amino acid modifications at the Fc domain generally do not affect the antigen binding properties (e.g., specificity, affinity, or avidity) of an antibody or antigen-binding fragment thereof.

### Human IgG Fc Domains

Humans have four IgG subtypes referred to as IgG1, IgG2, IgG3, and IgG4. Each of these subtypes corresponds to a distinct heavy chain constant region comprising an Fc domain. The amino acid and DNA sequences for these heavy chains constant regions are known in the art and can be accessed using the UniProt database. Exemplary amino acid sequence of human IgG1 heavy chain constant region can be accessed using UniProt Accession No. P01857. Exemplary amino acid sequence of human IgG2 heavy chain constant region can be accessed using UniProt Accession No. P01859. Exemplary amino acid sequence of human IgG3 heavy chain constant region can be accessed using UniProt Accession No. P01860. Exemplary amino acid sequence of human IgG4 heavy chain constant region can be accessed using UniProt Accession No. P01861. The anti-TNFRSF, anti-TNFSF, anti-CD28, or anti-ICOS antibodies or antigen-binding fragments thereof of the disclosure may have an IgG1, IgG2, IgG3, or IgG4 subtype. In some embodiments, the anti-TNFRSF, anti-TNFSF, anti-CD28, or anti-ICOS antibody or antigen-binding fragment thereof of the disclosure has an IgG4 subtype. In some embodiments, the anti-TNFRSF, anti-TNFSF, anti-CD28, or anti-ICOS antibody or antigen-binding fragment thereof of the disclosure is an IgG4 antibody or antigen-binding fragment thereof.

The Fc domain of human IgGs comprises a CH2 domain and a CH3 domain. The amino acid sequences of the CH2 and CH3 domains of human IgGs are relatively conserved across different IgG subtypes. Exemplary amino acid residues at each position of human IgG CH2 domains are shown in Table 15 below, and exemplary amino acid residues at each position of human IgG CH3 domains are shown in Table 16 below. It is known to a person of ordinary skill in the art that isoforms of these sequences exist, where one or more amino acid residue modifications (e.g., substitutions, deletions, or insertions) are present. Unless otherwise specified, the amino acid positions at the Fc domain are numbered according to the EU index throughout the disclosure.

TABLE 15
Amino acid residues at each position of human IgG CH2 domains.
CH2

| EU Number | Human IgG1 | Human IgG2 | Human IgG3 | Human IgG4 |
|---|---|---|---|---|
| 231 | A | A | A | A |
| 232 | P | P | P | P |
| 233 | E | P | E | E |
| 234 | L | V | L | F |
| 235 | L | A | L | L |
| 236 | G | | G | G |
| 237 | G | G | G | G |
| 238 | P | P | P | P |
| 239 | S | S | S | S |
| 240 | V | V | V | V |
| 241 | F | F | F | F |
| 242 | L | L | L | L |
| 243 | F | F | F | F |
| 244 | P | P | P | P |
| 245 | P | P | P | P |
| 246 | K | K | K | K |
| 247 | P | P | P | P |
| 248 | K | K | K | K |
| 249 | D | D | D | D |
| 250 | T | T | T | T |
| 251 | L | L | L | L |
| 252 | M | M | M | M |
| 253 | I | I | I | I |
| 254 | S | S | S | S |
| 255 | R | R | R | R |
| 256 | T | T | T | T |
| 257 | P | P | P | P |
| 258 | E | E | E | E |
| 259 | V | V | V | V |
| 260 | T | T | T | T |
| 261 | C | C | C | C |
| 262 | V | V | V | V |
| 263 | V | V | V | V |
| 264 | V | V | V | V |
| 265 | D | D | D | D |
| 266 | V | V | V | V |
| 267 | S | S | S | S |
| 268 | H | H | H | Q |
| 269 | E | E | E | E |
| 270 | D | D | D | D |
| 271 | P | P | P | P |
| 272 | E | E | E | E |
| 273 | V | V | V | V |
| 274 | K | Q | Q | Q |
| 275 | F | F | F | F |
| 276 | N | N | K | N |
| 277 | W | W | W | W |
| 278 | Y | Y | Y | Y |
| 279 | V | V | V | V |
| 280 | D | D | D | D |
| 282 | G | G | G | G |
| 282 | V | V | V | V |
| 283 | E | E | E | E |
| 284 | V | V | V | V |
| 285 | H | H | H | H |
| 286 | N | N | N | N |
| 287 | A | A | A | A |
| 288 | K | K | K | K |
| 289 | T | T | T | T |
| 290 | K | K | K | K |
| 291 | P | P | P | P |
| 292 | R | R | R | R |
| 293 | E | E | E | E |
| 294 | E | E | E | E |
| 295 | Q | Q | Q | Q |
| 296 | Y | F | Y | F |
| 297 | N | N | N | N |
| 298 | S | S | S | S |
| 299 | T | T | T | T |
| 300 | Y | F | F | Y |
| 301 | R | R | R | R |
| 302 | V | V | V | V |
| 303 | V | V | V | V |
| 304 | S | S | S | S |
| 305 | V | V | V | V |
| 306 | L | L | L | L |
| 307 | T | T | T | T |
| 308 | V | V | V | V |
| 309 | L | V | L | L |
| 310 | H | H | H | H |
| 311 | Q | Q | Q | Q |
| 312 | D | D | D | D |
| 313 | W | W | W | W |
| 314 | L | L | L | L |
| 315 | N | N | N | N |
| 316 | G | G | G | G |
| 317 | K | K | K | K |
| 318 | E | E | E | E |
| 319 | Y | Y | Y | Y |
| 320 | K | K | K | K |
| 321 | C | C | C | C |
| 322 | K | K | K | K |
| 323 | V | V | V | V |
| 324 | S | S | S | S |
| 325 | N | N | N | N |
| 326 | K | K | K | K |
| 327 | A | G | A | G |
| 328 | L | L | L | L |
| 329 | P | P | P | P |
| 330 | A | A | A | S |
| 331 | P | P | P | S |
| 332 | I | I | I | I |
| 333 | E | E | E | E |
| 334 | K | K | K | K |
| 335 | T | T | T | T |
| 336 | I | I | I | I |
| 337 | S | S | S | S |
| 338 | K | K | K | K |
| 339 | A | T | T | A |
| 340 | K | K | K | K |

TABLE 16
Amino acid residues at each position of human IgG CH3 domains.
CH3

| EU number | Human IgG1 | Human IgG2 | Human IgG3 | Human IgG4 |
|---|---|---|---|---|
| 341 | G | G | G | G |
| 342 | Q | Q | Q | Q |
| 343 | P | P | P | P |
| 344 | R | R | R | R |
| 345 | E | E | E | E |
| 346 | P | P | P | P |
| 347 | Q | Q | Q | Q |
| 348 | V | V | V | V |
| 349 | Y | Y | Y | Y |
| 350 | T | T | T | T |
| 351 | L | L | L | L |
| 352 | P | P | P | P |
| 353 | P | P | P | P |
| 354 | S | S | S | S |
| 355 | R | R | R | Q |
| 356 | D | E | E | E |
| 357 | E | E | E | E |
| 358 | L | M | M | M |
| 359 | T | T | T | T |
| 360 | K | K | K | K |
| 361 | N | N | N | N |
| 362 | Q | Q | Q | Q |
| 363 | V | V | V | V |
| 364 | S | S | S | S |
| 365 | L | L | L | L |
| 366 | T | T | T | T |
| 367 | C | C | C | C |
| 368 | L | L | L | L |
| 369 | V | V | V | V |
| 370 | K | K | K | K |
| 371 | G | G | G | G |
| 372 | F | F | F | F |
| 373 | Y | Y | Y | Y |
| 374 | P | P | P | P |
| 375 | S | S | S | S |
| 376 | D | D | D | D |
| 377 | I | I | I | I |
| 378 | A | A | A | A |
| 379 | V | V | V | V |
| 380 | E | E | E | E |
| 381 | W | W | W | W |
| 382 | E | E | E | E |
| 383 | S | S | S | S |
| 384 | N | N | S | N |
| 385 | G | G | G | G |
| 386 | Q | Q | Q | Q |
| 387 | P | P | P | P |
| 388 | E | E | E | E |
| 389 | N | N | N | N |
| 390 | N | N | N | N |
| 391 | Y | Y | Y | Y |
| 392 | K | K | N | K |
| 393 | T | T | T | T |
| 394 | T | T | T | T |
| 395 | P | P | P | P |
| 396 | P | P | P | P |
| 397 | V | M | M | V |
| 398 | L | L | L | L |
| 399 | D | D | D | D |
| 400 | S | S | S | S |
| 401 | D | D | D | D |
| 402 | G | G | G | G |
| 403 | S | S | S | S |
| 404 | F | F | F | F |
| 405 | F | F | F | F |
| 406 | L | L | L | L |
| 407 | Y | Y | Y | Y |
| 408 | S | S | S | S |
| 409 | K | K | K | R |
| 410 | L | L | L | L |
| 411 | T | T | T | T |
| 412 | V | V | V | V |
| 413 | D | D | D | D |
| 414 | K | K | K | K |
| 415 | S | S | S | S |
| 416 | R | R | R | R |
| 417 | W | W | W | W |
| 418 | Q | Q | Q | Q |
| 419 | Q | Q | Q | E |
| 420 | G | G | G | G |
| 421 | N | N | N | N |
| 422 | V | V | I | V |
| 423 | F | F | F | F |
| 424 | S | S | S | S |
| 425 | C | C | C | C |
| 426 | S | S | S | S |
| 427 | V | V | V | V |
| 428 | M | M | M | M |
| 429 | H | H | H | H |
| 430 | E | E | E | E |
| 431 | A | A | A | A |
| 432 | L | L | L | L |
| 433 | H | H | H | H |
| 434 | N | N | N | N |
| 435 | H | H | R | H |
| 436 | Y | Y | F | Y |
| 437 | T | T | T | T |
| 438 | Q | Q | Q | Q |
| 439 | K | K | K | K |
| 440 | S | S | S | S |
| 441 | L | L | L | L |
| 442 | S | S | S | S |
| 443 | L | L | L | L |
| 444 | S | S | S | S |
| 445 | P | P | P | L |
| 446 | G | G | G | G |
| 447 | K | K | K | K |

### Human IgG Hinge Regions

The CH1 domain and CH2 domain of a human IgG are connected with a hinge region. The amino acid sequences of the hinge regions of human IgGs are known in the art and are described in, e.g., Kabat et al, Sequences of Proteins of Immunological Interest (National Institute of Health, Bethesda, Md. 1987), the disclosure of which is incorporated herein by reference. Exemplary amino acid residues at each position of human IgG hinge regions are shown in Table 17 below. It is known to a person of ordinary skill in the art that isoforms of these sequences exist, where one or more amino acid residue modifications (e.g., substitutions, deletions, or insertions) are present. Unless otherwise specified, the amino acid positions at the Fc domain are numbered according to the EU index throughout the disclosure.

TABLE 17
Amino acid residues at each position of human IgG hinge regions.
Hinge Region

| EU number | Human IgG1 | Human IgG2 | Human IgG3 | Human IgG4 |
|---|---|---|---|---|
| 216 | E | E | E | E |
| 217 | P | R | L | S |
| 218 | K | K | K | K |
| | | | T | Y |
| | | | P | G |
| 219 | S | C | L | |
| 220 | C | C | G | |
| 221 | D | | D | |
| 222 | K | V | T | |
| 223 | T | | T | |
| 224 | H | E | H | P |
| 225 | T | | T | P |
| 226 | C | C | C | C |
| 227 | P | P | P | P |
| 228 | P | P | R | S |
| 229 | C | C | C | C |
| 230 | P | P | P | P |

Amino Acid Modifications that Decrease or Eliminate Fc Function

It has long been thought that functional antibodies (e.g., agonistic or antagonistic antibodies, such as agonistic or antagonistic antibodies that specifically bind TNFRSF members, TNFSF members, CD28, or ICOS) are more potent when they have a functional Fc receptor region. This is because the Fc region can bind to adjacent cells and thus facilitate the recruitment of effector cells. This adjacent cell-to-cell crosslinking with the antibody as the bridge is often referred to as ADCC (antibody dependent cellular cytotoxicity). This cell-to-cell crosslinking increases the signal strength of the antibody through the antigen binding regions opposite to the Fc regions, on two different cells. Also, a functional antibody usually binds a single antigen with both antigen-binding arms. Antibody engineering is commonly performed to increase the function of the Fc region of the antibody, as this was commonly expected to increase cell-to-cell crosslinking and, thus, to enhance the potency of the antibody. There are known Fc mutations for the improvement of effector functions, such as those shown in Table 18 below. It has long been thought that:
(a) strong Fc receptor function is critical for the function of the antibody;
(b) the crosslinking mediated by an antibody that has been modified to improve Fc receptor effector function is between two cells; and
(c) an antibody, such as one modified to improve Fc receptor effector function usually binds a single antigen with both antigen-binding arms.

In contrast to this traditional thinking regarding Fc receptor effector function of antibodies, I discovered a different approach to enhancing the function of antibodies and antigen-binding fragments thereof. Functional antibodies and antigen-binding fragments thereof generated by the approaches described herein (e.g., agonistic or antagonistic antibodies, such as agonistic or antagonistic antibodies that specifically bind TNFRSF members, TNFSF members, CD28, or ICOS) are unique in that the removal of the Fc receptor region from the entire antibody (or modifications that reduce, block, or ablate Fc effector function) does not have an apparent effect that alters antigen-binding and does not destroy the agonistic or antagonistic functions of the antibodies or antigen-binding fragments thereof. Receptor mapping of these antibodies shows that these antibodies consistently bind to two adjacent antigens on the same cell. These antibodies also exhibit cell-surface-only crosslinking through what is believed to be hexagonal networks—where antibodies and antigens are all on the surface of a cell. The traits of an antibody or antigen-binding fragment thereof generated using my approach include:
(a) the removal of the Fc region of the antibody (or changes that reduce, block, or eliminate Fc region effector function) does not harm the agonist or antagonist function of the antibody or antigen binding fragment thereof;
(b) the antibody or antigen-binding fragment thereof crosslinks at the cell surface, thereby pulling together two adjacent receptors; and
(c) the antibody or antigen-binding fragment thereof only exhibits single cell crosslinking at the cell surface.

Therefore, I attempted to create potent functional antibodies by testing the following hypothesis: that ADCC function-enhanced cell-surface crosslinking antibodies would be the most potent, and that ADCC function-removed cell-surface crosslinking antibodies would be weaker. The results that I obtained and disclosed herein are surprising and paradoxical to what was known in the art for years. When I removed the Fc receptor function from cell-surface crosslinking only antibodies, I created antibodies with high potency not only at very low doses but also across very wide dose ranges (e.g., the antibodies were functional at very low doses and also at very high doses).

ADCC crosslinking antibodies and cell-surface crosslinking antibodies have been characterized by (and were often limited by bi-model dose response curves, resulting in a narrow dose range when used to treat patients. When I retained cell-surface crosslinking and removed ADCC function of antibodies, the resulting antibodies no longer exhibited bi-model dose response curves. This observation suggests, surprisingly, that retained or enhanced Fc receptor function is harmful for the function of an antibody. Therefore, the present disclosure teaches that expanded dose response curves for functional antibodies are best achieved by cell-surface crosslinking only antibodies with Fc receptor inactivation.

Antibodies of the disclosure (e.g., anti-TNFRSF, anti-TNFSF, anti-CD28, or anti-ICOS antibodies or antigen-binding fragments thereof of the disclosure) with amino acid modifications (e.g., substitutions) that decrease or eliminate (e.g., inactivate) Fc function can be administered at a higher dose, at a higher dosing frequency, or across a wider dose range relative to corresponding antibodies that do not have such amino acid modifications. In some embodiments, the anti-TNFRSF, anti-TNFSF, anti-CD28, or anti-ICOS antibodies or antigen-binding fragments thereof of the disclosure may comprise one or more amino acid modifications (e.g., substitutions, deletions, insertions, or chemical modifications) at the Fc domain that decrease or eliminate (e.g., inactivate) the function of the Fc domain. For example, the amino acid modifications may decrease or eliminate (e.g., inactivate) the binding affinity of the antibody or antigen-binding fragment thereof for an Fc receptor (e.g., FcγRI, FcγRII, or FcγRIII) and/or an effector function of the antibody or antigen-binding fragment thereof. The effector function may be one that is mediated by an Fc receptor, such as ADCC or ADCP. In some embodiments, the amino acid modifications may be located within the hinge region of the antibody or antigen-binding fragment thereof.

In some embodiments, the Fc domain is a human IgG1 Fc domain. In some embodiments, the one or more amino acid modifications are one or more amino acid substitutions selected from the group consisting of:
(i) L235E;
(ii) L234A and L235A;
(iii) L234A, L235A, and P329G
(iv) L234E, L235F, and P331S;
(v) D265A;
(vi) G237A;
(vii) E318A;
(viii) E233P
(ix) G236R and L328R;
(x) L234A, L235A, G237A, P238S, H268A, A330S, and P331S; and
(xi) N297G or N297Q,
in which the amino acid positions are numbered according to the EU index.

In some embodiments, the Fc domain is a human IgG2 Fc domain. In some embodiments, the one or more amino acid modifications are one or more amino acid substitutions selected from the group consisting of:
(i) D265A;
(ii) G237A;
(iii) E318A;
(iv) H268Q, V309L, A330S, and P331S;
(v) V234A, G237A, P238S, H268A, V309L, A330S, and P331S; and
(vi) N297G or N297Q,
in which the amino acid positions are numbered according to the EU index.

In some embodiments, the Fc domain is a human IgG3 Fc domain. In some embodiments, the one or more amino acid modifications are one or more amino acid substitutions selected from the group consisting of:
(i) L235E;
(ii) L234A and L235A;
(iii) L234A, L235A, and P329G
(iv) L234E, L235F, and P331S;
(v) D265A;
(vi) G237A;
(vii) E318A;
(viii E233P
(ix) G236R and L328R;
(x) L234A, L235A, G237A, P238S, H268A, A330S, and P331S; and
(xi) N297G or N297Q,
in which the amino acid positions are numbered according to the EU index.

In some embodiments, the Fc domain is a human IgG4 Fc domain. In some embodiments, the one or more amino acid modifications are one or more amino acid substitutions selected from the group consisting of:
(i) L235E;
(ii) D265A;
(iii) G237A;
(iv) E318A;
(v) E233P
(vi) G236R and L328R; and
(vii) N297G or N297Q,
in which the amino acid positions are numbered according to the EU index.

In some embodiments, the antibody or antigen-binding fragment thereof comprises a human IgG4 hinge region having the amino acid substitution S228P (S241P according to Kabat). In some embodiments, the one or more amino acid modifications are one or more amino acid substitutions selected from the group consisting of:
(i) L235E; and
(ii) F234A and L235A,
in which the amino acid positions are numbered according to the EU index.

In some embodiments, the antibody or antigen-binding fragment thereof is an IgG4 antibody or antigen-binding fragment thereof. In some embodiments, the modified antibody or antigen-binding fragment thereof comprises the amino acid substitution S228P.

In some embodiments, the one or more amino acid modifications are one or more amino acid deletions. In some embodiments, the one or more amino acid deletions are deletions of all amino acid residues of the Fc domain. In some embodiments, the antibody or antigen-binding fragment thereof lacks an Fc domain. The absence of an Fc domain leads to the loss of effector function mediated by ADCC, which is dependent on the Fc domain. However, the effector function due to cell surface crosslinking of the antibody or antigen-binding fragment thereof is not affected and is a basis for the enhanced function of antibodies or antigen-binding fragments thereof described herein that lack an Fc domain or Fc effector function. The resulting antibody or antigen-binding fragment thereof binds two adjacent antigens, a function that does not involve linking of two adjacent cells by mechanisms such as ADCC. In some embodiments, the antibody or antigen-binding fragment thereof is selected from the group consisting of a single-chain Fv molecule (scFv), a diabody, a triabody, an antibody-like protein scaffold, a Fv fragment, a Fab fragment, a F(ab′)₂ molecule, and a tandem scFv (taFv). A person of ordinary skill in the art would expect that antibodies or antigen-binding fragments thereof lacking an Fc domain, such as the ones described herein, retain antigen binding activity, because the binding domains of the antibodies or antigen-binding fragments are still intact.

Additional amino acid modifications that decrease or eliminate (e.g., inactivate) Fc function are known in the art and can be found, e.g., in Saunders. Front. Immunol. 10:1296, 2019; incorporated herein by reference.

In certain embodiments, the modified human IgG4 Fc domain (including the IgG4 Fc hinge region) comprising an amino acid substitution of a proline (P) for the serine(S) at amino acid position 228, according to the EU index. In certain embodiments, the modified human IgG4 Fc domain comprises an amino acid sequence having at least 80%, at least 85%, at least 90%, at least 95%, at least 96%, at least 97%, at least 98%, at least 99%, or 100% identity to the amino acid sequence of SEQ ID NO: 1547. The modified human IgG4 Fc domain can, for example, comprise the amino acid sequence of SEQ ID NO: 1547.

In certain embodiments, the modified human IgG4 Fc domain is an IgG4*01 allotype, an IgG4*02 allotype, an IgG4*03 allotype, an IgG4*05 allotype, or an IgG4*06 allotype.

In certain embodiments, the modified human IgG4 Fc domain comprises a CH2 domain according to the human IgG4 amino acid sequence provided in Table 15. In certain embodiments, the modified human IgG4 Fc domain comprises a CH3 region according to the human IgG4 amino acid sequence provided in Table 16. In certain embodiments, the modified human IgG4 Fc domain comprises a hinge region according to the human IgG4 amino acid sequence provided in Table 17. In certain embodiments, the modified human IgG4 Fc domain comprises a hinge region according to the human IgG4 amino acid sequence provided in Table 17, with the exception of an amino acid substitution of a proline (P) for a serine(S) at amino acid position 228, according to the EU index. In certain embodiments, the modified human IgG4 Fc domain results in little to no effector function, i.e., little to no antibody-dependent cellular cytotoxicity (ADCC) or antibody dependent cellular phagocytosis (ADCP).

IgG4 antibodies by their nature have less effector function than IgG1 or IgG2 antibodies. The effector function can, for example, be referred to as ADCC. IgG4 antibodies have a number of desirable traits naturally and other isotype antibodies can be modified to have these traits. IgG4 naturally, or other isotype antibodies can be modified to, have less Fc receptor function to prevent ADCC or modified to have less ability to bind complement, and, thus, kill the target cell. Also, antibodies can be modified to remove the Fc region of the antibody, thus creating Fab′2 fragments that still have both arms but no longer have any effector function. As outlined herein, mutations in the hinge of antibodies can result in different distances between the antibody arms that affects the stabilization of the cross linking for optimal agonism.

The modified IgG4 antibodies disclosed herein have the following advantageous properties. In certain embodiments, the modified IgG4 antibodies have enhanced agonism. Reference to enhanced agonism indicates the modified IgG4 antibody is less susceptible to inhibition at a higher dose compared to a corresponding antibody having greater effector function (e.g., ADCC) (e.g., an IgG1 or IgG2 isotype or an IgG4 isotype with one or more mutations increasing ADCC). In certain embodiments, the modified human IgG4 Fc domain on the modified IgG4 antibody decreases or inactivates an effector function of the modified IgG4 antibody as compared to a control antibody (e.g., a modified human IgG1 or IgG2 antibody which comprises an IgG1 or IgG2 Fc domain or an unmodified IgG4 antibody which comprises an IgG4 Fc domain). The effector function can, for example, be selected from antibody dependent cellular cytotoxicity (ADCC) or antibody-dependent cellular phagocytosis (ADCP). In certain embodiments, the modified human IgG4 Fc domain of the modified IgG4 antibody reduces complement binding function as compared to a control antibody (e.g., a modified human IgG1 or IgG2 antibody which comprises an IgG1 or IgG2 Fc domain or an unmodified IgG4 antibody which comprises an IgG4 Fc domain). In certain embodiments, the modified IgG4 antibody is capable of being administered to a subject at a higher dose range and/or frequency than a control antibody (e.g., a modified human IgG1 or IgG2 antibody which comprises an IgG1 or IgG2 Fc domain or an unmodified IgG4 antibody which comprises an IgG4 Fc domain).

In certain embodiments, the modified IgG4 antibody comprises one or more Fc substitutions further reducing or eliminating effector function (e.g., ADCC or ADCP) as compared to a corresponding antibody having an unmodified IgG4 Fc.

In certain embodiments, the modified IgG4 antibody is capable of maintaining or increasing effectiveness when administered to a subject at a higher dose range than a control antibody (e.g., a modified human IgG1 or IgG2 antibody which comprises an IgG1 or IgG2 Fc domain). Maintaining or increasing effectiveness at a higher dose can, for example, refer to maintaining or increasing the proliferation of Treg cells when administered to a subject or patient in need thereof. Maintaining or increasing the effectiveness at a higher dose can also be monitored by measuring an increase (e.g., a 1×, 2×, 3×, or 4× increase) of soluble TNFR2 (sTNFR2) in the blood. The sTNFR2 would optimally be increased to levels at or above 2 ng/ml and up to levels of 6 ng/ml. The effectiveness of the modified IgG4 antibody can be maintained or increased, for example, by subcutaneous (SQ) administration. An advantage of SQ administration over intravenous (IV) administration is that SQ administration allows the correct complex formation by the modified IgG4 antibody. SQ administration can be performed at intervals as short as 3 days and up to intervals of 4 weeks.

In certain embodiments, the modified IgG4 antibody has an enhanced binding efficacy over a longer duration compared with a control antibody (e.g., a modified human IgG1 or IgG2 antibody which comprises an IgG1 or IgG2 Fc domain).

In certain embodiments, the modified IgG4 antibody is further conjugated to a therapeutic agent. The therapeutic agent can, for example, be selected from the group consisting of a chemotherapy agent, an immunotherapy agent, and an agonist of TNFR2.

Amino Acid Modifications that Increase Fc Function

In some embodiments, the anti-TNFRSF, anti-TNFSF, anti-CD28, or anti-ICOS antibodies or antigen-binding fragments thereof of the disclosure do not comprise any of the amino acid modifications (e.g., substitutions, deletions, insertions, or chemical modifications) at the Fc domain that increase the function of the Fc domain. For example, the amino acid modifications may increase the binding affinity of the antibody or antigen-binding fragment thereof for an Fc receptor (e.g., FcγRI, FcγRII, or FcγRIII) and/or an effector function of the antibody or antigen-binding fragment thereof. The effector function may be one that is mediated by an Fc receptor, such as ADCC or ADCP. Exemplary amino acid modifications at the Fc domain that increase Fc function are shown in Table 18 below. Additional amino acid modifications that decrease Fc function are known in the art and can be found, e.g., in Saunders. Front. Immunol. 10:1296, 2019; incorporated herein by reference.

TABLE 18
Amino acid modifications at the Fc
domain that decrease Fc function.

| Amino acid modifications | Alias or abbreviated name |
|---|---|
| S298A, E333A, K334A | AAA |
| S239D, A330L, I332E | DLE |
| S239D, I332E | DE |
| G236A, S239D, A330L, I332E | GASDALIE |
| G236A | GA |
| G236A, S239D, I332E | DAE |
| L234Y, G236W, S298A | YWA |
| F243L, R292P, Y300L, V305I, P396L | Variant 18 |
| Biantennary glycan at N297 | |
| Afucosylated glycan at N297 | |
| R435H | His435 |
| N434A | A |
| M252Y, S254T, T256E | YTE |
| M428L, N434S | LS |
| T252L, T253S, T254F | LSF |
| E294 deletion, T307P, N434Y | C6A-66 |
| T256N, A378V, S383N, N434Y | C6A-78 |
| E294 deletion | Del |

### Dimeric Binding of Antibodies to TNFRSF Member Protein, CD28, or ICOS

The present disclosure is based on the surprising finding that anti-TNFRSF, anti-CD28, or anti-ICOS antibodies or antigen-binding fragments thereof with amino acid modifications at the Fc domain that decrease Fc function do not lose biological activity (e.g., TNFRSF, CD28, or ICOS agonistic or antagonistic activity) when administered at a high dose or across a wide dose range. This is in direct contrast to prior publications describing methods of enhancing the activity of therapeutic antibodies by enhancing their Fc function, such as effector functions mediated by the Fc domain (e.g., ADCC or ADCP).

Therapeutic antibodies have been observed to lose activity when used at a high concentration or dose (see, e.g., Mayes et al. Nat. Rev. Drug Discov. 17:509-527, 2018; incorporated herein by reference), which limits their dose range when administered to human patients. This phenomenon is caused by monomeric binding of the antibody to its target, where only one of the two antigen-binding arms of the antibody interact with the target. It is also a result of the suboptimal clustering of antibodies due to ADCC.

The anti-TNFRSF, anti-CD28, or anti-ICOS antibodies or antigen-binding fragments thereof of the disclosure comprise amino acid modifications at the Fc domain that decrease Fc function. The lack of Fc effector function (e.g., lack of ADCC) leads to the favorable hexagonal clustering pattern of the antibodies or antigen-binding fragments and dimeric binding of the antibodies or antigen-binding fragments with a TNFRSF member protein, CD28, or ICOS, where each arm of an antibody interacts with a TNFRSF member protein, CD28, or ICOS.

### Nucleic Acids and Expression Systems

Anti-TNFRSF, anti-TNFSF, anti-CD28, or anti-ICOS antibodies or antigen-binding fragments thereof of the disclosure can be prepared by any of a variety of established techniques. For instance, an anti-TNFRSF, anti-TNFSF, anti-CD28, or anti-ICOS antibody or antigen-binding fragment thereof of the disclosure can be prepared by recombinant expression of one or more immunoglobulin light and heavy chain genes in a host cell. For instance, to express an antibody recombinantly, a host cell can be transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N. Y., 1989), Current Protocols in Molecular Biology (Ausubel et al., eds., Greene Publishing Associates, 1989), and in U.S. Pat. No. 4,816,397; incorporated herein by reference.

### Vectors for Expressing Antibodies

Viral genomes provide a rich source of vectors that can be used for the efficient delivery of exogenous genes into the genome of a cell (e.g., a eukaryotic or prokaryotic cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments of the disclosure include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030); incorporated herein by reference.

### Polynucleotide Sequence Elements

To express the anti-TNFRSF, anti-TNFSF, anti-CD28, or anti-ICOS antibodies or antigen-binding fragments thereof of the disclosure, polynucleotides encoding partial or full-length light and heavy chains, or CDRs thereof, e.g., obtained as described above, can be inserted into expression vectors such that the genes are operatively linked to transcriptional and translational control sequences. The expression vector and expression control sequences are chosen to be compatible with the expression host cell used. Polynucleotides encoding, e.g., the light chain gene and the heavy chain of anti-TNFRSF, anti-TNFSF, anti-CD28, or anti-ICOS antibody or antigen-binding fragment thereof can be inserted into separate vectors, or, optionally, both polynucleotides can be incorporated into the same expression vector using established techniques described herein or known in the art.

In addition to polynucleotides encoding the heavy and light chains of an antibody (or a polynucleotide encoding a single-chain polypeptide or an antibody fragment, such as a scFv molecule), the recombinant expression vectors of the disclosure may carry regulatory sequences that control the expression of the antibody chain genes in a host cell. The design of the expression vector, including the selection of regulatory sequences, may depend on such factors as the choice of the host cell to be transformed or the level of expression of protein desired. For instance, suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. Nos. 5,168,062, 4,510,245, and 4,968,615.

In addition to the antibody chain or CDR genes and regulatory sequences, the recombinant expression vectors of the disclosure can carry additional sequences, such as sequences that regulate replication of the vector in host cells (e.g., origins of replication) and selectable marker genes. A selectable marker gene facilitates selection of host cells into which the vector has been introduced (see e.g., U.S. Pat. Nos. 4,399,216; 4,634,665; and 5,179,017). For example, typically the selectable marker gene confers resistance to cytotoxic drugs, such as G418, puromycin, blasticidin, hygromycin, or methotrexate, to a host cell into which the vector has been introduced. Suitable selectable marker genes include the dihydrofolate reductase (DHFR) gene (for use in DHFR-host cells with methotrexate selection/amplification) and the neo gene (for G418 selection). To express the light and heavy chains of an anti-TNFRSF, anti-TNFSF, anti-CD28, or anti-ICOS antibody or antigen-binding fragment thereof, the expression vector(s) containing polynucleotides encoding the heavy and light chains can be transfected into a host cell by standard techniques.

### Host Cells for Expressing Antibodies

It is possible to express the anti-TNFRSF, anti-TNFSF, anti-CD28, or anti-ICOS antibodies or antigen-binding fragments thereof of the disclosure in either prokaryotic or eukaryotic host cells. In some embodiments, expression of polypeptides (e.g., single-chain polypeptides, antibodies, or antigen-binding fragments thereof) is performed in eukaryotic cells, e.g., mammalian host cells, for optimal secretion of a properly folded and immunologically active antibody. Exemplary mammalian host cells for expressing the recombinant polypeptides (e.g., single-chain polypeptides, antibodies, or antigen-binding fragments thereof) of the disclosure include Chinese Hamster Ovary (CHO cells) (including DHFR-CHO cells, described in Urlaub and Chasin, Proc. Natl. Acad. Sci. USA 77:4216-4220, 1980, used with a DHFR selectable marker, e.g., as described in Kaufman and Sharp, Mol. Biol. 159:601-621, 1982, NSO myeloma cells, COS cells, 293 cells, and SP2/0 cells. Additional cell types that may be useful for the expression of single-chain polypeptides, antibodies, and fragments thereof include bacterial cells, such as BL-21 (DE3) E. coli cells, which can be transformed with vectors containing foreign DNA according to established protocols. Additional eukaryotic cells that may be useful for expression of polypeptides include yeast cells, such as auxotrophic strains of S. cerevisiae, which can be transformed and selectively grown in incomplete media according to established procedures known in the art. When recombinant expression vectors encoding antibody genes (e.g., genes encoding one or more CDRs, an antibody heavy chain, or an antibody light chain) are introduced into mammalian host cells, the antibodies are produced by culturing the host cells for a period of time sufficient to allow for expression of the antibody in the host cells or secretion of the antibody into the culture medium in which the host cells are grown.

Anti-TNFRSF, anti-TNFSF, anti-CD28, or anti-ICOS antibodies or antigen-binding fragments thereof can be recovered from the culture medium using standard protein purification methods. Host cells can also be used to produce portions of intact antibodies, such as Fab fragments or scFv molecules. The disclosure also includes methods in which the above procedure is varied according to established protocols known in the art. For example, it can be desirable to transfect a host cell with DNA encoding either the light chain or the heavy chain (but not both) of an anti-TNFRSF, anti-TNFSF, anti-CD28, or anti-ICOS antibody or antigen-binding fragment thereof of this disclosure in order to produce an antigen-binding fragment of the antibody.

Once an anti-TNFRSF, anti-TNFSF, anti-CD28, or anti-ICOS antibody or antigen-binding fragment thereof of the disclosure has been produced by recombinant expression, it can be purified by any method known in the art, such as a method useful for purification of an immunoglobulin molecule, for example, by chromatography (e.g., ion exchange, affinity, particularly by affinity for the corresponding TNFRSF member protein, the corresponding TNFSF member protein, CD28, or ICOS after Protein A or Protein G selection, and sizing column chromatography), centrifugation, differential solubility, or by any other standard technique for the purification of proteins. Further, the anti-TNFRSF, anti-TNFSF, anti-CD28, or anti-ICOS antibody or antigen-binding fragment thereof of the disclosure can be fused to heterologous polypeptide sequences described herein or otherwise known in the art to facilitate purification or to produce therapeutic conjugates.

Once isolated, an anti-TNFRSF, anti-TNFSF, anti-CD28, or anti-ICOS antibody or antigen-binding fragment thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a SUPERDEX™ 75 column (Pharmacia Biotech AB, Uppsala, Sweden).

### Antibody Conjugates

Prior to administration of the anti-TNFRSF, anti-TNFSF, anti-CD28, or anti-ICOS antibodies or antigen-binding fragments thereof of the disclosure to a mammalian subject (e.g., a human), it may be desirable to conjugate the polypeptide (e.g., single-chain polypeptide, antibody, or antigen-binding fragment thereof) to a second molecule, e g., to modulate the activity of the polypeptide in vivo. The anti-TNFRSF, anti-TNFSF, anti-CD28, or anti-ICOS antibodies or antigen-binding fragments thereof of the disclosure can be conjugated to other molecules at either the N-terminus or C-terminus of a light or heavy chain of the polypeptide using any one of a variety of established conjugation strategies that are well-known in the art. Examples of pairs of reactive functional groups that can be used to covalently tether an anti-TNFRSF, anti-TNFSF, anti-CD28, or anti-ICOS single-chain polypeptide, antibody, or fragment thereof to another molecule include, without limitation, thiol pairs, carboxylic acids and amino groups, ketones and amino groups, aldehydes and amino groups, thiols and alpha, beta-unsaturated moieties (such as maleimides or dehydroalanine), thiols and alpha-halo amides, carboxylic acids and hydrazides, aldehydes and hydrazides, and ketones and hydrazides.

The anti-TNFRSF, anti-TNFSF, anti-CD28, or anti-ICOS antibodies or antigen-binding fragments thereof of the disclosure can be covalently appended directly to another molecule by chemical conjugation as described. Alternatively, fusion proteins containing anti-TNFRSF, anti-TNFSF, anti-CD28, or anti-ICOS single-chain polypeptides, antibodies, and fragments thereof of the disclosure can be expressed recombinantly from a cell (e.g., a eukaryotic cell or prokaryotic cell). This can be accomplished, for example, by incorporating a polynucleotide encoding the fusion protein into the nuclear genome of a cell (e.g., using techniques described herein or known in the art). Optionally, single-chain polypeptides, antibodies, and fragments thereof of the disclosure (e.g., anti-TNFRSF, anti-TNFSF, anti-CD28, or anti-ICOS antibodies or antigen-binding fragments thereof of the disclosure) can be joined to a second molecule by forming a covalent bond between the antibody and a linker. This linker can then be subsequently conjugated to another molecule, or the linker can be conjugated to another molecule prior to ligation to the anti-TNFRSF, anti-TNFSF, anti-CD28, or anti-ICOS single-chain polypeptide, antibody, or fragment thereof of the disclosure. Examples of linkers that can be used for the formation of a conjugate include polypeptide linkers, such as those that contain naturally occurring or non-naturally occurring amino acids. In some cases, it may be desirable to include D-amino acids in the linker, as these residues are not present in naturally occurring proteins and are thus more resistant to degradation by endogenous proteases. Fusion proteins containing polypeptide linkers can be made using chemical synthesis techniques, such as those described herein, or through recombinant expression of a polynucleotide encoding the fusion protein in a cell (e.g., a prokaryotic or eukaryotic cell). Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

### Drug-Antibody Conjugates

The anti-TNFRSF, anti-TNFSF, anti-CD28, or anti-ICOS antibodies or antigen-binding fragments thereof of the disclosure can additionally be conjugated to, admixed with, or administered separately from a therapeutic agent, such as a cytotoxic molecule. Conjugates of the disclosure may be applicable to the treatment or prevention of a disease associated with aberrant cell proliferation, such as a cancer described herein. Exemplary cytotoxic agents that can be conjugated to, admixed with, or administered separately from an anti-TNFRSF, anti-TNFSF, anti-CD28, or anti-ICOS antibody or antigen-binding fragment thereof include, without limitation, antineoplastic agents such as: acivicin; aclarubicin; acodazole hydrochloride; acronine; adozelesin; adriamycin; aldesleukin; altretamine; ambomycin; a. metantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; camptothecin; caracemide; carbetimer; carboplatin; carmustine; carubicin hydrochloride; carzelesin; cedefingol; chlorambucil; cirolemycin; cisplatin; cladribine; combretestatin a-4; crisnatol mesylate; cyclophosphamide; cytarabine; dacarbazine; daca (n-[2-(dimethyl-amino) ethyl] acridine-4-carboxamide); dactinomycin; daunorubicin hydrochloride; daunomycin; decitabine; dexormaplatin; dezaguanine; dezaguanine mesylate; diaziquone; docetaxel; dolasatins; doxorubicin; doxorubicin hydrochloride; droloxifene; droloxifene citrate; dromostanolone propionate; duazomycin; edatrexate; eflornithine hydrochloride; ellipticine; elsamitrucin; enloplatin; enpromate; epipropidine; epirubicin hydrochloride; erbulozole; esorubicin hydrochloride; estramustine; estramustine phosphate sodium; etanidazole; ethiodized oil i 131; etoposide; etoposide phosphate; etoprine; fadrozole hydrochloride; fazarabine; fenretinide; floxuridine; fludarabine phosphate; fluorouracil; 5-fdump; flurocitabine; fosquidone; fostriecin sodium; gemcitabine; gemcitabine hydrochloride; gold au 198; homocamptothecin; hydroxyurea; idarubicin hydrochloride; ifosfamide; ilmofosine; interferon alfa-2a; interferon alfa-2b; interferon alfa-nl; interferon alfa-n3; interferon beta-i a; interferon gamma-i b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peploycinsulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; rhizoxin; rhizoxin d; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; strontium chloride sr 89; sulofenur; talisomycin; taxane; taxoid; tecogalan sodium; tegafur; teloxantrone hydrochloride; temoporfin; teniposide; teroxirone; testolactone; thiamiprine; thioguanine; thiotepa; thymitaq; tiazofurin; tirapazamine; tomudex; top53; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine; vinblastine sulfate; vincristine; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 2-chlorodeoxyadenosine; 2′ deoxyformycin; 9-aminocamptothecin; raltitrexed; N-propargyl-5,8-dideazafolic acid; 2chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlor ethamine); cyclophosphamide; melphalan; chlorambucil; ifosfamide; busulfan; N-methyl-N-nitrosourea (MNU); N,N′-Bis (2-chloroethyl)-N-nitrosourea (BCNU); N-(2-chloroethyl)-N′ cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nitrosourea (fotemustine); streptozotocin; diacarbazine (DTIC); mitozolomide; temozolomide; thiotepa; mitomycin C; AZQ; adozelesin; cisplatin; carboplatin; ormaplatin; oxaliplatin; C1-973; DWA 2114R; JM216; JM335; Bis (platinum); tomudex; azacitidine; cytarabine; gemcitabine; 6-mercaptopurine; 6-thioguanine; hypoxanthine; teniposide 9-amino camptothecin; topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-hydroxyphenyl) retinamide; 13-cis retinoic acid; 3-methyl TTNEB; 9-cis retinoic acid; fludarabine (2-F-ara-AMP); or 2-chlorodeoxyadenosine (2-Cda).

Other therapeutic compounds that can be conjugated to, admixed with, or administered separately from an anti-TNFRSF, anti-TNFSF, anti-CD28, or anti-ICOS single-chain polypeptide, antibody, or antigen-binding fragment thereof of the disclosure in order to treat, prevent, or study the progression of a disease associated with aberrant cell proliferation include, but are not limited to, cytotoxic agents such as 20-pi-1,25 dihydroxyvitamin D3; 5-ethynyluracil; abiraterone; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatyrosine; baccatin Ill derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones (A, R=H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; rnerbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single-chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

### Labeled Antibodies

Anti-TNFRSF, anti-TNFSF, anti-CD28, or anti-ICOS single-chain polypeptides, antibodies, or antigen-binding fragments thereof of the disclosure may be conjugated to another molecule (e.g., an epitope tag) for the purpose of purification or detection. Examples of such molecules that are useful in protein purification include those that present structural epitopes capable of being recognized by a second molecule. This is a common strategy that is employed in protein purification by affinity chromatography, in which a molecule is immobilized on a solid support and exposed to a heterogeneous mixture containing a target protein conjugated to a molecule capable of binding the immobilized compound. Examples of epitope tag molecules that can be conjugated to the anti-TNFRSF, anti-TNFSF, anti-CD28, or anti-ICOS single-chain polypeptides, antibodies, or fragments thereof for the purposes of molecular recognition include, without limitation, maltose-binding protein, glutathione-S-transferase, a poly-histidine tag, a FLAG-tag, a myc-tag, human influenza hemagglutinin (HA) tag, biotin, streptavidin. Conjugates containing the epitopes presented by these molecules are capable of being recognized by such complementary molecules as maltose, glutathione, a nickel-containing complex, an anti-FLAG antibody, an anti-myc antibody, an anti-HA antibody, streptavidin, or biotin, respectively. For example, one can purify an anti-TNFRSF, anti-TNFSF, anti-CD28, or anti-ICOS single-chain polypeptide, antibody, or fragment thereof of the disclosure that has been conjugated to an epitope tag from a complex mixture of other proteins and biomolecules (e.g., DNA, RNA, carbohydrates, phospholipids, etc.) by treating the mixture with a solid-phase resin containing an complementary molecule that can selectively recognize and bind the epitope tag of the anti-TNFRSF, anti-TNFSF, anti-CD28, or anti-ICOS antibody or antigen-binding fragment thereof. Examples of solid-phase resins include agarose beads, which are compatible with purifications in aqueous solution.

An anti-TNFRSF, anti-TNFSF, anti-CD28, or anti-ICOS antibody or antigen-binding fragment thereof of the disclosure can also be covalently appended to a fluorescent molecule, e.g., to detect the antibody or antigen-binding fragment thereof by fluorimetry and/or by direct visualization using fluorescence microscopy. Exemplary fluorescent molecules that can be conjugated to polypeptides of the disclosure include green fluorescent protein, cyan fluorescent protein, yellow fluorescent protein, red fluorescent protein, phycoerythrin, allophycocyanin, Hoechst, 4′,6-diamidino-2-phenylindole (DAPI), propidium iodide, fluorescein, coumarin, rhodamine, tetramethylrhodamine, and cyanine. Additional examples of fluorescent molecules suitable for conjugation to polypeptides of the disclosure are well-known in the art and have been described in detail in, e.g., U.S. Pat. Nos. 7,417,131 and 7,413,874, each of which is incorporated by reference herein.

Anti-TNFRSF, anti-TNFSF, anti-CD28, or anti-ICOS antibodies or antigen-binding fragments thereof of the disclosure containing a fluorescent molecule are particularly useful for monitoring the cell-surface localization properties of the antibodies or antigen-binding fragments. For instance, one can expose cultured mammalian cells (e.g., T-reg cells, T cells, B cells, monocytes, neutrophils, platelets, granulocytes, bone marrow-derived lymphoid cells, and parenchymal cells) to the anti-TNFRSF, anti-TNFSF, anti-CD28, or anti-ICOS single-chain polypeptides, antibodies, or fragments thereof of the disclosure that have been covalently conjugated to a fluorescent molecule and subsequently analyze these cells using conventional fluorescent microscopy techniques known in the art. Confocal fluorescent microscopy is a particularly powerful method for determining cell-surface localization of the anti-TNFRSF, anti-TNFSF, anti-CD28, or anti-ICOS single-chain polypeptides, antibodies, or fragments thereof, as individual planes of a cell can be analyzed in order to distinguish antibodies or fragments thereof that have been internalized into a cell's interior, e.g., by receptor-mediated endocytosis, from those that are bound to the external face of the cell membrane. Additionally, cells can be treated with anti-TNFRSF, anti-TNFSF, anti-CD28, or anti-ICOS antibodies or antigen-binding fragments thereof of conjugated to a fluorescent molecule that emits visible light of a particular wavelength (e.g., fluorescein, which fluoresces at about 535 nm) and an additional fluorescent molecule that is known to localize to a particular site on the T-reg cell surface and that fluoresces at a different wavelength (e.g., a molecule that localizes to CD25 and that fluoresces at about 599 nm). The resulting emission patterns can be visualized by confocal fluorescence microscopy and the images from these two wavelengths can be merged in order to reveal information regarding the location of the anti-TNFRSF, anti-TNFSF, anti-CD28, or anti-ICOS single-chain polypeptide, antibody, or antigen-binding fragment thereof on the T-reg cell surface with respect to other receptors.

Bioluminescent proteins can also be incorporated into a fusion protein for the purposes of detection and visualization of an Anti-TNFRSF, anti-TNFSF, anti-CD28, or anti-ICOS antibody or antigen-binding fragment thereof of the disclosure. Bioluminescent proteins, such as Luciferase and aequorin, emit light as part of a chemical reaction with a substrate (e.g., luciferin and coelenterazine). Exemplary bioluminescent proteins suitable for use as a diagnostic sequence and methods for their use are described in, e.g., U.S. Pat. Nos. 5,292,658; 5,670,356; 6,171,809; and 7,183,092, each of which is herein incorporated by reference. Anti-TNFRSF, anti-TNFSF, anti-CD28, or anti-ICOS single-chain polypeptides, antibodies, or fragments thereof labeled with bioluminescent proteins are a useful tool for the detection of antibodies of the disclosure following an in vitro assay. For instance, the presence of an anti-TNFRSF, anti-TNFSF, anti-CD28, or anti-ICOS antibody or antigen-binding fragment thereof of the disclosure that has been conjugated to a bioluminescent protein can be detected among a complex mixture of additional proteins by separating the components of the mixture using gel electrophoresis methods known in the art (e.g., native gel analysis) and subsequently transferring the separated proteins to a membrane to perform a Western blot. Detection of the anti-TNFRSF, anti-TNFSF, anti-CD28, or anti-ICOS antibody or antigen-binding fragment thereof of the disclosure among the mixture of other proteins can be achieved by treating the membrane with an appropriate Luciferase substrate and subsequently visualizing the mixture of proteins on film using established protocols.

The anti-TNFRSF, anti-TNFSF, anti-CD28, or anti-ICOS antibodies or antigen-binding fragments thereof of the disclosure can also be conjugated to a molecule comprising a radioactive nucleus, such that an antibody or fragment thereof of the disclosure can be detected by analyzing the radioactive emission pattern of the nucleus. Alternatively, an anti-TNFRSF, anti-TNFSF, anti-CD28, or anti-ICOS antibody or antigen-binding fragment thereof of the disclosure can be modified directly by incorporating a radioactive nucleus within the antibody during the preparation of the protein. Radioactive isotopes of methionine (³⁵S), nitrogen (¹⁵N), or carbon (¹³C) can be incorporated into antibodies or fragments thereof of the disclosure by, e.g., culturing bacteria in media that has been supplemented with nutrients containing these isotopes. Optionally, tyrosine derivatives containing a radioactive halogen can be incorporated into an anti-TNFRSF, anti-TNFSF, anti-CD28, or anti-ICOS antibody or antigen-binding fragment thereof of the disclosure by, e.g., culturing bacterial cells in media supplemented with radiolabeled tyrosine. It has been shown that tyrosine functionalized with a radioactive halogen at the C2 position of the phenol system are rapidly incorporated into elongating polypeptide chains using the endogenous translation enzymes in vivo (U.S. Pat. No. 4,925,651; incorporated herein by reference). Halogens include fluorine, chlorine, bromine, iodine, and astatine. Additionally, anti-TNFRSF, anti-TNFSF, anti-CD28, or anti-ICOS antibodies or antigen-binding fragments thereof of the disclosure can be modified following isolation and purification from cell culture by functionalizing antibodies or fragments thereof of the disclosure with a radioactive isotope. The halogens represent a class of isotopes that can be readily incorporated into a purified protein by aromatic substitution at tyrosine or tryptophan, e.g., via reaction of one or more of these residues with an electrophilic halogen species. Examples of radioactive halogen isotopes include ¹⁸F, ⁷⁵Br, ⁷⁷Br, ¹²²I, ¹²³I, ¹²⁴I, ¹²⁵I, ¹²⁹I, ¹³¹I, or ²¹¹At.

Another alternative strategy for the incorporation of a radioactive isotope is the covalent attachment of a chelating group to the anti-TNFRSF, anti-TNFSF, anti-CD28, or anti-ICOS antibodies or antigen-binding fragments thereof of the disclosure. Chelating groups can be covalently appended to an anti-TNFRSF, anti-TNFSF, anti-CD28, or anti-ICOS antibody or antigen-binding fragment thereof of the disclosure by attachment to a reactive functional group, such as a thiol, amino group, alcohol, or carboxylic acid. The chelating groups can then be modified to contain any of a variety of metallic radioisotopes, including, without limitation, such radioactive nuclides as ¹²⁵I, ⁶⁷Ga, ¹¹¹In, ⁹⁹Tc, ¹⁶⁹Yb, ¹⁸⁶Re, ¹²³I, ¹²⁴I, ¹²⁵I, ¹³¹I, ^{99m}Tc, ¹¹¹In, ⁶⁴Cu, ⁶⁷Cu, ¹⁸⁶Re, ¹⁸⁸Re, ¹⁷⁷Lu, ⁹⁰Y, ⁷⁷As, ⁷²As, ⁸⁶Y, ⁸⁹Zr, ²¹¹At, ²¹²Bi, ²¹³Bi, or ²²⁵Ac.

In some embodiments, it may be desirable to covalently conjugate the anti-TNFRSF, anti-TNFSF, anti-CD28, or anti-ICOS antibodies or antigen-binding fragments thereof of the disclosure with a chelating group capable of binding a metal ion from heavy elements or rare earth ions, such as Gd³⁺, Fe³⁺, Mn³⁺, or Cr²⁺. Conjugates containing chelating groups that are coordinated to such paramagnetic metals are useful as in magnetic resonance imaging (MRI) applications. Paramagnetic metals include, but are not limited to, chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III), and ytterbium (III). In this way, anti-TNFRSF, anti-TNFSF, anti-CD28, or anti-ICOS antibodies or antigen-binding fragments thereof of the disclosure can be detected by MRI spectroscopy. For instance, one can administer anti-TNFRSF, anti-TNFSF, anti-CD28, or anti-ICOS antibodies or antigen-binding fragments thereof of the disclosure conjugated to chelating groups bound to paramagnetic ions to a mammalian subject (e.g., a human subject) to monitor the distribution of the antibody following administration. This can be achieved by administration of the antibody to a subject by any of the administration routes described herein, such as intravenously, and subsequently analyzing the location of the administered antibody by recording an MRI of the subject according to established protocols.

Anti-TNFRSF, anti-TNFSF, anti-CD28, or anti-ICOS antibodies or antigen-binding fragments thereof of the disclosure can additionally be conjugated to other molecules for the purpose of improving the solubility and stability of the protein in aqueous solution. Examples of such molecules include polyethylene glycol (PEG), PSA, bovine serum albumin (BSA), and human serum albumin (HSA), among others. For instance, one can conjugate an anti-TNFRSF, anti-TNFSF, anti-CD28, or anti-ICOS antibody or antigen-binding fragment thereof of the disclosure to carbohydrate moieties to evade detection of the antibody or fragment thereof by the immune system of the subject receiving treatment. This process of hyperglycosylation reduces the immunogenicity of therapeutic proteins by sterically inhibiting the interaction of the protein with B-cell receptors in circulation. Alternatively, anti-TNFRSF, anti-TNFSF, anti-CD28, or anti-ICOS antibodies or antigen-binding fragments thereof of the disclosure can be conjugated to molecules that prevent clearance from human serum and improve the pharmacokinetic profile of antibodies of the disclosure. Exemplary molecules that can be conjugated to or inserted within anti-TNFRSF, anti-TNFSF, anti-CD28, or anti-ICOS antibodies or antigen-binding fragments thereof of the disclosure to attenuate clearance and improve the pharmacokinetic profile of these antibodies and fragments include salvage receptor binding epitopes. These epitopes are found within the Fc region of an IgG immunoglobulin and have been shown to bind Fc receptors and prolong antibody half-life in human serum. The insertion of salvage receptor binding epitopes into anti-TNFRSF, anti-TNFSF, anti-CD28, or anti-ICOS antibodies or antigen-binding fragments thereof of the disclosure can be achieved, e.g., as described in U.S. Pat. No. 5,739,277; incorporated herein by reference.

### Pharmaceutical Compositions

Therapeutic compositions containing an anti-TNFRSF, anti-TNFSF, anti-CD28, or anti-ICOS antibody or antigen-binding fragment thereof of the disclosure can be prepared using methods known in the art. For example, such compositions can be prepared using, e.g., physiologically acceptable carriers, excipients, or stabilizers (Remington's Pharmaceutical Sciences 16^{th} edition, Osol, A. Ed. (1980); incorporated herein by reference), and in a desired form, e.g., in the form of lyophilized formulations or aqueous solutions. The compositions can also be prepared to contain the active agent (e.g., an anti-TNFRSF, anti-TNFSF, anti-CD28, or anti-ICOS antibody or antigen-binding fragment thereof of the disclosure) at a desired concentration. For example, a pharmaceutical composition of the disclosure may contain at least 10% (e.g., 10%, 20%, 30%, 40%, 50%, 60%, 70%, 80%, 90%, 95%, 97%, 98%, 99%, 99.5%, 99.9%, or 100%) active agent by weight (w/w).

Additionally, an active agent (e.g., an anti-TNFRSF, anti-TNFSF, anti-CD28, or anti-ICOS antibody or antigen-binding fragment thereof of the disclosure) that can be incorporated into a pharmaceutical formulation can itself have a desired level of purity. For example, an antibody or antigen-binding fragment thereof of the disclosure may be characterized by a certain degree of purity after isolating the antibody from cell culture media or after chemical synthesis, e.g., of a single-chain antibody fragment (e.g., scFv) by established solid-phase peptide synthesis methods or native chemical ligation as described herein. An anti-TNFRSF, anti-TNFSF, anti-CD28, or anti-ICOS antibody or antigen-binding fragment thereof of the disclosure may be at least 10% pure prior to incorporating the antibody into a pharmaceutical composition (e.g., 10%, 20%, 30%, 40%, 50%, 60%, 70%, 80%, 90%, 95%, 97%, 98%, 99%, 99.5%, 99.9%, 99.99%, or 100% pure).

Pharmaceutical compositions of anti-TNFRSF, anti-TNFSF, anti-CD28, or anti-ICOS antibodies or antigen-binding fragments thereof of the disclosure of the disclosure can be prepared for storage as lyophilized formulations or aqueous solutions by mixing the antibody having the desired degree of purity with optional pharmaceutically acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences, 16^{th} edition (Osol, ed. 1980; incorporated herein by reference). Such additives must be nontoxic to the recipients at the dosages and concentrations employed.

### Compositions for Combination Therapy

Pharmaceutical compositions of the disclosure may optionally include more than one active agent. For instance, compositions of the disclosure may contain an anti-TNFRSF, anti-TNFSF, anti-CD28, or anti-ICOS antibody or antigen-binding fragment thereof of the disclosure conjugated to, admixed with, or administered separately from another pharmaceutically active molecule, e.g., a cytotoxic agent, an antibiotic, or a T-lymphocyte (e.g., a gene-edited T-lymphocyte for use in CAR-T therapy). For instance, an anti-TNFRSF, anti-TNFSF, anti-CD28, or anti-ICOS antibody or antigen-binding fragment thereof of the disclosure, or a therapeutic conjugate thereof (e.g., a drug-antibody conjugate described herein), may be admixed with one or more additional active agents that can be used to treat cancer or another cell proliferation disorder (e.g., neoplasm). Alternatively, pharmaceutical compositions of the disclosure may be formulated for co-administration or sequential administration with one or more additional active agents that can be used to treat cancer or other cell proliferation disorders. Examples of additional active agents that can be used to treat cancer and other cell proliferation disorders and that can be conjugated to, admixed with, or administered separately from an anti-TNFRSF, anti-TNFSF, anti-CD28, or anti-ICOS single-chain polypeptide, antibody, or antibody fragment of the disclosure include cytotoxic agents (e.g., those described herein), as well as antibodies that exhibit reactivity with a tumor antigen or a cell-surface protein that is overexpressed on the surface of a cancer cell. Exemplary antibodies that can be conjugated to, admixed with, or administered separately from anti-TNFRSF, anti-TNFSF, anti-CD28, or anti-ICOS antibodies or antigen-binding fragments thereof of the disclosure include, without limitation, Trastuzamb (HERCEPTIN®), Bevacizumab (AVASTIN®), Cetuximab (ERBITUX®), Panitumumab (VECTIBIX®), Ipilimumab (YERVOY®), Rituximab (RITUXAN® and MABTHERA®), Alemtuzumab (CAMPATH®), Ofatumumab (ARZERRA®), Gemtuzumab ozogamicin (MYLOTARG®), Brentuximab vedotin (ADCETRIS®), ⁹⁰Y-Ibritumomab Tiuxetan (ZEVALIN®), and ¹³¹I-Tositumomab (BEXXAR®), which are described in detail in Scott et al. Cancer Immun., 12:14-21, 2012; incorporated herein by reference.

Additional agents that can be conjugated to, admixed with, or administered separately from anti-TNFRSF, anti-TNFSF, anti-CD28, or anti-ICOS antibodies or antigen-binding fragments thereof of the disclosure of the disclosure include T-lymphocytes that exhibit reactivity with a specific antigen associated with a particular pathology. For instance, anti-TNFRSF, anti-TNFSF, anti-CD28, or anti-ICOS antibodies or antigen-binding fragments thereof of the disclosure of the disclosure can be formulated for administration with a T-cell that expresses a chimeric antigen receptor (CAR-T) in order to treat a cell proliferation disorder, such as a cancer described herein. Anti-TNFRSF, anti-TNFSF, anti-CD28, or anti-ICOS antibodies or antigen-binding fragments thereof of the disclosure can synergize with CAR-T therapy by preventing T-reg cells from deactivating T-lymphocytes that have been genetically modified so as to express tumor-reactive antigen receptors. In this way, CAR-T cells can be administered to a subject prior to, concurrently with, or after administration of an anti-TNFRSF, anti-TNFSF, anti-CD28, or anti-ICOS single-chain polypeptide, antibody, or antigen-binding fragment thereof in order to treat a mammalian subject (e.g., a human) suffering from a cell proliferation disorder, such as cancer.

CAR-T therapy is a particularly robust platform for targeting cancer cells in view of the ability to genetically engineer T-lymphocytes to express an antigen receptor specific to a tumor-associated antigen. For instance, identification of antigens overexpressed on the surfaces of tumors and other cancer cells can inform the design and discovery of chimeric T-cell receptors, which are often composed of cytoplasmic and transmembrane domains derived from a naturally occurring T-cell receptor operatively linked to an extracellular scFv fragment that specifically binds to a particular antigenic peptide. T-cells can be genetically modified in order to express an antigen receptor that specifically binds to a particular tumor antigen by any of a variety of genome editing techniques described herein or known in the art. Exemplary techniques for modifying a T-cell genome so as to incorporate a gene encoding a chimeric antigen receptor include the CRISPR/Cas, zinc finger nuclease, TALEN, ARCUS™ platforms described herein. Methods for the genetic engineering of CAR-T lymphocytes have been described, e.g., in WO 2014/127261, WO 2014/039523, WO 2014/099671, and WO 2012/079000; the disclosures of each of which are incorporated by reference herein.

CAR-T cells useful in the compositions and methods of the disclosure include those that have been genetically modified such that the cell does not express the endogenous T-cell receptor. For instance, a CAR-T cell may be modified by genome-editing techniques, such as those described herein, so as to suppress expression of the endogenous T-cell receptor in order to prevent graft-versus-host reactions in a subject receiving a CAR-T infusion. Additionally or alternatively, CAR-T cells can be genetically modified so as to reduce the expression of one or more endogenous MHC proteins. This is a particularly useful technique for the infusion of allogeneic T-lymphocytes, as recognition of foreign MHC proteins represents one mechanism that promotes allograft rejection. One of skill in the art can also modify a T-lymphocyte so as to suppress the expression of immune suppressor proteins, such as programmed cell death protein 1 (PD-1) and cytotoxic T-lymphocyte-associated protein 4 (CTLA-4). These proteins are cell surface receptors that, when activated, attenuate T-cell activation. Infusion of CAR-T cells that have been genetically modified so as to diminish the expression of one or more immunosuppressor proteins represents one strategy that can be used to prolong the T-lymphocyte-mediated cytotoxicity in vivo.

In addition to deleting specific genes, one can also modify CAR-T cells in order to express a T-cell receptor with a desired antigen specificity. For instance, one can genetically modify a T-lymphocyte in order to express a T-cell receptor that specifically binds to a tumor-associated antigen in order to target infused T-cells to cancerous cells. An exemplary T-cell receptor that may be expressed by a CAR-T cell is one that binds PD-L1, a cell surface protein that is often overexpressed on various tumor cells. As PD-L1 activates PD-1 on the surface of T-lymphocytes, targeting this tumor antigen with CAR-T therapy can synergize with the anti-TNFRSF, anti-TNFSF, anti-CD28, or anti-ICOS antibodies or antigen-binding fragments thereof of the disclosure in order to increase the duration of an immune response mediated by a T-lymphocyte in vivo. CAR-T cells can also be modified so as to express a T-cell receptor that specifically binds an antigen associated with one or more infectious diseases, such as an antigen derived from a viral protein, a bacterial cell, a fungus, or other parasitic organism.

Other pharmaceutical compositions of the disclosure include those that contain an anti-TNFRSF, anti-TNFSF, anti-CD28, or anti-ICOS antibody or antigen-binding fragment thereof of the disclosure, interferon alpha, and/or one or more antibiotics that can be administered to a subject (e.g., a human subject) suffering from an infectious disease. For instance, an anti-TNFRSF, anti-TNFSF, anti-CD28, or anti-ICOS antibody or antigen-binding fragment thereof of the disclosure can be conjugated to, admixed with, or administered separately from an antibiotic useful for treating one or more infectious diseases, such as amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, meropenem, cefadroxil, cefazolin, cefazlexin, cefaclor, cefoxitin, cefprozil, cefuroxime, cefdinir, cefditoren, cefoperazone, clindamycin, lincomycin, daptomycin, erythromycin, linezolid, torezolid, amoxicillin, ampicillin, bacitracin, ciprofloxacin, doxycycline, and tetracycline, among others.

### Immunotherapy Agents

An anti-TNFRSF, anti-TNFSF, anti-CD28, or anti-ICOS antibody or antigen-binding fragment thereof described herein may be admixed with, conjugated to, administered with, or administered separately from, an immunotherapy agent, for instance, for the treatment of a cancer or infectious disease, such as a cancer or infectious disease described herein. Exemplary immunotherapy agents useful in conjunction with the compositions and methods described herein include, without limitation, an anti-CTLA-4 agent, an anti-PD-1 agent, an anti-PD-L1 agent, an anti-PD-L2 agent, an anti-TNF-α cross-linking agent, an anti-TRAIL cross-linking agent, and an anti-TWEAKR agent, as well as, for example, agents directed toward the immunological targets described in Table 1 of Mahoney et al., Cancer Immunotherapy, 14:561-584, 2015, the disclosure of which is incorporated herein by reference in its entirety. For example, the immunotherapy agent may be an anti-CTLA-4 antibody or antigen-binding fragment thereof, such as ipilimumab and tremelimumab. The immunotherapy agent may be an anti-PD-1 antibody or antigen-binding fragment thereof, such as nivolumab, pembrolizumab, avelumab, durvalumab, and atezolizumab. The immunotherapy agent may be an anti-PD-L1 antibody or antigen-binding fragment thereof, such as atezolizumab or avelumab. As other examples, immunological target TL1A may be targeted with an anti-TL1A antibody; immunological target LIGHT may be targeted with an anti-LIGHT antibody; immunological target BTLA may be targeted with an anti-BTLA antibody; immunological target LAG3 may be targeted with an anti-LAG3 antibody; immunological target TIM3 may be targeted with an anti-TIM3 antibody; immunological target Singlecs may be targeted with an anti-Singlecs antibody; immunological target ICOS ligand may be targeted with an anti-ICOS ligand antibody; immunological target B7-H3 may be targeted with an anti-B7-H3 antibody; immunological target B7-H4 may be targeted with an anti-B7-H4 antibody; immunological target VISTA may be targeted with an anti-VISTA antibody; immunological target TMIGD2 may be targeted with an anti-TMIGD2 antibody; immunological target BTNL2 may be targeted with an anti-BTNL2 antibody; immunological target CD48 may be targeted with an anti-CD48 antibody; immunological target KIR may be targeted with an anti-KIR antibody; immunological target LIR may be targeted with an anti-LIR antibody; immunological target ILT may be targeted with an anti-ILT antibody; immunological target NKG2D may be targeted with an anti-NKG2D antibody; immunological target NKG2A may be targeted with an anti-NKG2A antibody; immunological target MICA may be targeted with an anti-MICA antibody; immunological target MICB may be targeted with an anti-MICB antibody; immunological target CD244 may be targeted with an anti-CD244 antibody; immunological target CSF1R may be targeted with an anti-CSF1R antibody; immunological target IDO may be targeted with an anti-IDO antibody; immunological target TGFβ may be targeted with an anti-TGFβ antibody; immunological target CD39 may be targeted with an anti-CD39 antibody; immunological target CD73 may be targeted with an anti-CD73 antibody; immunological target CXCR4 may be targeted with an anti-CXCR4 antibody; immunological target CXCL12 may be targeted with an anti-CXCL12 antibody; immunological target SIRPA may be targeted with an anti-SIRPA antibody; immunological target CD47 may be targeted with an anti-CD47 antibody; immunological target VEGF may be targeted with an anti-VEGF antibody; and immunological target neuropilin may be targeted with an anti-neuropilin antibody.

Immunotherapy agents that may be used in conjunction with the compositions and methods described herein include, for instance, an anti-TWEAK agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-LIGHT agent, an anti-DR4 agent, an anti-CR5 agent, an anti-CD95 agent, an anti-TRAIL agent, an anti-RANKL agent, an anti-BCMA agent, an anti-TACI agent, and an anti-BAFFR agent. For instance, the immunotherapy agent may be an anti-TWEAK antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-LIGHT antibody or antigen-binding fragment thereof, an anti-DR4 antibody or antigen-binding fragment thereof, an anti-CR5 antibody or antigen-binding fragment thereof, an anti-CD95 antibody or antigen-binding fragment thereof, an anti-TRAIL antibody or antigen-binding fragment thereof, an anti-RANKL antibody or antigen-binding fragment thereof, an anti-BCMA antibody or antigen-binding fragment thereof, an anti-TACI antibody or antigen-binding fragment thereof, or an anti-BAFFR antibody or antigen-binding fragment thereof.

In some embodiments, the immunotherapy agent is an anti-cell surface lymphocyte protein antibody or antigen-binding fragment thereof, such as an antibody or antigen-binding fragment thereof that binds one or more of CD1, CD2, CD3, CD4, CD5, CD6, CD7, CD8, CD9, CD10, CD11, CD12, CD13, CD14, CD15, CD16, CD17, CD18, CD19, CD20, CD21, CD22, CD23, CD24, CD25, CD26, CD28, CD29, CD31, CD32, CD33, CD34, CD35, CD36, CD37, CD38, CD39, CD41, CD42, CD43, CD44, CD45, CD46, CD47, CD48, CD49, CD50, CD51, CD52, CD53, CD54, CD55, CD56, CD57, CD58, CD59, CD60, CD61, CD62, CD63, CD64, CD65, CD66, CD67, CD68, CD69, CD70, CD71, CD72, CD73, CD74, CD75, CD76, CD77, CD78, CD79, CD80, CD81, CD82, CD83, CD84, CD85, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD108, CD109, CD110, CD111, CD112, CD113, CD114, CD115, CD116, CD117, CD118, CD119, CD120, CD121, CD122, CD123, CD124, CD125, CD126, CD127, CD128, CD129, CD130, CD131, CD132, CD133, CD134, CD135, CD136, CD138, CD139, CD140, CD141, CD142, CD143, CD144, CD145, CD146, CD147, CD148, CD149, CD150, CD151, CD152, CD153, CD154, CD155, CD156, CD157, CD158, CD159, CD160, CD161, CD162, CD163, CD164, CD165, CD166, CD167, CD168, CD169, CD170, CD171, CD172, CD173, CD174, CD175, CD176, CD177, CD178, CD179, CD180, CD181, CD182, CD183, CD184, CD185, CD186, CD187, CD188, CD189, CD190, CD191, CD192, CD193, CD194, CD195, CD196, CD197, CD198, CD199, CD200, CD201, CD202, CD203, CD204, CD205, CD206, CD207, CD208, CD209, CD210, CD211, CD212, CD213, CD214, CD215, CD216, CD217, CD218, CD219, CD220, CD221, CD222, CD223, CD224, CD225, CD226, CD227, CD228, CD229, CD230, CD231, CD232, CD233, CD234, CD235, CD236, CD237, CD238, CD239, CD240, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD285, CD286, CD287, CD288, CD289, CD290, CD291, CD292, CD293, CD294, CD295, CD296, CD297, CD298, CD299, CD300, CD301, CD302, CD303, CD304, CD305, CD306, CD307, CD308, CD309, CD310, CD311, CD312, CD313, CD314, CD315, CD316, CD317, CD318, CD319, and/or CD320.

In some embodiments, the immunotherapy agent is an agent (e.g., a polypeptide, antibody, antigen-binding fragment thereof, a single-chain polypeptide, or construct thereof) that binds a chemokine or lymphokine, such as a chemokine or lymphokine involved in tumor growth. For instance, exemplary immunotherapy agents that may be used in conjunction with the compositions and methods described herein include agents (e.g., polypeptides, antibodies, antigen-binding fragments thereof, single-chain polypeptides, and constructs thereof) that bind and inhibit the activity of one or more, or all, of CXCL1, CXCL2, CXCL3, CXCL8, CCL2 and CCL5. Exemplary chemokines involved in tumor growth and that may be targeted using an immunotherapy agent as described herein include those described, for instance, in Chow et al., Cancer Immunol. Res., 2:1125-1131, 2014, the disclosure of which is incorporated herein by reference. Exemplary immunotherapy agents that may be used in conjunction with the compositions and methods described herein additionally include agents (e.g., polypeptides, antibodies, antigen-binding fragments thereof, single-chain polypeptides, and constructs thereof) that bind and inhibit the activity of one or more, or all, of CCL3, CCL4, CCL8, and CCL22, which are described, for instance, in Balkwill, Nat. Rev. Cancer, 4:540-550, 2004, the disclosure of which is incorporated herein by reference.

Additional examples of immunotherapy agents that can be used in conjunction with the compositions and methods described herein include Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies such as Vorinostat.

Using the methods described herein, an anti-TNFRSF, anti-TNFSF, anti-CD28, or anti-ICOS antibody or antigen-binding fragment thereof described herein may be co-administered with (e.g., admixed with) or administered separately from an immunotherapy agent. For example, an anti-TNFRSF, anti-TNFSF, anti-CD28, or anti-ICOS antibody or antigen-binding fragment thereof described herein may be administered to a subject, such as a human subject suffering from a cancer or infectious disease, simultaneously or at different times. In some embodiments, the anti-TNFRSF, anti-TNFSF, anti-CD28, or anti-ICOS antibody or antigen-binding fragment thereof of the disclosure is administered to the subject prior to administration of an immunotherapy agent to the subject. Alternatively, the anti-TNFRSF, anti-TNFSF, anti-CD28, or anti-ICOS antibody or antigen-binding fragment thereof of the disclosure may be administered to the subject after an immunotherapy agent. For example, the anti-TNFRSF, anti-TNFSF, anti-CD28, or anti-ICOS antibody or antigen-binding fragment thereof of the disclosure may be administered to the subject after a failed immunotherapy treatment. A physician of skill in the art can monitor the efficacy of immunotherapy treatment to determine whether the therapy has successfully ameliorated the pathology being treated (such as a cancer or infectious disease, e.g., a cancer or infectious disease described herein) using methods described herein and known in the art.

For instance, a physician of skill in the art may monitor the quantity of cancer cells in a sample isolated from a subject (e.g., a blood sample or biopsy sample), such as a human subject, for instance, using flow cytometry or FACS analysis. Additionally, or alternatively, a physician of skill in the art can monitor the progression of a cancerous disease in a subject, for instance, by monitoring the size of one or more tumors in the subject, for example, by computed tomography (CT) scan, MRI, or X-ray analysis. A physician of skill in the art may monitor the progression of a cancer, such as a cancer described herein, by evaluating the quantity and/or concentration of tumor biomarkers in the subject, such as the quantity and/or concentration of cell surface-bound tumor associated antigens or secreted tumor antigens present in the blood of the subject as an indicator of tumor presence. A finding that the quantity of cancer cells, the size of a tumor, and/or the quantity or concentration of one or more tumor antigens present in the subject or in a sample isolated from the subject has not decreased, for instance, by a statistically significant amount following administration of the immunotherapy agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the immunotherapy treatment has failed to ameliorate the cancer. Based on this indication, a physician of skill in the art may administer an anti-TNFRSF, anti-TNFSF, anti-CD28, or anti-ICOS antibody or antigen-binding fragment thereof described herein. Similarly, a physician a physician of skill in the art may monitor the quantity of bacterial, fungal, or parasitic cells, or the quantity of viral particles in a sample isolated from a subject suffering from an infectious disease, such as an infectious disease described herein. Additionally, or alternatively, a physician of skill in the art may monitor the progression of an infectious disease by evaluating the symptoms of a subject suffering from such a pathology. For instance, a physician may monitor the subject by determining whether the frequency and/or severity of one or more symptoms of the infectious disease have stabilized (e.g., remained the same) or decreased following treatment with an immunotherapy agent. A finding that the quantity of bacterial, fungal, or parasitic cells or viral particles in a sample isolated from the subject and/or a finding that the frequency or severity of one or more symptoms of the infectious disease have not decreased, for instance, by a statistically significant amount following administration of the immunotherapy agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the immunotherapy treatment has failed to ameliorate the infectious disease. Based on this indication, a physician of skill in the art may administer an anti-TNFRSF, anti-TNFSF, anti-CD28, or anti-ICOS antibody or antigen-binding fragment thereof described herein.

### Chemotherapy Agents

An anti-TNFRSF, anti-TNFSF, anti-CD28, or anti-ICOS antibody or antigen-binding fragment thereof described herein may be admixed with, conjugated to, administered with, or administered separately from, a chemotherapy agent, for instance, for the treatment of a cancer or infectious disease, such as a cancer or infectious disease described herein. Exemplary chemotherapy agents useful in conjunction with the compositions and methods of the disclosure include, without limitation, Abiraterone Acetate, ABITREXATE® (Methotrexate), ABRAXANE® (Paclitaxel Albumin), ADRIAMYCIN®, bleomycin, vinblastine, and dacarbazine (ABVD), ADRIAMYCIN®, bleomycin, vincristine sulfate, and etoposide phosphate (ABVE), ADRIAMYCIN®, bleomycin, vincristine sulfate, etoposide phosphate, prednisone, and cyclophosphamide (ABVE-PC), doxorubicin and cyclophosphamide (AC), doxorubicin, cyclophosphamide, and paclitaxel or docetaxel (AC-T), ADCETRIS® (Brentuximab Vedotin), cytarabine, daunorubicin, and etoposide (ADE), ado-trastuzumab emtansine, ADRIAMYCIN® (doxorubicin hydrochloride), afatinib dimaleate, AFINITOR® (Everolimus), AKYNZEO® (netupitant and palonosetron hydrochloride), ALDARA® (imiquimod), aldesleukin, ALECENSA® (alectinib), alectinib, alemtuzumab, ALKERAN® for Injection (Melphalan Hydrochloride), ALKERAN® tablets (melphalan), ALIMTA® (pemetrexed disodium), ALOXI® (palonosetron hydrochloride), AMBOCHLORIN® (chlorambucil), AMBOCLORIN® (Chlorambucil), aminolevulinic acid, anastrozole, aprepitant, AREDIA® (pamidronate disodium), ARIMIDEX® (anastrozole), AROMASIN® (exemestane), ARRANON® (nelarabine), arsenic trioxide, ARZERRA® (ofatumumab), asparaginase Erwinia chrysanthemi, AVASTIN® (bevacizumab), axitinib, azacitidine, BEACOPP Becenum (carmustine), BELEODAQ® (Belinostat), belinostat, bendamustine hydrochloride, bleomycin, etoposide, and cisplatin (BEP), bevacizumab, bexarotene, BEXXAR® (tositumomab and iodine ¹³¹I tositumomab), bicalutamide, BiCNU (carmustine), bleomycin, blinatumomab, BLINCYTO® (blinatumomab), bortezomib, BOSULIF® (bosutinib), bosutinib, brentuximab vedotin, busulfan, BUSULFEX® (busulfan), cabazitaxel, cabozantinib-S-malate, CAF, CAMPATH® (alemtuzumab), CAMPTOSAR® (irinotecan hydrochloride), capecitabine, CAPOX, CARAC® (fluorouracil), carboplatin, CARBOPLATIN-TAXOL®, carfilzomib, CARMUBRIS® (carmustine), carmustine, carmustine implant, CASODEX® (bicalutamide), CEENU (lomustine), cisplatin, etoposide, and methotrexate (CEM), ceritinib, CERUBIDINE® (daunorubicin hydrochloride), CERVARIX® (recombinant HPV bivalent vaccine), cetuximab, chlorambucil, chlorambucil-prednisone, CHOP, cisplatin, CLAFEN® (cyclophosphamide), clofarabine, CLOFAREX® (clofarabine), CLOLAR® (Clofarabine), CMF, cobimetinib, cometriq (cabozantinib-S-malate), COPDAC, COPP, COPP-ABV, COSMEGEN® (dactinomycin), COTELLIC® (cobimetinib), crizotinib, CVP, cyclophosphamide, CYFOS® (ifosfamide), CYRAMZA® (ramucirumab), cytarabine, cytarabine liposome, CYTOSAR-U® (cytarabine), CYTOXAN® (cyclophosphamide), dabrafenib, dacarbazine, DACOGEN® (decitabine), dactinomycin, daratumumab, DARZALEX® (daratumumab), dasatinib, daunorubicin hydrochloride, decitabine, degarelix, denileukin diftitox, denosumab, DEPOCYT® (cytarabine liposome), dexamethasone, dexrazoxane hydrochloride, dinutuximab, docetaxel, DOXIL® (doxorubicin hydrochloride), doxorubicin hydrochloride, DOX-SL® (doxorubicin hydrochloride), DTIC-DOME® (dacarbazine), EFUDEX (fluorouracil), ELITEK® (rasburicase), ELLENCE® (epirubicin hydrochloride), elotuzumab, ELOXATIN® (oxaliplatin), eltrombopag olamine, EMEND® (aprepitant), EMPLICITI® (elotuzumab), enzalutamide, epirubicin hydrochloride, EPOCH, ERBITUX® (cetuximab), eribulin mesylate, ERIVEDGE® (vismodegib), erlotinib hydrochloride, ERWINAZE® (asparaginase Erwinia chrysanthemi), ETOPOPHOS® (etoposide phosphate), etoposide, etoposide phosphate, EVACET® (doxorubicin hydrochloride liposome), everolimus, EVISTA® (raloxifene hydrochloride), EVOMELA® (melphalan hydrochloride), exemestane, 5-FU (5-fluorouracil), FARESTON® (toremifene), FARYDAK® (panobinostat), FASLODEX® (fulvestrant), FEC, FEMARA® (letrozole), filgrastim, FLUDARA® (fludarabine phosphate), fludarabine phosphate, FLUOROPLEX® (fluorouracil), fluorouracil injection, flutamide, FOLEX® (methotrexate), FOLEX® PFS (methotrexate), FOLFIRI, FOLFIRI-bevacizumab, FOLFIRI-cetuximab, FOLFIRINOX, FOLFOX, FOLOTYN® (pralatrexate), FU-LV, fulvestrant, GARDASIL® (recombinant HPV quadrivalent vaccine), GARDASIL 9® (recombinant HPV nonavalent vaccine), GAZYVA® (obinutuzumab), gefitinib, gemcitabine hydrochloride, gemcitabine-cisplatin, gemcitabine-oxaliplatin, gemtuzumab ozogamicin, GEMZAR® (gemcitabine hydrochloride), GILOTRIF® (afatinib dimaleate), GLEEVEC® (imatinib mesylate), GLIADEL® (carmustine implant), GLIADEL® wafer (carmustine implant), glucarpidase, goserelin acetate, HALAVEN® (eribulin mesylate), HERCEPTIN® (trastuzumab), HPV bivalent vaccine, HYCAMTIN® (topotecan hydrochloride), Hyper-CVAD, IBRANCE (palbociclib), IBRITUMOMAB® tiuxetan, ibrutinib, ICE, ICLUSIG® (ponatinib hydrochloride), IDAMYCIN® (idarubicin hydrochloride), idarubicin hydrochloride, idelalisib, IFEX® (ifosfamide), ifosfamide, ifosfamidum, IL-2 (aldesleukin), imatinib mesylate, IMBRUVICA® (ibrutinib), ilmiquimod, IMLYGIC® (talimogene laherparepvec), INLYTA (axitinib), recombinant interferon alpha-2b, intron A, tositumomab, such as ¹³¹I tositumomab, ipilimumab, IRESSA® (gefitinib), irinotecan hydrochloride, ISTODAX® (romidepsin), ixabepilone, ixazomib citrate, IXEMPRA® (ixabepilone), JAKAFI® (ruxolitinib phosphate), JEVTANA® (cabazitaxel), KADCYLA® (ado-trastuzumab emtansine), KEOXIFENE® (raloxifene hydrochloride), KEPIVANCE® (palifermin), KEYTRUDA® (pembrolizumab), KYPROLIS® (carfilzomib), lanreotide acetate, lapatinib ditosylate, lenalidomide, lenvatinib mesylate, LENVIMA® (lenvatinib mesylate), letrozole, leucovorin calcium, leukeran (chlorambucil), leuprolide acetate, levulan (aminolevulinic acid), LINFOLIZIN® (chlorambucil), LIPODOX® (doxorubicin hydrochloride liposome), lomustine, LONSURF® (trifluridine and tipiracil hydrochloride), LUPRON® (leuprolide acetate), LYNPARZA® (olaparib), MARQIBO® (vincristine sulfate liposome), MATULANE® (procarbazine hydrochloride), mechlorethamine hydrochloride, megestrol acetate, MEKINIST® (trametinib), melphalan, melphalan hydrochloride, mercaptopurine, MESNEX® (mesna), METHAZOLASTONE® (temozolomide), methotrexate, methotrexate LPF, MEXATE® (methotrexate), MEXATE-AQ® (methotrexate), mitomycin C, mitoxantrone hydrochloride, MITOZYTREX® (mitomycin C), MOPP, MOZOBIL® (plerixafor), MUSTARGEN® (mechlorethamine hydrochloride), MUTAMYCIN® (mitomycin C), MYLERAN® (busulfan), MYLOSAR® (azacitidine), MYLOTARG® (gemtuzumab ozogamicin), nanoparticle paclitaxel, NAVELBINE® (vinorelbine tartrate), NECITUMUMAB, nelarabine, NEOSAR® (cyclophosphamide), netupitant and palonosetron hydrochloride, NEUPOGEN® (filgrastim), NEXAVAR® (sorafenib tosylate), NILOTINIB, NINLARO® (ixazomib citrate), nivolumab, NOLVADEX® (tamoxifen citrate), NPLATE® (romiplostim), obinutuzumab, ODOMZO® (sonidegib), OEPA, ofatumumab, OFF, olaparib, omacetaxine mepesuccinate, ONCASPAR® (pegaspargase), ondansetron hydrochloride, ONIVYDE® (irinotecan hydrochloride liposome), ONTAK® (denileukin diftitox), OPDIVO® (nivolumab), OPPA, osimertinib, oxaliplatin, paclitaxel, paclitaxel albumin-stabilized nanoparticle formulation, PAD, palbociclib, palifermin, palonosetron hydrochloride, palonosetron hydrochloride and netupitant, pamidronate disodium, panitumumab, panobinostat, PARAPLAT® (carboplatin), PARPLATIN® (carboplatin), pazopanib hydrochloride, PCV, pegaspargase, peginterferon alpha-2b, PEG-INTRON® (peginterferon alpha-2b), pembrolizumab, pemetrexed disodium, PERJETA® (pertuzumab), pertuzumab, PLATINOL® (cisplatin), PLATINOL-AQ® (cisplatin), plerixafor, pomalidomide, POMALYSTR (pomalidomide), ponatinib hydrochloride, PORTRAZZA® (necitumumab), pralatrexate, prednisone, procarbazine hydrochloride, PROLEUKIN® (aldesleukin), PROLIA® (denosumab), PROMACTA (eltrombopag olamine), PROVENGE® (sipuleucel-T), PURINETHOL® (mercaptopurine), PURIXAN® (mercaptopurine), ²²³Ra dichloride, raloxifene hydrochloride, ramucirumab, rasburicase, R—CHOP, R-CVP, recombinant human papillomavirus (HPV), recombinant interferon alpha-2b, regorafenib, R-EPOCH, REVLIMID® (lenalidomide), RHEUMATREX® (methotrexate), RITUXAN® (rituximab), rolapitant hydrochloride, romidepsin, romiplostim, rubidomycin (daunorubicin hydrochloride), ruxolitinib phosphate, SCLEROSOL® intrapleural aerosol (talc), siltuximab, sipuleucel-T, somatuline depot (lanreotide acetate), sonidegib, sorafenib tosylate, SPRYCEL® (dasatinib), STANFORD V, sterile talc powder (talc), STERITALC® (talc), STIVARGA® (regorafenib), sunitinib malate, SUTENT® (sunitinib malate), SYLATRON® (peginterferon alpha-2b), SYLVANT® (siltuximab), SYNOVIR® (thalidomide), SYNRIBO® (omacetaxine mepesuccinate), thioguanine, TAC, TAFINLAR® (dabrafenib), TAGRISSO® (osimertinib), talimogene laherparepvec, tamoxifen citrate, tarabine PFS (cytarabine), TARCEVA (erlotinib hydrochloride), TARGRETIN® (bexarotene), TASIGNA® (nilotinib), TAXOL® (paclitaxel), TAXOTERER (docetaxel), TEMODAR® (temozolomide), temsirolimus, thalidomide, THALOMID® (thalidomide), thioguanine, thiotepa, TOLAK® (topical fluorouracil), topotecan hydrochloride, toremifene, TORISEL® (temsirolimus), TOTECT® (dexrazoxane hydrochloride), TPF, trabectedin, trametinib, TREANDA® (bendamustine hydrochloride), trifluridine and tipiracil hydrochloride, TRISENOX® (arsenic trioxide), TYKERB® (lapatinib ditosylate), UNITUXIN® (dinutuximab), uridine triacetate, VAC, vandetanib, VAMP, VARUBI® (rolapitant hydrochloride), vectibix (panitumumab), VelP, VELBAN® (vinblastine sulfate), VELCADE® (bortezomib), VELSAR (vinblastine sulfate), VEMURAFENIB, VIADUR (leuprolide acetate), VIDAZA (azacitidine), vinblastine sulfate, VINCASAR® PFS (vincristine sulfate), vincristine sulfate, vinorelbine tartrate, VIP, vismodegib, VISTOGARD® (uridine triacetate), VORAXAZE® (glucarpidase), vorinostat, VOTRIENT® (pazopanib hydrochloride), WELLCOVORIN® (leucovorin calcium), XALKORI® (crizotinib), XELODA® (capecitabine), XELIRI, XELOX, XGEVA® (denosumab), XOFIGO® (²²³Ra dichloride), XTANDI® (enzalutamide), YERVOY® (ipilimumab), YONDELIS® (trabectedin), ZALTRAP® (ziv-aflibercept), ZARXIO® (filgrastim), ZELBORAF® (vemurafenib), ZEVALIN® (ibritumomab tiuxetan), ZINECARD® (dexrazoxane hydrochloride), ziv-aflibercept, ZOFRAN® (ondansetron hydrochloride), ZOLADEX® (gGoserelin acetate), zoledronic acid, ZOLINZA® (vorinostat), ZOMETA® (zoledronic acid), ZYDELIG® (idelalisib), ZYKADIA® (ceritinib), and ZYTIGA (abiraterone acetate).

### Methods of Treatment

A physician of ordinary skill in the art can readily determine an effective amount of an anti-TNFRSF, anti-TNFSF, anti-CD28, or anti-ICOS antibody or antigen-binding fragment thereof of the disclosure for administration to a mammalian subject (e.g., a human) in need thereof. For example, a physician could start prescribing doses of an anti-TNFRSF, anti-TNFSF, anti-CD28, or anti-ICOS antibody or antigen-binding fragment thereof of the disclosure at levels lower than that required to achieve the desired therapeutic effect and gradually increase the dosage until the desired effect is achieved. Examples of the desired effect, without limitation, includes alleviation of symptoms, diminishment of extent of disease, stabilized (i.e., not worsening) state of disease, delay or slowing of disease progression, amelioration or palliation of the disease state, and remission (whether partial or total). Alternatively, a physician may begin a treatment regimen by administering an anti-TNFRSF, anti-TNFSF, anti-CD28, or anti-ICOS antibody or antigen-binding fragment thereof of the disclosure at a high dose and subsequently administer progressively lower doses until a therapeutic effect is achieved (e.g., a reduction in the proliferation of a population of CD8+ T cells, CD4+ T cells, and/or B cells) or a decrease in the peripheral secretion of IFN-v). In general, a suitable daily dose of an anti-TNFRSF, anti-TNFSF, anti-CD28, or anti-ICOS antibody or antigen-binding fragment thereof of the disclosure will be an amount of the antibody which is the lowest dose effective to produce a therapeutic effect. An anti-TNFRSF, anti-TNFSF, anti-CD28, or anti-ICOS antibody or antigen-binding fragment thereof of the disclosure may be administered by injection, e.g., by intravenous, intramuscular, intraperitoneal, or subcutaneous injection, optionally proximal to the site of a target tissue. A daily dose of a therapeutic composition of an anti-TNFRSF, anti-TNFSF, anti-CD28, or anti-ICOS antibody or antigen-binding fragment thereof of the disclosure may be administered as a single dose or as two, three, four, five, six or more doses administered separately at appropriate intervals throughout the day, week, month, or year, optionally, in unit dosage forms. While it is possible for an anti-TNFRSF, anti-TNFSF, anti-CD28, or anti-ICOS antibody or antigen-binding fragment thereof of the disclosure to be administered alone, it may also be administered as a pharmaceutical formulation in combination with excipients, carriers, and optionally, additional therapeutic agents.

Table 19 below lists specific TNFRSF member proteins, the function of their natural ligand or agonists, the function of their antagonists, as well as diseases that may be treated by their agonists or antagonists (e.g., agonistic or antagonistic antibodies or antigen-binding fragment thereof that specifically bind the TNFRSF member protein). Additional diseases or disorders related to TNFRSF member proteins that may be treated by agonists or antagonists of TNFRSF member proteins can be found, e.g., in Dostert et al. Physiol. Rev. 99:115-160, 2019; incorporated herein by reference.

TABLE 19
TNFRSF member proteins and diseases that may be
treated by an antagonist or agonist thereof.

| | Effect on | | | |
|---|---|---|---|---|
| | receptor by a | Effect on | | |
| TNFRSF | natural ligand | receptor by an | Diseases that | Diseases that |
| member | or an agonist | antagonist | may be treated | may be treated |
| protein | agent | agent | by an antagonist | by an agonist |
| TRAIL-R1 | Death of cells | Suppresses | Autoimmunity | Cancer, infectious |
| (TNFRSF10A) | (e.g., tumor | apoptosis of, | | disease |
| | cells) | e.g., T-reg cells, | | |
| | | MDSCs | | |
| TRAIL-R2 | Death of cells | Suppresses | Autoimmunity | Cancer, infectious |
| (TNFRSF10B) | (e.g., tumor | apoptosis of, | | disease |
| | cells) | e.g., T-reg cells, | | |
| | | MDSCs | | |
| TRAMP | Stimulates T- | Suppresses | Cancer, infectious | Autoimmunity |
| | regs | apoptosis of, | diseases | |
| | | e.g., CD8+ | | |
| | | cytotoxic T cells | | |
| DR6 | Causes cell | Suppresses | Neuron-specific | Autoimmunity, |
| | death | apoptosis of, | diseases (e.g., | inflammation |
| | | e.g., CD8+ | Alzheimer's | |
| | | cytotoxic T cells, | disease), cancer, | |
| | | B cells, neural | infectious | |
| | | cells | diseases | |
| NGFR | Maintenance of | Suppresses | Cancer | Autoimmunity |
| | neurons | apoptosis of, | | |
| | | e.g., CD8+ | | |
| | | cytotoxic T cells, | | |
| | | B cells | | |
| TNFR1 | Increase | Suppresses | Autoimmunity, | Cancer |
| | inflammation | apoptosis of, | inflammation, | |
| | | e.g., T-reg cells, | transplant | |
| | | MDSCs | rejections, asthma | |
| Fas | Kills cells (e.g., | Suppresses | Obesity, diabetes | Cancer |
| | tumor cells) | apoptosis of, | (e.g., type 2 | |
| | | e.g., T-reg cells, | diabetes), | |
| | | MDSCs | hyperlipidemia | |
| EDAR | Developmental | — | — | — |
| | biology, e.g., | | | |
| | embryonic | | | |
| | signaling | | | |
| | pathway that | | | |
| | allows | | | |
| | development of | | | |
| | ectoderm | | | |
| | structures | | | |
| | including skin, | | | |
| | hair, nails, teeth, | | | |
| | and sweat | | | |
| | glands | | | |
| TRAIL-R3 | Decoy receptor | Inhibits | Cancer, infectious | Autoimmunity |
| | | proliferation of, | diseases | |
| | | e.g., T-reg cells, | | |
| | | tumor cells, | | |
| | | MDSCs | | |
| TRAIL-R4 | Incomplete | Inhibits | Cancer, infectious | Autoimmunity |
| | death domain | proliferation of, | diseases | |
| | | e.g., T-reg cells, | | |
| | | tumor cells, | | |
| | | MDSCs | | |
| RANK | Regulates | Inhibits | Osteoporosis, | Stroke |
| | osteoclast | proliferation of, | tumor metastatic | |
| | differentiation | e.g., osteoclast | disease, bone | |
| | | cells | loss | |
| FN14 | Stimulate brain | Inhibits | Glioblastoma, | Autoimmunity, |
| | tumors | proliferation of, | brain tumors | inflammation |
| | | e.g., T-reg cells, | | |
| | | tumor cells, | | |
| | | MDSCs | | |
| TNFR2 | Stimulation of T- | Inhibits | Cancer, infectious | Autoimmunity |
| | regs; death of T | proliferation of, | diseases | |
| | effectors | e.g., T-reg cells, | | |
| | | tumor cells, | | |
| | | MDSCs | | |
| LT beta | Lymphoid | Inhibits | Cancer (e.g., B | Autoimmunity, |
| receptor | development, | proliferation of, | cell lymphomas, | inflammation |
| | tumor growth | e.g., T-reg cells, | melanomas, liver | |
| | | cancer cells, | cancer, renal | |
| | | MDSCs | cancer, | |
| | | | fibrosarcomas) | |
| HVEM | Limits | Inhibits | Cancer, infectious | Autoimmunity, |
| | inflammation, | proliferation of, | diseases | inflammation |
| | TCR activation, | e.g., T-reg cells, | | |
| | B cells | cancer cells, | | |
| | | MDSCs | | |
| CD40 | Proliferates T | Inhibits | Autoimmunity, | Cancer, infectious |
| | effectors | proliferation of, | allergies, asthma | disease |
| | | e.g., CD8+ | | |
| | | cytotoxic T cells, | | |
| | | B cells, platelets, | | |
| | | macrophages, | | |
| | | dendritic cells | | |
| CD30 | Proliferation | Inhibits | Cancer (e.g., | Autoimmunity, |
| | | proliferation of, | Hodgkin | transplant |
| | | e.g., T-reg cells, | lymphoma, | rejection |
| | | cancer cells, | chronic Hodgkin | |
| | | MDSCs | lymphoma, | |
| | | | refractory Hodgkin | |
| | | | lymphoma, and | |
| | | | anaplastic large | |
| | | | cell lymphoma) | |
| CD27 | NFkB activation | Inhibits | Autoimmunity, | Cancer, infectious |
| | CD8 memory T | proliferation of, | allergies, asthma | disease |
| | cells | e.g., CD4+ and | | |
| | | CD8+ cytotoxic | | |
| | | T cells | | |
| 4-1BB | Activate CD8 T, | Inhibits | Autoimmunity, | Cancer, infectious |
| | NK cells | proliferation of, | asthma, transplant | disease |
| | increased | e.g., CD4+ and | rejection | |
| | cytokines, | CD8+ cytotoxic | | |
| | cytotoxicity | T cells, NK cells | | |
| OX40 | Activate CD8+ | Inhibits | Autoimmunity, | Cancer, infectious |
| | T, NK cells | proliferation of, | asthma, transplant | disease |
| | increased | e.g., CD4+ and | rejection | |
| | cytokines, | CD8+ cytotoxic | | |
| | cytotoxicity | T cells, B cells | | |
| GITR | Prevents cell | Inhibits | Transplant | Cancer |
| | growth | proliferation of, | rejection, | |
| | | e.g., CD8+ | autoimmunity, | |
| | | cytotoxic T cells, | asthma, allergies | |
| | | NK cells | | |
| BCMA | NK and B cell | Inhibits | Cancer for B cell | Infectious disease |
| (TNFRSF17) | activation | proliferation of, | tumors, e.g., | |
| | | e.g., T-reg cells, | multiple myeloma | |
| | | B cells, MDSCs | (e.g., refractory | |
| | | | multiple myeloma) | |
| TACI | NK and B cell | Inhibits | Multiple myeloma | Infectious disease |
| | activation | proliferation of, | (e.g., refractory | |
| | | e.g., T-reg cells, | multiple | |
| | | B cells, MDSCs | myeloma), breast | |
| | | | cancer | |
| BAFF-R | B cell | Inhibits | Cancer for B cell | Infectious disease |
| | development | proliferation of, | tumors, e.g., B | |
| | | e.g., T-reg cells, | cell acute | |
| | | B cells, MDSCs | lymphoblastic | |
| | | | leukemia, multiple | |
| | | | myeloma | |
| XEDAR | B cell | Inhibits | Allergies, | Cancer |
| | maintenance | proliferation of, | autoimmunity, | |
| | | e.g., CD8+ | transplant | |
| | | cytotoxic T cells, | rejection | |
| | | B cells | | |
| TROY | Cell | Inhibits | Cancer, e.g., | Regeneration of |
| | maintenance | proliferation of, | colon cancer and | the intestinal |
| | (e.g., in the | e.g., T-reg cells, | glioblastoma | epithelia |
| | colon) | cancer cells, | | |
| | | MDSCs | | |
| RELT | Cell death | Inhibits | Nerve/CNS | Autoimmune |
| (TNFRSF19L) | | proliferation of, | tumors, infectious | disease, |
| | | e.g., T-reg cells, | diseases | inflammation |
| | | MDSCs, cancer | | |
| | | cells | | |

### Methods of Treating Cell Proliferation Disorders

Anti-TNFRSF, anti-TNFSF, anti-CD28, or anti-ICOS antibodies or antigen-binding fragments thereof of the disclosure are useful therapeutics for the treatment of a wide array of cancers and cell proliferation disorders. Certain anti-TNFRSF, anti-TNFSF, anti-CD28, or anti-ICOS antibodies or antigen-binding fragments thereof of the disclosure can be administered to a mammalian subject, such as a human, suffering from a cell proliferation disorder, such as cancer, e.g., to enhance the effectiveness of the adaptive immune response against the target cancer cells.

In particular, anti-TNFRSF, anti-TNFSF, anti-CD28, or anti-ICOS antibodies or antigen-binding fragments thereof of the disclosure can be administered to a mammalian subject, such as a human, to reduce or inhibit T-reg cell growth and activation, which allows tumor-infiltrating T-lymphocytes to localize to cells presenting tumor-associated antigens and to promote cytotoxicity. In addition, anti-TNFRSF, anti-TNFSF, anti-CD28, or anti-ICOS antibodies or antigen-binding fragments thereof of the disclosure may synergize with existing adoptive T-cell therapy platforms, as one of the limitations on the effectiveness of this strategy has been the difficulty of prolonging cytotoxicity of tumor-reactive T-cells following infusion into a mammalian subject (e.g., a human). In some embodiments, the antibody or antigen-binding fragment thereof is an agonistic antibody or antigen-binding fragment thereof that specifically binds a TNFRSF member protein selected from the group consisting of TRAIL-R1 (TNFRSF10A), TRAIL-R2 (TNFRSF10B), TNFR1, Fas, CD40, CD27, 4-1BB, OX40, GITR, and XEDAR. In some embodiments, the antibody or antigen-binding fragment thereof is an antagonistic antibody or antigen-binding fragment thereof that specifically binds a TNFRSF member protein selected from the group consisting of TRAMP, NGFR, TRAIL-R4, TNFR2, HVEM, CD30, TROY, and RELT. In some embodiments, the modified antibody or antigen-binding fragment thereof is an antagonistic antibody or antigen-binding fragment thereof that specifically binds TNFR2 comprising a CDR-H1, a CDR-H2, a CDR-H3, a CDR-L1, a CDR-L2, and a CDR-L3 set forth in Table 4. In some embodiments, the modified antibody or antigen-binding fragment thereof is an antagonistic antibody or antigen-binding fragment thereof that specifically binds TNFR2 comprising a VH and a VL having amino acid sequences that are at least 80% identical to the sequences set forth in Table 5.

Anti-TNFRSF, anti-TNFSF, anti-CD28, or anti-ICOS antibodies or antigen-binding fragments thereof of the disclosure can mitigate the T-reg-mediated depletion of tumor-reactive T-cells by suppressing the growth and proliferation of T-reg cells that typically accompanies T-cell infusion. For instance, anti-TNFRSF, anti-TNFSF, anti-CD28, or anti-ICOS antibodies or antigen-binding fragments thereof of the disclosure may be capable of reducing the growth of a population of T-reg cells by about 50% to about 200% relative to untreated cells (e.g., 50%, 75%, 100%, 125%, 150%, 175%, or 200%). The reduction in cellular growth occurs even in the presence of a cognate or natural ligand of the receptor. In some embodiments, anti-TNFRSF, anti-TNFSF, anti-CD28, or anti-ICOS antibodies or antigen-binding fragments thereof of the disclosure may be capable of restricting the growth of a population of T-reg cells in the presence of the natural ligand of the TNFRSF member protein, the TNFSF member protein, CD28, or ICOS to between 90% and 150% relative to untreated cells (e.g., 90%, 100%, 110%, 120%, 130%, 140%, or 150%). Anti-TNFRSF, anti-TNFSF, anti-CD28, or anti-ICOS antibodies or antigen-binding fragments thereof of the disclosure are also capable of restricting the proliferation of a population of T-reg cells to less than 70% (e.g., 60%, 50%, 40%, 30%, 20%, 10%, 5%, or 1%) of that of an untreated population of T-reg cells. Anti-TNFRSF, anti-TNFSF, anti-CD28, or anti-ICOS antibodies or antigen-binding fragments thereof of the disclosure are also capable of decreasing the survival of a population of T-reg cells by about 10% (e.g., by about 20%, 30%, 40%, or 50%, or more) relative to an untreated population of T-reg cells.

Anti-TNFRSF, anti-TNFSF, anti-CD28, or anti-ICOS antibodies or antigen-binding fragments thereof of the disclosure can mitigate the MDSC-mediated depletion of tumor-reactive T-cells by suppressing the growth and proliferation of MDSCs that may accompany T-cell infusion. For instance, anti-TNFRSF, anti-TNFSF, anti-CD28, or anti-ICOS antibodies or antigen-binding fragments thereof of the disclosure may be capable of reducing the growth of a population of MDSCs by about 50% to about 200% relative to untreated cells (e.g., 50%, 75%, 100%, 125%, 150%, 175%, or 200%). The reduction in cellular growth occurs even in the presence of a cognate or natural ligand of the receptor. In some embodiments, anti-TNFRSF, anti-TNFSF, anti-CD28, or anti-ICOS antibodies or antigen-binding fragments thereof of the disclosure may be capable of restricting the growth of a population of MDSCs in the presence of the natural ligand of the TNFRSF member protein, the TNFSF member protein, CD28, or ICOS to between 90% and 150% relative to untreated cells (e.g., 90%, 100%, 110%, 120%, 130%, 140%, or 150%). anti-TNFRSF, anti-TNFSF, anti-CD28, or anti-ICOS antibodies or antigen-binding fragments thereof of the disclosure are also capable of restricting the proliferation of a population of MDSCs to less than 70% (e.g., 60%, 50%, 40%, 30%, 20%, 10%, 5%, or 1%) of that of an untreated population of MDSCs. anti-TNFRSF, anti-TNFSF, anti-CD28, or anti-ICOS antibodies or antigen-binding fragments thereof of the disclosure are also capable of decreasing the survival of a population of MDSCs by about 10% (e.g., by about 20%, 30%, 40%, or 50%, or more) relative to an untreated population of MDSCs.

Anti-TNFRSF, anti-TNFSF, anti-CD28, or anti-ICOS antibodies or antigen-binding fragments thereof of the disclosure can directly kill cells expressing the TNFRSF member protein, the TNFSF member protein, CD28, or ICOS, such as B cells, parenchymal cells, dendritic cells, platelets, and granulocytes. For instance, anti-TNFRSF, anti-TNFSF, anti-CD28, or anti-ICOS antibodies or antigen-binding fragments thereof of the disclosure may be capable of reducing the growth of a population of TNFRSF-, TNFSF-, CD28-, or ICOS-expressing cells by about 50% to about 200% relative to untreated cells (e.g., 50%, 75%, 100%, 125%, 150%, 175%, or 200%). The reduction in cellular growth may occur even in the presence of a cognate or natural ligand of the receptor. In some embodiments, anti-TNFRSF, anti-TNFSF, anti-CD28, or anti-ICOS antibodies or antigen-binding fragments thereof of the disclosure may be capable of restricting the growth of a population of TNFRSF-, TNFSF-, CD28-, or ICOS-expressing cells in the presence of the natural ligand of the TNFRSF member protein, the TNFSF member protein, CD28, or ICOS to between 90% and 150% relative to untreated cells (e.g., 90%, 100%, 110%, 120%, 130%, 140%, or 150%). Anti-TNFRSF, anti-TNFSF, anti-CD28, or anti-ICOS antibodies or antigen-binding fragments thereof of the disclosure are also capable of restricting the proliferation of a population of TNFRSF-, TNFSF-, CD28-, or ICOS-expressing cells to less than 70% (e.g., 60%, 50%, 40%, 30%, 20%, 10%, 5%, or 1%) of that of an untreated population of TNFRSF-, TNFSF-, CD28-, or ICOS-expressing cells. Anti-TNFRSF, anti-TNFSF, anti-CD28, or anti-ICOS antibodies or antigen-binding fragments thereof of the disclosure are also capable of decreasing the survival of a population of TNFRSF-, TNFSF-, CD28-, or ICOS-expressing cells by about 10% (e.g., by about 20%, 30%, 40%, or 50%, or more) relative to an untreated population of TNFRSF-, TNFSF-, CD28-, or ICOS-expressing cells.

Anti-TNFRSF, anti-TNFSF, anti-CD28, or anti-ICOS antibodies or antigen-binding fragments thereof of the disclosure can be administered to a mammalian subject (e.g., a human) suffering from cancer in order to improve the condition of the subject by promoting the immune response against cancer cells and tumorigenic material. Anti-TNFRSF, anti-TNFSF, anti-CD28, or anti-ICOS antibodies or antigen-binding fragments thereof of the disclosure can be administered to a subject, e.g., via any of the routes of administration described herein. Anti-TNFRSF, anti-TNFSF, anti-CD28, or anti-ICOS antibodies or antigen-binding fragments thereof of the disclosure can also be formulated with excipients, biologically acceptable carriers, and may be optionally conjugated to, admixed with, or co-administered separately (e.g., sequentially) with additional therapeutic agents, such as anti-cancer agents. Cancers that can be treated by administration of anti-TNFRSF, anti-TNFSF, anti-CD28, or anti-ICOS antibodies or antigen-binding fragments thereof of the disclosure include such cancers as leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer. Particular cancers that can be treated by administration of anti-TNFRSF, anti-TNFSF, anti-CD28, or anti-ICOS antibodies or antigen-binding fragments thereof of the disclosure include, without limitation, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, Ewing sarcoma family, osteosarcoma and malignant fibrous histiocytoma, central nervous system embryonal tumors, central nervous system germ cell tumors, craniopharyngioma, ependymoma, bronchial tumors, Burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, Langerhans cell histiocytosis, Hodgkin lymphoma, hypopharyngeal cancer, islet cell tumors, pancreatic neuroendocrine tumors, Wilms tumor and other childhood kidney tumors, Langerhans cell histiocytosis, small cell lung cancer, cutaneous T-cell lymphoma, intraocular melanoma, Merkel cell carcinoma, mesothelioma, metastatic squamous neck cancer, midline tract carcinoma, multiple endocrine neoplasia syndromes, multiple myeloma/plasma cell neoplasm (e.g., multiple myeloma or refractory multiple myeloma), myelodysplastic syndromes, nasal cavity and paranasal sinus cancer, nasopharyngeal cancer, neuroblastoma, non-Hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi sarcoma, rhabdomyosarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

Anti-TNFRSF, anti-TNFSF, anti-CD28, or anti-ICOS antibodies or antigen-binding fragments thereof of the disclosure can also be co-administered with a therapeutic antibody that exhibits reactivity towards a cancer cell. In this way, anti-TNFRSF, anti-TNFSF, anti-CD28, or anti-ICOS antibodies or antigen-binding fragments thereof of the disclosure may synergize not only with the adaptive immune response, e.g., by prolonging T-lymphocyte tumor reactivity, but also with other inhibitors of tumor cell growth. Examples of additional therapeutic antibodies that can be used to treat cancer and other cell proliferation disorders include those that exhibit reactivity with a tumor antigen or a cell-surface protein that is overexpressed on the surface of a cancer cell. Exemplary antibodies that can be admixed, co-administered, or sequentially administered with the anti-TNFRSF, anti-TNFSF, anti-CD28, or anti-ICOS antibodies or antigen-binding fragments thereof of the disclosure include, without limitation, Trastuzumab (HERCEPTIN®), Bevacizumab (AVASTIN®), Cetuximab (ERBITUX®), Panitumumab (VECTIBIX®), Ipilimumab (YERVOY®), Rituximab (RITUXAN® and MABTHERA®), Alemtuzumab (CAMPATH®), Ofatumumab (ARZERRA®), Gemtuzumab ozogamicin (MYLOTARG®), Brentuximab vedotin (ADCETRIS®), ⁹⁰Y-Ibritumomab Tiuxetan (ZEVALIN®), and ¹³¹I-Tositumomab (BEXXAR®), which are described in detail in Scott et al. Cancer Immun. 12:14-21, 2012; incorporated herein by reference.

Anti-TNFRSF, anti-TNFSF, anti-CD28, or anti-ICOS antibodies or antigen-binding fragments thereof of the disclosure can be monitored for their ability to attenuate the progression of a cell proliferation disease, such as cancer, by any of a variety of methods known in the art. For instance, a physician may monitor the response of a mammalian subject (e.g., a human) to treatment with an anti-TNFRSF, anti-TNFSF, anti-CD28, or anti-ICOS antibody or antigen-binding fragment thereof of the disclosure by analyzing the volume of one or more tumors in the subject. For example, anti-TNFRSF, anti-TNFSF, anti-CD28, or anti-ICOS antibodies or antigen-binding fragments thereof of the disclosure may be capable of reducing tumor volume by between 1% and 100% (e.g., 1%, 5%, 10%, 20%, 30%, 40%, 50%, 60%, 70%, 80%, 90%, 95%, 99%, or 100%). Alternatively, a physician may monitor the responsiveness of a subject (e.g., a human) to treatment with anti-TNFRSF, anti-TNFSF, anti-CD28, or anti-ICOS antibodies or antigen-binding fragments thereof of the disclosure by analyzing the T-reg cell population in the lymph of a particular subject. For instance, a physician may withdraw a sample of blood from a mammalian subject (e.g., a human who was administered the antagonist) and determine the quantity or density of a population of T-reg cells (e.g., CD4+ CD25+ FOXP3+ T-reg cells or CD17+ T-reg cells) using established procedures, such as fluorescence activated cell sorting.

### Methods of Treating Autoimmune Diseases and Transplant Rejections

Anti-TNFRSF, anti-TNFSF, anti-CD28, or anti-ICOS antibodies or antigen-binding fragments thereof of the disclosure may be administered to a subject (e.g., a human) to treat an autoimmune disease. Anti-TNFRSF, anti-TNFSF, anti-CD28, or anti-ICOS antibodies or antigen-binding fragments thereof of the disclosure may, additionally or alternatively, directly kill T effector cells, such as CD8+ T effector cells, and may promote the proliferation, regeneration, healing, and/or protection of TNFRSF-, TNFSF-, CD28-, or ICOS-expressing parenchymal cells, as described herein.

Anti-TNFRSF, anti-TNFSF, anti-CD28, or anti-ICOS antibodies or antigen-binding fragments thereof of the disclosure can be administered to a subject, e.g., a mammalian subject, such as a human, in order to treat such conditions as autoimmune diseases, neurological diseases, metabolic diseases (e.g., diabetes), macular diseases (e.g., macular degeneration), muscular atrophy, diseases related to miscarriage, vascular diseases (e.g., atherosclerosis), diseases related to bone loss (e.g., bone loss as a result of menopause or osteoporosis), allergies, asthma, a blood disorder (e.g., hemophilia), a musculoskeletal disorder, a disease related to growth receptor expression or activity, obesity, graft-versus-host disease (GVHD), or a transplant rejection (e.g., an allograft rejection), among other indications.

Anti-TNFRSF, anti-TNFSF, anti-CD28, or anti-ICOS antibodies or antigen-binding fragments thereof of the disclosure can be administered to a subject, e.g., a mammalian subject, such as a human, suffering from a graft rejection. Examples of graft rejections that can be treated by administration of the anti-TNFRSF, anti-TNFSF, anti-CD28, or anti-ICOS antibodies or antigen-binding fragments thereof of the disclosure include, without limitation, skin graft rejection, bone graft rejection, vascular tissue graft rejection, ligament graft rejection (e.g., cricothyroid ligament graft rejection, periodontal ligament graft rejection, suspensory ligament of the lens graft rejection, palmar radiocarpal ligament graft rejection, dorsal radiocarpal ligament graft rejection, ulnar collateral ligament graft rejection, radial collateral ligament graft rejection, suspensory ligament of the breast graft rejection, anterior sacroiliac ligament graft rejection, posterior sacroiliac ligament graft rejection, sacrotuberous ligament graft rejection, sacrospinous ligament graft rejection, inferior pubic ligament graft rejection, superior pubic ligament graft rejection, anterior cruciate ligament graft rejection, lateral collateral ligament graft rejection, posterior cruciate ligament graft rejection, medial collateral ligament graft rejection, cranial cruciate ligament graft rejection, caudal cruciate ligament graft rejection, patellar ligament graft rejection) and organ graft rejection (e.g., heart, lung, kidney, liver, pancreas, intestine, and thymus graft rejection, among others).

Anti-TNFRSF, anti-TNFSF, anti-CD28, or anti-ICOS antibodies or antigen-binding fragments thereof of the disclosure may, additionally or alternatively, be administered to a subject, e.g., a mammalian subject, such as a human) suffering from a graft-versus-host disease (GVHD). Exemplary graft-versus-host diseases that can be treated using the compositions and methods of the disclosure include those that arises from a bone marrow transplant, as well as from the transplantation of blood cells, such as hematopoietic stem cells, common myeloid progenitor cells, common lymphoid progenitor cells, megakaryocytes, monocytes, basophils, eosinophils, neutrophils, macrophages, T-cells, B-cells, natural killer cells, and/or dendritic cells.

Diseases that can be treated by administration of the anti-TNFRSF, anti-TNFSF, anti-CD28, or anti-ICOS antibodies or antigen-binding fragments thereof of the disclosure include autoimmune diseases, such as type I diabetes, alopecia areata, ankylosing spondylitis, anti-N-methyl-D-aspartate receptor (NMDAR) encephalitis, antiphospholipid syndrome, autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, limited scleroderma (CREST Syndrome), cold agglutinin disease, Crohn's Disease, discoid lupus, essential mixed cryoglobulinemia, fibromyalgia-fibromyositis, Graves' Disease, Guillain-Barre Syndrome, Hashimoto's Thyroiditis, hypothyroidism, Inflammatory Bowel Disease, autoimmune lymphoproliferative syndrome (ALPS), idiopathic pulmonary fibrosis, idiopathic thrombocytopenia purpura (ITP), IgA nephropathy, juvenile arthritis, lichen planus, lupus, Menière's Disease, mixed connective tissue disease, multiple sclerosis, myasthenia gravis, Pemphigus vulgaris, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, Stiff-Man syndrome, Takayasu Arteritis, temporal arteritis/giant cell arteritis, ulcerative colitis, uveitis, vasculitis, vitiligo, and Wegener's Granulomatosis.

Anti-TNFRSF, anti-TNFSF, anti-CD28, or anti-ICOS antibodies or antigen-binding fragments thereof of the disclosure can additionally be used to treat subjects in need of organ repair or regeneration. For instance, anti-TNFRSF, anti-TNFSF, anti-CD28, or anti-ICOS antibodies or antigen-binding fragments thereof of the disclosure may be used to stimulate organ repair or regeneration. Examples of tissues and organs that may be induced to regenerate by administration of an anti-TNFRSF, anti-TNFSF, anti-CD28, or anti-ICOS antibody or antigen-binding fragment thereof of the disclosure to a subject (e.g., a mammalian subject, such as a human) include the pancreas, salivary gland, pituitary gland, kidney, heart, lung, hematopoietic system, cranial nerves, heart, blood vessels including the aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes.

Anti-TNFRSF, anti-TNFSF, anti-CD28, or anti-ICOS antibodies or antigen-binding fragments thereof of the disclosure can be monitored for their ability to attenuate the progression of an immunological disease, such as an autoimmune disease, by any of a variety of methods known in the art. For instance, a physician may monitor the responsiveness of a subject (e.g., a human who was administered the antagonist) to treatment with the anti-TNFRSF, anti-TNFSF, anti-CD28, or anti-ICOS antibodies or antigen-binding fragments thereof of the disclosure by analyzing the T-reg cell population in the lymph of a particular subject. For instance, a physician may withdraw a sample of blood from a mammalian subject (e.g., a human who was administered the antagonist) and determine the quantity or density of a population of T-reg cells (e.g., CD4+ CD25+ FOXP3+ T-reg cells or CD17+ T-reg cells) using established procedures, such as fluorescence activated cell sorting. In these cases, high counts of T-reg cells can be indicative of efficacious therapy, while lower T-reg cell counts may indicate that the subject is to be prescribed or administered higher dosages of the anti-TNFRSF, anti-TNFSF, anti-CD28, or anti-ICOS antibodies or antigen-binding fragments thereof of the disclosure until, e.g., an ideal T-reg cell count is achieved. In addition, a physician of skill in the art may monitor the effect of treatment by administration of the anti-TNFRSF, anti-TNFSF, anti-CD28, or anti-ICOS antibodies or antigen-binding fragments thereof of the disclosure to a subject suffering from an immunological disorder, such as an autoimmune disease described herein, by analyzing the quantity of autoreactive CD8+ T-cells within a lymph sample isolated from the subject.

Anti-TNFRSF, anti-TNFSF, anti-CD28, or anti-ICOS antibodies or antigen-binding fragments thereof of the disclosure may attenuate the proliferation of autoreactive T-cells by about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% relative to a reference level of the proliferation of autoreactive T-cells. The level of proliferation of autoreactive T-cells can be determined by isolating said cells from a subject (e.g., a subject having an autoimmune disease, such as type I diabetes) and performing a proliferation assay which is known in the art (see, e.g., Seyfert-Margolis et al. Diabetes 55:2588-2594, 2006; incorporated herein by reference). Treatment with the anti-TNFRSF, anti-TNFSF, anti-CD28, or anti-ICOS antibodies or antigen-binding fragments thereof of the disclosure can lead to reduced quantities of autoreactive T-cells within the lymph isolated from a subject receiving treatment, and a rapid decline in the population of autoreactive T-cells in a lymph sample isolated from such a subject indicates effective treatment. In cases where a lymph sample isolated from a subject exhibits an autoreactive T-cell count that has not declined in response to anti-TNFRSF, anti-TNFSF, anti-CD28, or anti-ICOS antibody therapy, a physician may prescribe the subject higher doses of the antibody or an antigen-binding fragment thereof or may administer the antibody or antigen-binding fragment thereof with higher frequency, e.g., multiple times per day, week, or month.

### Methods of Treating Infectious Diseases

Anti-TNFRSF, anti-TNFSF, anti-CD28, or anti-ICOS antibodies or antigen-binding fragments thereof of the disclosure can be used for treating infectious diseases, such as those caused by any one or more of a virus, a bacterium, a fungus, or a parasite. Anti-TNFRSF, anti-TNFSF, anti-CD28, or anti-ICOS antibodies or antigen-binding fragments thereof of the disclosure can be administered to a mammalian subject (e.g., a human) suffering from an infectious disease in order to treat the disease, as well as to alleviate one or more symptoms of the disease.

For example, anti-TNFRSF, anti-TNFSF, anti-CD28, or anti-ICOS antibodies or antigen-binding fragments thereof of the disclosure can be used for treating, or alleviating one or more symptoms of, viral infections in a mammalian subject, such as a human, that are caused by, e.g., a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus; a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabia virus, Tacaribe virus, Tamiami virus, Whitewater Arroyo virus, Chapare virus, and Lujo virus; a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, California encephalitis virus, and Crimean-Congo hemorrhagic fever (CCHF) virus; a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively); Friend Leukemia Virus; and transmissible spongiform encephalopathy, such as chronic wasting disease. Particularly, methods of the disclosure include administering an anti-TNFRSF, anti-TNFSF, anti-CD28, or anti-ICOS antibody or antigen-binding fragment thereof of the disclosure to a human in order to treat an HIV infection (such as a human suffering from AIDS).

Anti-TNFRSF, anti-TNFSF, anti-CD28, or anti-ICOS antibodies or antigen-binding fragments thereof of the disclosure can also be used for treating, or alleviating one or more symptoms of, bacterial infections in a mammalian subject (e.g., a human). Examples of bacterial infections that may be treated by administration of the anti-TNFRSF, anti-TNFSF, anti-CD28, or anti-ICOS antibodies or antigen-binding fragments thereof of the disclosure include, without limitation, those caused by bacteria within the genera Streptococcus, Bacillus, Listeria, Corynebacterium, Nocardia, Neisseria, Actinobacter, Moraxella, Enterobacteriacece (e.g., E. coli, such as O157:H7), Pseudomonas (such as Pseudomonas aeruginosa), Escherichia, Klebsiella, Serratia, Enterobacter, Proteus, Salmonella, Shigella, Yersinia, Haemophilus, Bordetella (such as Bordetella pertussis), Legionella, Pasteurella, Francisella, Brucella, Bartonella, Clostridium, Vibrio, Campylobacter, Staphylococcus, Mycobacterium (such as Mycobacterium tuberculosis and Mycobacterium avium paratuberculosis, and Helicobacter (such as Helicobacter pylori and Helicobacter hepaticus). Particularly, methods of the disclosure include administering an anti-TNFRSF, anti-TNFSF, anti-CD28, or anti-ICOS antibody or antigen-binding fragment thereof of the disclosure to a human or a non-human mammal in order to treat a Mycobacterium tuberculosis infection.

Anti-TNFRSF, anti-TNFSF, anti-CD28, or anti-ICOS antibodies or antigen-binding fragments thereof of the disclosure can also be administered to a mammalian subject (e.g., a human) for treating, or alleviating one or more symptoms of, parasitic infections caused by a protozoan parasite (e.g., an intestinal protozoa, a tissue protozoa, or a blood protozoa) or a helminthic parasite (e.g., a nematode, a helminth, an adenophorea, a secementea, a trematode, a fluke (blood flukes, liver flukes, intestinal flukes, and lung flukes), or a cestode). Exemplary protozoan parasites that can be treated according to the methods of the disclosure include, without limitation, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Leishmania major, Toxoplasma gondii, Plasmodium vivax, Plasmodium ovale, Plasmodium malariae, Plasmodium falciparum, Plasmodium yoelli, Trichomonas vaginalis, and Histomonas meleagridis. Exemplary helminthic parasites include Richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus. Additional parasitic infections that can be treated according to the methods of the disclosure include Onchocercas volvulus.

Anti-TNFRSF, anti-TNFSF, anti-CD28, or anti-ICOS antibodies or antigen-binding fragments thereof of the disclosure can also be administered to a mammalian subject (e.g., a human) in order to treat, or to alleviate one or more symptoms of, fungal infections. Examples of fungal infections that may be treated according to the methods of the disclosure include, without limitation, those caused by, e.g., Aspergillus, Candida, Malassezia, Trichosporon, Fusarium, Acremonium, Rhizopus, Mucor, Pneumocystis, and Absidia. Exemplary fungal infections that can be treated according to the methods of the disclosure also include Pneumocystis carinii, Paracoccidioides brasiliensis and Histoplasma capsulatum.

### Methods of Treating Inflammatory Diseases

Anti-TNFRSF, anti-TNFSF, anti-CD28, or anti-ICOS antibodies or antigen-binding fragments thereof of the disclosure can also be used for treating inflammatory diseases, such as those caused by chronic or acute inflammation. The anti-TNFRSF, anti-TNFSF, anti-CD28, or anti-ICOS antibodies or antigen-binding fragments thereof of the disclosure can be administered to a mammalian subject (e.g., a human) suffering from an inflammatory disease in order to treat the disease, as well as to alleviate one or more symptoms of the disease.

Anti-TNFRSF, anti-TNFSF, anti-CD28, or anti-ICOS antibodies or antigen-binding fragments thereof of the disclosure can also be administered to a mammalian subject (e.g., a human) for treating, or alleviating one or more symptoms of, inflammatory diseases such as cardiac fibrosis, lung fibrosis, osteoarthritis, rheumatoid arthritis, atherosclerosis, type I diabetes, type II diabetes, graft-versus-host disease, multiple sclerosis, osteomyelitis, psoriasis, Crohn's disease, Sjögren's syndrome, lupus erythematosus, and ulcerative colitis, among others.

### Methods of Treating Neurological Diseases or Disorders

Anti-TNFRSF, anti-TNFSF, anti-CD28, or anti-ICOS antibodies or antigen-binding fragments thereof of the disclosure may be administered to a subject (e.g., a human) to treat a neurological disease or disorder such as a brain tumor, a brain metastasis, a brain injury, a spinal cord injury, a nerve injury, schizophrenia, epilepsy, Parkinson's disease, autism, Huntington's disease, stroke, Alzheimer's disease, multiple sclerosis, amyotrophic lateral sclerosis (ALS), and myasthenia gravis. Anti-TNFRSF, anti-TNFSF, anti-CD28, or anti-ICOS antibodies or antigen-binding fragments thereof of the disclosure can be used as a neuroprotective agent, e.g., by attenuating the neuroinflammatory response (see Ots et al. Int. J. Mol. Sci. 23:4115, 2022, which is incorporated herein by reference). A physician of skill in the art may monitor the effect of treatment by administration of the anti-TNFRSF, anti-TNFSF, anti-CD28, or anti-ICOS antibodies or antigen-binding fragments thereof of the disclosure to a subject suffering from a neurological disease or disorder, by analyzing the quantity of a soluble TNFRSF member protein (e.g., soluble TNFR2 (sTNFR2), among others) within a blood sample isolated from the subject, which can be used as a biomarker of TNFRSF-mediated inflammatory activity.

### Measuring Levels of Soluble TNFRSF Member Protein as a Biomarker

Soluble forms of TNFRSF member proteins are generated by enzymatic cleavage of membrane bound TNFRSF member proteins, and usually work similar to the decoy receptor, which lacks an intracellular domain and acts as TNFSF ligand inhibitors. Elevated levels of soluble TNFRSF member proteins are often indicative of immune activation. Levels of secreted soluble TNFRSF member proteins (e.g., TNFR2, TNFR1, 4-1BB, CD27, CD30, CD40, DR6, EDAR, Fas, GITR, HVEM, LT-β receptor, NGFR, OPG, OX40, RANK, RELT (TNFRSF19L), TRAIL-R1 (TNFRSF10A), TRAIL-R2 (TNFRSF10B), TRAIL-R4, TRAMP, TROY, XEDAR, or DCR3, among others) can be measured in a biological sample (e.g., serum or plasma) of a subject to determine disease progression and/or efficacy of treatment. The level of a secreted soluble TNFRSF member protein (e.g., soluble TNFR2 (sTNFR2), such as in the treatment of type 1 diabetes) can be measured again after administration of the anti-TNFRSF antibodies or antigen-binding fragments thereof of the disclosure to a subject to determine if administration has caused a decrease in the level of the secreted soluble TNFRSF member protein. For example, the level of a secreted soluble TNFRSF member protein in a subject can be measured one day, two days, three days, four days, five days, six days, one week, eight days, nine days, ten days, eleven days, twelve days, thirteen days, fourteen days, fifteen days, sixteen days, seventeen days, eighteen days, nineteen days, twenty days, three weeks, four weeks, five weeks, six weeks, seven weeks, eight weeks, or more after administration of the anti-TNFRSF antibodies or antigen-binding fragments thereof of the disclosure. For example, a subject can be administered one or more additional doses and/or treatment periods of an antagonistic anti-TNFRSF antibodies or antigen-binding fragments thereof of the disclosure if the post-treatment level of the secreted soluble TNFRSF member protein is about the same or higher than a reference level (e.g., the level of the secreted soluble TNFRSF member protein measured in a healthy, untreated human) or a previously measured level of the secreted soluble TNFRSF member protein (a pre-treatment level). Conversely, a subject can be administered one or more additional doses and/or treatment periods of an agonistic anti-TNFRSF antibodies or antigen-binding fragments thereof of the disclosure if the post-treatment level of the secreted soluble TNFRSF member protein is about the same or lower than a reference level or a pre-treatment level of the secreted soluble TNFRSF member protein.

The level of a soluble TNFRSF member protein in a biological sample (e.g., serum or plasma) of a subject can also be used for the diagnosis or screening of a disease or disorder (e.g., a cell proliferation disorder, an autoimmune disease, an infectious disease, an inflammatory disease, a neurological disease, an allergy, a transplant rejection (e.g., an allograft rejection), or a graft-versus-host disease disclosed herein, such as a cancer). A level of a soluble TNFRSF member protein in a biological sample (e.g., serum or plasma) of a subject that is higher than a reference level of the soluble TNFRSF member protein in a healthy human is indicative of a disease or disorder in the subject, such as a cancer, an autoimmune disease, an infectious disease, an inflammatory disease, or a transplant rejection. For example, elevated level of soluble OX40 is correlated with bacterial infection; elevated level of soluble TNFR2 is correlated with influenza and cancer (e.g., glioblastoma, pancreatic cancer, and non-Hodgkin lymphoma); elevated level of soluble CD40 is correlated with rheumatoid arthritis (RA), systemic lupus erythematosus (SLE), and Alzheimer's disease; elevated level of soluble 4-1BB is correlated with RA, multiple sclerosis (MS), and primary biliary cirrhosis; elevated level of soluble CD30 is correlated with chronic infection and transplant rejection; and elevated level of soluble CD27 is correlated with SLE (see, e.g., Croft et al. Trends Immunol. 33:144-152, 2012; Ward-Kavanagh et al. Immunity 44:1005-1019, 2016; and Takahashi et al. Cells 11:1952, 2022; incorporated herein by reference).

The level of a soluble TNFRSF member protein in a biological sample (e.g., serum or plasma) of a subject can also be used for determining the treatment group to which the subject is assigned. For example, a subject having a level of a soluble TNFRSF member protein that is lower than a reference level can be assigned to a treatment group that is administered an agonistic antibody or antigen-binding fragment thereof that specifically binds the TNFRSF member protein. A subject having a level of a soluble TNFRSF member protein that is higher than a reference level can be assigned to a treatment group that is administered an antagonistic antibody or antigen-binding fragment thereof that specifically binds the TNFRSF member protein.

In some embodiments, the level of a soluble TNFRSF member protein in a biological sample (e.g., serum or plasma) of a subject is used for determining the appropriate dosing of a pharmaceutical composition of the disclosure (e.g., an antibody or antigen-binding fragment thereof that specifically binds a TNFRSF member protein, or a construct, polynucleotide, vector, or host cell encoding thereof). For example, when administering an agonistic antibody or antigen-binding fragment thereof that specifically binds a TNFRSF member protein to a subject, a higher dose and/or dosing frequency of the antibody or antigen-binding fragment thereof should be given to the subject if the post-treatment level of the soluble TNFRSF member protein is about the same or lower than a reference level or the pre-treatment level of the soluble TNFRSF member protein; a lower dose and/or dosing frequency of the antibody or antigen-binding fragment thereof should be given to the subject if the post-treatment level of the soluble TNFRSF member protein is about the same or higher than a reference level or the pre-treatment level of the soluble TNFRSF member protein. Conversely, when administering an antagonistic antibody or antigen-binding fragment thereof that specifically binds a TNFRSF member protein to a subject, a higher dose and/or dosing frequency of the antibody or antigen-binding fragment thereof should be given to the subject if the post-treatment level of the soluble TNFRSF member protein is about the same or higher than a reference level or the pre-treatment level of the soluble TNFRSF member protein; a lower dose and/or dosing frequency of the antibody or antigen-binding fragment thereof should be given to the subject if the post-treatment level of the soluble TNFRSF member protein is about the same or lower than a reference level or the pre-treatment level of the soluble TNFRSF member protein. These adjustments in dosing can also be used to guide the administration of the antibody or antigen-binding fragment thereof over a narrower dose range.

### Routes of Administration and Dosing

Anti-TNFRSF, anti-TNFSF, anti-CD28, or anti-ICOS antibodies or antigen-binding fragments thereof of the disclosure can be administered to a mammalian subject (e.g., a human) by a variety of routes such as orally, transdermally, subcutaneously, intranasally, intravenously, intramuscularly, intraocularly, intratumorally, parenterally, topically, intrathecally and intracerebroventricularly. The most suitable route for administration in any given case will depend on the particular antibody or antigen-binding fragment administered, the subject, pharmaceutical formulation methods, administration methods (e.g., administration time and administration route), the subject's age, body weight, sex, severity of the diseases being treated, the subject's diet, and the subject's excretion rate. Due to the exceptional antagonistic or agonistic activity of the antibodies or antigen-binding fragments thereof of the disclosure, the antibodies or antigen-binding fragments thereof may be administered to a subject subcutaneously. In some embodiments, the anti-TNFRSF, anti-TNFSF, anti-CD28, or anti-ICOS antibody or antigen-binding fragment thereof of the disclosure (e.g., an anti-TNFR2 antibody or antigen-binding fragment thereof) is administered subcutaneously.

The effective dose of an anti-TNFRSF, anti-TNFSF, anti-CD28, or anti-ICOS antibody or antigen-binding fragment thereof of the disclosure can range from about 0.0001 to about 100 mg/kg of body weight per single (e.g., bolus) administration, multiple administrations or continuous administration, or to achieve a serum concentration of 0.0001-5000 μg/mL per single (e.g., bolus) administration, multiple administrations or continuous administration, or any effective range or value therein depending on the condition being treated, the route of administration and the age, weight, and condition of the subject. A single dose of an anti-TNFRSF, anti-TNFSF, anti-CD28, or anti-ICOS antibody or antigen-binding fragment thereof of the disclosure can range from about 0.001 to about 500000 mg or from about 0.0001 to about 100 mg/kg of body weight of the subject. In certain examples, e.g., for the treatment of cancer, each dose can range from about 0.0001 mg to about 500 mg/kg of body weight. For instance, a pharmaceutical composition of the disclosure may be administered in a daily dose in the range of 0.001-100 mg/kg (body weight). The dose may be administered one or more times (e.g., 2-10 times) per day, week, month, or year to a mammalian subject (e.g., a human) in need thereof. In some embodiments, the anti-TNFRSF antibodies or antigen-binding fragments thereof of the disclosure (e.g., an anti-TNFR2 antibody or antigen-binding fragment thereof) are administered once every week at a dose of from about 0.8 mg/kg to about 1.2 mg/kg (e.g., about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, or about 1.2 mg/kg). In some embodiments, the anti-TNFRSF antibodies or antigen-binding fragments thereof of the disclosure are administered once every two weeks at a dose of from about 1.65 mg/kg to about 2.5 mg/kg (e.g., about 1.65 mg/kg, about 1.7 mg/kg, about 1.75 mg/kg, about 1.8 mg/kg, about 1.85 mg/kg, about 1.9 mg/kg, about 1.95 mg/kg, about 2.0 mg/kg, about 2.05 mg/kg, about 2.1 mg/kg, about 2.15 mg/kg, about 2.2 mg/kg, about 2.25 mg/kg, about 2.3 mg/kg, about 2.35 mg/kg, about 2.4 mg/kg, about 2.45 mg/kg, or about 2.5 mg/kg). In some embodiments, the anti-TNFRSF antibodies or antigen-binding fragments thereof of the disclosure are administered once every three weeks at a dose of from about 2.2 mg/kg to about 3.0 mg/kg (e.g., about 2.2 mg/kg, about 2.25 mg/kg, about 2.3 mg/kg, about 2.35 mg/kg, about 2.4 mg/kg, about 2.45 mg/kg, about 2.5 mg/kg, about 2.55 mg/kg, about 2.6 mg/kg, about 2.65 mg/kg, about 2.7 mg/kg, about 2.75 mg/kg, about 2.8 mg/kg, about 2.85 mg/kg, about 2.9 mg/kg, about 2.95 mg/kg, or about 3.0 mg/kg). In some embodiments, the anti-TNFRSF antibodies or antigen-binding fragments thereof of the disclosure are administered once every four weeks at a dose of from about 2.5 mg/kg to about 4.2 mg/kg (e.g., about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, or about 4.2 mg/kg).

Therapeutic compositions can be administered with medical devices known in the art. For example, in some embodiments, a therapeutic composition of the disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the disclosure include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

### Kits Containing Antibodies or Antigen-Binding Fragments Thereof

This disclosure also includes kits that contain anti-TNFRSF, anti-TNFSF, anti-CD28, or anti-ICOS antibodies or antigen-binding fragments thereof of the disclosure (e.g., an anti-TNFR2 antibody or antigen-binding fragment thereof). The kits provided herein may contain any of the anti-TNFRSF, anti-TNFSF, anti-CD28, or anti-ICOS antibodies or antigen-binding fragments thereof described above, as well as any of the polynucleotides encoding these antibodies or antigen-binding fragments thereof, vectors containing these antibodies or antigen-binding fragments thereof, or cells engineered to express and secrete the antibodies or antigen-binding fragments thereof of the disclosure (e.g., prokaryotic or eukaryotic cells). A kit of this disclosure may include reagents that can be used to produce the compositions of the disclosure (e.g., anti-TNFRSF, anti-TNFSF, anti-CD28, or anti-ICOS antibodies or antigen-binding fragments thereof, polynucleotides encoding said antibodies or antigen-binding fragments thereof, or vectors containing said polypeptides). Optionally, kits of the disclosure may include reagents that can induce the expression of the anti-TNFRSF, anti-TNFSF, anti-CD28, or anti-ICOS antibodies or antigen-binding fragments thereof of the disclosure within cells (e.g., mammalian cells), such as doxycycline or tetracycline. In other cases, a kit of the disclosure may contain a compound capable of binding and detecting a fusion protein that contains an anti-TNFRSF, anti-TNFSF, anti-CD28, or anti-ICOS antibody or antigen-binding fragment thereof of the disclosure and an epitope tag. For instance, in such cases a kit of the disclosure may contain maltose, glutathione, a nickel-containing complex, an anti-FLAG antibody, an anti-myc antibody, an anti-HA antibody, biotin, or streptavidin.

Kits of the disclosure may also include reagents that can detect an anti-TNFRSF, anti-TNFSF, anti-CD28, or anti-ICOS antibody or antigen-binding fragment thereof of the disclosure directly. Examples of such reagents include secondary antibodies that selectively recognize and bind particular structural features within the Fc region of an anti-TNFRSF, anti-TNFSF, anti-CD28, or anti-ICOS antibody or antigen-binding fragment thereof of the disclosure. Kits of the disclosure may contain secondary antibodies that recognize the Fc region of anti-TNFRSF, anti-TNFSF, anti-CD28, or anti-ICOS antibody or antigen-binding fragment thereof of the disclosure and that are conjugated to a fluorescent molecule. These antibody-fluorophore conjugates provide a tool for analyzing the localization of the anti-TNFRSF, anti-TNFSF, anti-CD28, or anti-ICOS antibodies or antigen-binding fragments thereof of the disclosure, e.g., in a particular tissue or cultured mammalian cell using established immunofluorescence techniques. In some embodiments, kits of the disclosure may include additional fluorescent compounds that exhibit known sub-cellular localization patterns. These reagents can be used in combination with another antibody-fluorophore conjugate, e.g., one that specifically recognizes a different receptor on the cell surface in order to analyze the localization of an anti-TNFRSF, anti-TNFSF, anti-CD28, or anti-ICOS antibody or antigen-binding fragment thereof of the disclosure relative to other cell-surface proteins.

Kits of the disclosure may also contain a reagent that can be used for the analysis of a subject's response to treatment by administration of anti-TNFRSF, anti-TNFSF, anti-CD28, or anti-ICOS antibodies or antigen-binding fragments thereof of the disclosure of the disclosure. For instance, kits of the disclosure may include an anti-TNFRSF, anti-TNFSF, anti-CD28, or anti-ICOS antibody or antigen-binding fragment thereof of the disclosure and one or more reagents that can be used to determine the quantity of T-reg cells in a blood sample withdrawn from a subject (e.g., a human who was administered the antagonist) that is undergoing treatment with an antibody of the disclosure. Such a kit may contain, e.g., antibodies that selectively bind cell-surface antigens presented by T-reg cells, such as CD4 and CD25. Optionally, these antibodies may be labeled with a fluorescent dye, such as fluorescein or tetramethylrhodamine, in order to facilitate analysis of a population of T-reg cells by fluorescence-activated cell sorting (FACS) methods known in the art. Kits of the disclosure may optionally contain one or more reagents that can be used to quantify a population of tumor-reactive T-lymphocytes in order to determine the effectiveness of an anti-TNFRSF, anti-TNFSF, anti-CD28, or anti-ICOS antibody or antigen-binding fragment thereof of the disclosure in restoring tumor-infiltrating lymphocyte proliferation. For instance, kits of the disclosure may contain an antibody that selectively binds cell-surface markers on the surface of a cytotoxic T-cell, such as CD8 or CD3. Optionally, these antibodies may be labeled with fluorescent molecules so as to enable quantitation by FACS analysis.

A kit of the disclosure may also contain one or more reagents useful for determining the affinity and selectivity of an anti-TNFRSF, anti-TNFSF, anti-CD28, or anti-ICOS antibody or antigen-binding fragment thereof of the disclosure for one or more peptides derived from the TNFRSF member protein, the TNFSF member protein, CD28, or ICOS. For instance, a kit may contain an anti-TNFRSF, anti-TNFSF, anti-CD28, or anti-ICOS antibody or antigen-binding fragment thereof of the disclosure and one or more reagents that can be used in an ELISA assay to determine the K_{d} of an antibody of the disclosure for one or more peptides that present a TNFRSF epitope, a TNFSF epitope, a CD28 epitope, or an ICOS epitope in a conformation similar to that of the epitope in the native protein. A kit may contain, e.g., a microtiter plate containing wells that have been previously conjugated to avidin, and may contain a library of TNFRSF-, TNFSF-, CD28-, or ICOS-derived peptides, each of which conjugated to a biotin moiety. Such a kit may optionally contain a secondary antibody that specifically binds to the Fc region of an anti-TNFRSF, anti-TNFSF, anti-CD28, or anti-ICOS antibody or antigen-binding fragment thereof of the disclosure, and the secondary antibody may be conjugated to an enzyme (e.g., horseradish peroxidase) that catalyzes a chemical reaction that results in the emission of luminescent light.

Kits of the disclosure may also contain anti-TNFRSF, anti-TNFSF, anti-CD28, or anti-ICOS antibodies or antigen-binding fragments thereof of the disclosure and reagents that can be conjugated to such a polypeptide, including those previously described (e.g., a cytotoxic agent, a fluorescent molecule, a bioluminescent molecule, a molecule containing a radioactive isotope, a molecule containing a chelating group bound to a paramagnetic ion, etc.). These kits may additionally contain instructions for how the conjugation of an anti-TNFRSF, anti-TNFSF, anti-CD28, or anti-ICOS antibody or antigen-binding fragment thereof of the disclosure to a second molecule, such as those described above, can be achieved.

A kit of the disclosure may also contain a vector containing a polynucleotide that encodes an anti-TNFRSF, anti-TNFSF, anti-CD28, or anti-ICOS antibody or antigen-binding fragment thereof of the disclosure, such as any of the vectors described herein. Alternatively, a kit may include mammalian cells (e.g., CHO cells) that have been genetically altered to express and secrete an anti-TNFRSF, anti-TNFSF, anti-CD28, or anti-ICOS antibody or antigen-binding fragment thereof of the disclosure from the nuclear genome of the cell. Such a kit may also contain instructions describing how expression of an anti-TNFRSF, anti-TNFSF, anti-CD28, or anti-ICOS antibody or antigen-binding fragment thereof of the disclosure from a polynucleotide can be induced, and may additionally include reagents (such as, e.g., doxycycline or tetracycline) that can be used to promote the transcription of these polynucleotides. Such kits may be useful for the manufacture of the anti-TNFRSF, anti-TNFSF, anti-CD28, or anti-ICOS antibodies or antigen-binding fragments thereof of the disclosure.

Other kits of the disclosure may include tools for engineering a prokaryotic or eukaryotic cell (e.g., a CHO cell or a BL21 (DE3) E. coli cell) so as to express and secrete an anti-TNFRSF, anti-TNFSF, anti-CD28, or anti-ICOS antibody or antigen-binding fragment thereof of the disclosure from the nuclear genome of the cell. For example, a kit may contain CHO cells stored in an appropriate medium and optionally frozen according to methods known in the art. The kit may also provide a vector containing a polynucleotide that encodes a nuclease (e.g., such as the CRISPR/Cas, zinc finger nuclease, TALEN, ARCUS™ nucleases described herein) as well as reagents for expressing the nuclease in the cell. The kit can additionally provide tools for modifying the polynucleotide that encodes the nuclease so as to enable one to alter the DNA sequence of the nuclease in order to direct the cleavage of a specific target DNA sequence of interest. Examples of such tools include primers for the amplification and site-directed mutagenesis of the polynucleotide encoding the nuclease of interest. The kit may also include restriction enzymes that can be used to selectively excise the nuclease-encoding polynucleotide from the vector and subsequently re-introduce the modified polynucleotide back into the vector once the user has modified the gene. Such a kit may also include a DNA ligase that can be used to catalyze the formation of covalent phosphodiester linkages between the modified nuclease-encoding polynucleotide and the target vector. A kit of the disclosure may also provide a polynucleotide encoding an anti-TNFRSF, anti-TNFSF, anti-CD28, or anti-ICOS antibody or antigen-binding fragment thereof of the disclosure, as well as a package insert describing the methods one can use to selectively cleave a particular DNA sequence in the genome of the cell in order to incorporate the polynucleotide encoding an anti-TNFRSF, anti-TNFSF, anti-CD28, or anti-ICOS antibody or antigen-binding fragment thereof of the disclosure into the genome at this site. Optionally, the kit may provide a polynucleotide encoding a fusion protein that contains an anti-TNFRSF, anti-TNFSF, anti-CD28, or anti-ICOS antibody or antigen-binding fragment thereof of the disclosure and an additional polypeptide, such as, e.g., those described herein.

A kit of the disclosure can be used to measure a secreted soluble TNFRSF member protein (e.g., present in a biological sample, such as a bodily fluid, e.g., blood, plasma, and serum). Such a kit may contain an anti-TNFRSF antibody or antigen-binding fragment thereof of the disclosure and reagents that can be conjugated to such a polypeptide. Optionally, these antibodies may include those previously described (e.g., a fluorescent molecule, a bioluminescent molecule, a molecule containing a radioactive isotope, a molecule containing a chelating group bound to a paramagnetic ion, etc.) to quantify the level of the TNFRSF member protein in a subject. The level of a secreted soluble TNFRSF member protein can be measured again after administration of the antibody of the disclosure to a subject to determine if administration has caused a decrease in the level of the secreted soluble TNFRSF member protein. For example, the level of a secreted soluble TNFRSF member protein in a subject can be measured one day, two days, three days, four days, five days, six days, one week, eight days, nine days, ten days, eleven days, twelve days, thirteen days, fourteen days, fifteen days, sixteen days, seventeen days, eighteen days, nineteen days, twenty days, three weeks, four weeks, five weeks, six weeks, seven weeks, eight weeks, or more after administration of the antibody of the disclosure. The subject can be administered another dose of the anti-TNFRSF antibody or antigen-binding fragment thereof of the disclosure if the level of the secreted soluble TNFRSF member protein is about the same or higher than the reference level of the secreted soluble TNFRSF member protein or a previously measured level of the secreted soluble TNFRSF member protein.

### Embodiments

Provided herein are the following non-limiting embodiments.

Embodiment 1 is a modified antibody or antigen-binding fragment thereof that specifically binds tumor necrosis factor receptor 2 (TNFR2), wherein the modified antibody or antigen-binding fragment thereof comprises a heavy chain variable region, a light chain variable region, and a modified human IgG4 Fc hinge region, wherein said modified human IgG4 Fc hinge region comprises an amino acid substitution of S228P, wherein the amino acid position is numbered according to the EU index.

Embodiment 2 is the modified antibody or antigen-binding fragment thereof of embodiment 1, wherein the TNFR2 is human TNFR2.

Embodiment 3 is the modified antibody or antigen-binding fragment thereof of embodiment 1 or 2, wherein the modified antibody or antigen-binding fragment thereof is a modified IgG4 antibody comprising a modified human IgG4 Fc domain comprising the amino acid substitution of S228P.

Embodiment 4 is the modified antibody or antigen-binding fragment thereof of embodiment 3, wherein the modified antibody or antigen-binding fragment thereof comprises a modified human IgG4 constant region comprising a modified human IgG4 Fc domain, wherein the modified human IgG4 Fc domain comprises the modified human IgG4 Fc hinge region, and wherein the modified human IgG4 constant region comprises an amino acid sequence having at least 95% identity to the amino acid sequence of SEQ ID NO: 1547.

Embodiment 5 is the modified antibody or antigen-binding fragment thereof of embodiment 4, wherein the modified human IgG4 constant region comprises the amino acid sequence of SEQ ID NO: 1547.

Embodiment 6 is the modified antibody or antigen-binding fragment thereof of any one of embodiments 3-5, wherein the heavy chain variable region comprises a heavy chain complementarity determining region 1 (CDR-H1), CDR-H2, and CDR-H3, and the light chain variable region comprises a light chain complementarity determining region 1 (CDR-L1), CDR-L2, and CDR-L3, wherein the CDR-H1, CDR-H2, CDR-H3, CDR-L1, CDR-L2, and CDR-L3 comprise the amino acid sequences of SEQ ID NOS: 1413, 1414 or 1420, 1415, 1416, 1417, and 1418, respectively.

Embodiment 7 is the modified antibody or antigen-binding fragment thereof of any one of embodiments 3-6, wherein the heavy chain variable region comprises an amino acid sequence at least 95%, at least 96%, at least 97%, at least 98%, or at least 99% identical to SEQ ID NO: 679, 681, 1485, 1486, 1487, 1488, 1489, or 1490, and the light chain variable region comprises an amino acid sequence at least 95%, at least 96%, at least 97%, at least 98%, or at least 99% identical to SEQ ID NO: 680, 682, 1491, 1492, 1493, 1494, or 1495.

Embodiment 8 is the modified antibody or antigen-binding fragment thereof of embodiment 7, wherein the heavy chain variable region comprises the amino acid sequence of SEQ ID NO: 679, 681, 1485, 1486, 1487, 1488, 1489, or 1490, and the light chain variable region comprises the amino acid sequence of SEQ ID NO: 680, 682, 1491, 1492, 1493, 1494, or 1495.

Embodiment 9 is the modified antibody or antigen-binding fragment thereof of embodiment 8, wherein:
(a) the heavy chain variable region comprises the amino acid sequence of SEQ ID NO: 679, and the light chain variable region comprises the amino acid sequence of SEQ ID NO: 680;
(b) the heavy chain variable region comprises the amino acid sequence of SEQ ID NO: 681, and the light chain variable region comprises the amino acid sequence of SEQ ID NO: 682; or
(c) the heavy chain variable region comprises the amino acid sequence of SEQ ID NO: 1488, and the light chain variable region comprises the amino acid sequence of SEQ ID NO: 1495.

Embodiment 10 is the modified antibody or antigen-binding fragment thereof of any one of embodiments 3-6, wherein the modified IgG4 antibody comprises a heavy chain comprising an amino acid sequence at least 95%, at least 96%, at least 97%, at least 98%, or at least 99% identical to SEQ ID NO: 1498, 1499, 1500, 1501, 1502, or 1503, and a light chain comprising an amino acid sequence at least 95%, at least 96%, at least 97%, at least 98%, or at least 99% identical to SEQ ID NO: 1504, 1505, 1506, 1507, or 1508.

Embodiment 11 is the modified antibody or antigen-binding fragment thereof of embodiment 10, wherein the heavy chain comprises the amino acid sequence of SEQ ID NO: 1498, 1499, 1500, 1501, 1502, or 1503, and the light chain comprises the amino acid sequence of SEQ ID NO: 1504, 1505, 1506, 1507, or 1508.

Embodiment 12 is the modified antibody or antigen-binding fragment thereof of embodiment 6, wherein the heavy chain variable region comprises the amino acid sequence of SEQ ID NO: 1488, the light chain variable region comprises the amino acid sequence of SEQ ID NO: 1495, and the IgG4 constant region comprises the amino acid sequence of SEQ ID NO: 1547.

Embodiment 13 is the modified antibody or antigen-binding fragment thereof of embodiment 6, wherein the modified IgG4 antibody comprises a heavy chain comprising the amino acid sequence of SEQ ID NO: 1501, a light chain comprising the amino acid sequence of SEQ ID NO: 1508.

Embodiment 14 is the modified antibody or antigen-binding fragment thereof of any one of embodiments 3-13, wherein the modified antibody is a monoclonal antibody.

Embodiment 15 is the modified antibody or antigen-binding fragment thereof of any one of embodiments 3-14, wherein the modified antibody is a humanized antibody.

Embodiment 16 is the modified antibody or antigen-binding fragment thereof of any one of embodiments 3-14, wherein the modified human IgG4 Fc domain further comprises one or more amino acid modifications reducing effector function selected from the group consisting of:
(i) L235E;
(ii) D265A;
(iii) G237A;
(iv) E318A;
(v) E233P;
(vi) G236R and L328R; and
(vii) N297G or N297Q;
   wherein the amino acid positions are numbered according to the EU index.

Embodiment 17 is the modified antibody or antigen-binding fragment thereof of any one of embodiments 3-14, wherein the modified human IgG4 Fc domain further comprises one or more amino acid modifications reducing effector function selected from:
(i) L235E; and
(ii) F234A and L235A;
   wherein the amino acid positions are numbered according to the EU index.

Embodiment 18 is the modified antibody or antigen-binding fragment thereof of any one of embodiments 3-17, wherein the modified antibody or antigen-binding fragment thereof is an agonistic antibody.

Embodiment 19 is the modified antibody or antigen-binding fragment thereof of embodiment 3, wherein the modified human IgG4 Fc domain is an IgG4*01 allotype, an IgG4*02 allotype, an IgG4*03 allotype, an IgG4*05 allotype, or an IgG4*06 allotype.

Embodiment 20 is the modified antibody or antigen-binding fragment thereof of any one of embodiments 6-18, wherein the modified human IgG4 Fc domain decreases or inactivates an effector function of the modified antibody or antigen-binding fragment thereof as compared to a control antibody or antigen-binding fragment thereof.

Embodiment 21 is the modified antibody or antigen-binding fragment thereof of embodiment 20, wherein the effector function is antibody-dependent cellular cytotoxicity (ADCC) or antibody-dependent cellular phagocytosis (ADCP).

Embodiment 22 is the modified antibody or antigen-binding fragment thereof of any one of embodiments 6-18, wherein the modified human IgG4 Fc domain reduces complement binding function of the modified antibody or antigen-binding fragment thereof as compared to a control antibody.

Embodiment 23 is the modified antibody or antigen-binding fragment thereof of any one of embodiments 6-18, wherein the modified antibody or antigen-binding fragment thereof is capable of being administered to a subject at a higher dose range and/or frequency than a control antibody.

Embodiment 24 is the modified antibody or antigen-binding fragment thereof of any one of embodiments 6-18, wherein the modified antibody or antigen-binding fragment thereof is capable of maintaining or increasing effectiveness when administered to a subject at a higher dose range than a control antibody.

Embodiment 25 is the modified antibody or antigen-binding fragment thereof of any one of embodiments 6-18, wherein the modified antibody or antigen-binding fragment thereof has an enhanced binding efficacy over a longer duration compared to a control antibody.

Embodiment 26 is the modified antibody or antigen-binding fragment thereof of any one of embodiments 4-18, wherein the modified antibody or antigen-binding fragment thereof is further conjugated to a therapeutic agent.

Embodiment 27 is the modified antibody or antigen-binding fragment thereof of embodiment 26, wherein the therapeutic agent is selected from the group consisting of a chemotherapy agent, an immunotherapy agent, and an agonist of TNFR2.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Evaluation of TNFR2 Antagonistic Antibody Activity In Vitro

### Objective

The objective of this study was to determine the therapeutic range of a TNFR2 antagonistic antibody in Jeko-1 tumor cells and to evaluate whether modifications in the TNFR2 antagonistic antibody can improve its activity across different concentrations.

### Materials and Methods

The study was carried out in accordance with the following protocol:
1. Culture Jeko-1 tumor cells in 96-well flat bottom plates at 100,000 cells per well in 200 μL of RPMI⁺⁺ media, which is made by combining 45 mL of Gibco RPMI 1640 medium with GLUTAMAX™ supplement (Cat #61870-036), 5 mL of Gibco heat inactivated Fetal Bovine Serum (FBS) (Cat #10082-147), and 0.5 mL of Penicillin-Streptomycin (100×).
2, Treat the cells with varying dose response concentrations of a TNFR2 antagonistic antibody.
3. After 72 hours, detach the cells from the plates and stain them with trypan blue (Sigma-Aldrich) to count viable cells.

### Results

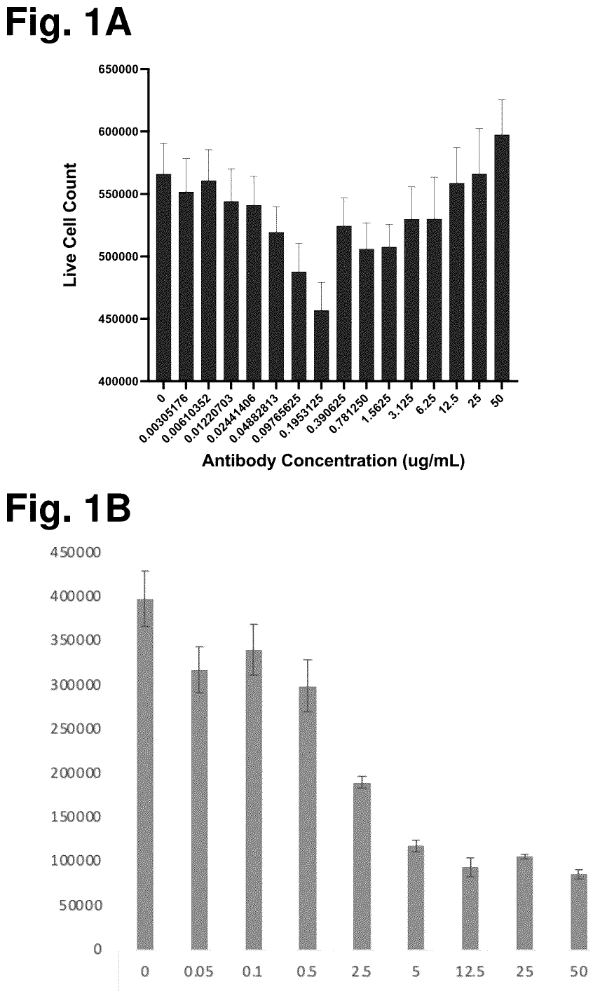

I observed that the fully humanized antagonistic antibody against TNFR2 is effective in reducing the live cell count of Jeko-1 tumor cells at low concentrations of up to 0.195 μg/mL, with the concentration of 0.195 μg/mL showing the largest reduction when compared to control condition of 0 μg/m (FIG. 1A). As the concentration increases beyond 0.195 μg/mL, the number of live Jeko-1 tumor cells increases as well, resulting in a bi-model shaped curve. The therapeutic range of the fully humanized antagonistic antibody in this data set is 0.003 μg/mL to 0.19 μg/mL.

When the experiment was repeated with the fully humanized antagonistic antibody and a mutated Fc receptor, I observed that as the antibody concentration increases, the amount of reduction in the live cell count of Jeko-1 tumor cells increases as well (FIG. 1B). The bi-model shape disappears, and the therapeutic range is expanded to 0.05 μg/mL to 50 μg/mL.

### Conclusion

One limitation of antibody-based therapies is that the window of therapeutic doses is narrow. The efficacy increases until a certain concentration, after which the efficacy begins to decline. This phenotype is demonstrated as a bi-model curve in dose-response experiments and has been a barrier to the development of effective therapeutic antibodies. However, the results in this study suggest that disrupting Fc receptor binding can help overcome this limitation of antibody-based therapies.

The results show that while an antagonistic antibody with intact Fc binding leads to the expected bi-model shaped curve, the same antagonistic antibody with mutated Fc receptor leads to disappearance of the bi-model shape. In addition, disrupting Fc receptor binding affects the dose-dependent efficacy of the antagonistic antibody in a way that widens the therapeutic range. Collectively, these results demonstrate that disrupting Fc receptor binding can be beneficial for the therapeutic activity of antagonistic TNFR2 antibodies. Consequently, antagonistic TNFR2 antibodies that are prepared with modifications to the Fc region that ablate or reduce Fc receptor binding can be used to improve the treatment of diseases and disorders described herein that are mediated by TNFR2 antagonist antibodies or antigen binding fragments thereof.

### Example 2. Evaluation of TNFR2 Agonistic Antibody Activity In Vitro

### Objective

The objective of this study was to determine the therapeutic range of a TNFR2 agonistic antibody in human T cells and to evaluate whether modifications in the TNFR2 agonistic antibody can improve its activity across different concentrations. Specifically, the effects of disruption of Fc receptor binding on TNFR2 agonism were examined.

### Materials and Methods

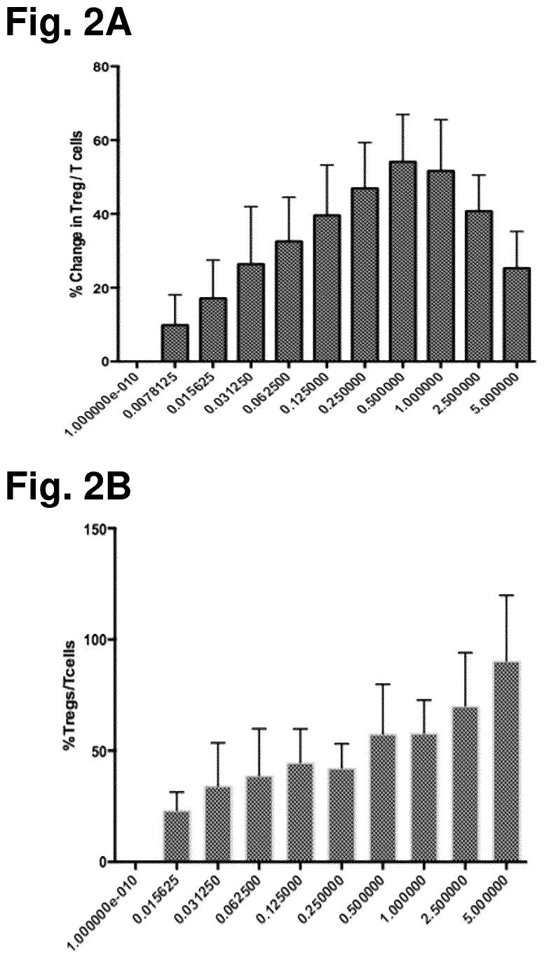

The isolation of human CD4+ T cells and incubation with varying concentrations of a TNFR2 agonistic antibody were carried out in accordance with the following protocol:
1. Isolate CD4⁺ cells from 2-4 tubes of human whole blood using the Stemcell Technologies EASYSEP™ Direct Human CD4⁺ T Cell Isolation Kit (Cat #19662) protocol.
2. Collect 12.5 μL of the isolate and load on a clean hemocytometer for counting.
3. Spin the cells down at 4,000 g for 5 min and aspirate out the supernatant.
4. Reconstitute the cell pellet:
   a. Add 1 mL of RPMI⁺⁺ media and gently pipette to resuspend the cell pellet.
   b. Add enough RPMI⁺⁺ media to obtain the concentration of 1 million cells/mL.
   c. Transfer 1 mL of the cell suspension to a new tube.
   d. Add 4 mL of RPMI⁺⁺ media to the tube to get to the desired concentration of 200 cells/μL (repeat steps 4b and 4c if more than 5 mL of cell suspension is needed).
5. Add 250 μL of RPMI⁺⁺ media to edge columns and rows of a 96-well round bottom plate to help prevent evaporation.
6. Add 100 μL of the cell suspension (20,000 cells) to every well.
7. Wash DYNABEADS® Human T-Activator CD3/CD28:
   a. Allow DYNABEADS® to warm for a few minutes.
   b. Resuspend the DYNABEADS® in the vial by vortexing at high speed for 30 sec.
   c. Transfer the desired volume of DYNABEADS® (1 μL per well) to an Eppendorf tube.
   d. Add 1 mL of RPMI⁺⁺ media to the tube and vortex for 10 sec.
   e. Place the tube on a magnet for 2 min and discard the supernatant.
   f. Reat steps 7c-7e.
   g. Resuspended the washed DYNABEADS® in the same volume of RPMI⁺⁺ media as the initial volume of DYNABEADS® taken from the vial in step 7c.
8. Vortex the washed DYNABEADS® for 5 sec.
9. Add 1 μL of the washed DYNABEADS® to each well containing cells.
10. Perform serial dilution of a TNFR2 agonistic antibody to obtain the desired dose response concentrations (see e.g., FIG. 2A).
11. Add 100 μL of each dose response concentration in duplicate to the wells.
12. Put the plate in the 37° C. incubator with 5% CO₂.

The activation and proliferation of the human T cells via CD3/CD28 stimulation were carried out in accordance with the following protocol:
1. Day 2-3: Pipette 100 μL of RPMI⁺⁺ media out of the wells without disturbing the cell pellet and discard. Replenish by adding fresh RPMI⁺⁺ media with 4 μL of IL-2 (Miltenyi Biotec Human IL-2 IS, Cat #130-097-742 or Cat #130-097-743) per mL of solution.
2. Day 4-5: Pipette 100 μL of RPMI⁺⁺ media out of the wells without disturbing the cell pellet and discard. Replenish by adding fresh RPMI⁺⁺ media with 2 μL of IL-2 per mL of solution.
3. Day 7: Replace old DYNABEADS® with new washed DYNABEADS®:
   a. Remove 100 μL of supernatant from every well containing cells.
   b. Set up Eppendorf tubes for each well and label them.
   c. Pipette down a couple times and transfer the contents of each well to the corresponding tube.
   d. Place all tubes in a magnet for 2 min and transfer the supernatant without the DYNABEADS® back into the corresponding wells on the plate.
   e. Wash DYNABEADS® as described above.
   f. Vortex the washed DYNABEADS® for 5 sec.
   g. Add 1 μL of the washed DYNABEADS® to every well containing cells.
   h. Replenish the wells with 120 μL of RPMI⁺⁺ media with 2 μL of IL-2 per mL of solution (the wells are usually at a lower volume at this point).
4. Day 9-10: Set up serial dilutions of the TNFR2 agonist antibody. Pipette 100 μL of RPMI⁺⁺ out of the wells without disturbing the cell pellet and discard. Add 100 μL of each of the dose response concentrations to the corresponding wells and gently mix by pipetting.
5. Day 11-12: Pipette 100 μL of RPMI⁺⁺ out of the wells without disturbing the cell pellet and discard. Replenish by adding fresh RPMI⁺⁺ media with 2 μL of IL-2 per mL of solution.
6. Day 14-15: Pipette 100 μL of RPMI⁺⁺ out of the wells without disturbing the cell pellet and discard. Replenish by adding 100 μL of fresh RPMI⁺⁺ media with 2 μL of IL-2 per mL of solution (up to 25 μL more of RPMI⁺⁺ media may be added if some of the liquid has evaporated).
7. Day 17:
   a. Collect 100 μL of the supernatant from one set of duplicate wells and add to labeled Eppendorf tubes without disturbing the cell pellet.
   b. Start staining procedure below for rows from which the supernatant was collected.

The CD25/CD127 surface staining for flow cytometry was carried out in accordance with the following protocol:
1. Add 200 μL of 2% FBS wash buffer (490 mL HBSS+10 mL FBS) into 1 row of wells and pipette up and down before transferring to their respectively labeled Eppendorf tubes.
2. Incubate the tubes for 2 min in the magnet and transfer the DYNABEADS®-free supernatant to a new set of respectively labeled tubes.
3. Centrifuge the tubes at 2,000 rpm for 2 min.
4. Aspirate the supernatant and discard. Get as close as possible to the cell pellet without disturbing it.
5. Add 50-80 μL of 2% FBS XF media (480 mL Agilent Seahorse XF RPMI medium, pH 7.4 (Cat #103576-100)+20 mL FBS) to resuspend the cell pellet.
6. Add 5 L of both CD25 PE and CD127 APC stains to the tubes.
7. Vortex the tubes for 5 sec to dissolve the cell pellet.
8. Incubate the tubes in the staining solution for 25 min on ice in the dark.
9. Add 1 mL of the 2% FBS wash buffer to the tubes.
10. Centrifuge the tubes at 2,000 rpm for 2 min.
11. Aspirate the supernatant without disturbing the cell pellet and discard.
12. Resuspend the cells in 200-500 μL of cold 2% FBS XF media by pipetting up and down.
13. Vortex the tubes for 5 sec.
14. Transfer the cell suspension to respectively labeled 5 mL flow tubes for flow cytometry.

### Results

I observed that the fully humanized agonistic antibody against TNFR2 is effective in proliferating Treg cells at low concentrations, with the concentration of 0.5 μg/mL showing the largest difference when compared to control condition (FIG. 2A). As the concentration increases further, the percentage of Treg cells decreases, resulting in a bi-model shaped curve. The therapeutic range of the fully humanized antagonistic antibody in this data set is 0.01 μg/mL to 0.5 μg/mL.

When the experiment was repeated with the same fully humanized agonistic antibody but with LALA mutations in the Fc region, which disrupt Fc receptor activity, I observed an increase in the percentage of Treg cells with an increase in the antibody concentration (FIG. 2B). The bi-model shape disappears, and the therapeutic range is expanded to 0.01 μg/mL to 5.0 μg/mL and above.

### Conclusion

Similar to antagonistic antibodies, agonistic antibodies also show the bi-model shape in their dose-response curves. The antibodies are effective until a certain concentration, after which they become less effective. The results in this study suggest that amino acid mutations in the Fc region of the agonistic antibody improve the potency of the antibody.

The results show that while an agonistic antibody with intact Fc binding leads to the expected bi-model shaped curve, the same agonistic antibody with mutations in the Fc region leads to disappearance of the bi-model shape. In addition, disrupting Fc receptor binding affects the dose-dependent efficacy of the agonistic antibody in a way that widens the therapeutic range. Collectively, these results demonstrate that inactive Fc receptor binding can be beneficial for the therapeutic activity of agonistic TNFR2 antibodies. Consequently, agonistic TNFR2 antibodies that are prepared with modifications to the Fc region that ablate or reduce Fc receptor binding can be used to improve the treatment of diseases and disorders described herein that are mediated by TNFR2 agonist antibodies or antigen binding fragments thereof.

### Example 3. Evaluation of the Significance of Fc Function in TNFR2 Agonism In Vitro

### Objective

The objective of this series of experiments was to evaluate the role of Fc domain and its binding to Fc receptor in mediating the effects of TNFR2 agonism.

### Materials and Methods

The isolation of human CD4+ T cells and incubation with varying concentrations of TNFR2 agonistic antibodies were carried out in accordance with the following protocol:
1. Isolate CD4⁺ cells from 2-4 tubes of human whole blood using the Stemcell Technologies EASYSEP™ Direct Human CD4⁺ T Cell Isolation Kit (Cat #19662) protocol.
2. Collect 12.5 μL of the isolate and load on a clean hemocytometer for counting.
3. Spin the cells down at 4,000 g for 5 min and aspirate out the supernatant.
4. Reconstitute the cell pellet:
   a. Add 1 mL of RPMI⁺⁺ media and gently pipette to resuspend the cell pellet.
   b. Add enough of RPMI⁺⁺ media to obtain the concentration of 1 million cells/mL.
   c. Transfer 1 mL of the cell suspension to a new tube.
   d. Add 4 mL of RPMI⁺⁺ media to the tube to get to the desired concentration of 200 cells/μL (repeat steps 4b and 4c if more than 5 mL of cell suspension is needed).
5. Add 250 μL of RPMI⁺⁺ media to edge columns and rows of a 96-well round bottom plate to help prevent evaporation.
6. Add 100 μL of the cell suspension (20,000 cells) to every well.
7. Wash DYNABEADS® Human T-Activator CD3/CD28:
   a. Allow DYNABEADS® to warm for a few minutes.
   b. Resuspend the DYNABEADS® in the vial by vortexing at high speed for 30 sec.
   c. Transfer the desired volume of DYNABEADS® (1 μL per well) to an Eppendorf tube.
   d. Add 1 mL of RPMI⁺⁺ media to the tube and vortex for 10 sec.
   e. Place the tube on a magnet for 2 min and discard the supernatant.
   f. Reat steps 7c-7e.
   g. Resuspended the washed DYNABEADS® in the same volume of RPMI⁺⁺ media as the initial volume of DYNABEADS® taken from the vial in step 7c.
8. Vortex the washed DYNABEADS® for 5 sec.
9. Add 1 μL of the washed DYNABEADS® to each well containing cells.
10. Perform serial dilution of TNFR2 agonistic antibodies to obtain the desired dose response concentrations (see e.g., FIG. 2A).
11. Add 100 μL of each dose response concentration in duplicate to the wells.
12. Put the plate in the 37° C. incubator with 5% CO₂.

The activation and proliferation of the human T cells via CD3/CD28 stimulation were carried out in accordance with the following protocol:
1. Day 2-3: Pipette 100 μL of RPMI⁺⁺ media out of the wells without disturbing the cell pellet and discard. Replenish by adding fresh RPMI⁺⁺ media with 4 μL of IL-2 (Miltenyi Biotec Human IL-2 IS, Cat #130-097-742 or Cat #130-097-743) per mL of solution.
2. Day 4-5: Pipette 100 μL of RPMI⁺⁺ media out of the wells without disturbing the cell pellet and discard. Replenish by adding fresh RPMI⁺⁺ media with 2 μL of IL-2 per mL of solution.
3. Day 7: Replace old DYNABEADS® with new washed DYNABEADS®:
   a. Remove 100 μL of supernatant from every well containing cells.
   b. Set up Eppendorf tubes for each well and label them.
   c. Pipette down a couple times and transfer the contents of each well to the corresponding tube.
   d. Place all tubes in a magnet for 2 min and transfer the supernatant without the DYNABEADS® back into the corresponding wells on the plate.
   e. Wash DYNABEADS® as described above.
   f. Vortex the washed DYNABEADS® for 5 sec.
   g. Add 1 μL of the washed DYNABEADS® to every well containing cells.
   h. Replenish the wells with 120 μL of RPMI⁺⁺ media with 2 μL of IL-2 per mL of solution (the wells are usually at a lower volume at this point).
4. Day 9-10: Set up serial dilutions of the TNFR2 agonist antibodies. Pipette 100 μL of RPMI⁺⁺ out of the wells without disturbing the cell pellet and discard. Add 100 μL of each of the dose response concentrations to the corresponding wells and gently mix by pipetting.
5. Day 11-12: Pipette 100 μL of RPMI⁺⁺ out of the wells without disturbing the cell pellet and discard. Replenish by adding fresh RPMI⁺⁺ media with 2 μL of IL-2 per mL of solution.
6. Day 14-15: Pipette 100 μL of RPMI⁺⁺ out of the wells without disturbing the cell pellet and discard. Replenish by adding 100 μL of fresh RPMI⁺⁺ media with 2 μL of IL-2 per mL of solution (up to 25 μL more of RPMI⁺⁺ media may be added if some of the liquid has evaporated).
7. Day 17:
   a. Collect 100 μL of the supernatant from one set of duplicate wells and add to labeled Eppendorf tubes without disturbing the cell pellet.
   b. Start staining procedure below for rows from which the supernatant was collected.

The CD25/CD127 surface staining for flow cytometry was carried out in accordance with the following protocol:
1. Add 200 μL of 2% FBS wash buffer (490 mL HBSS+10 mL FBS) into 1 row of wells and pipette up and down before transferring to their respectively labeled Eppendorf tubes.
2. Incubate the tubes for 2 min in the magnet and transfer the DYNABEAD®-free supernatant to a new set of respectively labeled tubes.
3. Centrifuge the tubes at 2,000 rpm for 2 min.
4. Aspirate the supernatant and discard. Get as close as possible to the cell pellet without disturbing it.
5. Add 50-80 μL of 2% FBS XF media (480 mL Agilent Seahorse XF RPMI medium, pH 7.4 (Cat #103576-100)+20 mL FBS) to resuspend the cell pellet.
6. Add 5 μL of both CD25 PE and CD127 APC stains to the tubes.
7. Vortex the tubes for 5 sec to dissolve the cell pellet.
8. Incubate the tubes in the staining solution for 25 min on ice in the dark.
9. Add 1 mL of the 2% FBS wash buffer to the tubes.
10. Centrifuge the tubes at 2,000 rpm for 2 min.
11. Aspirate the supernatant without disturbing the cell pellet and discard.
12. Resuspend the cells in 200-500 μL of cold 2% FBS XF media by pipetting up and down.
13. Vortex the tubes for 5 sec.
14. Transfer the cell suspension to respectively labeled 5 mL flow tubes for flow cytometry.

### Results

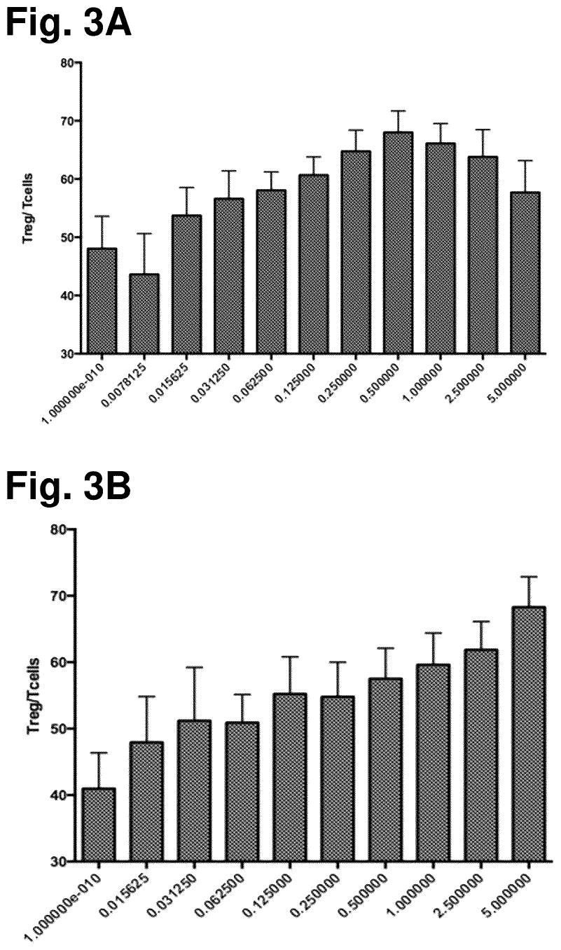

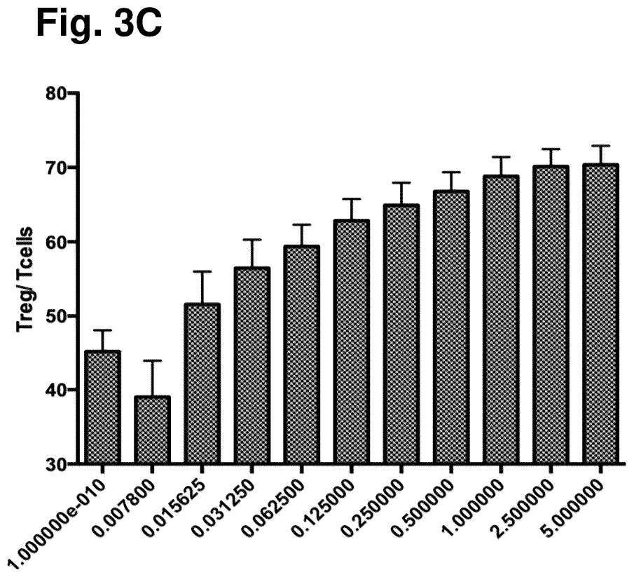

I observed that when human T cells are treated with a human anti-human TNFR2 agonist antibody, there is expansion of Treg cells at concentrations up to 0.5 g/mL but not at concentrations of 1.0 μg/mL and above (FIG. 3A). When human T cells are treated with a human anti-human TNFR2 agonist with LALA double mutations in the Fc region, the percentage of Treg cells increases with increasing concentrations of the agonist, even beyond 1.0 μg/ml (FIG. 3B). Lastly, when human T cells are treated with a mouse anti-human TNFR2 agonist, there is an increase in the percentage of Treg cells (FIG. 3C). This increase is greater at higher concentrations of the mouse anti-human TNFR2 agonist.

### Conclusion

One mechanism that antibodies rely on for exerting their therapeutic effects is the hexagonal organization of the target protein receptors. In the case of TNFR2, efficient signaling of TNFR2 agonism requires TNFR2 receptors to form parallel dimers with exposed ligand-binding sites. These parallel dimers then form trimers with each other, resulting in a tight hexagonal complex and a dense network of TNFR2 receptors on the cell surface. When agonistic antibodies bind, they are able to activate multiple receptors at once and trigger the downstream effects. On the other hand, efficient signaling of TNFR2 antagonism requires antiparallel dimerization of TNFR2 receptors. This conformation buries the ligand-binding sites and results in the formation of a loose, or open, hexagonal complex. Antagonistic antibodies are able to stabilize this loose hexagonal network of TNFR2 receptors, thereby disrupting receptor activation and downstream signaling activity. Given these differences, proper organization of the hexagonal structure is critical for agonistic and antagonistic antibodies.

Another mechanism by which antibodies exert their therapeutic effects is ADCC, which is initiated when an antibody binds a target cell and an effector cell. Specifically, the Fc region of an antibody binds the Fc receptors that are expressed on the effector cells. Upon binding at the Fc region, effector cells are recruited and a signaling mechanism is triggered, which leads to apoptosis of the target cell. It is known in the art that ADCC is required for effective therapeutic activity of antibodies. Thus, a common strategy that has been utilized to improve the efficacy of antibodies is to enhance ADCC by modifying Fc binding. In this series of experiments, the role of the Fc region in TNFR2 agonism was explored. While the human anti-human TNFR2 agonist results in a bi-model shaped curve, both the human anti-human TNFR2 agonist with LALA mutations and mouse anti-human TNFR2 agonist show a positive relationship between the proliferation of Treg cells and antibody concentration. The difference in these results may be attributed to ADCC clustering via Fc receptor binding, which is intact in the former but disrupted in the latter. For human anti-human TNFR2 agonist with LALA mutations, the mutations disrupt the function of Fc receptors. For the mouse anti-human TNFR2 agonist, Fc receptor binding is essentially inactive, since antibodies raised in mice rarely bind human Fc receptors.

Intact Fc function of the human anti-human TNFR2 agonist promotes ADCC clustering via Fc receptor binding. It was understood that ADCC and the Fc binding, which promotes ADCC, are necessary for improving the efficacy of antibodies. The results of this study suggest, however, that at higher concentrations, ADCC may become less specific and begin to interfere with the hexagonal organization of TNFR2 receptors, resulting in reduced potency and a narrow window of therapeutic doses. On the other hand, disrupted Fc function of the human anti-human TNFR2 agonist with LALA mutations and mouse anti-human TNFR2 agonist prevents ADCC clustering. However, there is still proliferation and expansion of Treg cells at higher concentrations of these agonists, suggesting that ADCC clustering is not required for the downstream effects of these antibodies to be propagated. Rather, proper hexagonal organization of TNFR2 receptors may be required, which may be achieved using antibodies that have mutated Fc receptors.

Collectively, these results demonstrate that disruption of Fc receptor function via mutations in the Fc region of TNFR2 agonist antibodies is an effective method for improving the potency and widening the therapeutic range of the antibodies. These results also suggest that ADCC and hexagonal clustering mechanisms may interfere with one another, and that hexagonal clustering is required for therapeutic antibodies to exert their effects over a broad range of doses.

### Example 4. Administration of a TNFRSF Antagonistic Antibody to a Patient Suffering from a Cancer or Infectious Disease

Using conventional methods known in the art, a TNFRSF antagonistic antibody comprising an Fc domain with amino acid modifications (such as those described herein) can be administered to a patient suffering from a cancer or infectious disease at a higher dose or frequency than previously known.

According to the methods disclosed herein, a physician of skill in the art can treat a patient, such as a human patient, so as to increase the immune response in the human patient to fight a cancer or infectious disease. To this end, a physician of skill in the art can administer to the human patient an antibody or antigen-binding fragment that binds a TNFRSF member protein and contains amino acid modifications in its Fc domain. Exemplary TNFRSF member proteins include TRAMP, NGFR, TRAIL-R4, TNFR2, HVEM, CD30, TROY, and RELT (TNFRSF19L). An effective dose of the composition may be between about 0.1 mg to about 5000 mg or between about 0.001 mg/kg to about 50 mg/kg of the antibody or antigen-binding fragment. The effective dose may be administered to the human patient at the frequency of one or more times a month, every three weeks, every two weeks, a week, every six days, every five days, every four days, every three days, every two days, or a day. Typically, a physician will determine the actual dosage which will be most suitable for an individual subject, and it will vary with the disease, age, weight, and response of the patient. The appropriate dosage can be determined by one skilled in the art. For example, the treatment may occur by administration (e.g., subcutaneous administration) of an antagonist anti-TNFR2 antibody or antigen-binding fragment thereof.

The TNFRSF antagonistic antibody comprising modified Fc domain can be administered to the patient in an amount sufficient to treat one or more features of a cancer or infectious disease, following which, the responsiveness to treatment can be measured. Administration of the TNFRSF antagonistic antibody with modified Fc domain described may, for example, alleviate the symptoms of a cancer or infectious disease. The level of the soluble TNFRSF member protein in the serum or plasma of the patient may be measured 1 month, 2 months, 3 months, 4 months, 5 months, 6 months, or more following administration of the compositions described herein to determine the patient's responsiveness to the treatment. Depending on the outcome of the evaluation, the patient may receive additional treatments.

The patient that is suitable for receiving the TNFRSF antagonistic antibody comprising modified Fc domain described may be suffering from a cancer (e.g., acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, renal cancer, retinoblastoma, rhabdomyosarcoma, salivary gland cancer, kaposi sarcoma, rhabdomyosarcoma, sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia) or an infectious disease.

### Example 5. Administration of a TNFRSF Agonistic Antibody to a Patient Suffering from an Autoimmune or Inflammatory Disease

Using conventional methods known in the art, a TNFRSF agonistic antibody comprising an Fc domain with amino acid modifications (such as those described herein) can be administered to a patient suffering from an autoimmune or inflammatory disease at a higher dose or frequency than previously known.

According to the methods disclosed herein, a physician of skill in the art can treat a patient, such as a human patient, so as to suppress the immune response in the human patient to fight an autoimmune or inflammatory disease. To this end, a physician of skill in the art can administer to the human patient an antibody or antigen-binding fragment that binds a TNFRSF member protein and contains amino acid modifications in its Fc domain. Exemplary TNFRSF member proteins include TRAMP, NGFR, TRAIL-R4, TNFR2, HVEM, CD30, TROY, and RELT (TNFRSF19L). An effective dose of the composition may be between about 0.1 mg to about 5000 mg or between about 0.001 mg/kg to about 50 mg/kg of the antibody or antigen-binding fragment. The effective dose may be administered to the human patient at the frequency of one or more times a month, every three weeks, every two weeks, a week, every six days, every five days, every four days, every three days, every two days, or a day. Typically, a physician will determine the actual dosage which will be most suitable for an individual subject, and it will vary with the disease, age, weight, and response of the patient. The appropriate dosage can be determined by one skilled in the art. For example, the treatment may occur by administration (e.g., subcutaneous administration) of an agonist anti-TNFR2 antibody or antigen-binding fragment thereof.

The TNFRSF agonistic antibody comprising modified Fc domain can be administered to the patient in an amount sufficient to treat one or more features of an autoimmune or inflammatory disease, following which, the responsiveness to treatment can be measured. Administration of the TNFRSF agonistic antibody with modified Fc domain described may, for example, alleviate the symptoms of an autoimmune or inflammatory disease. The level of the soluble TNFRSF member protein in the serum or plasma of the patient may be measured 1 month, 2 months, 3 months, 4 months, 5 months, 6 months, or more following administration of the compositions described herein to determine the patient's responsiveness to the treatment. Depending on the outcome of the evaluation, the patient may receive additional treatments.

The patient that is suitable for receiving the TNFRSF agonistic antibody comprising modified Fc domain described may be suffering from an autoimmune disease (e.g., Type I diabetes, alopecia areata, ankylosing spondylitis, antiphospholipid syndrome, autoimmune Addison's disease, autoimmune hemolytic anemia, autoimmune hepatitis, Behçet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, limited scleroderma (CREST syndrome), cold agglutinin disease, Crohn's disease, discoid lupus, essential mixed cryoglobulinemia, fibromyalgia-fibromyositis, Graves' disease, Guillain-Barre syndrome, Hashimoto's thyroiditis, hypothyroidism, inflammatory bowel disease, autoimmune lymphoproliferative syndrome (ALPS), idiopathic pulmonary fibrosis, idiopathic thrombocytopenia purpura (ITP), IgA nephropathy, juvenile arthritis, lichen planus, lupus, lupus nephritis, Ménière's disease, mixed connective tissue disease, multiple sclerosis, myasthenia gravis, Pemphigus vulgaris, Pemphigus foliaceus, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Takayasu's arteritis, temporal arteritis/giant cell arteritis, ulcerative colitis, uveitis, vasculitis, vitiligo, Wegener's granulomatosis, Goodpasture syndrome, transplant rejection, celiac disease, esophagitis, and inflammatory central nervous system disorders (e.g., Alzheimer's disease)) or an inflammatory disease (e.g., acute or chronic inflammation, cardiac fibrosis, lung fibrosis, osteoarthritis, rheumatoid arthritis, atherosclerosis, type I diabetes, type II diabetes, graft-versus-host disease, multiple sclerosis, osteomyelitis, psoriasis, Crohn's disease, Sjögren's syndrome, lupus erythematosus, and ulcerative colitis).

### Example 6. Administration of a TNFRSF Agonist Antibody to a Patient Suffering from an Autoimmune or Inflammatory Disease

Using conventional methods known in the art, a TNFRSF agonist antibody comprising an Fc domain with amino acid modifications (such as those described herein) can be administered to a patient suffering from an autoimmune or inflammatory disease at a higher dose or frequency than previously known.

According to the methods disclosed herein, a physician of skill in the art can treat a patient, such as a human patient, so as to suppress the immune response in the human patient to fight an autoimmune or inflammatory disease. To this end, a physician of skill in the art can administer to the human patient an antibody or antigen-binding fragment that binds a TNFRSF member protein and contains amino acid modifications in its Fc domain. Exemplary TNFRSF member proteins include TRAIL-R1 (TNFRSF10A), TRAIL-R2 (TNFRSF10B), TNFR1, Fas, CD40, CD27, 4-1BB, OX40, GITR, and XEDAR. An effective dose of the composition may be between about 0.1 mg to about 5000 mg or between about 0.001 mg/kg to about 50 mg/kg of the antibody or antigen-binding fragment. The effective dose may be administered to the human patient at the frequency of one or more times a month, every three weeks, every two weeks, a week, every six days, every five days, every four days, every three days, every two days, or a day. Typically, a physician will determine the actual dosage which will be most suitable for an individual subject, and it will vary with the disease, age, weight, and response of the patient. The appropriate dosage can be determined by one skilled in the art.

The TNFRSF agonist antibody comprising modified Fc domain can be administered to the patient in an amount sufficient to treat one or more features of an autoimmune or inflammatory disease, following which, the responsiveness to treatment can be measured. Administration of the TNFRSF antagonistic antibody with modified Fc domain described may, for example, alleviate the symptoms of an autoimmune or inflammatory disease. The level of the soluble TNFRSF member protein in the serum or plasma of the patient may be measured 1 month, 2 months, 3 months, 4 months, 5 months, 6 months, or more following administration of the compositions described herein to determine the patient's responsiveness to the treatment. Depending on the outcome of the evaluation, the patient may receive additional treatments.

The patient that is suitable for receiving the TNFRSF antagonistic antibody comprising modified Fc domain described may be suffering from an autoimmune disease (e.g., Type I diabetes, alopecia areata, ankylosing spondylitis, antiphospholipid syndrome, autoimmune Addison's disease, autoimmune hemolytic anemia, autoimmune hepatitis, Behçet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, limited scleroderma (CREST syndrome), cold agglutinin disease, Crohn's disease, discoid lupus, essential mixed cryoglobulinemia, fibromyalgia-fibromyositis, Graves' disease, Guillain-Barre syndrome, Hashimoto's thyroiditis, hypothyroidism, inflammatory bowel disease, autoimmune lymphoproliferative syndrome (ALPS), idiopathic pulmonary fibrosis, idiopathic thrombocytopenia purpura (ITP), IgA nephropathy, juvenile arthritis, lichen planus, lupus, lupus nephritis, Ménière's disease, mixed connective tissue disease, multiple sclerosis, myasthenia gravis, Pemphigus vulgaris, Pemphigus foliaceus, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Takayasu's arteritis, temporal arteritis/giant cell arteritis, ulcerative colitis, uveitis, vasculitis, vitiligo, Wegener's granulomatosis, Goodpasture syndrome, transplant rejection, celiac disease, esophagitis, and inflammatory central nervous system disorders (e.g., Alzheimer's disease)) or an inflammatory disease (e.g., acute or chronic inflammation, cardiac fibrosis, lung fibrosis, osteoarthritis, rheumatoid arthritis, atherosclerosis, type I diabetes, type II diabetes, graft-versus-host disease, multiple sclerosis, osteomyelitis, psoriasis, Crohn's disease, Sjögren's syndrome, lupus erythematosus, and ulcerative colitis).

### Example 7. Administration of a TNFRSF Agonistic Antibody to a Patient Suffering from a Cancer or Infectious Disease

Using conventional methods known in the art, a TNFRSF agonistic antibody comprising an Fc domain with amino acid modifications (such as those described herein) can be administered to a patient suffering from a cancer or infectious disease at a higher dose or frequency than previously known.

According to the methods disclosed herein, a physician of skill in the art can treat a patient, such as a human patient, so as to increase the immune response in the human patient to fight a cancer or infectious disease. To this end, a physician of skill in the art can administer to the human patient an antibody or antigen-binding fragment that binds a TNFRSF member protein and contains amino acid modifications in its Fc domain. Exemplary TNFRSF member proteins include TRAIL-R1 (TNFRSF10A), TRAIL-R2 (TNFRSF10B), TNFR1, Fas, CD40, CD27, 4-1BB, OX40, GITR, and XEDAR. An effective dose of the composition may be between about 0.1 mg to about 5000 mg or between about 0.001 mg/kg to about 50 mg/kg of the antibody or antigen-binding fragment. The effective dose may be administered to the human patient at the frequency of one or more times a month, every three weeks, every two weeks, a week, every six days, every five days, every four days, every three days, every two days, or a day. Typically, a physician will determine the actual dosage which will be most suitable for an individual subject, and it will vary with the disease, age, weight, and response of the patient. The appropriate dosage can be determined by one skilled in the art.

The TNFRSF agonistic antibody comprising modified Fc domain can be administered to the patient in an amount sufficient to treat one or more features of a cancer or infectious disease, following which, the responsiveness to treatment can be measured. Administration of the TNFRSF agonistic antibody with modified Fc domain described may, for example, alleviate the symptoms of a cancer or infectious disease. The level of the soluble TNFRSF member protein in the serum or plasma of the patient may be measured 1 month, 2 months, 3 months, 4 months, 5 months, 6 months, or more following administration of the compositions described herein to determine the patient's responsiveness to the treatment. Depending on the outcome of the evaluation, the patient may receive additional treatments. The patient that is suitable for receiving the TNFRSF agonistic antibody comprising modified Fc domain described may be suffering from a cancer (e.g., acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, renal cancer, retinoblastoma, rhabdomyosarcoma, salivary gland cancer, kaposi sarcoma, rhabdomyosarcoma, sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia) or an infectious disease.

Throughout this application, the term “agonist” with respect to an antibody refers to an antibody that promotes the proliferation, promotes the cell growth, and/or enhances the biological function of Treg cells. As data in this application demonstrates, an agonist antibody can, for example, also have effector function on cytotoxic T lymphocytes (CTLs) or effector T cells. For example, the agonist antibody can kill population of activated cells, and, thus, could also be referred to as an “antagonist” antibody. To prevent confusion, an agonist antibody can, for example, refer to an antibody that promotes the proliferation, promotes the cell growth, and/or enhances the biological function of Treg cells.

### Example 8. Modified IgG4 Antibody

As demonstrated herein, a way to eliminate the bi-modal curve of agonism, which is characteristic of agonistic antibodies and explains the reasons for clinical trial failures, has been found. Three modifications to the antibodies disclosed herein resulted in the elimination of the bi-modal dose response curve of agonistic antibodies. An antibody isotype with the least amount of natural effector function, i.e., the least Fc function, the least Clq function, and the least glycosylation, was selected. The antibody was genetically modified to improve the hinge of the antibody to fix the separation of the antibody arms into a rigid structure. Then, it was demonstrated with the desired variable sequences in the genetically modified IgG4 isotypic antibody that the well know bi-modal dose response curve was eliminated.

There are two major ways for antibodies to crosslink cell surface receptors such as TNFRSF member proteins (e.g., TNFR2). One way is ADCC and the other way is hexagonal networks.

ADCC. ADCC is one mechanism for cross linking cell surface receptors for agonism. For cell surface receptors to activate signaling, cross linking of surface receptors is necessary. In ADCC, the FAB region of an antibody binds to a cell surface target (e.g., a cell surface receptor) while the Fc region of the antibody binds to an adjacent cell. ADCC at a minimum requires two cells and one cross-linking antibody. Over the years, many methods have been devised to improve ADCC, which commonly involve enhancing the affinity of the FAB to the target antigen and also involve enhancing the affinity of the Fc receptor for the reciprocal two cell binding. This type of agonism is often associated with a dose response curve with a narrow range of antibody concentration of efficacy, with lower doses having better efficacy and higher doses diminishing efficacy of the antibody. Most agonist antibodies have progressed to the clinic as IgG1 antibodies, since IgG1 antibodies bind to almost all types of human Fc receptors.

It has been shown that increasing the dose of the agonist antibody to the point of 100% receptor occupancy results in a decrease in the effect of the agonist antibody, i.e., higher concentrations of the antibody result in a decrease in agonist function. Thus, when an agonist antibody concentration is increased using the ADCC/Fc mechanism of action, the effective dose is limited since the efficacy disappears as doses are increased. The bi-modal curve for agonist antibodies, therefore, has been the reason why few agonist antibodies in the clinic have succeeded.

Hexagonal networks. A new way of enhancing cross linking of surface receptors to achieve agonism was discovered, which involves one antibody and one cell, and the antibody on the cell surface having one FAB arm binding to one cell surface target protein (e.g., a cell surface receptor) and the other FAB arm binding an adjacent but different cell surface target protein on the same cell. The cross linking is all on the cell surface, and this creates a very stable hexagonal network of antibodies that results in enhanced agonism.

It was hypothesized that ADCC in combination with hexagonal network would result in superior, strong antibody agonism. If both ADCC and hexagonal networks crosslink cell surface receptors, then combining the two methods would be assumed to provide even better agonism. As indicated herein, this hypothesis was found to be incorrect. For the best agonism, and also the best antagonism, the goal should always be for an antibody to form a hexagonal network but to eliminate or minimize ADCC, such as by eliminating or minimizing Fc receptor function. The ADCC cross linking not only fails but also harms to enhance the agonism or antagonism as a result of hexagonal networks. Additionally, it was demonstrated that “pure” agonism with hexagonal networks was able to eliminate the bi-modal response curve as described above, a problem that has plagued the field of agonist antibodies for at least 20 years.

It was determined that the hexagonal network alone without ADCC (Fc receptor binding) resulted in optimized agonism. It was determined that ADCC in combination with hexagonal network results in worsening agonist function. It was determined that hexagonal networks are good. It was determined that hexagonal network alone without ADCC was the best. It is apparent that the ADCC crosslink impairs the symmetry of cell surface hexagonal networks. Cell surface hexagonal networks with a tight “beehives” conformation promote antibody agonism; cell surface hexagonal networks with larger “beehives” conformation (e.g., as a result of interference by ADCC) promote antibody antagonism.

In general, human IgG antibodies have four major subclasses, IgG1, IgG2, IgG3, and IgG4. Each one of these antibody subclasses has different degrees of Fc receptor binding. Thus, if enhanced Fc receptor binding is desired for an antibody, the best subclass for the antibody is an IgG1; and if less Fc receptor function is desired, the best subclass for the antibody is an IgG4. Also, IgG4 eliminates C1q binding, which is involved in complement activation. As a result, IgG4 antibodies have no or minimal ability to kill cells that they bind through the complement system. Since it was desired to enhance agonism, it would be desired to have an antibody that does not inadvertently activate complement system and kill the cells to which it binds.

Therefore, the preferred antibody sequence, namely an IgG4-based antibody sequence, was used in an experiment involving different antibody frameworks to test how to minimize ADCC, i.e., Fc receptor binding. Additionally, it was desired for the IgG4 antibody to not undergo Fab arm exchange, which could alter its threshold for binding to a cell and forming the hexagonal network. Thus, a single point mutation (S228) was introduced to prevent Fab arm exchange of the IgG4 antibody. This mutation is in the IgG4 Fc hinge region of the antibody, and this region is also known to control the distance between the Fab arms of the antibody. This mutation may also confer superior distances for pulling cell surface receptors closer together on the cell surface, e.g., by reducing the distance between the Fab arms of the resulting antibody.

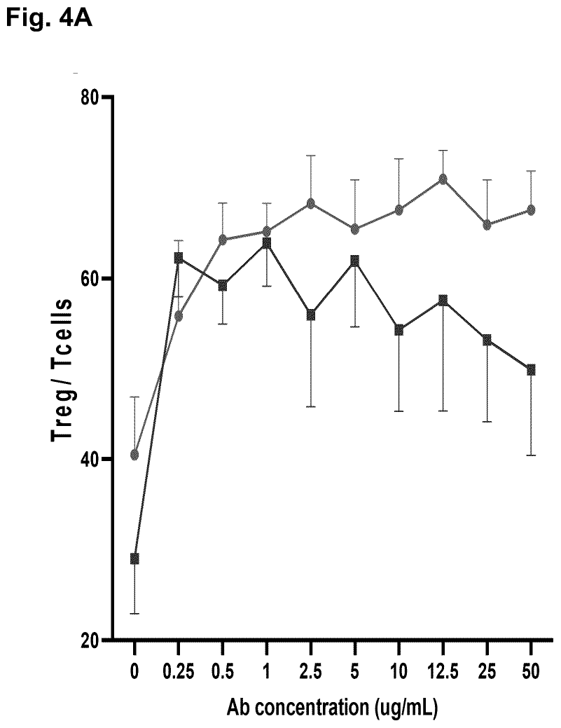

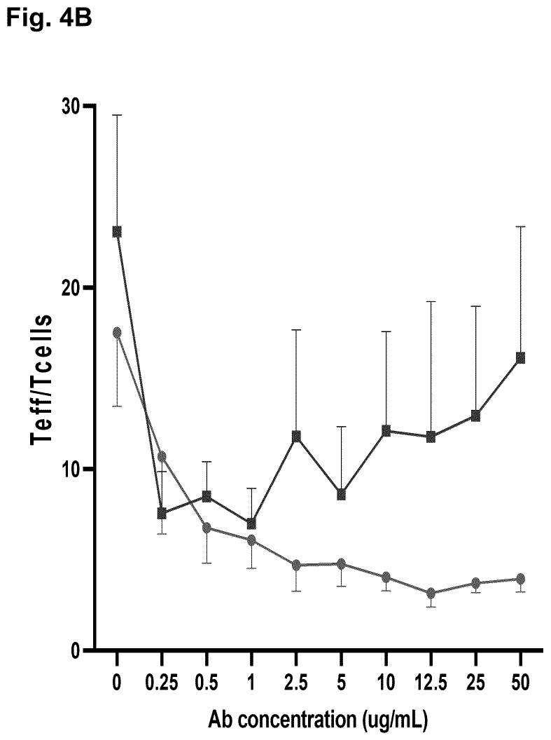

Although it was predicted that IgG1 with the best Fc receptor function (e.g., ADCC) would be optimal for agonism, it was demonstrated that the IgG4 antibody with no ADCC worked the best for agonism. All assays were performed using freshly purified human CD4+ T cells. Treg assays were performed to determine Treg cell proliferation and death of activated cells (e.g., effector T cells). The loss of agonism caused by ADCC for an IgG1 antibody, which has a functionally intact Fc domain that promotes ADCC, was demonstrated by the well-known bimodal response curve of the IgG1 antibody (FIG. 4A). This bimodal response curve was eliminated with the modified IgG4 antibody, which retained high potency (as demonstrated by the enhancement of Treg proliferation) at very high doses. In addition, very low doses of the modified IgG4 antibody also performed with increased potency.

In a 21-day Treg assay using a humanized TNFR2 agonist antibody (an antibody having CDR-H1, CDR-H2, CDR-H3, CDR-L1, CDR-L2, and CDR-L3 sequences of SEQ ID NOs: 1413, 1414 or 1420, 1415, 1416, 1417, and 1418, respectively; VH and VL sequences of SEQ ID NOs: 1488 and 1495, respectively; and HC and LC sequences of SEQ ID NOs: 1501 and 1508, respectively), an IgG4 TNFR2 agonist antibody was demonstrated to be the best performer for promoting Treg cell proliferation (FIG. 4A). This antibody also maintained excellent effector T cell function through activated CTL killing, which was also important, since the best antibodies for inflammatory disease should have both Treg activation and reciprocal effector T cell killing. Therefore, it was demonstrated that the IgG4 agonist antibody had both Treg proliferation and effector T cell killing. In the 21-day Treg assay, the IgG1 TNFR2 agonist antibody produced the expected bi-modal response curve (i.e., lower efficacy at both low and high doses) for Treg proliferation and also produced the bi-modal curve for effector T cell death (FIGS. 4A and 4B). These data indicate that the IgG4 TNFR2 agonist antibody was the first agonist antibody to eliminate the bi-modal functional curves with increasing antibody concentrations, i.e., the agonist antibodies were able to maximize the hexagonal networks and eliminate/minimize Fc binding/C-activation. This could, for example, also be achieved by taking Fc functions away with mutations in other antibody isotypes. For example, other antibody isotypes can be modified to produce antibody fragments, in which the Fc domain of the antibody is removed (e.g., creating an FAB′2 fragment).

The other function of a TNFR2 agonist antibody is that it can also kill effector T cells or inactivate effector T cell function. If the antibody loses the reciprocal effect on effector T cell function, the antibody does not achieve optimal agonism. In an experiment designed to test effector T cell function, it was shown that the modified IgG4 agonist antibody demonstrated continuous killing or growth inhibition of effector T cells (FIG. 4B). The IgG1 agonist antibody, which has both ADCC and hexagonal mechanisms, retained the undesirable bi-modal response curve that makes therapeutic dosing difficult to achieve. Thus, modified IgG4 antibodies having an Fc domain that minimizes or lacks Fc functions (e.g., ADCC) are best candidates for the development of agonist antibodies.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Although specific dosing regimens have been disclosed, a number of other embodiments and modifications may be made without departing from the spirit and scope of the invention. It should be apparent, however, to those skilled in the art that many more modifications besides those already described are possible without departing from the inventive concepts described herein. The inventive subject matter, therefore, is not to be restricted except in the spirit of the disclosure.

All patents, patent publications, and publications mentioned herein are incorporated herein by reference in their entirety to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

Other embodiments are within the claims.

1-234. (canceled)

235. A modified antibody or antigen-binding fragment thereof that specifically binds tumor necrosis factor receptor 2 (TNFR2), wherein the modified antibody or antigen-binding fragment thereof comprises a heavy chain variable region, a light chain variable region, and a modified human IgG4 constant region comprising a modified human IgG4 Fc hinge region, wherein the modified human IgG4 constant region comprises an amino acid sequence having at least 95% identity to the amino acid sequence of SEQ ID NO: 1547, wherein the modified human IgG4 Fc hinge region comprises the amino acid substitution of S228P, and wherein the amino acid position is numbered according to the EU index.

236. The modified antibody or antigen-binding fragment thereof of claim 235, wherein the TNFR2 is human TNFR2.

237. The modified antibody or antigen-binding fragment thereof of claim 235, wherein the modified antibody or antigen-binding fragment thereof is a modified human IgG4 antibody or antigen-binding fragment thereof comprising a modified human IgG4 Fc domain comprising the amino acid substitution of S228P.

238. The modified antibody or antigen-binding fragment thereof of claim 237, wherein the modified human IgG4 constant region comprises the amino acid sequence of SEQ ID NO: 1547.

239. The modified antibody or antigen-binding fragment thereof of claim 237, wherein the heavy chain variable region comprises a heavy chain complementarity determining region 1 (CDR-H1), CDR-H2, and CDR-H3, and the light chain variable region comprises a light chain complementarity determining region 1 (CDR-L1), CDR-L2, and CDR-L3, wherein the CDR-H1, CDR-H2, CDR-H3, CDR-L1, CDR-L2, and CDR-L3 comprise the amino acid sequences of SEQ ID NOs: 1413, 1414 or 1420, 1415, 1416, 1417, and 1418, respectively.

240. The modified antibody or antigen-binding fragment thereof of claim 239, wherein the heavy chain variable region comprises an amino acid sequence having at least 95% identical identity to the amino acid sequence of SEQ ID NO: 1488, and the light chain variable region comprises an amino acid sequence having at least 95% identical identity to the amino acid sequence of SEQ ID NO: 1495.

241. The modified antibody or antigen-binding fragment thereof of claim 240, wherein the heavy chain variable region comprises the amino acid sequence of SEQ ID NO: 1488, and the light chain variable region comprises the amino acid sequence of SEQ ID NO: 1495.

242. The modified antibody or antigen-binding fragment thereof of claim 239, wherein the modified antibody or antigen-binding fragment thereof comprises a heavy chain comprising an amino acid sequence having at least 95% identical identity to the amino acid sequence of SEQ ID NO: 1501, and a light chain comprising an amino acid sequence having at least 95% identical identity to the amino acid sequence of SEQ ID NO: 1508.

243. The modified antibody or antigen-binding fragment thereof of claim 242, wherein the heavy chain comprises the amino acid sequence of SEQ ID NO: 1501, and the light chain comprises the amino acid sequence of SEQ ID NO: 1508.

244. The modified antibody or antigen-binding fragment thereof of claim 239, wherein the heavy chain variable region comprises the amino acid sequence of SEQ ID NO: 1488, the light chain variable region comprises the amino acid sequence of SEQ ID NO: 1495, and the modified human IgG4 constant region comprises the amino acid sequence of SEQ ID NO: 1547.

245. The modified antibody or antigen-binding fragment thereof of claim 239, wherein the modified antibody or antigen-binding fragment thereof comprises a heavy chain comprising the amino acid sequence of SEQ ID NO: 1501 and a light chain comprising the amino acid sequence of SEQ ID NO: 1508.

246. The modified antibody or antigen-binding fragment thereof of claim 237, wherein the modified antibody is a monoclonal antibody.

247. The modified antibody or antigen-binding fragment thereof of claim 237, wherein the modified antibody is a humanized antibody.

248. The modified antibody or antigen-binding fragment thereof of claim 237, wherein the modified antibody or antigen-binding fragment thereof is an agonistic antibody.

249. The modified antibody or antigen-binding fragment thereof of claim 237, wherein the modified antibody or antigen-binding fragment thereof is formulated for subcutaneous administration.
